SEQUENCE LISTING (447 sequences)

SEQ ID NO: 1 - protein, 152 aa, Homo sapiens
MANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 2 - protein, 152 aa, Homo sapiens
MANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 3 - protein, 362 aa, Homo sapiens
MAAAEEEPKPKKLKVEAPQALRENILFGMGNPLLDISAVVDKDFLDKYSLKPNDQILAEDKHKELFDELVKKFKVEYHAGGSTQNSIKVAQWMIQQPHKAATFFGCIGIDKFGEILKRKAAEAHVDAHYYEQNEQPTGTCAACITGDNRSLIANLAAANCYKKEKHLDLEKNWMLVEKARVCYIAGFFLTVSPESVLKVAHHASENNRIFTLNLSAPFISQFYKESLMKVMPYVDILFGNETEAATFAREQGFETKDIKEIAKKTQALPKMNSKRQRIVIFTQGRDDTIMATESEVTAFAVLDQDQKEIIDTNGAGDAFVGGFLSQLVSDKPLTECIRAGHYAASIIIRRTGCTFPEKPDFH

SEQ ID NO: 4 - protein, 132 aa, Homo sapiens
MCDAFVGTWKLVSSENFDDYMKEVGVGFATRKVAGMAKPNMIISVNGDVITIKSESTFKNTEISFILGQEFDEVTADDRKVKSTITLDGGVLVHVQKWDGKSTTIKRKREDDKLVVECVMKGVTSTRVYERA

SEQ ID NO: 5 - protein, 132 aa, Mus musculus
MCDAFVGTWKLVSSENFDDYMKEVGVGFATRKVAGMAKPNMIISVNGDLVTIRSESTFKNTEISFKLGVEFDEITADDRKVKSIITLDGGALVQVQKWDGKSTTIKRKRDGDKLVVECVMKGVTSTRVYERA

SEQ ID NO: 6 - protein, 11 aa, Homo sapiens
KEVGVGFATRK

SEQ ID NO: 7 - DNA, 399 bp, Homo sapiens
  source 1..399: mol_type = other DNA
atgtgtgatgcttttgtaggtacctggaaacttgtctccagtgaaaactttgatgattatatgaaagaagtaggagtgggctttgccaccaggaaagtggctggcatggccaaacctaacatgatcatcagtgtgaatggggatgtgatcaccattaaatctgaaagtacctttaaaaatactgagatttccttcatactgggccaggaatttgacgaagtcactgcagatgacaggaaagtcaagagcaccataaccttagatgggggtgtcctggtacatgtgcagaaatgggatggaaaatcaaccaccataaagagaaaacgagaggatgataaactggtggtggaatgcgtcatgaaaggcgtcacttccacgagagtttatgagagagcataa

SEQ ID NO: 8 - DNA, 399 bp, Mus musculus
  source 1..399: mol_type = other DNA
atgtgtgatgcctttgtgggaacctggaagcttgtctccagtgaaaacttcgatgattacatgaaagaagtgggagtgggctttgccacaaggaaagtggcaggcatggccaagcccaacatgatcatcagcgtaaatggggatttggtcaccatccggtcagagagtacttttaaaaacaccgagatttccttcaaactgggcgtggaattcgatgaaatcaccgcagacgacaggaaggtgaagagcatcataaccctagatggcggggccctggtgcaggtgcagaagtgggatggaaagtcgaccacaataaagagaaaacgagatggtgacaagctggtggtggaatgtgttatgaaaggcgtgacttccacaagagtttatgaaagggcatga

SEQ ID NO: 9 - protein, 23 aa, Homo sapiens
DIQMTQSPSSLSASVGDRVTITC

SEQ ID NO: 10 - protein, 15 aa, Homo sapiens
WYQQKPGKAPKLLIY

SEQ ID NO: 11 - protein, 32 aa, Homo sapiens
GVPSRFSGSGSGTDFTLTISSLQPEDFATYYC

SEQ ID NO: 12 - protein, 23 aa, Homo sapiens
DIQMTQSPSSLSASVGDRVTITC

SEQ ID NO: 13 - protein, 15 aa, Homo sapiens
WYQQKPGKAPKLLIY

SEQ ID NO: 14 - protein, 32 aa, Homo sapiens; identical to SEQ ID NO: 11

SEQ ID NO: 15 - protein, 23 aa, Homo sapiens
DIQMTQSPSSLSASVGDRVTITC

SEQ ID NO: 16 - protein, 32 aa, Homo sapiens
GVPSRFSGSGSGTDFTFTISSLQPEDIATYYC

SEQ ID NO: 17 - protein, 32 aa, Homo sapiens; identical to SEQ ID NO: 16

SEQ ID NO: 18 - protein, 23 aa, Homo sapiens
DIQMTQSPSSLSASVGDRVTITC

SEQ ID NO: 19 - protein, 15 aa, Homo sapiens
WYQQKPGKAPKLLIY

SEQ ID NO: 20 - protein, 32 aa, Homo sapiens; identical to SEQ ID NO: 16

SEQ ID NO: 21 - protein, 23 aa, Homo sapiens
DIQMTQSPSSLSASVGDRVTITC

SEQ ID NO: 22 - protein, 15 aa, Homo sapiens
WYQQKPGKVPKLLIY

SEQ ID NO: 23 - protein, 32 aa, Homo sapiens
GVPSRFSGSGSGTDFTLTISSLQPEDVATYYC

SEQ ID NO: 24 - protein, 23 aa, Homo sapiens
DIQMTQSPSSLSASVGDRVTITC

SEQ ID NO: 25 - protein, 15 aa, Homo sapiens
WYQQKPGKAPKRLIY

SEQ ID NO: 26 - protein, 32 aa, Homo sapiens
GVPSRFSGSGSGTEFTLTISSLQPEDFATYYC

SEQ ID NO: 27 - protein, 23 aa, Homo sapiens
NIQMTQSPSAMSASVGDRVTITC

SEQ ID NO: 28 - protein, 15 aa, Homo sapiens
WFQQKPGKVPKHLIY

SEQ ID NO: 29 - protein, 32 aa, Homo sapiens; identical to SEQ ID NO: 26

SEQ ID NO: 30 - protein, 23 aa, Homo sapiens
DIQMTQSPSSLSASVGDRVTITC

SEQ ID NO: 31 - protein, 15 aa, Homo sapiens
WFQQKPGKAPKSLIY

SEQ ID NO: 32 - protein, 32 aa, Homo sapiens; identical to SEQ ID NO: 11

SEQ ID NO: 33 - protein, 23 aa, Homo sapiens
DIQMTQSPSSLSASVGDRVTITC

SEQ ID NO: 34 - protein, 15 aa, Homo sapiens
WYQQKPEKAPKSLIY

SEQ ID NO: 35 - protein, 32 aa, Homo sapiens; identical to SEQ ID NO: 11

SEQ ID NO: 36 - protein, 23 aa, Homo sapiens
AIQLTQSPSSLSASVGDRVTITC

SEQ ID NO: 37 - protein, 15 aa, Homo sapiens
WYQQKPGKAPKLLIY

SEQ ID NO: 38 - protein, 32 aa, Homo sapiens; identical to SEQ ID NO: 11

SEQ ID NO: 39 - protein, 23 aa, Homo sapiens
AIQLTQSPSSLSASVGDRVTITC

SEQ ID NO: 40 - protein, 15 aa, Homo sapiens
WYQQKPGKAPKLLIY

SEQ ID NO: 41 - protein, 32 aa, Homo sapiens; identical to SEQ ID NO: 11

SEQ ID NO: 42 - protein, 23 aa, Homo sapiens
DIQMTQSPSSVSASVGDRVTITC

SEQ ID NO: 43 - protein, 15 aa, Homo sapiens
WYQQKPGKAPKLLIY

SEQ ID NO: 44 - protein, 32 aa, Homo sapiens; identical to SEQ ID NO: 11

SEQ ID NO: 45 - protein, 23 aa, Homo sapiens
DIQMTQSPSSVSASVGDRVTITC

SEQ ID NO: 46 - protein, 15 aa, Homo sapiens
WYQQKPGKAPKLLIY

SEQ ID NO: 47 - protein, 32 aa, Homo sapiens; identical to SEQ ID NO: 11

SEQ ID NO: 48 - protein, 23 aa, Homo sapiens
DIQLTQSPSFLSASVGDRVTITC

SEQ ID NO: 49 - protein, 15 aa, Homo sapiens
WYQQKPGKAPKLLIY

SEQ ID NO: 50 - protein, 32 aa, Homo sapiens; identical to SEQ ID NO: 26

SEQ ID NO: 51 - protein, 23 aa, Homo sapiens
AIRMTQSPFSLSASVGDRVTITC

SEQ ID NO: 52 - protein, 15 aa, Homo sapiens
WYQQKPAKAPKLFIY

SEQ ID NO: 53 - protein, 32 aa, Homo sapiens
GVPSRFSGSGSGTDYTLTISSLQPEDFATYYC

SEQ ID NO: 54 - protein, 23 aa, Homo sapiens
AIRMTQSPSSFSASTGDRVTITC

SEQ ID NO: 55 - protein, 15 aa, Homo sapiens
WYQQKPGKAPKLLIY

SEQ ID NO: 56 - protein, 32 aa, Homo sapiens
GVPSRFSGSGSGTDFTLTISCLQSEDFATYYC

SEQ ID NO: 57 - protein, 23 aa, Homo sapiens
VIWMTQSPSLLSASTGDRVTISC

SEQ ID NO: 58 - protein, 15 aa, Homo sapiens
WYQQKPGKAPELLIY

SEQ ID NO: 59 - protein, 32 aa, Homo sapiens; identical to SEQ ID NO: 56

SEQ ID NO: 60 - protein, 23 aa, Homo sapiens
AIQMTQSPSSLSASVGDRVTITC

SEQ ID NO: 61 - protein, 15 aa, Homo sapiens
WYQQKPGKAPKLLIY

SEQ ID NO: 62 - protein, 32 aa, Homo sapiens; identical to SEQ ID NO: 11

SEQ ID NO: 63 - protein, 23 aa, Homo sapiens
DIQMTQSPSTLSASVGDRVTITC

SEQ ID NO: 64 - protein, 15 aa, Homo sapiens
WYQQKPGKAPKLLIY

SEQ ID NO: 65 - protein, 32 aa, Homo sapiens
GVPSRFSGSGSGTEFTLTISSLQPDDFATYYC

SEQ ID NO: 66 - protein, 23 aa, Homo sapiens
DIVMTQTPLSLPVTPGEPASISC

SEQ ID NO: 67 - protein, 15 aa, Homo sapiens
WYLQKPGQSPQLLIY

SEQ ID NO: 68 - protein, 32 aa, Homo sapiens
GVPDRFSGSGSGTDFTLKISRVEAEDVGVYYC

SEQ ID NO: 69 - protein, 23 aa, Homo sapiens
DIVMTQTPLSLPVTPGEPASISC

SEQ ID NO: 70 - protein, 15 aa, Homo sapiens
WYLQKPGQSPQLLIY

SEQ ID NO: 71 - protein, 32 aa, Homo sapiens; identical to SEQ ID NO: 68

SEQ ID NO: 72 - protein, 23 aa, Homo sapiens
DVVMTQSPLSLPVTLGQPASISC

SEQ ID NO: 73 - protein, 15 aa, Homo sapiens
WFQQRPGQSPRRLIY

SEQ ID NO: 74 - protein, 32 aa, Homo sapiens; identical to SEQ ID NO: 68

SEQ ID NO: 75 - protein, 23 aa, Homo sapiens
DVVMTQSPLSLPVTLGQPASISC

SEQ ID NO: 76 - protein, 15 aa, Homo sapiens
WFQQRPGQSPRRLIY

SEQ ID NO: 77 - protein, 32 aa, Homo sapiens; identical to SEQ ID NO: 68

SEQ ID NO: 78 - protein, 23 aa, Homo sapiens
DIVMTQTPLSLSVTPGQPASISC

SEQ ID NO: 79 - protein, 15 aa, Homo sapiens
WYLQKPGQSPQLLIY

SEQ ID NO: 80 - protein, 32 aa, Homo sapiens; identical to SEQ ID NO: 68

SEQ ID NO: 81 - protein, 23 aa, Homo sapiens
DIVMTQTPLSLSVTPGQPASISC

SEQ ID NO: 82 - protein, 15 aa, Homo sapiens
WYLQKPGQPPQLLIY

SEQ ID NO: 83 - protein, 32 aa, Homo sapiens; identical to SEQ ID NO: 68

SEQ ID NO: 84 - protein, 23 aa, Homo sapiens
DIVMTQSPLSLPVTPGEPASISC

SEQ ID NO: 85 - protein, 15 aa, Homo sapiens
WYLQKPGQSPQLLIY

SEQ ID NO: 86 - protein, 32 aa, Homo sapiens; identical to SEQ ID NO: 68

SEQ ID NO: 87 - protein, 23 aa, Homo sapiens
DIVMTQSPLSLPVTPGEPASISC

SEQ ID NO: 88 - protein, 15 aa, Homo sapiens
WYLQKPGQSPQLLIY

SEQ ID NO: 89 - protein, 32 aa, Homo sapiens; identical to SEQ ID NO: 68

SEQ ID NO: 90 - protein, 23 aa, Homo sapiens
DIVMTQTPLSSPVTLGQPASISC

SEQ ID NO: 91 - protein, 15 aa, Homo sapiens
WLQQRPGQPPRLLIY

SEQ ID NO: 92 - protein, 32 aa, Homo sapiens
GVPDRFSGSGAGTDFTLKISRVEAEDVGVYYC

SEQ ID NO: 93 - protein, 23 aa, Homo sapiens
EIVLTQSPGTLSLSPGERATLSC

SEQ ID NO: 94 - protein, 15 aa, Homo sapiens
WYQQKPGQAPRLLIY

SEQ ID NO: 95 - protein, 32 aa, Homo sapiens
GIPDRFSGSGSGTDFTLTISRLEPEDFAVYYC

SEQ ID NO: 96 - protein, 23 aa, Homo sapiens
EIVLTQSPATLSLSPGERATLSC

SEQ ID NO: 97 - protein, 15 aa, Homo sapiens
WYQQKPGLAPRLLIY

SEQ ID NO: 98 - protein, 32 aa, Homo sapiens; identical to SEQ ID NO: 95

SEQ ID NO: 99 - protein, 23 aa, Homo sapiens
EIVMTQSPATLSVSPGERATLSC

SEQ ID NO: 100 - protein, 15 aa, Homo sapiens
WYQQKPGQAPRLLIY

SEQ ID NO: 101 - protein, 32 aa, Homo sapiens
GIPARFSGSGSGTEFTLTISSLQSEDFAVYYC

SEQ ID NO: 102 - protein, 23 aa, Homo sapiens
EIVMTQSPATLSVSPGERATLSC

SEQ ID NO: 103 - protein, 15 aa, Homo sapiens
WYQQKPGQAPRLLIY

SEQ ID NO: 104 - protein, 32 aa, Homo sapiens; identical to SEQ ID NO: 101

SEQ ID NO: 105 - protein, 23 aa, Homo sapiens
EIVLTQSPATLSLSPGERATLSC

SEQ ID NO: 106 - protein, 15 aa, Homo sapiens
WYQQKPGQAPRLLIY

SEQ ID NO: 107 - protein, 32 aa, Homo sapiens
GIPARFSGSGSGTDFTLTISSLEPEDFAVYYC

SEQ ID NO: 108 - protein, 23 aa, Homo sapiens
EIVLTQSPATLSLSPGERATLSC

SEQ ID NO: 109 - protein, 15 aa, Homo sapiens
WYQQKPGQAPRLLIY

SEQ ID NO: 110 - protein, 32 aa, Homo sapiens
GIPARFSGSGPGTDFTLTISSLEPEDFAVYYC

SEQ ID NO: 111 - protein, 23 aa, Homo sapiens
EIVMTQSPATLSLSPGERATLSC

SEQ ID NO: 112 - protein, 15 aa, Homo sapiens
WYQQKPGQAPRLLIY

SEQ ID NO: 113 - protein, 32 aa, Homo sapiens
GIPARFSGSGSGTDFTLTISSLQPEDFAVYYC

SEQ ID NO: 114 - protein, 23 aa, Homo sapiens
DIVMTQSPDSLAVSLGERATINC

SEQ ID NO: 115 - protein, 15 aa, Homo sapiens
WYQQKPGQPPKLLIY

SEQ ID NO: 116 - protein, 32 aa, Homo sapiens
GVPDRFSGSGSGTDFTLTISSLQAEDVAVYYC

SEQ ID NO: 117 - protein, 23 aa, Homo sapiens
ETTLTQSPAFMSATPGDKVNISC

SEQ ID NO: 118 - protein, 15 aa, Homo sapiens
WYQQKPGEAAIFIIQ

SEQ ID NO: 119 - protein, 32 aa, Homo sapiens
GIPPRFSGSGYGTDFTLTINNIESEDAAYYFC

SEQ ID NO: 120 - protein, 23 aa, Homo sapiens
EIVLTQSPDFQSVTPKEKVTITC

SEQ ID NO: 121 - protein, 15 aa, Homo sapiens
WYQQKPDQSPKLLIK

SEQ ID NO: 122 - protein, 32 aa, Homo sapiens
GVPSRFSGSGSGTDFTLTINSLEAEDAATYYC

SEQ ID NO: 123 - protein, 23 aa, Homo sapiens
EIVLTQSPDFQSVTPKEKVTITC

SEQ ID NO: 124 - protein, 15 aa, Homo sapiens
WYQQKPDQSPKLLIK

SEQ ID NO: 125 - protein, 32 aa, Homo sapiens; identical to SEQ ID NO: 122

SEQ ID NO: 126 - protein, 23 aa, Homo sapiens
DVVMTQSPAFLSVTPGEKVTITC

SEQ ID NO: 127 - protein, 15 aa, Homo sapiens
WYQQKPDQAPKLLIK

SEQ ID NO: 128 - protein, 32 aa, Homo sapiens
GVPSRFSGSGSGTDFTFTISSLEAEDAATYYC

SEQ ID NO: 129 - protein, 10 aa, Homo sapiens
FGQGTKVEIK

SEQ ID NO: 130 - protein, 10 aa, Homo sapiens
FGQGTKLEIK

SEQ ID NO: 131 - protein, 10 aa, Homo sapiens
FGPGTKVDIK

SEQ ID NO: 132 - protein, 10 aa, Homo sapiens
FGGGTKVEIK

SEQ ID NO: 133 - protein, 10 aa, Homo sapiens
FGQGTRLEIK

SEQ ID NO: 134 - protein, 22 aa, Homo sapiens
QSVLTQPPSVSEAPRQRVTISC

SEQ ID NO: 135 - protein, 15 aa, Homo sapiens
WYQQLPGKAPKLLIY

SEQ ID NO: 136 - protein, 32 aa, Homo sapiens
GVSDRFSGSKSGTSASLAISGLQSEDEADYYC

SEQ ID NO: 137 - protein, 22 aa, Homo sapiens
QSVLTQPPSVSGAPGQRVTISC

SEQ ID NO: 138 - protein, 15 aa, Homo sapiens
WYQQLPGTAPKLLIY

SEQ ID NO: 139 - protein, 32 aa, Homo sapiens
GVPDRFSGSKSGTSASLAITGLQAEDEADYYC

SEQ ID NO: 140 - protein, 22 aa, Homo sapiens
QSVLTQPPSASGTPGQRVTISC

SEQ ID NO: 141 - protein, 15 aa, Homo sapiens
WYQQLPGTAPKLLIY

SEQ ID NO: 142 - protein, 32 aa, Homo sapiens
GVPDRFSGSKSGTSASLAISGLQSEDEADYYC

SEQ ID NO: 143 - protein, 22 aa, Homo sapiens
QSVLTQPPSASGTPGQRVTISC

SEQ ID NO: 144 - protein, 15 aa, Homo sapiens
WYQQLPGTAPKLLIY

SEQ ID NO: 145 - protein, 32 aa, Homo sapiens
GVPDRFSGSKSGTSASLAISGLRSEDEADYYC

SEQ ID NO: 146 - protein, 22 aa, Homo sapiens
QSVLTQPPSVSAAPGQKVTISC

SEQ ID NO: 147 - protein, 15 aa, Homo sapiens
WYQQLPGTAPKLLIY

SEQ ID NO: 148 - protein, 32 aa, Homo sapiens
GIPDRFSGSKSGTSATLGITGLQTGDEADYYC

SEQ ID NO: 149 - protein, 22 aa, Homo sapiens
QSALTQPPSASGSPGQSVTISC

SEQ ID NO: 150 - protein, 15 aa, Homo sapiens
WYQQHPGKAPKLMIY

SEQ ID NO: 151 - protein, 32 aa, Homo sapiens
GVPDRFSGSKSGNTASLTVSGLQAEDEADYYC

SEQ ID NO: 152 - protein, 22 aa, Homo sapiens
QSALTQPRSVSGSPGQSVTISC

SEQ ID NO: 153 - protein, 15 aa, Homo sapiens
WYQQHPGKAPKLMIY

SEQ ID NO: 154 - protein, 32 aa, Homo sapiens
GVPDRFSGSKSGNTASLTISGLQAEDEADYYC

SEQ ID NO: 155 - protein, 22 aa, Homo sapiens
QSALTQPASVSGSPGQSITISC

SEQ ID NO: 156 - protein, 15 aa, Homo sapiens
WYQQHPGKAPKLMIY

SEQ ID NO: 157 - protein, 32 aa, Homo sapiens
GVSNRFSGSKSGNTASLTISGLQAEDEADYYC

SEQ ID NO: 158 - protein, 22 aa, Homo sapiens
QSALTQPPSVSGSPGQSVTISC

SEQ ID NO: 159 - protein, 15 aa, Homo sapiens
WYQQPPGTAPKLMIY

SEQ ID NO: 160 - protein, 32 aa, Homo sapiens; identical to SEQ ID NO: 154

SEQ ID NO: 161 - protein, 22 aa, Homo sapiens
QSALTQPASVSGSPGQSITISC

SEQ ID NO: 162 - protein, 15 aa, Homo sapiens
WYQQHPGKAPKLMIY

SEQ ID NO: 163 - protein, 32 aa, Homo sapiens; identical to SEQ ID NO: 157

SEQ ID NO: 164 - protein, 22 aa, Homo sapiens
SYELTQPPSVSVSPGQTASITC

SEQ ID NO: 165 - protein, 15 aa, Homo sapiens
WYQQKPGQSPVLVIY

SEQ ID NO: 166 - protein, 32 aa, Homo sapiens
GIPERFSGSNSGNTATLTISGTQAMDEADYYC

SEQ ID NO: 167 - protein, 22 aa, Homo sapiens
SYELTQPLSVSVALGQTARITC

SEQ ID NO: 168 - protein, 15 aa, Homo sapiens
WYQQKPGQAPVLVIY

SEQ ID NO: 169 - protein, 32 aa, Homo sapiens
GIPERFSGSNSGNTATLTISRAQAGDEADYYC

SEQ ID NO: 170 - protein, 22 aa, Homo sapiens
SYELTQPPSVSVSPGQTARITC

SEQ ID NO: 171 - protein, 15 aa, Homo sapiens
WYQQKSGQAPVLVIY

SEQ ID NO: 172 - protein, 32 aa, Homo sapiens
GIPERFSGSSSGTMATLTISGAQVEDEADYYC

SEQ ID NO: 173 - protein, 22 aa, Homo sapiens
SYELTQPPSVSVSLGQMARITC

SEQ ID NO: 174 - protein, 15 aa, Homo sapiens
WYQQKPGQFPVLVIY

SEQ ID NO: 175 - protein, 32 aa, Homo sapiens
GIPERFSGSSSGTIVTLTISGVQAEDEADYYC

SEQ ID NO: 176 - protein, 22 aa, Homo sapiens
SSELTQDPAVSVALGQTVRITC

SEQ ID NO: 177 - protein, 15 aa, Homo sapiens
WYQQKPGQAPVLVIY

SEQ ID NO: 178 - protein, 32 aa, Homo sapiens
GIPDRFSGSSSGNTASLTITGAQAEDEADYYC

SEQ ID NO: 179 - protein, 22 aa, Homo sapiens
SYVLTQPPSVSVAPGKTARITC

SEQ ID NO: 180 - protein, 15 aa, Homo sapiens
WYQQKPGQAPVLVIY

SEQ ID NO: 181 - protein, 32 aa, Homo sapiens
GIPERFSGSNSGNTATLTISRVEAGDEADYYC

SEQ ID NO: 182 - protein, 22 aa, Homo sapiens
SYELTQLPSVSVSPGQTARITC

SEQ ID NO: 183 - protein, 15 aa, Homo sapiens
WYQQKPGQAPELVIY

SEQ ID NO: 184 - protein, 32 aa, Homo sapiens
GIPERFSGSTSGNTTTLTISRVLTEDEADYYC

SEQ ID NO: 185 - protein, 22 aa, Homo sapiens
SYELMQPPSVSVSPGQTARITC

SEQ ID NO: 186 - protein, 15 aa, Homo sapiens
WYQQKPGQAPVLVIY

SEQ ID NO: 187 - protein, 32 aa, Homo sapiens
GIPERFSGSSSGTTVTLTISGVQAEDEADYYC

SEQ ID NO: 188 - protein, 22 aa, Homo sapiens
SYELTQPSSVSVSPGQTARITC

SEQ ID NO: 189 - protein, 15 aa, Homo sapiens
WFQQKPGQAPVLVIY

SEQ ID NO: 190 - protein, 32 aa, Homo sapiens
GIPERFSGSSSGTTVTLTISGAQVEDEADYYC

SEQ ID NO: 191 - protein, 22 aa, Homo sapiens
LPVLTQPPSASALLGASIKLTC

SEQ ID NO: 192 - protein, 15 aa, Homo sapiens
WYQQRPGRSPQYIMK

SEQ ID NO: 193 - protein, 32 aa, Homo sapiens
GIPDRFMGSSSGADRYLTFSNLQSDDEAEYHC

SEQ ID NO: 194 - protein, 22 aa, Homo sapiens
QPVLTQSSSASASLGSSVKLTC

SEQ ID NO: 195 - protein, 15 aa, Homo sapiens
WHQQQPGKAPRYLMK

SEQ ID NO: 196 - protein, 32 aa, Homo sapiens
GVPDRFSGSSSGADRYLTISNLQLEDEADYYC

SEQ ID NO: 197 - protein, 22 aa, Homo sapiens
QLVLTQSPSASASLGASVKLTC

SEQ ID NO: 198 - protein, 15 aa, Homo sapiens
WHQQQPEKGPRYLMK

SEQ ID NO: 199 - protein, 32 aa, Homo sapiens
GIPDRFSGSSSGAERYLTISSLQSEDEADYYC

SEQ ID NO: 200 - protein, 22 aa, Homo sapiens
QPVLTQPPSSSASPGESARLTC

SEQ ID NO: 201 - protein, 15 aa, Homo sapiens
WYQQKPGSPPRYLLY

SEQ ID NO: 202 - protein, 34 aa, Homo sapiens
GVPSRFSGSKDASANTGILLISGLQSEDEADYYC

SEQ ID NO: 203 - protein, 22 aa, Homo sapiens
QAVLTQPASLSASPGASASLTC

SEQ ID NO: 204 - protein, 15 aa, Homo sapiens
WYQQKPGSPPQYLLR

SEQ ID NO: 205 - protein, 34 aa, Homo sapiens
GVPSRFSGSKDASANAGILLISGLQSEDEADYYC

SEQ ID NO: 206 - protein, 22 aa, Homo sapiens
QPVLTQPSSHSASSGASVRLTC

SEQ ID NO: 207 - protein, 15 aa, Homo sapiens
WYQQKPGNPPRYLLY

SEQ ID NO: 208 - protein, 34 aa, Homo sapiens
GVPSRFSGSNDASANAGILRISGLQPEDEADYYC

SEQ ID NO: 209 - protein, 22 aa, Homo sapiens
NFMLTQPHSVSESPGKTVTISC

SEQ ID NO: 210 - protein, 15 aa, Homo sapiens
WYQQRPGSSPTTVIY

SEQ ID NO: 211 - protein, 34 aa, Homo sapiens
GVPDRFSGSIDSSSNSASLTISGLKTEDEADYYC

SEQ ID NO: 212 - protein, 22 aa, Homo sapiens
QTVVTQEPSLTVSPGGTVTLTC

SEQ ID NO: 213 - protein, 15 aa, Homo sapiens
WFQQKPGQAPRALIY

SEQ ID NO: 214 - protein, 32 aa, Homo sapiens
WTPARFSGSLLGGKAALTLSGVQPEDEAEYYC

SEQ ID NO: 215 - protein, 22 aa, Homo sapiens
QAVVTQEPSLTVSPGGTVTLTC

SEQ ID NO: 216 - protein, 15 aa, Homo sapiens
WFQQKPGQAPRTLIY

SEQ ID NO: 217 - protein, 32 aa, Homo sapiens
WTPARFSGSLLGGKAALTLSGAQPEDEAEYYC

SEQ ID NO: 218 - protein, 22 aa, Homo sapiens
QTVVTQEPSFSVSPGGTVTLTC

SEQ ID NO: 219 - protein, 15 aa, Homo sapiens
WYQQTPGQAPRTLIY

SEQ ID NO: 220 - protein, 32 aa, Homo sapiens
GVPDRFSGSILGNKAALTITGAQADDESDYYC

SEQ ID NO: 221 - protein, 22 aa, Homo sapiens
QPVLTQPPSASASLGASVTLTC

SEQ ID NO: 222 - protein, 15 aa, Homo sapiens
WYQQRPGKGPRFVMR

SEQ ID NO: 223 - protein, 32 aa, Homo sapiens
GIPDRFSVLGSGLNRYLTIKNIQEEDESDYHC

SEQ ID NO: 224 - protein, 22 aa, Homo sapiens
QAGLTQPPSVSKGLRQTATLTC

SEQ ID NO: 225 - protein, 15 aa, Homo sapiens
WLQQHQGHPPKLLSY

SEQ ID NO: 226 - protein, 32 aa, Homo sapiens
GISERLSASRSGNTASLTITGLQPEDEADYYC

SEQ ID NO: 227 - protein, 10 aa, Homo sapiens
FGTGTKVTVL

SEQ ID NO: 228 - protein, 10 aa, Homo sapiens
FGGGTKLTVL

SEQ ID NO: 229 - protein, 10 aa, Homo sapiens
FGGGTKLTVL

SEQ ID NO: 230 - protein, 10 aa, Homo sapiens
FGGGTQLTVL

SEQ ID NO: 231 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 233 - protein, 25 aa, Homo sapiens [sequence omitted]

SEQ ID NO: 234 - protein, 14 aa, Homo sapiens
WVRQAPGQGLEWMG

SEQ ID NO: 235 - protein, 32 aa, Homo sapiens
RVTMTRDTSISTAYMELSRLRSDDTAVYYCAR

SEQ ID NO: 236 - protein, 25 aa, Homo sapiens [sequence omitted]

SEQ ID NO: 237 - protein, 14 aa, Homo sapiens
WVRQAPGQRLEWMG

SEQ ID NO: 238 - protein, 32 aa, Homo sapiens
RVTITRDTSASTAYMELSSLRSEDTAVYYCAR

SEQ ID NO: 239 - protein, 25 aa, Homo sapiens [sequence omitted]

SEQ ID NO: 240 - protein, 14 aa, Homo sapiens
WVRQATGQGLEWMG

SEQ ID NO: 241 - protein, 32 aa, Homo sapiens
RVTMTRNTSISTAYMELSSLRSEDTAVYYCAR

SEQ ID NO: 242 - protein, 25 aa, Homo sapiens [sequence omitted]

SEQ ID NO: 243 - protein, 14 aa, Homo sapiens
WVRQAPGQGLEWMG

SEQ ID NO: 244 - protein, 32 aa, Homo sapiens
RVTMTTDTSTSTAYMELRSLRSDDTAVYYCAR

SEQ ID NO: 245 - protein, 25 aa, Homo sapiens
QVQLVQSGAEVKKPGASVKVSCKVS

SEQ ID NO: 246 - protein, 14 aa, Homo sapiens
WVRQAPGKGLEWMG

SEQ ID NO: 247 - protein, 32 aa, Homo sapiens
RVTMTEDTSTDTAYMELSSLRSEDTAVYYCAT

SEQ ID NO: 248 - protein, 25 aa, Homo sapiens
QMQLVQSGAEVKKTGSSVKVSCKAS

SEQ ID NO: 249 - protein, 14 aa, Homo sapiens
WVRQAPGQALEWMG

SEQ ID NO: 250 - protein, 32 aa, Homo sapiens
RVTITRDRSMSTAYMELSSLRSEDTAMYYCAR

SEQ ID NO: 251 - protein, 25 aa, Homo sapiens [sequence omitted]

SEQ ID NO: 252 - protein, 14 aa, Homo sapiens
WVRQAPGQGLEWMG

SEQ ID NO: 253 - protein, 32 aa, Homo sapiens
RVTMTRDTSTSTVYMELSSLRSEDTAVYYCAR

SEQ ID NO: 254 - protein, 25 aa, Homo sapiens
QMQLVQSGPEVKKPGTSVKVSCKAS

SEQ ID NO: 255 - protein, 14 aa, Homo sapiens
WVRQARGQRLEWIG

SEQ ID NO: 256 - protein, 32 aa, Homo sapiens
RVTITRDMSTSTAYMELSSLRSEDTAVYYCAA

SEQ ID NO: 257 - protein, 25 aa, Homo sapiens [sequence omitted]

SEQ ID NO: 258 - protein, 14 aa, Homo sapiens
WVRQAPGQGLEWMG

SEQ ID NO: 259 - protein, 32 aa, Homo sapiens
RVTITADESTSTAYMELSSLRSEDTAVYYCAR

SEQ ID NO: 260 - protein, 25 aa, Homo sapiens [sequence omitted]

SEQ ID NO: 261 - protein, 14 aa, Homo sapiens
WVRQAPGQGLEWMG

SEQ ID NO: 262 - protein, 32 aa, Homo sapiens
RVTITADKSTSTAYMELSSLRSEDTAVYYCAR

SEQ ID NO: 263 - protein, 25 aa, Homo sapiens
EVQLVQSGAEVKKPGATVKISCKVS

SEQ ID NO: 264 - protein, 14 aa, Homo sapiens
WVQQAPGKGLEWMG

SEQ ID NO: 265 - protein, 32 aa, Homo sapiens
RVTITADTSTDTAYMELSSLRSEDTAVYYCAT

SEQ ID NO: 266 - protein, 25 aa, Homo sapiens
QITLKESGPTLVKPTQTLTLTCTFS

SEQ ID NO: 267 - protein, 14 aa, Homo sapiens
WIRQPPGKALEWLA

SEQ ID NO: 268 - protein, 33 aa, Homo sapiens
RLTITKDTSKNQVVLTMTNMDPVDTATYYCAHR

SEQ ID NO: 269 - protein, 25 aa, Homo sapiens
QVTLKESGPVLVKPTETLTLTCTVS

SEQ ID NO: 270 - protein, 14 aa, Homo sapiens
WIRQPPGKALEWLA

SEQ ID NO: 271 - protein, 33 aa, Homo sapiens
RLTISKDTSKSQVVLTMTNMDPVDTATYYCARI

SEQ ID NO: 272 - protein, 25 aa, Homo sapiens
QVTLKESGPALVKPTQTLTLTCTFS

SEQ ID NO: 273 - protein, 14 aa, Homo sapiens
WIRQPPGKALEWLA

SEQ ID NO: 274 - protein, 33 aa, Homo sapiens
RLTISKDTSKNQVVLTMTNMDPVDTATYYCARI

SEQ ID NO: 275 - protein, 25 aa, Homo sapiens [sequence omitted]

SEQ ID NO: 276 - protein, 14 aa, Homo sapiens
WVRQAPGKGLEWVA

SEQ ID NO: 277 - protein, 32 aa, Homo sapiens
RFTISRDNAKNSLYLQMNSLRAEDTAVYYCAR

SEQ ID NO: 278 - protein, 25 aa, Homo sapiens [sequence omitted]

SEQ ID NO: 279 - protein, 14 aa, Homo sapiens
WVRQAPGKGLEWVS

SEQ ID NO: 280 - protein, 33 aa, Homo sapiens
RFTISRDNAKNSLYLQMNSLRAEDTALYYCAKD

SEQ ID NO: 281 - protein, 25 aa, Homo sapiens
QVQLVESGGGLVKPGGSLRLSCAAS

SEQ ID NO: 282 - protein, 14 aa, Homo sapiens
WIRQAPGKGLEWVS

SEQ ID NO: 283 - protein, 32 aa, Homo sapiens; identical to SEQ ID NO: 277

SEQ ID NO: 284 - protein, 25 aa, Homo sapiens [sequence omitted]

SEQ ID NO: 285 - protein, 14 aa, Homo sapiens
WVRQATGKGLEWVS

SEQ ID NO: 286 - protein, 32 aa, Homo sapiens
RFTISRENAKNSLYLQMNSLRAGDTAVYYCAR

SEQ ID NO: 287 - protein, 25 aa, Homo sapiens
EVQLVESGGGLVKPGGSLRLSCAAS

SEQ ID NO: 288 - protein, 14 aa, Homo sapiens
WVRQAPGKGLEWVG

SEQ ID NO: 289 - protein, 32 aa, Homo sapiens
RFTISRDDSKNTLYLQMNSLKTEDTAVYYCTT

SEQ ID NO: 290 - protein, 25 aa, Homo sapiens
EVQLVESGGGVVRPGGSLRLSCAAS

SEQ ID NO: 291 - protein, 14 aa, Homo sapiens
WVRQAPGKGLEWVS

SEQ ID NO: 292 - protein, 32 aa, Homo sapiens
RFTISRDNAKNSLYLQMNSLRAEDTALYHCAR

SEQ ID NO: 293 - protein, 25 aa, Homo sapiens
EVQLVESGGGLVKPGGSLRLSCAAS

SEQ ID NO: 294 - protein, 14 aa, Homo sapiens
WVRQAPGKGLEWVS

SEQ ID NO: 295 - protein, 32 aa, Homo sapiens; identical to SEQ ID NO: 277

SEQ ID NO: 296 - protein, 25 aa, Homo sapiens
EVQLLESGGGLVQPGGSLRLSCAAS

SEQ ID NO: 297 - protein, 14 aa, Homo sapiens
WVRQAPGKGLEWVS

SEQ ID NO: 298 - protein, 32 aa, Homo sapiens
RFTISRDNSKNTLYLQMNSLRAEDTAVYYCAK

SEQ ID NO: 299 - protein, 25 aa, Homo sapiens
QVQLVESGGGVVQPGRSLRLSCAAS

SEQ ID NO: 300 - protein, 14 aa, Homo sapiens
WVRQAPGKGLEWVA

SEQ ID NO: 301 - protein, 32 aa, Homo sapiens; identical to SEQ ID NO: 298

SEQ ID NO: 302 - protein, 25 aa, Homo sapiens
QVQLVESGGGVVQPGRSLRLSCAAS

SEQ ID NO: 303 - protein, 14 aa, Homo sapiens
WVRQAPGKGLEWVA

SEQ ID NO: 304 - protein, 32 aa, Homo sapiens
RFTISRDNSKNTLYLQMNSLRAEDTAVYYCAR

SEQ ID NO: 305 - protein, 25 aa, Homo sapiens
QVQLVESGGGVVQPGRSLRLSCAAS

SEQ ID NO: 306 - protein, 14 aa, Homo sapiens
WVRQAPGKGLEWVA

SEQ ID NO: 307 - protein, 32 aa, Homo sapiens; identical to SEQ ID NO: 298

SEQ ID NO: 308 - protein, 25 aa, Homo sapiens
QVQLVESGGGVVQPGRSLRLSCAAS

SEQ ID NO: 309 - protein, 14 aa, Homo sapiens
WVRQAPGKGLEWVA

SEQ ID NO: 310 - protein, 32 aa, Homo sapiens; identical to SEQ ID NO: 304

SEQ ID NO: 311 - protein, 25 aa, Homo sapiens
EVQLVESGGVVVQPGGSLRLSCAAS

SEQ ID NO: 312 - protein, 14 aa, Homo sapiens
WVRQAPGKGLEWVS

SEQ ID NO: 313 - protein, 33 aa, Homo sapiens
RFTISRDNSKNSLYLQMNSLRTEDTALYYCAKD

SEQ ID NO: 314 - protein, 25 aa, Homo sapiens [sequence omitted]

SEQ ID NO: 315 - protein, 14 aa, Homo sapiens
WVRQAPGKGLEWVS

SEQ ID NO: 316 - protein, 32 aa, Homo sapiens
RFTISRDNAKNSLYLQMNSLRDEDTAVYYCAR

SEQ ID NO: 317 - protein, 25 aa, Homo sapiens
EVQLVESGGGLVQPGRSLRLSCTAS

SEQ ID NO: 318 - protein, 14 aa, Homo sapiens
WFRQAPGKGLEWVG

SEQ ID NO: 319 - protein, 32 aa, Homo sapiens
RFTISRDGSKSIAYLQMNSLKTEDTAVYYCTR

SEQ ID NO: 320 - protein, 25 aa, Homo sapiens
EVQLVETGGGLIQPGGSLRLSCAAS

SEQ ID NO: 321 - protein, 14 aa, Homo sapiens
WVRQAPGKGLEWVS

SEQ ID NO: 322 - protein, 32 aa, Homo sapiens; identical to SEQ ID NO: 304

SEQ ID NO: 323 - protein, 25 aa, Homo sapiens [sequence omitted]

SEQ ID NO: 324 - protein, 14 aa, Homo sapiens
WVRQAPGKGLEYVS

SEQ ID NO: 325 - protein, 32 aa, Homo sapiens
RFTISRDNSKNTLYLQMGSLRAEDMAVYYCAR

SEQ ID NO: 326 - protein, 25 aa, Homo sapiens [sequence omitted]

SEQ ID NO: 327 - protein, 14 aa, Homo sapiens
WVRQAPGKGLEWVS

SEQ ID NO: 328 - protein, 32 aa, Homo sapiens; identical to SEQ ID NO: 304

SEQ ID NO: 329 - protein, 25 aa, Homo sapiens [sequence omitted]

SEQ ID NO: 330 - protein, 14 aa, Homo sapiens
WVRQAPGKGLEWVG

SEQ ID NO: 331 - protein, 32 aa, Homo sapiens
RFTISRDDSKNSLYLQMNSLKTEDTAVYYCAR

SEQ ID NO: 332 - protein, 25 aa, Homo sapiens
EVQLVESGGGLVQPGGSLKLSCAAS

SEQ ID NO: 333 - protein, 14 aa, Homo sapiens
WVRQASGKGLEWVG

SEQ ID NO: 334 - protein, 32 aa, Homo sapiens
RFTISRDDSKNTAYLQMNSLKTEDTAVYYCTR

SEQ ID NO: 335 - protein, 25 aa, Homo sapiens [sequence omitted]

SEQ ID NO: 336 - protein, 14 aa, Homo sapiens
WVRQAPGKGLVWVS

SEQ ID NO: 337 - protein, 32 aa, Homo sapiens
RFTISRDNAKNTLYLQMNSLRAEDTAVYYCAR

SEQ ID NO: 338 - protein, 25 aa, Homo sapiens
EVQLVESRGVLVQPGGSLRLSCAAS

SEQ ID NO: 339 - protein, 14 aa, Homo sapiens
WVRQAPGKGLEWVS

SEQ ID NO: 340 - protein, 32 aa, Homo sapiens
RFTISRDNSKNTLHLQMNSLRAEDTAVYYCKK

SEQ ID NO: 341 - protein, 25 aa, Homo sapiens
QVQLQESGPGLVKPSGTLSLTCAVS

SEQ ID NO: 342 - protein, 14 aa, Homo sapiens
WVRQPPGKGLEWIG

SEQ ID NO: 343 - protein, 32 aa, Homo sapiens
RVTISVDKSKNQFSLKLSSVTAADTAVYYCAR

SEQ ID NO: 344 - protein, 25 aa, Homo sapiens
QVQLQESGPGLVKPSDTLSLTCAVS

SEQ ID NO: 345 - protein, 14 aa, Homo sapiens
WIRQPPGKGLEWIG

SEQ ID NO: 346 - protein, 32 aa, Homo sapiens
RVTMSVDTSKNQFSLKLSSVTAVDTAVYYCAR

SEQ ID NO: 347 - protein, 25 aa, Homo sapiens
QVQLQESGPGLVKPSQTLSLTCTVS

SEQ ID NO: 348 - protein, 14 aa, Homo sapiens
WIRQHPGKGLEWIG

SEQ ID NO: 349 - protein, 32 aa, Homo sapiens
RVTISVDTSKNQFSLKLSSVTAADTAVYYCAR

SEQ ID NO: 350 - protein, 25 aa, Homo sapiens
QLQLQESGSGLVKPSQTLSLTCAVS

SEQ ID NO: 351 - protein, 14 aa, Homo sapiens
WIRQPPGKGLEWIG

SEQ ID NO: 352 - protein, 32 aa, Homo sapiens
RVTISVDRSKNQFSLKLSSVTAADTAVYYCAR

SEQ ID NO: 353 - protein, 25 aa, Homo sapiens
QVQLQESGPGLVKPSQTLSLTCTVS

SEQ ID NO: 354 - protein, 14 aa, Homo sapiens
WIRQPPGKGLEWIG

SEQ ID NO: 355 - protein, 32 aa, Homo sapiens; identical to SEQ ID NO: 349

SEQ ID NO: 356 - protein, 25 aa, Homo sapiens
QVQLQESGPGLVKPSQTLSLTCTVS

SEQ ID NO: 357 - protein, 14 aa, Homo sapiens
WIRQHPGKGLEWIG

SEQ ID NO: 358 - protein, 32 aa, Homo sapiens; identical to SEQ ID NO: 349

SEQ ID NO: 359 - protein, 25 aa, Homo sapiens [sequence omitted]

SEQ ID NO: 360 - protein, 14 aa, Homo sapiens
WIRQPPGKGLEWIG

SEQ ID NO: 361 - protein, 32 aa, Homo sapiens; identical to SEQ ID NO: 349

SEQ ID NO: 362 - protein, 25 aa, Homo sapiens
QLQLQESGPGLVKPSETLSLTCTVS

SEQ ID NO: 363 - protein, 14 aa, Homo sapiens
WIRQPPGKGLEWIG

SEQ ID NO: 364 - protein, 32 aa, Homo sapiens; identical to SEQ ID NO: 349

SEQ ID NO: 365 - protein, 25 aa, Homo sapiens
QVQLQESGPGLVKPSETLSLTCTVS

SEQ ID NO: 366 - protein, 14 aa, Homo sapiens
WIRQPPGKGLEWIG

SEQ ID NO: 367 - protein, 32 aa, Homo sapiens; identical to SEQ ID NO: 349

SEQ ID NO: 368 - protein, 25 aa, Homo sapiens
QVQLQESGPGLVKPSETLSLTCTVS

SEQ ID NO: 369 - protein, 14 aa, Homo sapiens
WIRQPPGKGLEWIG

SEQ ID NO: 370 - protein, 32 aa, Homo sapiens; identical to SEQ ID NO: 349

SEQ ID NO: 371 - protein, 25 aa, Homo sapiens
QVQLQESGPGLVKPSETLSLTCAVS

SEQ ID NO: 372 - protein, 14 aa, Homo sapiens
WIRQPPGKGLEWIG

SEQ ID NO: 373 - protein, 32 aa, Homo sapiens; identical to SEQ ID NO: 349

SEQ ID NO: 374 - protein, 25 aa, Homo sapiens
EVQLVQSGAEVKKPGESLKISCKGS

SEQ ID NO: 375 - protein, 14 aa, Homo sapiens
WVRQMPGKGLEWMG

SEQ ID NO: 376 - protein, 32 aa, Homo sapiens
QVTISADKSISTAYLQWSSLKASDTAMYYCAR

SEQ ID NO: 377 - protein, 25 aa, Homo sapiens
EVQLVQSGAEVKKPGESLRISCKGS

SEQ ID NO: 378 - protein, 14 aa, Homo sapiens
WVRQMPGKGLEWMG

SEQ ID NO: 379 - protein, 32 aa, Homo sapiens
HVTISADKSISTAYLQWSSLKASDTAMYYCAR

SEQ ID NO: 380 - protein, 25 aa, Homo sapiens
QVQLQQSGPGLVKPSQTLSLTCAIS

SEQ ID NO: 381 - protein, 14 aa, Homo sapiens
WIRQSPSRGLEWLG

SEQ ID NO: 382 - protein, 32 aa, Homo sapiens
RITINPDTSKNQFSLQLNSVTPEDTAVYYCAR

SEQ ID NO: 383 - protein, 25 aa, Homo sapiens
QVQLVQSGSELKKPGASVKVSCKAS

SEQ ID NO: 384 - protein, 14 aa, Homo sapiens
WVRQAPGQGLEWMG

SEQ ID NO: 385 - protein, 32 aa, Homo sapiens
RFVFSLDTSVSTAYLQICSLKAEDTAVYYCAR

SEQ ID NO: 386 - protein, 11 aa, Homo sapiens
WGQGTLVTVSS

SEQ ID NO: 387 - protein, 11 aa, Homo sapiens
WGRGTLVTVSS

SEQ ID NO: 388 - protein, 11 aa, Homo sapiens
WGQGTMVTVSS

SEQ ID NO: 389 - protein, 11 aa, Homo sapiens
WGQGTLVTVSS

SEQ ID NO: 390 - protein, 11 aa, Homo sapiens
WGQGTLVTVSS

SEQ ID NO: 391 - protein, 11 aa, Homo sapiens
WGQGTTVTVSS

SEQ ID NO: 392 - protein, 102 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCP

SEQ ID NO: 393 - protein, 18 aa, Homo sapiens
PSTPPTPSPSTPPTPSPS

SEQ ID NO: 394 - protein, 101 aa, Homo sapiens
CCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKS

SEQ ID NO: 395 - protein, 111 aa, Homo sapiens
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLA

SEQ ID NO: 396 - protein, 102 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCP

SEQ ID NO: 397 - protein, 5 aa, Homo sapiens
PPPPP

SEQ ID NO: 398 - protein, 101 aa, Homo sapiens
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKS

SEQ ID NO: 399 - protein, 111 aa, Homo sapiens; identical to SEQ ID NO: 395

SEQ ID NO: 400 - protein, 101 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWP

SEQ ID NO: 401 - protein, 58 aa, Homo sapiens
ESPKAQASSVPTAQPQAEGSLAKATTAPATTRNTGRGGEEKKKEKEKEEQEERETKTP

SEQ ID NO: 402 - protein, 108 aa, Homo sapiens
ECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREP

SEQ ID NO: 403 - protein, 108 aa, Homo sapiens
AAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVS

SEQ ID NO: 404 - protein, 103 aa, Homo sapiens
ASTQSPSVFPLTRCCKNIPSNATSVTLGCLATGYFPEPVMVTCDTGSLNGTTMTLPATTLTLSGHYATISLLTVSGAWAKQMFTCRVAHTPSSTDWVDNKTFS

SEQ ID NO: 405 - protein, 107 aa, Homo sapiens
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 406 - protein, 108 aa, Homo sapiens
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 407 - protein, 108 aa, Homo sapiens
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNP

SEQ ID NO: 408 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 409 - protein, 15 aa, Homo sapiens
EPKSCDKTHTCPPCP

SEQ ID NO: 410 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 411 - protein, 105 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 412 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 413 - protein, 12 aa, Homo sapiens
ERKCCVECPPCP

SEQ ID NO: 414 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 415 - protein, 105 aa, Homo sapiens
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 416 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 417 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 418 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 419 - protein, 105 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSP

SEQ ID NO: 420 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 421 - protein, 12 aa, Homo sapiens
ESKYGPPCPSCP

SEQ ID NO: 422 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 423 - protein, 105 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 424 - protein, 104 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLP

SEQ ID NO: 425 - protein, 112 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVP

SEQ ID NO: 426 - protein, 106 aa, Homo sapiens
DQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPK

SEQ ID NO: 427 - protein, 110 aa, Homo sapiens
GVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKST

SEQ ID NO: 428 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 429 - protein, 15 aa, Homo sapiens
MANCERTFIAIKPDG

SEQ ID NO: 430 - protein, 15 aa, Homo sapiens
IKPDGVQRGLVGEII

SEQ ID NO: 431 - protein, 15 aa, Homo sapiens
VGEIIKRFEQKGFRL

SEQ ID NO: 432 - protein, 15 aa, Homo sapiens
KGFRLVGLKFMQASE

SEQ ID NO: 433 - protein, 15 aa, Homo sapiens
MQASEDLLKEHYVDL

SEQ ID NO: 434 - protein, 15 aa, Homo sapiens
HYVDLKDRPFFAGLV

SEQ ID NO: 435 - protein, 15 aa, Homo sapiens
FAGLVKYMHSGPVVA

SEQ ID NO: 436 - protein, 15 aa, Homo sapiens
GPVVAMVWEGLNVVK

SEQ ID NO: 437 - protein, 15 aa, Homo sapiens
LNVVKTGRVMLGETN

SEQ ID NO: 438 - protein, 15 aa, Homo sapiens
LGETNPADSKPGTIR

SEQ ID NO: 439 - protein, 15 aa, Homo sapiens
PGTIRGDFCIQVGRN

SEQ ID NO: 440 - protein, 15 aa, Homo sapiens
QVGRNIIHGSDSVES

SEQ ID NO: 441 - protein, 15 aa, Homo sapiens
DSVESAEKEIGLWFH

SEQ ID NO: 442 - protein, 15 aa, Homo sapiens
GLWFHPEELVDYTSC

SEQ ID NO: 443 - protein, 15 aa, Homo sapiens
ELVDYTSCAQNWIYE

SEQ ID NO: 444 - protein, 5 aa, Homo sapiens
MVWEG

SEQ ID NO: 445 - protein, 6 aa, Homo sapiens
IIKRFE

SEQ ID NO: 446 - protein, 221 aa, Homo sapiens
QSVEESGGRLVTPGTPLTLTCTVSGFSLSTYYMSWVRQAPGKGLEWIGIIYPSGSTYCASWAKGRFTISKASTTVDLKITSPTTEDTATYFCARPDNDGTSGYLSGFGLWGQGLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 447 - protein, 219 aa, Homo sapiens
DVVMTQTPASVSEPVGGTVTIKCGASEDISRYLVWYQQKPGQPPKRLIYKASTLASGVPSRFKGSGSGTDFTLTISDLECDDAATYYCQCTYGTYAGSFFYSFGGGTEVVVERTDAAPTVSIFPPSSEQLTSGGASVVCFLNNFYPKDINVKWKIDGSERQNGVLNSWTDQDSKDCTYSMSSTLTLTKDEYERHNSYTCEATHKTSTSPIVKSFNRNEC

(19) United States
(12) Patent Application Publication
(10) Pub. No.: US 2023/0203142 A1
(43) Pub. Date: Jun. 29, 2023

(54) TREATMENT OF METABOLIC DISORDERS THROUGH THE TARGETING OF A NOVEL CIRCULATING HORMONE COMPLEX

(71) Applicant: The President and Fellows of Harvard College, Cambridge, MA (US)

(72) Inventors: Gökhan S. Hotamisligil, Weston, MA (US); Ediz Calay, Cambridge, MA (US); Kacey Prentice, Cambridge, MA (US); Jani Pertti Kristian Saksi, Cambridge, MA (US)

(21) Appl. No.: 17/983,098

(22) Filed: Nov. 8, 2022

Related U.S. Application Data
(63) Continuation of application No. PCT/US2021/031643, filed on May 10, 2021.
(60) Provisional application No. 63/022,235, filed on May 8, 2020.

(51) Int. Cl. C07K 16/18; G01N 33/573; G01N 33/68

(52) U.S. Cl. CPC C07K 16/18; G01N 33/5735; G01N 33/6893; C07K 2317/92

(57) ABSTRACT

The present invention provides a method to identify compounds useful in inhibiting the adverse effects of excessive FABP4 on the modulation of NDPK-ADK agonism of G protein-coupled receptors (GPCR) and channels in FABP4-mediated disorders. It has been surprisingly discovered that the fatty acid binding protein 4 (FABP4) inhibits the ability of the nucleoside diphosphate kinase (NDPK) and adenosine kinase (ADK) complex to agonize GPCRs on target cells by forming an NDPK-ADK/FABP4 complex, resulting in, amongst other things, impaired or reduced insulin secretion in islet β-cells and an increase in glucose levels in the bloodstream. By inhibiting the formation of the NDPK-ADK/FABP4 complex, or inhibiting FABP4 downregulation of NDPK-ADK complex modulation of GPCRs, it has been discovered that FABP4-medited effects can be blunted, including the modulation of islet β-cell insulin secretion, providing for a reduction in glucose levels and the attenuation of metabolic dysfunction.

### CROSS-REFERENCE TO RELATED APPLICATIONS

This application is a continuation of International Patent Application No. PCT/US2021/031643, filed in the U.S. Receiving Office on May 10, 2021, which claims the benefit of U.S. Provisional Patent Application 63/022,235, filed May 8, 2020. The entirety of each of these applications is hereby incorporated by reference for all purposes.

### FIELD OF THE INVENTION

The present invention provides methods for targeting a novel circulating hormone complex comprising fatty acid binding protein 4 (FABP4), nucleoside-diphosphate kinase (NDPK), and adenosine kinase (ADK) for the treatment and prevention of metabolic disorders including dysregulated insulin secretion and elevated blood glucose levels.

### INCORPORATION BY REFERENCE

The contents of the XML file named “15020-027WO1US1_SequenceListing_ST26.xml” which was created on Nov. 7, 2022 and is 386 KB in size, are hereby incorporated by reference in their entirety.

### BACKGROUND OF THE INVENTION

Hormones are required for the integrated function of various organ systems, enabling an orchestrated response to the fundamental needs of the organism including growth, feeding, reproduction, stress and metabolic adaptations. The dysregulation or absence of an individual hormone can have profound effects on physiology, which in large part are attributed to vital alterations in metabolism. The appropriate management of energy stores is a process and involves the function of numerous signals derived from multiple tissue sources including the pancreas, hypothalamus, adrenal glands, and thyroid. The coordinated actions of these systems are critical for both adaptive and homeostatic response systems and the maintenance of organismal health and survival.

Adipose tissue is the primary site for energy storage in the body. At times of energy deficit, adipocytes initiate a program to release energy by breaking down triglyceride stores through lipolysis. Once energy balance is restored, lipolysis is terminated and adipocytes replenish their energy storage capacity. While this is a critical function to support survival and escape food shortages and life-threatening stress conditions, uncontrolled or chronic lipolysis is also detrimental for the body and disrupts metabolic homeostasis. Despite being a highly secretory tissue, producing numerous adipokines, the secretion of only one recently identified hormone, fatty acid binding protein 4 (FABP4), is coupled to the breakdown of lipid stores during lipolysis (Ertunc et al., 2015; Schlottmann et al., 2014; Villeneuve et al., 2018). This places FABP4 in the unique position of being able to communicate the energy status of the body to distant metabolic tissues, critical for maintaining appropriate energy balance and overall metabolic health.

In both rodent and human studies, circulating FABP4 levels have been strongly associated with obesity and cardiometabolic diseases (Hotamisligil and Bernlohr, 2015; Prentice et al., 2019). It is also established that pharmacological targeting or reduced expression through either genetic ablation in mice, or low-expression genetic variant in multiple independent cohorts of humans, is protective against type 2 diabetes (T2D) and cardiovascular disease (Saksi et al., 2014; Tuncman et al., 2006; Zhao et al., 2017). Hence, rapidly accumulating evidence strongly supports a model where some of the main systemic physiological and pathophysiological actions of FABP4 are mediated primarily by its secreted hormonal form, stimulating a new set of considerations related to its biology that are distinct from the intracellular pools of this molecule.

While the majority of studies examining hormonal FABP4 biology in vivo have focused on hepatic glucose and lipid metabolism, a few reports have provided evidence that hormonal FABP4 may have a yet undefined role in the regulation of pancreatic islet beta cell function. Early studies demonstrated that acute lipolysis-induced insulin secretion is reduced in FABP4 deficient mice, but not in response to the secretagogues arginine and glyburide (Scheja et al., 1999). A follow-up study further demonstrated that in vivo glucose stimulated insulin secretion (GSIS) is enhanced in genetically obese (ob/ob)-Fabp4-deficient mice compared to controls (Uysal et al., 2000). Whether this functionality is also mediated by the hormonal FABP4 remained unresolved, and a few studies utilizing recombinant FABP4 have been unable to demonstrate a consistent impact on GSIS, even at super-physiological levels, ex vivo or in vivo (Kralisch et al., 2015; Wu et al., 2014). Two studies quantifying plasma FABP4 in human subjects have reported a potential positive correlation between serum FABP4 and insulin levels in non-diabetic human subjects, though they have limitations in terms of low sample sizes, and not accounting for the likely confounding effect of FABP4 levels correlating with BMI (Nakamura et al., 2017; Wu et al., 2014). Despite the characterization of hormonal FABP4 secretion and strong links with human disease, the mechanism of hormonal FABP4 action remains essentially completely unknown. This has presented important limitations to exploring potential targets for therapeutic treatment.

Accordingly, it is an object of the invention to provide methods useful in treating FABP4-mediated disorders. It is another objection of the invention to provide compounds, and methods of identifying compounds, useful in treating disorders associated with FABP4-mediated disorders, including, but not limited to diabetes (both type 1 and type 2) obesity, cardiovascular disease, fatty liver disease, and/or cancer, among others.

### SUMMARY OF THE INVENTION

The present invention is based on the surprising discovery that fatty-acid binding protein 4 (FABP4) is capable of mediating the effects of nucleoside diphosphate kinase (NDPK) and adenosine kinase (ADK) activity by binding the NDPK-ADK complex extracellularly, modulating NDPK-ADK complex modulation of purinergic G protein-coupled receptors on target cells, such as P2Y1 (P2Y1R) on pancreatic islet β-cells. NDPK and ADK regulate the activity of GPCRs, such as P2Y1R, on the surface of target cells to potentiate a variety of intracellular effects and alter extracellular adenosine triphosphate (ATP) and adenosine diphosphate (ADP) levels. In particular, the formation of the NDPK-ADK complex agonizes the activity of P2Y1R on pancreatic islet (3-cells increasing extracellular ADP levels and inducing the secretion of insulin in the presence of glucose. This process is known as glucose-stimulated insulin secretion (GSIS). In conditions wherein circulating FABP4 is abundant, for example in obesity and other metabolic disorders, FABP4 binds with the NDPK-ADK complex to form an NDPK-ADK/FABP4 complex that inhibits the agonistic stimulatory effect of the NDPK-ADK complex on the P2Y1R, reducing GSIS, impairing the function of β-cells including their ability to secrete insulin, and ultimately impairing β-cell survival. The formation of the NDPK-ADK/FABP4 complex and antagonism of the P2Y1R results in reduced insulin secretion, increased blood glucose levels and a decrease in pancreatic islet β-cell mass and function, directly contributing to metabolic dysfunction, including the development of type I diabetes.

Hormones are traditionally investigated as solitary entities, with a single hormone released to act through a defined receptor. Described herein is a novel mechanism of hormone biology, wherein circulating FABP4 forms a functional complex with two extracellular nucleoside kinases, NDPK and ADK to mediate biological activity. This activity is independent of a defined complex receptor, and instead is regulated through metabolite signaling via purinergic receptors to regulate downstream effects. This unique mechanism of action allows for FABP4, the only known hormone to be released from adipose tissue upon stimulation of lipolysis to have a diverse activity profile, establishing a novel endocrine axis of metabolic regulation.

It has been surprisingly found that, by inhibiting the interaction of FABP4 with NDPK and/or the NDPK-ADK complex, or by sequestering the NDPK-ADK/FABP4 complex away from interactions with the P2Y1R, the adverse effects associated with FABP4 binding to the NDPK-ADK complex can be reduced, improving metabolic outcomes including the improvement of GSIS. Furthermore, by inhibiting the formation of the novel NDPK-ADK/FABP4 complex, it has been discovered that pancreatic islet β-cell integrity and survival can be preserved, and the onset of disorders mediated by FABP4, including for example, type I diabetes, can be delayed and/or prevented. Thus, the discovery of the interaction of FABP4 with the NDPK-ADK complex and modulation of the P2Y1R on pancreatic islet β-cells provides a new treatment pathway for modulating FABP4-mediated disorders, and an important model for identifying and/or developing compounds capable of modulating FABP4-mediated disorders without significant off-target effects.

Cells expressing purinergic GPCRs may be key targets for the NDPK-ADK/FABP4 complex. In particular, pancreatic islet β-cells may be a particularly key target tissue for the NDPK-ADK/FABP4 complex, due to the unusually high extracellular ATP and ADP concentrations in the intra-islet space, acting as substrates for the kinases. These adenosine nucleosides are charged, and thus do not freely diffuse across membranes. Both ATP and ADP accumulate in insulin granules and are co-secreted along with insulin (Leitner et al., 1975; Sakamoto et al., 2014). Impairment of vesicular ATP release improves insulin secretion and whole-body insulin sensitivity, consistent with the observed effects of reducing extracellular ATP through the inhibition of the NDPK-ADK/FABP4 complex. Because purinergic receptors are broadly expressed, the NDPK-ADK/FABP4 complex can be targeted to treat a number of FABP4-mediated disorders.

Using the teachings herein, compounds for the treatment of FABP4-mediated disorders can be identified and selected that have advantageous properties for human treatment. The selection of compounds capable of inhibiting the formation of the NDPK-ADK/FABP4 complex or the inhibition of purinergic GPCR antagonism by the NDPK-ADK/FABP4 complex provides a critical target for therapeutic treatments.

Accordingly, provided herein are methods for treating or preventing an FABP4-mediated disorder in a subject by (i) identifying a compound capable of neutralizing the ability of FABP4 to associate with the NDPK-ADK complex or prevents or inhibits the NDPK-ADK/FABP4 complex from generating increased levels of ATP to antagonize purinergic G protein-coupled receptors (GPCRs) or channels on target cells, and (ii) administering to the subject an effective amount of the compound. In some embodiments, the administered compound prevents FABP4 from associating with the NDPK-ADK complex, allowing the NDPK-ADK complex alone to increase ADP production to agonize the purinergic GPCRs or channels and their downstream signaling activities. In some embodiments, the administered compound prevents the NDPK-ADK/FABP4 complex from generating increased levels of ATP to antagonize the purinergic GPCRs or channels and their downstream signaling activities. In some embodiments, the target cell is selected from pancreatic islet β-cells, hepatic cells, endothelial cells, macrophages, smooth muscle cells, neurons, glial cells and epithelial cells.

In one aspect, provided herein is a method of treating or preventing a FABP4-mediated disorder in a subject by (i) identifying a compound capable of selectively binding to FABP4, thus preventing the formation of the NDPK-ADK/FABP4 complex, and (ii) administering to the subject an effective amount of the compound. In some embodiments, the compound is capable of inducing glucose-stimulated insulin secretion in a human pancreatic islet β-cell or insulin secreting cell line assay in the presence of glucose, FABP4, NDPK, and ADK. In some embodiments, the compound is a monoclonal antibody or an antigen binding agent. In some embodiments, the compound is a monoclonal antibody or an antigen binding agent that binds to FABP4 with a K_{d} (the equilibrium dissociation constant between the antibody and its antigen) of ≤10⁻⁷, for example, a K_{d} in the nanomolar (10⁻⁷ to 10⁻⁹) or picomolar (10⁻⁹ to 10⁻¹²) range, or less. In some embodiments, the antibody or antigen binding agent binds to FABP4 preferentially, that is with a greater affinity or K_{d}, than it binds to NDPK, ADK, and/or NDPK-ADK complex. In some embodiments, the antibody or antigen binding agent does not bind to NDPK, ADK, and/or NDPK-ADK complex.

In one aspect, provided herein is a method of treating or preventing a FABP4-mediated disorder in a subject by (i) identifying a compound capable of selectively binding to NDPK, thus preventing the formation of the NDPK-ADK/FABP4 complex, and (ii) administering to the subject an effective amount of the compound. In some embodiments, the antibody or antigen binding agent binds to the NDPK-A isoform. In one embodiment, the compound neutralizes the ability of NDPK-A to form a complex with FABP4 and thus agonizing a P2Y1 receptor by binding to NDPK-A at amino acid Met75, Va176, Trp77, Glu78, and Gly79 of SEQ ID NO: 1. In one embodiment, the compound neutralizes the ability of NDPK-A to form a complex with FABP4 and thus agonizing a P2Y1 receptor by binding to NDPK-A at amino acid Ile24, Ile25, Lys26, Arg27, Phe28, and Glu29 of SEQ ID NO: 1. In one embodiment, the compound neutralizes the ability of NDPK-A to form a complex with FABP4 and thus agonizing a P2Y1 receptor by binding to NDPK-A at amino acid Met75, Va176, Trp77, Glu78, and Gly79 of SEQ ID NO: 1 and amino acid Ile24, Ile25, Lys26, Arg27, Phe28, and Glu29 of SEQ ID NO: 1. In some embodiments, the antibody or antigen binding agent binds to the NDPK-B isoform. In some embodiments, the antibody or antigen binding agent is capable of inducing glucose-stimulated insulin secretion in a human pancreatic islet β-cell or insulin secreting cell line assay in the presence of glucose, FABP4, NDPK, and ADK. In some embodiments, the compound does not inhibit the ability of the NDPK-ADK complex from increasing ADP to agonize the activity of purinergic GPCRs or channels, for example P2Y1R on human pancreatic islet β-cells or insulin-secreting cell lines. In some embodiments, the compound is a monoclonal antibody or an antigen binding agent. In some embodiments, the compound is a monoclonal antibody or an antigen binding agent that binds to NDPK with a K_{d} of ≤10⁻⁸, for example, a K_{d} of 10⁻⁸, 10⁻⁹, 10⁻¹⁰, 10⁻¹¹, 10⁻¹², or less. In some embodiments, the antibody of antigen binding agent binds to NDPK preferentially, that is with a greater affinity or K_{d}, than it binds to FABP4 and/or ADK. In some embodiments, the antibody of antigen binding agent does not bind to uncomplexed ADK and/or FABP4.

In one aspect, provided herein is a method of treating or preventing a FABP4-mediated disorder in a subject by (i) identifying a compound capable of selectively binding to ADK, thus preventing the formation of the NDPK-ADK/FABP4 complex, and (ii) administering to the subject an effective amount of the compound. In some embodiments, the compound is capable of inducing glucose-stimulated insulin secretion in a human pancreatic islet β-cell or insulin secreting cell line assay in the presence of glucose, FABP4, NDPK, and ADK. In some embodiments, the compound does not inhibit the ability of the NDPK-ADK complex from generating ADP to agonize the activity of purinergic GPCRs or channels, for example P2Y1R on human pancreatic islet β-cells or insulin-secreting cell lines. In some embodiments, the compound is a monoclonal antibody or an antigen binding agent. In some embodiments, the compound is a monoclonal antibody or an antigen binding agent that binds to ADK with a K_{d} of ≤10⁻⁷, for example, a K_{d} of 10⁻⁷, 10⁻⁸, 10⁻⁹, 10⁻¹⁰, 10⁻¹¹, 10⁻¹², or less. In some embodiments, the antibody of antigen binding agent binds to ADK preferentially, that is with a greater affinity or K_{d}, than it binds to FABP4 and/or NDPK. In some embodiments, the antibody of antigen binding agent does not bind to uncomplexed NDPK and/or FABP4.

In one aspect, provided herein is a method of treating or preventing a FABP4-mediated disorder in a subject by (i) identifying a compound capable of inhibiting the NDPK-ADK/FABP4 complex from generating ATP to antagonize purinergic GPCRs or channels on target cells, and (ii) administering to the subject an effective amount of the compound. In some embodiments, the compound is capable of inducing glucose-stimulated insulin secretion in a human pancreatic islet β-cell or insulin secreting cell line assay in the presence of glucose, FABP4, NDPK, and ADK. In some embodiments, the compound does not inhibit the ability of the NDPK-ADK complex from generating ADP to agonize the activity of purinergic GPCRs or channels, for example P2Y1R on human pancreatic islet β-cells or insulin-secreting cell lines. In some embodiments, the compound is a monoclonal antibody or an antigen binding agent. In some embodiments, the compound is a monoclonal antibody or an antigen binding agent that binds to the NDPK-ADK/FABP4 complex with an affinity greater than that for NDPK-ADK complex. In some embodiments, the compound is a monoclonal antibody or an antigen binding agent that binds to the NDPK-ADK/FABP4 complex with an affinity greater than that for NDPK and/or ADK uncomplexed. In some embodiments, the antibody of antigen binding agent does not bind to NDPK and/or ADK uncomplexed.

In one aspect, provided herein is a method of treating or preventing a FABP4-mediated disorder in a subject by (i) identifying a compound capable of, in the presence of an NDPK-ADK/FABP4 complex, providing one or more of the following biological effects in a pancreatic islet β-cell or insulin secreting cell: a) in increase of extracellular ADP compared to ATP, b) an increase in the measurement of insulin secretion in response to glucose, c) an increase in GTP, d) a decrease in intracellular free Ca′ concentrations or an increase in extracellular calcium influx, e) a decrease in cyclic AMP (cAMP) generation, f) a decrease in inositol 1,4,5-trisphosphate (IP3) generation, g) a decrease in IP3R phosphorylation, h) a decrease in the level of cleaved caspase 3 (CC3), i) a decrease in the level of cleaved caspase 3/7 (CC3/7), j) a decrease in the level of c-Jun N-terminal kinase (JNK) phosphorylation, k) a decrease in the level of endoplasmic reticulum stress marker immunoglobulin heavy chain-binding protein (Bip (Grp78)), 1) a decrease in the level of endoplasmic reticulum stress marker C/-EBP homologous protein (CHOP), m) a decrease in the accumulation of misprocessed or misfolded proteins, and/or n) a decrease in diacylglycerol (DAG) generation, or a combination thereof, compared to the biological effects in a pancreatic islet β-cell or insulin secreting cell in the presence of NDPK-ADK/FABP4 without the compound; and (ii) administering to the subject the compound.

In embodiments of any of the aspects and embodiments described herein, the FABP4-mediated disorder to be prevented or treated is a metabolic disorder, a cardiovascular disorder, a fatty liver disease, an inflammatory disorder, a neurodegenerative disorder, a cancer, or other FABP-4-mediated disorder. In some embodiments, the FABP4-mediated disorder is selected from diabetes type 1 (T1D), diabetes type 2 (T2D), hyperglycemia, diabetic ketoacidosis, hyperglycemic hyperosmolar syndrome, cardiovascular disease, diabetic nephropathy or kidney failure, diabetic retinopathy, impaired fasting glucose, impaired glucose tolerance, dyslipidemia, obesity, cataracts, stroke, atherosclerosis, impaired wound healing, hyperglycemia, perioperative hyperglycemia, insulin resistance syndrome, metabolic syndrome, liver fibrosis, lung fibrosis, non-alcoholic fatty liver disease (NAFLD), nonalcoholic steatohepatitis (NASH), hepatocellular carcinoma, cirrhosis, glucagonoma, Necrolytic migratory erythema (NME), hyperglucanemia, hyperinsulinemia, asthma, autoimmune diseases, preeclampsia, or other FABP4-mediated disorders described herein.

In one aspect, provided herein is a method of treating or preventing type I diabetes in a subject comprising (i) identifying a compound capable of neutralizing the ability of FABP4 to associate with the NDPK-ADK complex or prevents or inhibits the NDPK-ADK/FABP4 complex from generating ATP to antagonize purinergic G protein-coupled receptors (GPCRs) or channels on target cells, and (ii) administering to the subject an effective amount of the compound.

In one aspect, provided herein is a method of reducing pancreatic islet β-cell mass loss in a subject with type I diabetes comprising (i) identifying a compound capable of neutralizing the ability of FABP4 to associate with the NDPK-ADK complex or prevents or inhibits the NDPK-ADK/FABP4 complex from generating ATP to antagonize purinergic G protein-coupled receptors (GPCRs) or channels on target cells, and (ii) administering to the subject an effective amount of the compound.

In one aspect, provided herein is a method of preserving pancreatic β-cell function in a subject with type I diabetes comprising (i) identifying a compound capable of neutralizing the ability of FABP4 to associate with the NDPK-ADK complex or prevents or inhibits the NDPK-ADK/FABP4 complex from generating ATP to antagonize purinergic G protein-coupled receptors (GPCRs) or channels on target cells, and (ii) administering to the subject an effective amount of the compound.

In one aspect, provided herein is a method of preventing the onset of type I diabetes in a subject comprising (i) identifying a compound capable of neutralizing the ability of FABP4 to associate with the NDPK-ADK complex or that prevents or inhibits the NDPK-ADK/FABP4 complex from generating ATP to antagonize purinergic G protein-coupled receptors (GPCRs) or channels on target cells, and (ii) administering to the subject an effective amount of the compound.

In one aspect, provided herein is a method of increasing pancreatic islet β-cell mass in a subject comprising (i) identifying a compound capable of neutralizing the ability of FABP4 to associate with the NDPK-ADK complex or prevents or inhibits the NDPK-ADK/FABP4 complex from generating ATP to antagonize purinergic G protein-coupled receptors (GPCRs) or channels on target cells, and (ii) administering to the subject an effective amount of the compound.

It has also been discovered that FABP4 serum levels are significantly elevated in human serum following the onset of type I diabetes (T1D). This elevation persists through the early phases of disease, despite significant reductions in body mass index (BMI), which has previously been associated with increased levels of serum FABP4 levels. In light of these findings, and the discovery that the onset of T1D diabetes may be inhibited by the methods described herein, the discovery provides a mechanism to monitor for and inhibit the development of T1D.

Accordingly, in one aspect, provided herein is a method of monitoring and inhibiting the development of the onset of type I diabetes in a subject comprising:

a. measuring an FABP4 baseline level in serum of a type I diabetes pre-onset subject at a first time point;

b. measuring the FABP4 level in serum of the subject at one or more subsequent time points;

c. if the subject's FABP4 levels in the one or more subsequent time points is significantly greater than the subject's FABP4 level at a first time point, administering to the subject an effective amount of a compound that neutralizes the ability of FABP4 to associate with the NDPK-ADK complex, prevents or inhibits the NDPK-ADK/FABP4 complex from generating ATP to antagonize purinergic G protein-coupled receptors (GPCRs) or channels on target cells, or generates ADP to agonize P2Y1Rs on pancreatic islet β-cells.

In an alternative aspect, through the discovery of the NDPK-ADK/FABP4 complex, compounds capable of inhibiting the binding of FABP4 to the NDPK-ADK complex or compounds capable of sequestering the NDPK-ADK/FABP4 complex allowing unbound NDPK-ADK complex to modulate purinergic GPCRs or channels on target cells, compounds that prevent the formation of the NDPK-ADK/FABP4 complex, can be identified or designed. In some embodiments, the compound is an antibody or antigen binding agent. In some embodiments, the antibody or antigen binding agent selectively binds to FABP4 over NDPK, ADK, or NDPK-ADK complex. In some embodiments, the antibody or antigen binding agent selectively binds to the NDPK-ADK/FABP4 complex over NDPK, ADK, or NDPK-ADK complex. In some embodiments, the antibody or antigen binding agent binds to NDPK or the NDPK-ADK complex, preventing FABP4 from interacting with the complex, while allowing the NDPK-ADK complex to generate ADP to agonize a purinergic GPCR on a target cell. Such antibodies and/or antigen binding agents are useful in the treatment of diseases mediated by FABP4 and attenuation of purinergic GPCR modulation on the surface of target cells.

Accordingly, in an aspect, a method of identifying a compound capable of binding FABP4 and inhibiting formation of NDPK-ADK/FABP4 complex is provided comprising:

i. determining whether the compound binds to FABP4;

ii. contacting the compound with FABP4 and NDPK-ADK complex (or alternatively NDPK and ADK uncomplexed); and,

iii. determining whether the compound inhibits the interaction of FABP4 with NDPK-ADK complex. In some embodiments, the assay is performed in vitro in the absence of cells. In some embodiments, the method further comprises introducing the compound into an ADP-kinase assay with FABP4, NDPK and ADK (or alternatively NDPK-ADK in complex), ATP, and GDP, wherein measurement of ADP production is indicative of a compound capable of binding FABP4 without inhibiting NDPK-ADK complex activity. In some embodiments the method further comprises introducing the compound into a cellular assay in the presence of FABP4, NDPK, ADK, and glucose, wherein the cellular assay includes a population of target cells expressing a purinergic GPCR or channels, for example an insulin secreting cell line expressing P2Y1R, and measuring the ratio of extracellular ADP to ATP, or alternatively insulin secretion, wherein an increase in the ratio of ADP to ATP or insulin secretion indicates a compound capable of binding FABP4 and inhibiting formation of NDPK-ADK/FABP4 complex. In some embodiments, the cell population is human pancreatic islet β-cells. In some embodiments, the ratio of ADP to ATP is measured in the cellular assay. In some embodiments, insulin secretion is measured in the cellular assay.

In an alternative aspect, a method of identifying a compound capable of binding NDPK and inhibiting the formation of NDPK-ADK/FABP4 complex without interfering with NDPK-ADK complex activity is provided comprising:

i. determining whether the compound binds to NDPK;

ii. contacting the compound with NDPK-ADK complex (or alternatively NDPK and ADK) and FABP4; and,

iii. determining whether the compound inhibits interaction of FABP4 with NDPK without inhibiting NDPK-ADK complex activity. In some embodiments, the assay is performed in vitro in the absence of cells. In some embodiments, the method further comprises introducing the compound into an ADP-kinase assay with FABP4, NDPK and ADK (or alternatively, NDPK-ADK complex), ATP, and GDP, wherein measurement of ADP production is indicative of a compound capable of binding NDPK without inhibiting NDPK-ADK complex activity. In some embodiments the method further comprises introducing the compound into a cellular assay in the presence of FABP4, NDPK, ADK, and glucose, wherein the cellular assay includes a population of target cells expressing a purinergic GPCR or channels, for example an insulin secreting cell line expressing P2Y1R, and measuring the ratio of extracellular ADP to ATP (or alternatively insulin secretion), wherein an increase in the ratio of ADP to ATP or insulin secretion indicates a compound capable of binding NDPK and inhibiting formation of NDPK-ADK/FABP4 complex. In some embodiments, the cell population is human pancreatic islet β-cells. In some embodiments, the ratio of ADP to ATP is measured in the cellular assay. In some embodiments, insulin secretion is measured in the cellular assay.

In an alternative aspect, a method of identifying a compound capable of binding ADK and inhibiting the formation of NDPK-ADK/FABP4 without interfering with NDPK-ADK complex activity is provided comprising:
i. determining whether the compound binds to ADK;
ii. contacting the compound with NDPK-ADK complex (or alternatively, NDPK and ADK), and FABP4; and,
ii. determining whether the compound inhibits interaction of FABP4 with NDPK-ADK complex without inhibiting NDPK-ADK complex activity. In some embodiments, the assay is performed in vitro in the absence of cells. In some embodiments, the method further comprises introducing the compound into an ADP-kinase assay with NDPK-ADK, ATP, and adenosine, wherein measurement of ADP production is indicative of a compound capable of binding ADK without inhibiting ADK activity. In some embodiments the method further comprises introducing the compound into a cellular assay in the presence of FABP4, NDPK, ADK, and glucose, wherein the cellular assay includes a population of target cells expressing a purinergic GPCR or channels, for example an insulin secreting cell line expressing P2Y1R, and measuring the ratio of extracellular ADP to ATP (or alternatively insulin secretion), wherein an increase in the ratio of ADP to ATP or insulin secretion indicates a compound capable of binding ADK and inhibiting formation of NDPK-ADK/FABP4 complex. In some embodiments, the cell population is human pancreatic islet β-cells. In some embodiments, the ratio of ADP to ATP is measured in the cellular assay. In some embodiments, insulin secretion is measured in the cellular assay.

In an alternative aspect, a method of identifying a compound capable of binding the NDPK-ADK complex and inhibiting the formation of NDPK-ADK/FABP4 without inhibiting NDPK-ADK complex activity is provided comprising:

i. determining whether the compound binds to the NDPK-ADK complex;

ii. contacting the compound with NDPK-ADK complex (or alternatively, NDPK and ADK), and FABP4; and,

ii. determining whether the compound inhibits interaction of FABP4 with NDPK-ADK complex without inhibiting NDPK-ADK complex activity. In some embodiments, the assay is performed in vitro in the absence of cells. In some embodiments, the method further comprises introducing the compound into an ADP-kinase assay with FABP4, NDPK and ADK (or alternatively, NDPK-ADK complex), ATP, and GDP, wherein measurement of ADP production is indicative of a compound capable of binding the NDPK-ADK complex and inhibiting formation of NDPK-ADK/FABP4 complex without inhibiting NDPK-ADK complex activity. In some embodiments the method further comprises introducing the compound into a cellular assay in the presence of FABP4, NDPK, ADK, and glucose, wherein the cellular assay includes a population of target cells expressing a purinergic GPCR or channels, for example an insulin secreting cell line expressing P2Y1R, and measuring the ratio of extracellular ADP to ATP (or alternatively insulin secretion), wherein an increase in the ratio of ADP to ATP or insulin secretion indicates a compound capable of binding the NDPK-ADK complex and inhibiting formation of the NDPK-ADK/FABP4 complex. In some embodiments, the cell population is human pancreatic islet (3-cells. In some embodiments, the ratio of ADP to ATP is measured in the cellular assay. In some embodiments, insulin secretion is measured in the cellular assay.

In another aspect, a method of identifying a compound capable of binding NDPK-ADK in complex with FABP4 (NDPK-ADK/FABP4 complex) and inhibiting ATP antagonism of a purinergic GPCR on a target cell is provided comprising:

i. contacting the compound with NDPK-ADK/FABP4 complex and,

ii. determining whether the compound binds to the NDPK-ADK/FABP4 complex.

In some embodiments, the assay is performed in vitro in the absence of cells. In some embodiments the method further comprises introducing the compound into a cellular assay in the presence of FABP4, NDPK, ADK, and glucose, wherein the cellular assay includes a population of target cells expressing a purinergic GPCR or channels, for example an insulin secreting cell line expressing P2Y1R, and measuring the ratio of extracellular ADP to ATP (or alternatively insulin secretion), wherein an increase in the ratio of ADP to ATP or insulin secretion indicates a compound capable of binding the NDPK-ADK/FABP4 complex and inhibiting ATP antagonism of a purinergic GPCR or channel. In some embodiments, the cell population is human pancreatic islet β-cells. In some embodiments, the ratio of ADP to ATP is measured in the cellular assay. In some embodiments, insulin secretion is measured in the cellular assay.

In an additional aspect, provided herein is a method of identifying a compound capable of increasing glucose-stimulated insulin secretion (GSIS) in the presence of FABP4 comprising:

i. contacting FABP4, NDPK, and ADK, NDPK-ADK complex, and/or NDPK-ADK/FABP4 complex with an insulin secreting cell which express GPCRs in the presence of a compound;

ii. contacting FABP4, NDPK, ADK, NDPK-ADK complex, and/or NDPK-ADK/FABP4 complex with an insulin secreting cell, which express GPCRs in the absence of a compound; and,

iii. measuring the level of insulin secretion in the presence of the compound and in the absence of the compound;

wherein an increase in the level of insulin secretion in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in insulin secretion by the insulin secreting cell. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii) for each of i) and ii) above, measuring one or more of: a) extracellular ADP and ATP levels, b) insulin secretion, c) GTP, d) intracellular free Ca2+ concentrations, e) cyclic AMP (cAMP) generation, f) inositol 1,4,5-trisphosphate (IP3) generation, g) IP3R phosphorylation, h) the level of cleaved caspase 3 (CC3), i) the level of cleaved caspase 3/7 (CC3/7), j) the level of c-Jun N-terminal kinase (JNK) phosphorylation, k) the level of endoplasmic reticulum stress marker immunoglobulin heavy chain-binding protein (Bip (Grp78)), 1) the level of endoplasmic reticulum stress marker C/-EBP homologous protein (CHOP), m) the accumulation of misprocessed or misfolded proteins, and/or n) diacylglycerol (DAG) generation;

wherein one or more of: a) in increase of extracellular ADP compared to ATP, b) an increase in the measurement of insulin secretion in response to glucose, c) an increase in GTP, d) a decrease in intracellular free Ca2+ concentrations, e) a decrease in cyclic AMP (cAMP) generation, f) a decrease in inositol 1,4,5-trisphosphate (IP3) generation, g) a decrease in IP3R phosphorylation, h) a decrease in the level of cleaved caspase 3 (CC3), i) a decrease in the level of cleaved caspase 3/7 (CC3/7), j) a decrease in the level of c-Jun N-terminal kinase (JNK) phosphorylation, k) a decrease in the level of endoplasmic reticulum stress marker immunoglobulin heavy chain-binding protein (Bip (Grp78)), 1) a decrease in the level of endoplasmic reticulum stress marker C/-EBP homologous protein (CHOP), m) a decrease in the accumulation of misprocessed or misfolded proteins, and/or n) a decrease in diacylglycerol (DAG) generation, or a combination thereof in i) compared to ii) is indicative of a compound capable of treating or preventing a FABP4-mediated disorder in a subject.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring the extracellular ADP and ATP levels in the presence of the compound and in the absence of the compound;

wherein an increase in the ratio of ADP to ATP in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in an increase in the ratio of ADP to ATP by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring insulin secretion in the presence of the compound and in the absence of the compound;

wherein an increase in insulin secretion in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in an increase in insulin secretion by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring GTP levels in the presence of the compound and in the absence of the compound;

wherein an increase in GTP levels in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in an increase in GTP by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring intracellular free Ca2+ concentration in the presence of the compound and in the absence of the compound;

wherein a decrease in intracellular free Ca2+ concentration in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in intracellular free Ca2+ concentration by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring cyclic AMP (cAMP) generation in the presence of the compound and in the absence of the compound;

wherein a decrease in cyclic AMP (cAMP) generation in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in cyclic AMP (cAMP) generation by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring adenylyl cyclase activation in the presence of the compound and in the absence of the compound;

wherein a decrease in adenylyl cyclase activation in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in adenylyl cyclase activation by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring inositol 1,4,5-trisphosphate (IP3) generation in the presence of the compound and in the absence of the compound;

wherein a decrease in inositol 1,4,5-trisphosphate (IP3) generation in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in inositol 1,4,5-trisphosphate (IP3) generation by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring inositol 1,4,5-trisphosphate receptor (IP3R) phosphorylation in the presence of the compound and in the absence of the compound;

wherein a decrease in inositol 1,4,5-trisphosphate receptor (IP3R) phosphorylation in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in inositol 1,4,5-trisphosphate receptor (IP3R) phosphorylation by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring the level of cleaved caspase 3 (CC3) in the presence of the compound and in the absence of the compound;

wherein a decrease in the level of cleaved caspase 3 (CC3) in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in the level of cleaved caspase 3 (CC3) by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring the level of cleaved caspase 3/7 (CC3/7) in the presence of the compound and in the absence of the compound;

wherein a decrease in the level of cleaved caspase 3/7 (CC3/7) in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in the level of cleaved caspase 3/7 (CC3/7) by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring the level of c-Jun N-terminal kinase (JNK) phosphorylation in the presence of the compound and in the absence of the compound;

wherein a decrease in the level of c-Jun N-terminal kinase (JNK) phosphorylation in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in the level of c-Jun N-terminal kinase (JNK) phosphorylation by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring the level of endoplasmic reticulum stress marker immunoglobulin heavy chain-binding protein (Bip (Grp78)) in the presence of the compound and in the absence of the compound;

wherein a decrease in the level of endoplasmic reticulum stress marker immunoglobulin heavy chain-binding protein (Bip (Grp78)) in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in the level of endoplasmic reticulum stress marker immunoglobulin heavy chain-binding protein (Bip (Grp78)) by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring the level of endoplasmic reticulum stress marker C/-EBP homologous protein (CHOP) in the presence of the compound and in the absence of the compound;

wherein a decrease in the level of endoplasmic reticulum stress marker C/-EBP homologous protein (CHOP) in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in the level of endoplasmic reticulum stress marker C/-EBP homologous protein (CHOP) by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring the accumulation of misprocessed or misfolded proteins in the presence of the compound and in the absence of the compound;

wherein a decrease in the accumulation of misprocessed or misfolded proteins in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in the accumulation of misprocessed or misfolded proteins by the insulin secreting cell and is indicative of a compound capable of treating or preventing a FABP4-mediated disorder in a subject. In some embodiments, the insulin secreting cell is a pancreatic islet β-cell.

In one aspect, provided herein is a method of identifying a compound capable of treating or preventing a FABP4-mediated disorder in a subject comprising:

i) contacting the compound with a) NDPK, ADK, and FABP4, b) FABP4 and NDPK-ADK complex, or c) an NDPK-ADK/FABP4 complex in the presence of a pancreatic islet β-cell or insulin secreting cell and glucose;

ii) contacting a) NDPK, ADK, and FABP4, b) FABP4 and NDPK-ADK complex, or c) an NDPK-ADK/FABP4 complex in the presence of a pancreatic islet β-cell or insulin secreting cell and glucose;

iii. measuring diacylglycerol (DAG) generation in the presence of the compound and in the absence of the compound;

wherein a decrease in diacylglycerol (DAG) generation in the presence of the compound is indicative of a compound capable of neutralizing the binding of FABP4 to the NDPK-ADK complex, or capable of sequestering the NDPK-ADK/FABP4 complex and allowing the unbound NDPK-ADK complex to modulate the P2Y1 receptor resulting in a decrease in diacylglycerol (DAG) generation by the insulin secreting cell and is indicative of a compound capable of treating or preventing a FABP4-mediated disorder in a subject. In some embodiments, the insulin secreting cell is a pancreatic islet β-cell. In any of the embodiments above, FABP4, NDPK, and/or ADK can be in the form of a recombinant protein, for example a human recombinant protein. In some embodiments, the NDPK is human NDPK-A. In some embodiments, the NDPK is human NDPK-B. In some embodiments, the assay includes both NDPK-A and NDPK-B.

In one aspect, a method of identifying a compound capable inhibiting formation of NDPK-ADK/FABP4 complex is provided comprising:

i. contacting the compound with recombinant FABP4, recombinant NDPK, and recombinant ADK and,

ii. determining whether the compound inhibits the formation of NDPK-ADK/FABP4 complex. In some embodiments, the assay is performed in vitro in the absence of cells. In some embodiments, the method further comprises introducing the compound into an ADP-kinase assay with FABP4, NDPK and ADK (or alternatively NDPK-ADK), ATP, and GDP, wherein measurement of ADP production is indicative of a compound capable of inhibiting NDPK-ADK/FABP4 complex formation. In some embodiments the method further comprises introducing the compound into a cellular assay in the presence of FABP4, NDPK, ADK, and glucose, wherein the cellular assay includes a population of target cells expressing a purinergic GPCR or channels, for example an insulin secreting cell line expressing P2Y1R, and measuring the ratio of extracellular ADP to ATP (or alternatively insulin secretion), wherein an increase in the ratio of ADP to ATP or insulin secretion indicates a compound capable of inhibiting formation of NDPK-ADK/FABP4 complex. In some embodiments, the cell population is human pancreatic islet β-cells.

Examples of insulin-secreting cell lines for use in the described screening methods include, but are not limited to, primary human islet cells, EndoC-βH1, INS-1 823/13, βTC, RIN, HIT, INS-1, βHC-9, or MING cells.

In one aspect, provided herein is an antibody, for example a monoclonal antibody, or an antigen binding agent, and methods of treatment using the antibody or antigen binding agent, wherein the antibody or antigen binding agent binds to an NDPK-ADK complex. In some embodiments, the antibody binds to the epitope MVWEG (SEQ ID NO: 444) on NDPK-A, which corresponds to which corresponds to amino acid residues Met75, Va176, Trp77, Glu78, and Gly79 of SEQ ID NO: 1. In some embodiments, the antibody binds to the epitope IIKRFE (SEQ ID NO: 445) on NDPK-A, which corresponds to amino acid residues Ile24, Ile25, Lys26, Arg27, Phe28, and Glu29 of SEQ ID NO: 1.

In some embodiments, the antibody binds to the epitopes MVWEG (SEQ ID NO: 444) and IIKRFE (SEQ ID NO: 445) on NDPK-A. In some embodiments, the antibody binds to the epitopes MVWEG (SEQ ID NO: 444) and IIKRFE (SEQ ID NO: 445) on NDPK, and does not bind to FABP4. In some embodiments, the antibody or antigen binding agent binds to the human form of NDPK-A (UniProtKB—P15531 (NDKA_HUMAN)).

In one aspect, provided herein is an antibody, for example a monoclonal antibody, or an antigen binding agent, and methods of treatment using the antibody or antigen binding agent, wherein the antibody or antigen binding agent binds to an NDPK-ADK/FABP4 complex.

In an alternative aspect, provided herein is a composition for use in an assay to identify a compound capable of treating or preventing a FABP4-mediated disorder comprising a recombinant FABP4 protein, a recombinant NDPK protein, and a recombinant ADK protein. In some embodiments, the composition is comprised of human recombinant proteins. In some embodiments, the recombinant NDPK is recombinant human NDPK-A. In some embodiments, the recombinant NDPK is recombinant human NDPK-B. In some embodiments, the assay includes both recombinant NDPK-A and recombinant NDPK-B. In one embodiment, a recombinant FABP4/NDPK-ADK complex composition is generated for use in an assay to identify a compound capable of treating or preventing a FABP4-mediated disorder.

In some embodiments, provided herein is a composition for use in an assay to identify a compound capable of treating or preventing a FABP4-mediated disorder comprising a recombinant FABP4 protein, a recombinant NDPK protein, and a recombinant ADK protein, wherein the recombinant FABP4 protein comprises an amino acid sequence comprising SEQ ID NO: 4, wherein the recombinant NDPK protein comprises an amino acid sequence comprising SEQ ID NO: 1 (NDPK-A) or SEQ ID NO: 2 (NDPK-B), and wherein the recombinant ADK protein comprises an amino acid sequence comprising SEQ ID NO: 3. In some embodiments, the recombinant NDPK in the composition comprises an amino acid sequence comprising SEQ ID NO: 1 (NDPK-A). In some embodiments, the recombinant NDPK in the composition comprises an amino acid sequence comprising SEQ ID NO: 2 (NDPK-B). In some embodiments, the composition further includes ADP, ATP, or glucose.

In some embodiments, provided herein is a composition for use in an assay to identify a compound capable of treating or preventing a FABP4-mediated disorder comprising a recombinant FABP4 protein comprising an amino acid sequence comprising SEQ ID NO: 4, a recombinant NDPK protein comprises an amino acid sequence comprising SEQ ID NO: 1 (NDPK-A), a recombinant NDPK protein comprises an amino acid sequence comprising or SEQ ID NO: 2 (NDPK-B), and a recombinant ADK protein comprising an amino acid sequence comprising SEQ ID NO: 3. In some embodiments, the composition further includes ADP, ATP, or glucose.

### BRIEF DESCRIPTION OF THE DRAWINGS

FIG. 1A shows gross pancreas images in lean FABP4+/+(wild type) and FABP4−/− mice in vivo.

FIG. 1B shows dithizone stained islets in vivo from FABP4−/− mice (representative image from N=3).

FIG. 1C shows immunohistochemical staining for insulin in pancreatic sections from 7-week-old wildtype (WT) or FABP4-deficient (FABP4−/−) mice (N=4/group).

FIG. 1D shows the quantification of percentage of insulin positive area per total pancreatic area based on IHC from 7-week-old WT and FABP4−/− mouse pancreata (N=4/group). *P<0.05, Statistical significance determined by Student's t-test. Data are presented as mean+/−SEM.

FIG. 1E shows the total pancreatic insulin content from 7-week-old WT (Black) and FABP4−/− (White) mouse pancreata (N=3/group). *P<0.05, Statistical significance determined by Student's t-test. Data are presented as mean+/−SEM.

FIG. 1F shows immunohistochemical staining for glucagon in pancreatic sections from 7-week-old WT or FABP4−/− mice (n=5/group).

FIG. 1G shows the quantification of percentage of glucagon positive area per total pancreatic area based on IHC from 7-week-old WT and FABP4−/− mouse pancreata (N=5/group). Data are presented as mean+/−SEM.

FIG. 1H shows glucose-stimulated insulin secretion (GSIS) from islets ex vivo under low glucose (2.8 mM; LG) and high glucose (16.7 mM; HG) conditions from 7-week-old WT (White) and FABP4−/− (Gray) mouse pancreata (N=8/group). **P<0.01. Statistical significance determined by Student's t-test. Data are presented as mean+/−SEM.

FIG. 1I shows immunofluorescent staining for insulin, FABP4, and nuclei (DAPI) in primary isolated mouse islets (N=15 islets).

FIG. 1J shows a Western blot for FABP4 and B-tubulin (B-Tubb) from INS1 cells with and without treatment with FABP4 (N=3).

FIG. 1K shows 6-hour fasting blood glucose from diet-induced obese (DIO) mice before treatment (wk. 0) and following 33 mg/kg a-Ab twice weekly for 3 wks. (N=10/group). ***P<0.001. Statistical significance determined by Student's t-test. Data are presented as mean+/−SEM.

FIG. 1L shows glucose tolerance test (GTT) in DIO mice treated for 2 weeks with PBS or a-Ab (N=10/group). **P<0.01, ***P<0.001. Statistical significance determined by Student's t-test. Data are presented as mean+/−SEM.

FIG. 1M shows immunohistochemical staining for insulin in pancreatic sections from DIO mice treated with PBS or 33 mg/kg a-Ab for 3 wks. (N=6/group).

FIG. 1N shows the quantification of total islet number per pancreatic section in pancreatic sections from DIO mice treated with PBS or a-Ab for 3 weeks (N=6/group). *P<0.05. Statistical significance determined by Student's t-test. Data are presented as mean+/−SEM.

FIG. 1O shows the percentage of insulin positive area per total pancreatic area in pancreatic sections from DIO mice treated with PBS or a-Ab for 3 weeks (N=4-8/group). Statistical significance determined by Student's t-test. Data are presented as mean+/−SEM.

FIG. 2A shows the quantification of plasma FABP4 levels (ng/mL) on the y-axis in autoantibody positive and negative normal glucose tolerant (NGT) individuals compared to new-onset T1D patients (<1-year duration; BABYDIAB cohort) shown on the x-axis (N=30/group). **P<0.01, ***P<0.001. Statistical significance determined by one way ANOVA. Data are presented as mean+/−SEM.

FIG. 2B shows the correlation of plasma FABP4 levels (ng/mL) on the x-axis with HbA1c percentage on the y-axis in established T1D patients (BRI cohort; N=50/group).

FIG. 2C shows plasma FABP4 levels (ng/mL) in NOD mice while non-diabetic (NGT), one week prior to hyperglycemia (Prior), or at time of second glucose reading >250 mg/dl (T1D) (N=10-35/group). *P<0.05, ***P<0.001. Statistical significance determined by one-way ANOVA. Data are presented as mean+/−SEM.

FIG. 2D shows baseline matching parameters for 10-week-old WT female NOD mice prior to the initiation of dosing (x-axis) showing no difference in body weight (y-axis). Data are presented as mean+/−SEM.

FIG. 2E shows baseline matching parameters for 10-week-old WT female NOD mice prior to the initiation of dosing (x-axis) showing no difference in plasma insulin (ng/ml) (y-axis). Data are presented as mean+/−SEM.

FIG. 2F shows baseline matching parameters for 10-week-old WT female NOD mice prior to the initiation of dosing (x-axis) showing no difference in blood glucose (mg/dl) (y-axis). Data are presented as mean+/−SEM.

FIG. 2G shows baseline matching parameters for 10-week-old WT female NOD mice prior to the initiation of dosing (x-axis) showing no difference in plasma FABP4 (ng/ml) (y-axis). Data are presented as mean+/−SEM.

FIG. 2H shows a survival curve for NOD model of T1D measured as a percentage diabetes free (y-axis) and age (weeks, x-axis) following twice weekly treatment with PBS, a-Ab, or c-Ab beginning at 10 wks. of age for the duration of the study (N=36/group). *P<0.05, **P<0.01. Statistical significance determined by one-way ANOVA. Data are presented as mean+/−SEM.

FIG. 2I shows the average blood glucose levels (mg/dl, y-axis) of NOD mice at the time of T1D diagnosis following twice weekly treatment with PBS, a-Ab, or c-Ab (x-axis) (N=11-23). *P<0.05, **P<0.01. Statistical significance determined by one-way ANOVA. Data are presented as mean+/−SEM.

FIG. 2J shows the average plasma insulin levels (ng/ml, y-axis) of NOD mice at the time of T1D diagnosis following twice weekly treatment with PBS, a-Ab, or c-Ab (x-axis) (N=11-23). *P<0.05. Statistical significance determined by one-way ANOVA. Data are presented as mean+/−SEM.

FIG. 2K shows 6-hour fasting blood glucose levels (mg/dl, y-axis) among NOD mice that remained non-diabetic for the duration of the treatment period (weeks, x-axis) (N=13-25). Data are presented as mean+/−SEM.

FIG. 2L shows body weight levels (g, y-axis) among NOD mice that remained non-diabetic for the duration of the treatment period (weeks, x-axis) (N=13-25). Data are presented as mean+/−SEM.

FIG. 2M shows plasma insulin levels (ng/ml, y-axis) among NOD mice that remained non-diabetic for the duration of the treatment period (weeks, x-axis) (N=13-25). Data are presented as mean+/−SEM.

FIG. 2N shows the results of glucose tolerance tests (GTT) (mg/dL, y-axis) over time (min., x-axis) in non-diabetic NOD mice treated with PBS or a-Ab (N=6/group). **P<0.01. Statistical significance determined by one-way ANOVA. Data are presented as mean+/−SEM.

FIG. 2O shows the plasma insulin values (Fold over basal, y-axis) over time (min., x-axis) in non-diabetic NOD mice treated with PBS or a-Ab (N=6/group). *P<0.05. Statistical significance determined by one-way ANOVA. Data are presented as mean+/−SEM.

FIG. 2P shows the results of a glucose-stimulated insulin secretion assay (ng/ml/ug DNA, y-axis) from islets isolated from NOD mice under different feeding conditions (LG, HG, HG+KCl, x-axis) treated with PBS, a-Ab, or c-Ab for 15 weeks (N=4/group).

FIG. 2Q shows immunohistochemical staining for insulin in pancreatic sections from mice treated with c-Ab (top panel) or a-Ab (bottom panel) for 5 wks. (N=4/group).

FIG. 2R shows percentage of insulin positive area (y-axis) per total pancreatic area in pancreatic sections from mice treated with PBS or a-Ab (x-axis) for 5 wks. (N=4/group). *P<0.05. Statistical significance determined by one-way ANOVA. Data are presented as mean+/−SEM.

FIG. 2S shows the quantification if islet number (y-axis) per pancreatic section in pancreatic sections from mice treated with PBS or a-Ab (x-axis) for 5 wks. (N=4/group). **P<0.01. Statistical significance determined by one-way ANOVA. Data are presented as mean+/−SEM.

FIG. 2T shows immunofluorescent staining for insulin and glucagon in pancreatic sections from NOD mice treated with c-Ab (left panel) or a-Ab (right panel) for 5 wks. (N=4/group).

FIG. 2U shows the quantification for insulin and glucagon staining (% Glucagon/Insulin+, y-axis) in pancreatic sections from NOD mice treated with PBS or a-Ab (x-axis) for 5 wks. (N=4/group). *P<0.05. Statistical significance determined by one-way ANOVA. Data are presented as mean+/−SEM.

FIG. 2V shows immunofluorescent staining for insulin and glucagon in pancreatic sections from DIO mice treated with PBS (left panel) or a-Ab (right panel) for 3 wks. (N=5/group).

FIG. 2W shows the quantification for insulin and glucagon staining (glucagon+(% islet), y-axis) in pancreatic sections from DIO mice treated with PBS or a-Ab (x-axis) for 3 wks. (N=26-30 islets from 5 mice/group). **P<0.01. Statistical significance determined by one-way ANOVA. Data are presented as mean+/−SEM.

FIG. 3A shows GSIS from islets from FABP4^{-/-} mice under different feeding conditions (LG, HG) treated with increasing doses of FABP4 (0 mg/ml, 5 ug/ml, 10 ug/ml, and 25 ug/ml) (N=3). FABP4 treatment concentrations are shown on the x-axis and insulin content (ng/mL/ug DNA) is shown on the y-axis. Data are presented as mean+/−SEM.

FIG. 3B shows plasma FABP4 levels (ng/ml, y-axis) over time (y-axis) following acute injection of 10 μg FABP4 (N=3-7/group).

FIG. 3C shows blood glucose levels (mg/dl, y-axis) 20-minutes post-injection with FABP4 or PBS (x-axis) (N=3-7/group). *P<0.05. Statistical significance determined by one-way ANOVA. Data are presented as mean+/−SEM.

FIG. 3D shows plasma insulin levels (ng/ml, y-axis) 20-minutes post-injection with FABP4 or PBS (x-axis) (N=3-7/group). *P<0.05. Statistical significance determined by one-way ANOVA. Data are presented as mean+/−SEM.

FIG. 3E shows the crystal structure showing FABP4 binding through the light chain of a-Ab.

FIG. 4A shows the results of three independent immunoprecipitation and mass spectrometry experiments from WT or FABP4−/− diet-induced obese (DIO) serum with a-Ab or c-Ab (control) with top hits based on enrichment and spectral counts.

FIG. 4B shows spectral counts of Adenosine Kinase (ADK) from Screen 1 (N=3/group) in WT and FABP4−/− DIO mice using a-Ab and c-Ab (x-axis). The y-axis shows the ADK spectral intensity in absorbance units (a.u.).

FIG. 4C shows spectral counts of nucleoside diphosphate kinase (NDPK) from Screen 1 (N=3/group) in WT and FABP4−/− mice using a-Ab and c-Ab (x-axis). The y-axis shows the NDPK spectral intensity in absorbance units (a.u.).

FIG. 4D shows the amino acid sequence for human NDPK-A, and indication of the location of each peptide generated for epitope mapping. Each peptide is 15 amino acids long with an N-terminal 6×His Tag for peptide labeling. Each peptide has a 5 amino acid overlap with the preceding peptide sequentially from the N-terminus of the protein.

FIG. 4E shows example MST binding curves for full-length NDPK-A (left panel), NDPK-A peptide 9 (center panel) and NDPK-A peptide 8 (right panel) with a-Ab.

FIG. 4F shows the crystal structure of human NDPK-A in hexamer conformation (source PDB:3L7U) with the potential binding region of peptide 8 and peptide 3 highlighted in the inset.

FIG. 4G shows the summary of microscale thermophoresis one-to-one binding experiments among proposed complex components (N=6/interaction).

FIG. 4H shows representative mesoscale thermophoresis experiment showing 1:1 protein interaction between FABP4-ADK (N=6/interaction). EC50 was calculated using Hill Slope.

FIG. 4I shows representative mesoscale thermophoresis experiment showing 1:1 protein interaction between NDPK-ADK (N=6/interaction). EC50 was calculated using Hill Slope.

FIG. 4J shows representative mesoscale thermophoresis experiment showing 1:1 protein interaction between ADK and a-Ab (N=6/interaction). EC50 was calculated using Hill Slope.

FIG. 4K shows representative mesoscale thermophoresis experiment showing 1:1 protein interaction between FABP4-NDPK (N=6/interaction). EC50 was calculated using Hill Slope.

FIG. 4L shows representative mesoscale thermophoresis experiment showing 1:1 protein interaction between NDPK and a-Ab (N=6/interaction). EC50 was calculated using Hill Slope.

FIG. 4M shows a Western blot of immunoprecipitation of GST-tagged NDPK with each of the proposed complex components and a-Ab (n=4).

FIG. 5A shows the results of an on-bead kinase assay for ADK activity from WT or FABP4^{-/-} mouse serum quantifying ATP production over time (N=5/group) in the presence of a-Ab or c-Ab. The x-axis shows time in minutes and the y-axis shows ATP production measured in relative luminescence units (RLU). *P<0.05. Statistical significance measured by two-way ANOVA. Data are presented as mean+/−SEM.

FIG. 5B shows ATP production from recombinant ADK in the presence of various complex components (N=3/group). The x-axis shows the various complex components and the y-axis shows ADK ATP production measured in RLU. *P<0.05. Statistical significance measured by one-way ANOVA. Data are presented as mean+/−SEM.

FIG. 5C shows kinase activity of recombinant ADK to generate ATP in the presence of complex components (N=3/group). The x-axis shows the substrate concentrations measured in mM and the y-axis shows ATP production measured in RLU. *P<0.05. Statistical significance measured by one-way ANOVA. Data are presented as mean+/−SEM.

FIG. 5D shows kinase activity of recombinant ADK to generate ADP in the presence of complex components (N=3/group). The x-axis shows the substrate concentrations measured in mM and the y-axis shows ADP production measured in RLU. *P<0.05. Statistical significance measured by one-way ANOVA. Data are presented as mean+/−SEM.

FIG. 5E shows the results of an on-bead kinase assay for NDPK activity from WT or FABP4^{-/-} mouse serum quantifying ADP production over time (N=5/group) in the presence of a-Ab or c-Ab. The x-axis shows time in minutes and the y-axis shows ADP production measured in RLU. *P<0.05. Statistical significance measured by two-way ANOVA. Data are presented as mean+/−SEM.

FIG. 5F shows ADP production from recombinant NDPK in the presence of various complex components (N=3/group). The x-axis shows the various complex components and the y-axis shows NDPK ADP production measured in RLU. *P<0.05, ***P<0.001. Statistical significance measured by one-way ANOVA. Data are presented as mean+/−SEM.

FIG. 5G shows kinase activity of recombinant NDPK to generate ADP in the presence of complex components (N=3/group). The x-axis shows the substrate concentration in mM and the y-axis shows ADP production measured in RLU. *P<0.05, **P<0.01, ***P<0.001. Statistical significance measured by one-way ANOVA. Data are presented as mean+/−SEM.

FIG. 5H shows kinase activity of recombinant NDPK to generate ATP in the presence of complex components (N=3/group). The x-axis shows the substrate concentration in mM and the y-axis shows ATP production measured in RLU. Data are presented as mean+/−SEM.

FIG. 5I shows kinase activity of recombinant ADK to generate ATP in the presence of a-Ab or c-Ab or alone (N=3/group). The x-axis shows the substrate concentration in mM and the y-axis shows ATP production measured in RLU. Data are presented as mean+/−SEM.

FIG. 5J shows kinase activity of recombinant NDPK to generate ADP in the presence of a-Ab or c-Ab or alone (N=3/group). The x-axis shows the substrate concentration in mM and the y-axis shows ADP production measured in RLU. Data are presented as mean+/−SEM.

FIG. 5K shows kinase activity of recombinant ADK to produce ATP in the presence of complex components with a-Ab or c-Ab (n=3/group). The x-axis shows the various complex components and the y-axis shows ADK ATP production measured in RLU. *P<0.05, **P<0.01, ***P<0.001. Statistical significance measured by one-way ANOVA. Data are presented as mean+/−SEM.

FIG. 5L shows kinase activity of recombinant NDPK to produce ADP in the presence of complex components with a-Ab or c-Ab (n=3/group). The x-axis shows the various complex components and the y-axis shows NDPK ADP production measured in RLU. *P<0.05, **P<0.01, ***P<0.001. Statistical significance measured by one-way ANOVA. Data are presented as mean+/−SEM.

FIG. 5M shows a proposed model of extracellular nucleoside regulation from the complex.

FIG. 6A shows GSIS from primary mouse islets from WT mice treated with either low glucose (LG) or high glucose (HG) and treated with NDPK-ADK or FABP4-ADK-NDPK (N=3/group). The treatment conditions are shown on the x-axis and insulin measured in (ng/mL/ug DNA) on the y-axis. Low glucose (LG) is 2.8 mM and high glucose (HG) is 16.7 mM glucose. ***P<0.001. Statistical significance measured by one-way ANOVA. Data are presented as mean+/−SEM.

FIG. 6B shows GSIS from human islets treated with NDPK-ADK or FABP4-ADK-NDPK with and without the addition of a-Ab or c-Ab (N=3). Treatment conditions are shown on the x-axis and insulin measured in (ng/mL/ug DNA) is shown on the y-axis. *P<0.05, **P<0.01. Statistical significance measured by one-way ANOVA. Data are presented as mean+/−SEM.

FIG. 6C shows GSIS from INS1 cells treated with either low glucose (LG) or high glucose (HG) and treated with NDPK-ADK or FABP4-ADK-NDPK with and without the addition of a-Ab or c-Ab (N=3). Treatment conditions are shown on the x-axis and insulin measured in (Fold HG/LG) is shown on the y-axis. Low glucose (LG) is 2.8 mM and high glucose (HG) is 16.7 mM glucose. **P<0.01, ***P<0.001. Statistical significance measured by one-way ANOVA. Data are presented as mean+/−SEM.

FIG. 6D shows GSIS from WT mouse islets treated with each of the proposed complex components alone (N=4/condition). The x-axis shows the conditions and insulin measured in (ng/mL/ug DNA) is shown on the y-axis. Data are presented as mean+/−SEM.

FIG. 6E shows GSIS from INS1 cells treated with NDPK-ADK with FABP4, lipid binding mutant (LBM) FABP4, or FABP4 pre-treated with inhibitor BMS-309403 (N=4/group). The treatment conditions are shown on the x-axis and insulin measured in (ng/mL/ug DNA) on the y-axis. *P<0.05, ***P<0.001. Statistical significance measured by one-way ANOVA. Data are presented as mean+/−SEM.

FIG. 6F shows a proposed model for the activity of extracellular nucleoside kinases on beta cell purinergic P2Y receptors.

FIG. 6G shows GSIS from WT mouse islets treated with high ratio (ATP degrading) or low ratio (ATP and ADP degrading) apyrase (N=3/group). The x-axis measures the concentration of apyrase and treatment conditions and insulin measured in (ng/mL/ug DNA) on the y-axis. *P<0.05, ***P<0.001. Statistical significance measured by one-way ANOVA. Data are presented as mean+/−SEM.

FIG. 6H shows GSIS in INS1 cells in response to the addition of increasing concentrations of ATP or ADP (N=4/group). The x-axis shows the concentration of nucleoside substrate measured in (04) and insulin measured in (ng/mL/ug DNA) is shown on the y-axis. *P<0.05. Statistical significance measured by Student's T-test. Data are presented as mean+/−SEM.

FIG. 6I shows GSIS in INS1 cells treated with NDPK-ADK or FABP4-ADK-NDPK in the presence of 5 μM exogenous ATP or non-metabolizable ATPyS (N=7/group). The x-axis shows the treatment conditions and insulin measured in (ng/mL/ug DNA) on the y-axis. *P<0.05, ***P<0.001. Statistical significance measured by one-way ANOVA. Data are presented as mean+/−SEM.

FIG. 6J shows GSIS in INS1 cells treated with NDPK-ADK or FABP4-ADK-NDPK in the presence of 5 μM exogenous ADP (N=3/group). The x-axis shows the treatment conditions and insulin measured in (ng/mL/ug DNA) on the y-axis. **P<0.01. Statistical significance measured by one-way ANOVA. Data are presented as mean+/−SEM.

FIG. 6K shows GSIS from INS1 cells in response to addition of ADP in the presence or absence of P2Y1 antagonist MRS2179 (N=4/group). The x-axis shows the concentration of ADP measured in (04) and insulin measured in (ng/mL/ug DNA) is shown on the y-axis. ***P<0.001. Statistical significance measured by one-way ANOVA. Data are presented as mean+/−SEM.

FIG. 6L shows GSIS from INS1 cells treated with NDPK-ADK or FABP4-ADK-NDPK in the presence of MRS2179 (N=3/group). The treatment groups are shown on the x-axis and insulin measured in (ng/mL/ug DNA) is shown on the y-axis. *P<0.05, **P<0.01. Statistical significance measured by one-way ANOVA. Data are presented as mean+/−SEM.

FIG. 6M shows kinase activity of WT NDPK or kinase dead H118N NDPK mutant to produce ADP (N=4/group). The substrate measured in mM is shown on the x-axis and ADP production measured in RLU is shown on the y-axis. **P<0.01. Statistical significance measured by one-way ANOVA. Data are presented as mean+/−SEM.

FIG. 6N shows GSIS from INS1 cells treated with WT NDPK or H118N NDPK in combination with ADK and FABP4 (N=4/group). The treatment conditions are shown on the x-axis and insulin measured in Fold difference HG/LG is shown on the y-axis. *P<0.05, **P<0.01, ***P<0.001. Statistical significance measured by one-way ANOVA. Data are presented as mean+/−SEM.

FIG. 6O shows kinase activity of ADK to generate ATP in the presence of ADK inhibitor (N=4/group). Substrate concentration is measured in (mM) and is shown on the x-axis and ATP production measured in relative luminescence units (RLU) is shown on the y-axis. **P<0.01, ***P<0.001. Statistical significance measured by one-way ANOVA. Data are presented as mean+/−SEM.

FIG. 6P shows GSIS from INS1 cells treated with NDPK-ADK or FABP4-ADK-NDPK in the presence of ADK inhibitor (N=3). The treatment groups are shown on the x-axis and insulin measured in (ng/mL/ug DNA) is shown on the y-axis. **P<0.01, ***P<0.001. Statistical significance measured by one-way ANOVA. Data are presented as mean+/−SEM.

FIG. 7A shows a Western blot (left panel) and quantification for pPKA substrate phosphorylation in human islets (N=6) treated with NDPK-ADK or FABP4-ADK-NDPK (right panel). ***P<0.001. Statistical significance measured by one-way ANOVA. Data are presented as mean+/−SEM.

FIG. 7B shows a Western blot (top panel) and quantification for pIP3R in INS1 cells treated with FABP4-ADK-NDPK (N=4). **P<0.01. Statistical significance measured by one-way ANOVA. Data are presented as mean+/−SEM.

FIG. 7C shows cytosolic calcium flux in INS1 cells from the extracellular space in response to 20 mM glucose as determined by Fura-2 AM staining (N=2-3 coverslips/condition; 50-100 cells) in control conditions or pretreatment with NDPK-ADK, FABP4-ADK-NDPK, and FABP4-ADK-NDPK-a-Ab. The x-axis is the number of frames and the y-axis is Fura-2 AM staining measured at excitations at 340 nm and 380 nm.

FIG. 7D shows the quantification of cytosolic calcium flux in INS1 cells from the extracellular space in response to 20 mM glucose as determined by Fura-2 AM staining (N=2-3 coverslips/condition; 50-100 cells) in control conditions or pretreatment with NDPK-ADK, FABP4-ADK-NDPK, and FABP4-ADK-NDPK-a-Ab. The x-axis is the treatment conditions and the y-axis is Fura-2 AM staining measured at excitations at 340 nm and 380 nm. ***P<0.001. Statistical significance measured by one-way ANOVA. Data are presented as mean+/−SEM.

FIG. 7E shows cytosolic calcium flux in INS1 cells from the endoplasmic reticulum in response to 1 μM thapsigargin as determined by Fura-2 AM staining (N=2-3 coverslips/condition; 50-100 cells) in control conditions or pretreatment with NDPK-ADK, FABP4-ADK-NDPK, and FABP4-ADK-NDPK-a-Ab. The x-axis is the number of frames and the y-axis is Fura-2 AM staining measured at excitations at 340 nm and 380 nm.

FIG. 7F shows the quantification of cytosolic calcium flux in INS1 cells from the endoplasmic reticulum in response to 1 μM thapsigargin as determined by Fura-2 AM staining (N=2-3 coverslips/condition; 50-100 cells) in control conditions or pretreatment with NDPK-ADK, FABP4-ADK-NDPK, and FABP4-ADK-NDPK-a-Ab. The x-axis is the treatment conditions and the y-axis is Fura-2 AM staining measured at excitations at 340 nm and 380 nm. ***P<0.001. Statistical significance measured by one-way ANOVA. Data are presented as mean+/−SEM.

FIG. 7G shows cytosolic calcium flux in INS1 cells from the extracellular space in response to 20 mM glucose followed by 30 mM KCl as determined by Fura-2 AM staining (N=2-3 coverslips/condition; 50-100 cells) in control conditions or pretreatment with FABP4-ADK-NDPK with or without MRS2365. The x-axis is the number of frames and the y-axis is Fura-2 AM staining measured at excitations at 340 nm and 380 nm.

FIG. 7H shows the quantification of cytosolic calcium flux in INS1 cells from the extracellular space in response to 20 mM glucose followed by 30 mM KCl as determined by Fura-2 AM staining (N=2-3 coverslips/condition; 50-100 cells) in control conditions or pretreatment with FABP4-ADK-NDPK with or without MRS2365. The x-axis is the treatment conditions and the y-axis is Fura-2 AM staining measured at excitations at 340 nm and 380 nm. ***P<0.001. Statistical significance measured by one-way ANOVA. Data are presented as mean+/−SEM.

FIG. 7I shows cytosolic calcium flux in INS1 cells from the endoplasmic reticulum in response to 1 μM thapsigargin as determined by Fura-2 AM staining (N=2-3 coverslips/condition; 50-100 cells) in control conditions or pretreatment with FABP4-ADK-NDPK with or without MRS2365. The x-axis is the number of frames and the y-axis is Fura-2 AM staining measured at excitations at 340 nm and 380 nm.

FIG. 7J shows the quantification of cytosolic calcium flux in INS1 cells from the endoplasmic reticulum in response to 1 μM thapsigargin as determined by Fura-2 AM staining (N=2-3 coverslips/condition; 50-100 cells) in control conditions or pretreatment with FABP4-ADK-NDPK with or without MRS2365. The x-axis is the treatment conditions and the y-axis is Fura-2 AM staining measured at excitations at 340 nm and 380 nm. **P<0.01, ***P<0.001. Statistical significance measured by one-way ANOVA. Data are presented as mean+/−SEM.

FIG. 7K shows cytosolic calcium flux in INS1 cells from the endoplasmic reticulum in response to 1 μM thapsigargin followed by 1.5 mM extracellular calcium as determined by Fura-2 AM staining (N=3-4 coverslips/treatment; 75-150 cells) under control conditions or pretreatment with FABP4-ADK-NDPK with or without the adenyl cyclase inhibitor NKY80. The x-axis is the number of frames and the y-axis is Fura-2 AM staining measured at excitations at 340 nm and 380 nm.

FIG. 7L shows a Western blot for cleaved caspase 3, pJNK, Chop, and B-Tubulin from INS1 cells treated with FABP4-ADK-NDPK for 24 hrs. (N=6).

FIG. 7M shows the quantification of Western blots from FIG. 7L for cleaved caspase 3 (CC3), pJNK, and Chop from INS1 cells treated with FABP4-ADK-NDPK for 24 hrs. (N=6). The x-axis is the proteins and the y-axis is the proteins measured in absorbance units (a.u.).

FIG. 7N shows gene expression of ER stress marker CHOP following 2 hr treatment with NDPK-ADK, FABP4-ADK-NDPK or FABP4-ADK-NDPK with a-Ab in the presence or absence of thapsigargin (Tg) (N=3). The x-axis shows the treatment conditions and the y-axis measures CHOP mRNA levels. **P<0.01, ***P<0.001. Statistical significance measured by one-way ANOVA. Data are presented as mean+/−SEM.

FIG. 7O shows gene expression of ER stress marker BIP following 2 hr treatment with NDPK-ADK, FABP4-ADK-NDPK or FABP4-ADK-NDPK with a-Ab in the presence or absence of thapsigargin (Tg) (N=3). The x-axis shows the treatment conditions and the y-axis measures BIP mRNA levels. **P<0.01, ***P<0.001. Statistical significance measured by one-way ANOVA. Data are presented as mean+/−SEM.

FIG. 7P and FIG. 7Q show cleaved caspase 3/7 activity in INS1 cells treated with increasing concentrations of cytokine cocktail TNFα, IFNγ, and IL-1B with or without FABP4-ADK-NDPK (N=4/group). *P<0.05. Statistical significance measured by Student's t-test. Data are presented as mean+/−SEM.

FIG. 8A shows the percentage of CD45+ cells in whole pancreas from mice treated with PBS, a-Ab, or c-Ab for 14 weeks (N=5-6 mice/group). The treatment conditions are shown on the x-axis and CD45+% is shown on the y-axis. Statistical significance measured by one-way ANOVA. Data are presented as mean+/−SEM.

FIG. 8B shows the percentage of regulatory T-cells in whole pancreas from mice treated with PBS, a-Ab, or c-Ab for 14 weeks (N=5-6 mice/group). The treatment conditions are shown on the x-axis and CD45+CD4+Foxp3+% is shown on the y-axis. Statistical significance measured by one-way ANOVA. Data are presented as mean+/−SEM.

FIG. 8C shows the percentage of cytotoxic T-cells in whole pancreas from mice treated with PBS, a-Ab, or c-Ab for 14 weeks (N=5-6 mice/group). The treatment conditions are shown on the x-axis and CD45+CD8+% is shown on the y-axis. Statistical significance measured by one-way ANOVA. Data are presented as mean+/−SEM.

FIG. 8D shows the percentage of T-helper cells in whole pancreas from mice treated with PBS, a-Ab, or c-Ab for 14 weeks (N=5-6 mice/group). The treatment conditions are shown on the x-axis and CD45+CD4+% is shown on the y-axis. Statistical significance measured by one-way ANOVA. Data are presented as mean+/−SEM.

FIG. 8E shows the percentage of B cells in whole pancreas from mice treated with PBS, a-Ab, or c-Ab for 14 weeks (N=5-6 mice/group). The treatment conditions are shown on the x-axis and CD45+CD3-CD19+% is shown on the y-axis. Statistical significance measured by one-way ANOVA. Data are presented as mean+/−SEM.

FIG. 8F shows the percentage of dendritic cells in whole pancreas from mice treated with PBS, a-Ab, or c-Ab for 14 weeks (N=5-6 mice/group). The treatment conditions are shown on the x-axis and CD45+CD11b-CD11c+% is shown on the y-axis. Statistical significance measured by one-way ANOVA. Data are presented as mean+/−SEM.

FIG. 8G shows the percentage of granulocytes cells in whole pancreas from mice treated with PBS, a-Ab, or c-Ab for 14 weeks (N=5-6 mice/group). The treatment conditions are shown on the x-axis and CD45+CD11b+Ly6C/6G+% is shown on the y-axis. Statistical significance measured by one-way ANOVA. Data are presented as mean+/−SEM.

FIG. 8H shows immunofluorescent staining for insulin, BrdU and nuclei of pancreatic sections from NOD mice treated with BrdU and a-Ab or c-Ab for 5 weeks (N=5 mice/group; 23-49 islets).

FIG. 8I shows the quantification of immunofluorescent staining for insulin, BrdU and nuclei of pancreatic sections from NOD mice treated with BrdU and a-Ab or c-Ab for 5 weeks (N=5 mice/group; 23-49 islets). The x-axis shows the treatment conditions and the y-axis shows the % BrdU+/INS+ cells. Statistical significance measured by Student's t-test. Data are presented as mean+/−SEM.

FIG. 8J shows immunofluorescent staining for insulin, BrdU and nuclei of pancreatic sections from NOD mice treated with PBS or a-Ab for 3 weeks (N=5 mice/group; 39-45 islets).

FIG. 8K shows the quantification of immunofluorescent staining for insulin, BrdU and nuclei of pancreatic sections from NOD mice treated with PBS or a-Ab for 3 weeks (N=5 mice/group; 39-45 islets). The x-axis shows the treatment conditions and the y-axis shows the % Ki67+/INS+ cells. Statistical significance measured by Student's t-test. Data are presented as mean+/−SEM.

FIG. 8L shows immunofluorescent staining for insulin, cleaved caspase 3 and nuclei of pancreatic sections from NOD mice treated with c-Ab or a-Ab for 3 weeks (N=8-10 islets/mouse from 5 mice/group).

FIG. 8M shows the quantification of immunofluorescent staining for insulin, cleaved caspase 3 and nuclei of pancreatic sections from NOD mice treated with c-Ab or a-Ab for 3 weeks (N=8-10 islets/mouse from 5 mice/group). Statistical significance measured by Student's t-test. Data are presented as mean+/−SEM.

FIG. 8N shows immunofluorescent staining for insulin, cleaved caspase 3 and nuclei of pancreatic sections from DIO mice treated with PBS or a-Ab for 3 weeks (N=25-34 islets from 5 mice/group).

FIG. 8O shows the quantification of immunofluorescent staining for insulin, cleaved caspase 3 and nuclei of pancreatic sections from DIO mice treated with PBS or a-Ab for 3 weeks (N=25-34 islets from 5 mice/group). Statistical significance measured by Student's t-test. Data are presented as mean+/−SEM.

### DETAILED DESCRIPTION

The present invention is based on the discovery that FABP4 is capable of modulating the ability of NDPK-ADK complex to modulate purinergic GPCRs through the formation of an NDPK-ADK/FABP4 complex which inhibits or reduces GPCR modulation. Under normal physiological conditions, NDPK-ADK complex is capable of modulating the activity of purinergic GPCRs on, for example, pancreatic islet β-cells, inducing the production and secretion of insulin in response to increased glucose levels. In the presence of excess FABP4, however, FABP4 binds to the NDPK-ADK complex, inhibiting or regulating its modulation of GPCRs, resulting in decreased glucose-stimulated insulin secretion (GSIS). In some embodiments, altering the ability of FABP4 from forming a complex with the NDPK-ADK complex, GSIS in pancreatic islet (3-cells can be restored, and thus, for example, elevated blood glucose levels reduced. Such a discovery provides new methods of addressing FABP4-mediated disorders, including for example chronic, elevated blood glucose levels in subjects, for example humans, and new methods for identifying compounds useful in treating disorders associated with FABP4-mediated disorders such as chronic, elevated blood glucose levels, including obesity and diabetes, for example, type I and type II diabetes.

Based on this discovery, methods, compounds, and methods for identifying compounds capable of interfering with the ability of NDPK-ADK/FABP4 complex formation are provided. By identifying and administering compounds capable of interfering with the formation of an NDPK-ADK/FABP4 complex, insulin production and secretion in pancreatic islet β-cells can be restored in response to elevated glucose blood levels. In some embodiments, the compound is an antibody, antibody-binding agent, or fragment. In some embodiments, the compound binds FABP4, NDPK, ADK, and/or the NDPK-ADK complex, inhibiting the formation of the NDPK-ADK/FABP4 complex, but not interfering with the ability of the NDPK-ADK complex from modulating or agonizing the activity of GPCRs. In some embodiments, the compound preferentially binds the NDPK-ADK/FABP4 complex over FABP4, the NDPK-ADK complex, NDPK, and/or ADK. In some embodiments, the compound preferentially binds to FABP4 over the NDPK-ADK complex, NDPK, and/or ADK.

When administered to a host in need thereof, the compound neutralizes the ability of FABP4 from modulating the activity of GPCRs by the NDPK-ADK complex, resulting in, for example, the restoration of GSIS, which would result in a decrease in blood glucose levels in vivo. Accordingly, conditions mediated by excess FABP4 including but not limited to chronic hyperglycemia can be treated by administering to a subject a compound described herein. Therefore, by targeting the interaction of FABP4 with NDPK-ADK complex, FABP4-mediated disorders, including metabolic disorders associated with increased blood glucose levels including, but not limited to, diabetes (both type 1 and type 2), hyperglycemia, diabetic ketoacidosis, hyperglycemic hyperosmolar syndrome, cardiovascular disease, diabetic nephropathy or kidney failure, diabetic retinopathy, impaired fasting glucose, impaired glucose tolerance, dyslipidemia, obesity, cataracts, stroke, impaired wound healing, perioperative hyperglycemia, hyperglycemia in the intensive care unit patient and insulin resistance syndrome can be treated. In certain embodiments, when administered to a subject in need thereof, the compound is useful to reduce fat mass, liver steatosis, improve serum lipid profiles, and/or reduce atherogenic plaque formation or maintenance in a subject. In certain embodiments, when administered to a subject in need thereof, the compound is useful to treat indications where ADK and NDPK have been previously implicated, such as cancer, atherosclerosis, and asthma. Therefore, in summary, the methods described herein are particularly useful to treat metabolic disorders associated with abnormal or excessive blood glucose levels, including, but not limited to, diabetes (both type 1 and type 2), hyperglycemia, obesity, fatty liver disease, and/or dyslipidemia. The compounds described herein are also useful in indications where ADK and NDPK have been previously implicated, such as atherosclerosis and asthma.

The present invention thus provides at least the following:
(a) Methods and compounds which modulate/affect, and preferably neutralize, the interaction of FABP4 on NDPK-ADK complex modulation of GPCRs for use in a therapy described herein.
(b) A method for identifying compounds which modulate/affect, and preferably neutralize, the activity of FABP4 on NDPK-ADK complex modulation of GPCRs for use in a therapy described herein.
(c) A method of modulating or restoring GPCR activity in the presence of FABP4 by administering a compound, for example an antibody, antigen-binding agent, or antibody-binding fragment, as described herein, or a described variant or conjugate thereof, that inhibits NDPK-ADK/FABP4 complex formation or sequesters the NDPK-ADK/FABP4 complex allowing NDPK-ADK complex modulation of GPCRs.
(d) A method of treating a subject, and in particular a human, with a disease or disorder associated with excess FABP4, for example dysregulated insulin secretion and elevated blood glucose levels by administering to the subject a compound, for example an antibody, antigen-binding agent or antibody-binding fragment, or a described variant or conjugate thereof, that inhibits NDPK-ADK/FABP4 complex formation or sequesters the NDPK-ADK/FABP4 complex away from the GPCRs allowing NDPK-ADK complex modulation of GPCRs on the surface of target cells.

### General Definitions

Unless otherwise defined, all technical and scientific terms used herein have the same meaning as commonly understood by one of ordinary skill in the art to which this invention belongs. Although methods and materials similar or equivalent to those described herein can be used in the practice or testing, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques are generally performed according to conventional methods well known in the art and as described in various general and more specific references that are cited and discussed throughout the present specification unless otherwise indicated. Enzymatic reactions and purification techniques may be performed according to manufacturer's specifications, as commonly accomplished in the art or as described herein. The nomenclatures used in connection with, and the laboratory procedures and techniques of, analytical chemistry, synthetic organic chemistry, and medicinal, and pharmaceutical chemistry described herein are those well-known and commonly used in the art. Standard techniques are used for chemical syntheses, chemical analyses, pharmaceutical preparation, formulation, and delivery, and treatment of patients.

That the present invention may be more readily understood, selected terms are defined below.

The term “host,” “subject,” or “patient” as used herein, typically refers to a human subject. In some embodiments, the host, subject, or patient is a human. Wherein a host to be treated is a human, and a compound administered is an antibody, in general a human or humanized antibody framework may be used as an acceptor structure. Where another host is treated, it is understood by those of skill in the art that an antibody or antigen binding agent may need to be tailored to that host to avoid rejection or to make more compatible. It is known how to use a CDR and engineer them into the proper framework or peptide sequence for desired delivery and function for a range of hosts. Other hosts may include other mammals or vertebrate species. The term “host,” therefore, can alternatively refer to animals such as mice, monkeys, dogs, pigs, rabbits, domesticated swine (pigs and hogs), ruminants, equine, poultry, felines, murines, bovines, canines, and the like, where the antibody or antigen binding agent, if necessary is suitably designed for compatibility with the host.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments, and polypeptide analogs of a protein sequence. A polypeptide may be monomeric or polymeric.

The term “human FABP4 protein” as used herein refers to the protein encoded by SEQ ID NO: 4 (UniProtKB—P15090 (FABP4 HUMAN)), and natural variants thereof, as described by Baxa, C. A., Sha, R. S., Buelt, M. K., Smith, A. J., Matarese, V., Chinander, L. L., Boundy, K. L., Bernlohr, A. Human adipocyte lipid-binding protein: purification of the protein and cloning of its complementary DNA. Biochemistry 28: 8683-8690, 1989.

The term “mouse FABP4 protein” as used herein, refers to the protein encoded by SEQ ID NO: 5 (UniProtKB—P04117 (FABP4_MOUSE)), and natural variants thereof.

“Antigen binding agents” as used herein include single chain antibodies (i.e. a full length heavy chain and light chain); Fab, modified Fab, Fab′, modified Fab′, F(ab′)2, Fv, Fab-Fv, Fab-dsFv, single domain antibodies (e.g. VH or VL or VHH) for example as described in WO 2001090190, scFv, bi, tri or tetra-valent antibodies, Bis-scFv, diabodies, tribodies, triabodies, tetrabodies and epitope-antigen binding agents of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). The Fab-Fv format was first disclosed in WO2009/040562 and the disulphide stabilised versions thereof, the Fab-dsFv was first disclosed in WO2010/035012. Other antibody fragments for use in the present invention include the Fab and Fab′ fragments described in International patent applications WO2005/003169, WO2005/003170, and WO2005/003171. Multi-valent antibodies may comprise multiple specificities e.g. bispecific or may be monospecific (see for example WO 92/22583 and WO05/113605). One such example of the latter is a Tri-Fab (or TFM) as described in WO92/22583.

A typical Fab′ molecule comprises a heavy and a light chain pair in which the heavy chain comprises a variable region VH, a constant domain CH1 and a natural or modified hinge region and the light chain comprises a variable region VL and a constant domain CL.

A dimer of a Fab′ to create a F(ab′)2 for example dimerization may be through a natural hinge sequence described herein, or derivative thereof, or a synthetic hinge sequence.

The terms “specific binding” or “specifically binding”, as used herein, in reference to the interaction of an antibody, a protein, or a peptide with a second chemical species, means that the interaction is dependent upon the presence of a particular structure (e.g., an “antigenic determinant” or “epitope” as defined below) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains at least some portion of the epitope binding features of an Ig molecule allowing it to, for example, specifically bind to FABP4. Such mutant, variant, or derivative antibody formats are known in the art and described below. Nonlimiting embodiments of which are discussed below. An antibody is said to be “capable of binding” a molecule if it is capable of specifically reacting with the molecule to thereby bind the molecule to the antibody.

A “monoclonal antibody” as used herein is intended to refer to a preparation of antibody molecules, which share a common heavy chain and common light chain amino acid sequence, or any functional fragment, mutant, variant, or derivation thereof which retains at least the light chain epitope binding features of an Ig molecule, in contrast with “polyclonal” antibody preparations that contain a mixture of different antibodies. Monoclonal antibodies can be generated by several known technologies like phage, bacteria, yeast or ribosomal display, as well as classical methods exemplified by hybridoma-derived antibodies (e.g., an antibody secreted by a hybridoma prepared by hybridoma technology, such as the standard Kohler and Milstein hybridoma methodology ((1975) Nature 256:495-497).

In a full-length antibody, each heavy chain is comprised of a heavy chain variable region (abbreviated herein as HCVR or VH) and a heavy chain constant region (CH). The heavy chain constant region is comprised of four domains—either CH1, Hinge, CH2, and CH3 (heavy chains γ, α and δ), or CH1, CH2, CH3, and CH4 (heavy chains μ and ε). Each light chain is comprised of a light chain variable region (abbreviated herein as LCVR or VL) and a light chain constant region (CL). The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “antibody construct” as used herein refers to a polypeptide comprising one or more of the antigen binding portions linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain, for example a human IgA, IgD, IgE, IgG or IgM constant domains. Heavy chain and light chain constant domain amino acid sequences are known in the art.

Still further, an antibody or antigen-binding portion thereof may be part of a larger immuno-adhesion molecule, formed by covalent or noncovalent association of the antibody or antibody portion with one or more other proteins or peptides. Examples of such immuno-adhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immuno-adhesion molecules can be obtained using standard recombinant DNA techniques, as described herein.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having human heavy and light chain variable regions in which one or more of the human CDRs (e.g., CDR3) has been replaced with murine CDR sequences.

The terms “Kabat numbering”, “Kabat definitions” and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31-35 (CDR-H1), residues 50-65 (CDR-H2) and residues 95-102 (CDR-H3) according to the Kabat numbering system. However, according to Chothia (Chothia et al., (1987) J. Mol. Biol., 196, 901-917 (1987)), the loop equivalent to CDR-H1 extends from residue 26 to residue 32. Thus, unless indicated otherwise “CDR-H1” as employed herein is intended to refer to residues 26 to 35, as described by a combination of the Kabat numbering system and Chothia's topological loop definition. For the light chain variable region, the hypervariable region ranges from amino acid positions 24 to 34 for CDRL1, amino acid positions 50 to 56 for CDRL2, and amino acid positions 89 to 97 for CDRL3.

As used herein, the terms “acceptor” and “acceptor antibody” refer to the antibody or nucleic acid sequence providing or encoding at least 80%, at least 85%, at least 90%, at least 95%, at least 98% or 100% of the amino acid sequences of one or more of the framework regions. In some embodiments, the term “acceptor” refers to the antibody amino acid or nucleic acid sequence providing or encoding the constant region(s). In yet another embodiment, the term “acceptor” refers to the antibody amino acid or nucleic acid sequence providing or encoding one or more of the framework regions and the constant region(s). In a specific embodiment, the term “acceptor” refers to a human antibody amino acid or nucleic acid sequence that provides or encodes at least 80%, preferably, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the term “CDR” refers to the complementarity determining region within antibody variable sequences. There are three CDRs in each of the variable regions of the heavy chain and the light chain, which are designated CDRH1, CDRH2 and CDRH3 for the heavy chain CDRs, and CDRL1, CDRL2, and CDRL3 for the light chain CDRs. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia, or a mixture thereof, defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone conformations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In a preferred embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

Human heavy chain and light chain acceptor sequences are known in the art. See, e.g., WO 2016/176656, incorporated herein by reference.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. See, e.g., Shapiro et al., Crit. Rev. Immunol. 22(3): 183-200 (2002); Marchalonis et al., Adv Exp Med Biol. 484:13-30 (2001). One of the advantages provided by various embodiments takes advantage of the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or 0-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

The term “humanized antibody” generally refers to antibodies which comprise heavy and light chain variable region sequences from a non-human species (e.g., a rabbit, mouse, etc.) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. Another type of humanized antibody is a CDR-grafted antibody, in which at least one non-human CDR is inserted into a human framework.

In particular, the term “humanized antibody” as used herein, is an antibody or a variant, derivative, analog or fragment thereof which immuno-specifically binds to an antigen of interest and which comprises a framework (FR) region having substantially the amino acid sequence of a human antibody and a complementarity determining region (CDR) having substantially the amino acid sequence of a non-human antibody. As used herein, the term “substantially” in the context of a CDR refers to a CDR having an amino acid sequence at least 50, 55, 60, 65, 70, 75 or 80%, preferably at least 85%, at least 90%, at least 95%, at least 98% or at least 99% identical to the amino acid sequence of a non-human antibody CDR. In some embodiments, the humanized antibody has a CDR region having one or more (for example 1, 2, 3 or 4) amino acid substitutions, additions and/or deletions in comparison to the non-human antibody CDR. Further, the non-human CDR can be engineered to be more “human-like” or compatible with the human body, using known techniques. A humanized antibody comprises substantially all of at least one, and typically two, variable domains (Fab, Fab′, F(ab′)2, F(ab′) c, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, and CH3, or CH1, CH2, CH3, and CH4 of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgY, IgM, IgG, IgD, IgA and IgE, and any isotype, including without limitation IgA1, IgA2, IgG1, IgG2, IgG3 and IgG4. The humanized antibody may comprise sequences from more than one class or isotype, and particular constant domains may be selected to optimize desired effector functions using techniques well known in the art. See, e.g., WO 2016/176656, incorporated herein by reference.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond exactly to either the donor antibody or the consensus framework. In a preferred embodiment, such mutations, however, will not be extensive. Usually, at least 50, 55, 60, 65, 70, 75 or 80%, preferably at least 85%, more preferably at least 90%, and most preferably at least 95%, 98% or 99% of the humanized antibody residues will correspond to those of the parental FR and CDR sequences. In some embodiments, one or more (for example 1, 2, 3 or 4) amino acid substitutions, additions and/or deletions may be present in the humanized antibody compared to the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

As used herein, “Vernier” zone refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and may impact on the structure of CDRs and the affinity of the antibody.

As used herein, the term “neutralizing” refers to neutralization of biological activity of FABP4 or the ability of FABP4 to modulate NDPK-ADK complex modulation of GPCRs or the ability of NDPK-ADK/FABP4 complex to antagonize a GPCR. Preferably a neutralizing binding protein, for example an antibody, is a binding protein who's binding to FABP4, NDPK, NDPK-ADK complex, and/or NDPK-ADK/FABP4 protein complex results in neutralization of the biological activity of FABP4 on the ability to inhibit NDPK-ADK complex modulation of GPCRs. Preferably the neutralizing binding protein decreases the ability of FABP4 to inhibit NDPK-ADK complex modulation by at least about 5%, 10%, 15%, 20%, 25%, 30%, 35%, 40%, 50%, 60%, 80%, 85%, or more. Neutralization of the biological activity of FABP4 on NDPK-ADK complex modulation of GPCRs can be assessed by measuring one or more indicators of NDPK-ADK complex biological activity described herein.

A “neutralizing monoclonal antibody” as used herein is intended to refer to a preparation of antibody molecules, which upon binding to FABP4, NDPK, ADK, or NDPK-ADK/FABP4 protein complex are able to inhibit or reduce the ability of FABP4 to inhibit or modulate NDPK-ADK complex modulation of GPCRs.

The term “blood glucose level” shall mean blood glucose concentration. In certain embodiments, a blood glucose level is a plasma glucose level. Plasma glucose may be determined in accordance with, e.g., Etgen et al., (2000) Metabolism, 49(5): 684-688 or calculated from a conversion of whole blood glucose concentration in accordance with D'Orazio et al., (2006) Clin. Chem. Lab. Med., 44(12): 1486-1490.

The term “normal glucose levels” refers to mean plasma glucose values in humans of less than about 100 mg/dL for fasting levels, and less than 145 mg/dL for 2-hour postprandial levels or 125 mg/dL for a random glucose. The term “elevated blood glucose level” or “elevated levels of blood glucose” shall mean an elevated blood glucose level such as that found in a subject demonstrating clinically inappropriate basal and postprandial hyperglycemia or such as that found in a subject in oral glucose tolerance test (oGTT), with “elevated levels of blood glucose” being greater than 100 mg/dL when tested under fasting conditions, and greater than about 200 mg/dL when tested at 1 hour.

As used herein, the term “attenuation,” “attenuate,” and the like refers to the lessening or reduction in the severity of a symptom or condition.

The term “epitope” or “antigenic determinant” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The term “K_{d}”, as used herein, is intended to refer to the Affinity (or Affinity constant), which is a measure of the rate of binding (association and dissociation) between the antibody and antigen, determining the intrinsic binding strength of the antibody binding reaction.

The terms “crystal”, and “crystallized” as used herein, refer to an antibody, or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 20 1-16, Oxford University Press, New York, N.Y., (1999).

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g. prophylactic or therapeutic agent).

### Nucleoside Diphosphate Kinase (NDPK)

Nucleotide diphosphate kinase (NDPK) is a ubiquitous enzyme that catalyzes the transfer of the γ-phosphate from nucleoside 5′-triphosphates (NTP) to nucleoside 5′-diphosphate (NDP) by a ping-pong mechanism involving the formation of a high energy phosphate intermediate on His118 (Morera et al. (1995) Biochemistry 34:11062-11070 and Tepper et al. (1994) J. Biol. Chem. 269:32175-32180). Eight isoforms of NDPK encoded by the nm23 genes in humans have been identified (Lacombe et al. (2000) J. Bionerg. Biomembr. 32:247-258). In mammalian tissues, the cytosolic enzyme forms heterohexamers of 17-21 kDa subunits (Gilles et al. (1991) J. Biol. Chem. 266:8784-8789). These are composed of different combinations of the three major isoforms, NDPK A, B, and C. In addition to NTP synthesis, NDPKs are involved in a variety of processes in cellular physiology, including tumor metastasis (Steeg et al. (1988) J. Natl. Cancer Inst. 80:200-204), development (Rosengard et al. (1989) Nature 342:177-180), gene regulation (Postel (2003) J. Bioenerg. Biomembranes 35:31-40), apoptosis (Fan et al. (2003) Cell 112:659-672), endocytosis (Krishnan et al. (2001) Neuron 30:197-210 and Palacios et al. (2002) Nat. Cell Biol. 4:929-936), vesicular transport from the endoplasmic reticulum (Kapetanovich et al. (2005) Mol. Biol. Cell 16:835-848), and regulation of the cystic fibrosis transmembrane conductance regulator (Crawford et al. (2006) Mol. Cell. Biol. 26:5921-5931). It has also been shown that NDPK can act as a direct activator of G protein signaling through protein histidine phosphorylation (Kimura et al. J. Bioenerg. Biomembranes (2003) 35:41-47, Wieland T. Naunyn Schmiedebergs Arch Pharmacol. (2007); 374:373-383, and Kowluru et al. Naunyn Schmiedebergs Arch Pharmacol. (October, 2011) 384:383-390).

As described herein for the first time, NDPK forms a complex with ADK capable of modulating GPCRs on, for example pancreatic islet β-cells, wherein such modulation is capable of being inhibited by FABP4's binding to the NDPK-ADK complex. The amino acid sequence of human NDPK-A and NDPK-B is described below.

TABLE 1
NDPK Amino Acid Sequence.

| | SEQ ID | |
|---|---|---|
| Protein | NO: | Sequence |
| Nucleoside | 1 | MANCERTFIAIKPDGVQRGLVGEIIKRFEQKGFRLVG |
| diphosphate kinase A | | LKFMQASEDLLKEHYVDLKDRPFFAGLVKYMHSGPVV |
| ((NDPK A) [H. | | AMVWEGLNVVKTGRVMLGETNPADSKPGTIRGDFCIQ |
| sapiens: UniProtKB - | | VGRNIIHGSDSVESAEKEIGLWFHPEELVDYTSCAQN |
| P15531 | | WIYE |
| (NDKA_HUMAN)] | | |
| Nucleoside | 2 | MANLERTFIAIKPDGVQRGLVGEIIKRFEQKGFRLVA |
| diphosphate kinase B | | MKFLRASEEHLKQHYIDLKDRPFFPGLVKYMNSGPVV |
| (NDPK B) [H. | | AMVWEGLNVVKTGRVMLGETNPADSKPGTIRGDFCIQ |
| sapiens: UniProtKB | | VGRNIIHGSDSVKSAEKEISLWFKPEELVDYKSCAHD |
| P22392 | | WVYE |
| (NDKB_HUMAN)] | | |

Anti-NDPK antibodies have been described in the art, and include: Li, Xue-ling; et al., Chinese journal of cellular and molecular immunology, 2004, 20(1), 86-8, incorporated herein by reference; Xi Bao Yu Fen Zi Mian Yi Xue Za Zhi. 2004, 20(1), 86-8, incorporated herein by reference; Aryee, D N; et al., International journal of cancer, 1995, 64(2), 104-11, incorporated herein by reference; Kraeft, Stine-Kathrein; et al., “Nuclear localization of nucleoside diphosphate kinase type B (nm23-H2) in cultured cells”, Experimental Cell Research, 1996, 227(1), 63-69, incorporated by reference; Yi X B, et al., “Neutralizing antibodies to nucleoside diphosphate kinase inhibit the enzyme in vitro and in vivo: evidence for two distinct mechanisms of activation of atrial currents by ATPgammaS”, Biochim Biophys Acta., 1996, 1310(3), 334-42, incorporated herein by reference; US 2002/0197253 incorporated herein by reference; US2016/0326263A1 incorporated herein by reference; US2017/0204196A1 incorporated herein by reference; US2018/0258186 incorporated herein by reference; US2019/0031778 incorporated herein by reference; US2019/0031779 incorporated herein by reference; US 2020/0078319 incorporated herein by reference; JP08333399.

### Adenosine Kinase (ADK)

Adenosine kinase (ADK) is an enzyme that catalyzes the transfer of gamma-phosphate from Adenosine triphosphate (ATP) to adenosine (Ado) leading to formation of adenosine monophosphate (AMP) and adenosine diphosphate (ADP). This enzymatic reaction plays a fundamental role as a metabolic regulator of energy homeostasis (Fredholm et al. Pharmacol. Rev. (2011a) 63:1-34). Adenosine thus controls important physiologic functions, such as blood supply, glucose homeostasis via interactions with both insulin and glucagon, and lipolysis (Hjemdahl and Fredholm Acta Physiol Scand. (1976) 96:170-179). Under conditions of stress or distress adenosine levels rapidly rise, largely by breakdown of adenine nucleotides (Fredholm et al. Cell Death Differ. (2007) 14:1315-1323). Under conditions of stress adenosine exerts a multitude of protective functions on many different levels (Fredholm et al. Cell Death Differ. (2007) 14:1315-1323). ADK dysfunction is involved in several pathologies, including diabetes, epilepsy, and cancer.

The amino acid sequence of ADK is provided below.

TABLE 2
ADK Amino Acid Sequence

| | SEQ ID | |
|---|---|---|
| Protein | NO: | Sequence |
| Adenosine Kinase | 3 | MAAAEEEPKPKKLKVEAPQALRENILFGMGNPLLDISAVV |
| (ADK) [H. sapiens: | | DKDFLDKYSLKPNDQILAEDKHKELFDELVKKFKVEYHAG |
| UniProtKB - P55263 | | GSTQNSIKVAQWMIQQPHKAATFFGCIGIDKFGEILKRKA |
| (ADKHUMAN)] | | AEAHVDAHYYEQNEQPTGTCAACITGDNRSLIANLAAANC |
| | | YKKEKHLDLEKNWMLVEKARVCYIAGFFLTVSPESVLKVA |
| | | HHASENNRIFTLNLSAPFISQFYKESLMKVMPYVDILFGN |
| | | ETEAATFAREQGFETKDIKEIAKKTQALPKMNSKRQRIVI |
| | | FTQGRDDTIMATESEVTAFAVLDQDQKEIIDTNGAGDAFV |
| | | GGFLSQLVSDKPLTECIRAGHYAASIIIRRTGCTFPEKPD |
| | | FH |

### Fatty Acid Binding Protein 4 (FABP4)

Human fatty-acid binding protein 4 (FABP4), also known as adipocyte lipid binding protein (aP2), belongs to a family of intra-cellular lipid-binding proteins involved in the transport and storage of lipids (Banzszak et al., (1994) Adv. Protein Chem. 45, 89-151). The FABP4 protein is involved in lipolysis and lipogenesis and has been indicated in diseases of lipid and energy metabolism such as diabetes, atherosclerosis, and metabolic syndromes. FABP4 has also been indicated in the integration of metabolic and inflammatory response systems. (Ozcan et al., (2006) Science 313(5790):1137-40; Makowski et al., (2005) J Biol Chem. 280 (13):12888-95; and Erbay et al., (2009) Nat Med. 15(12):1383-91). More recently, FABP4 has been shown to be differentially expressed in certain soft tissue tumors such as certain liposarcomas (Kashima et al., (2013) Virchows Arch. 462, 465-472).

FABP4 is highly expressed in adipocytes and regulated by peroxisome-proliferator-activated receptor-gamma (PPAR-gamma) agonists, insulin, and fatty acids (Hertzel et al., (2000) Trends Endocrinol. Metab. 11, 175-180; Hunt et al., (1986) PNAS USA 83, 3786-3790; Melki et al., (1993) J. Lipid Res. 34, 1527-1534; Distel et al., (1992) J. Biol. Chem. 267, 5937-5941). Studies in FABP4 deficient mice (FABP4^{-/-}) indicate protection against the development of insulin resistance associated with genetic or diet-induced obesity and improved lipid profile in adipose tissue with increased levels of C16:1n7-palmitoleate, reduced hepatosteatosis, and improved control of hepatic glucose production and peripheral glucose disposal (Hotamisligil et al., (1996) Science 274, 1377-1379; Uysal et al., (2000) Endocrinol. 141, 3388-3396; Cao et al., (2008) Cell 134, 933-944).

In addition, genetic deficiency or pharmacological blockade of FABP4 reduces both early and advanced atherosclerotic lesions in an apolipoprotein E-deficient (ApoE^{-/-}) mouse model (Furuhashi et al., (2007) Nature, June 21; 447 (7147):959-65; Makowski et al., (2001) Nature Med. 7, 699-705; Layne et al., (2001) FASEB 15, 2733-2735; Boord et al., (2002) Arteriosclerosis, Thrombosis, and Vas. Bio. 22, 1686-1691). Furthermore, FABP4-deficiency leads to a marked protection against early and advanced atherosclerosis in apolipoprotein E-deficient (ApoE^{-/-}) mice (Makowski et al., (2001) Nature Med. 7, 699-705; Fu et al., (2000) J. Lipid Res. 41, 2017-2023). Hence, FABP4 plays a critical role in many aspects of development of metabolic disease in preclinical models.

In the past two decades, the biological functions of FABPs in general and FABP4 in particular have primarily been attributed to their action as intracellular proteins. Since the abundance of FABP4 protein in the adipocytes is extremely high, accounting for up to a few percent of the total cellular protein (Cao et al., (2013) Cell Metab. 17 (5):768-78), therapeutically targeting FABP4 with traditional approaches has been challenging, and the promising success obtained in preclinical models (Furuhashi et al., (2007) Nature 447, 959-965; Won et al., (2014) Nature Mat. 13, 1157-1164; Cai et al., (2013) Acta Pharm. Sinica 34, 1397-1402; Hoo et al., (2013) J. of Hepat. 58, 358-364) has been slow to progress toward clinical translation.

In addition to its presence in the cytoplasm, it has recently been shown that FABP4 is actively secreted from adipose tissue through a non-classical regulated pathway (Cao et al., (2013) Cell Metab. 17(5), 768-778; Ertunc et al., (2015) J. Lipid Res. 56, 423-424). The secreted form of FABP4 acts as a novel adipokine and regulates hepatic glucose production and systemic glucose homeostasis in mice in response to fasting and fasting-related signals. Serum FABP4 levels are significantly elevated in obese mice and blocking circulating FABP4 improves glucose homeostasis in mice with diet-induced obesity (Cao et al., (2013) Cell Metab. 17(5):768-78). Importantly, the same patterns are also observed in human populations where secreted FABP4 levels are increased in obesity and strongly correlate with metabolic and cardiovascular diseases in multiple independent human studies (Xu et al., (2006) Clin. Chem. 53, 405-413; Yoo et al., (2011) J. Clin. Endocrin. & Metab. 96, 488-492; von Eynatten et al., (2012) Arteriosclerosis, Thrombosis, and Vas. Bio. 32, 2327-2335; Suh et al., (2014) Scandinavian J. Gastro. 49, 979-985; Furuhashi et al., (2009) Metabolism: Clinical and Experimental 58, 1002-1007; Kaess et al., (2012) J. Endocrin. & Metab. 97, E1943-47; Cabre et al., (2007) Atherosclerosis 195, e150-158). Finally, humans carrying a haploinsufficiency allele which results in reduced FABP4 expression are protected against diabetes and cardiovascular disease (Tuncman et al., (2006) PNAS USA 103, 6970-6975; Saksi et al., (2014) Circulation, Cardiovascular Genetics 7, 588-598). US20120134998, titled Secreted aP2 and Methods of Inhibiting Same, to President and Fellows of Harvard University and US20160319003, titled Anti-AP2 Antibodies and Antigen Binding Agents to Treat Metabolic Disorders (both incorporated herein by reference in their entirety), to President and Fellows of Harvard University, describe the use of antibodies targeting circulating FABP4 in order to modulate metabolic disorders.

Fatty acid-binding proteins (FABPs) are members of the superfamily of lipid-binding proteins (LBP). Nine different FABPs have to date been identified, each showing relative tissue enrichment: L (liver), I (intestinal), H (muscle and heart), A (adipocyte), E (epidermal), Il (ileal), B (brain), M (myelin) and T (testis). The primary role of all the FABP family members is regulation of fatty acid uptake and intracellular transport. The structures of all FABPs are similar the basic motif characterizing these proteins is B-barrel, and a fatty acid ligand or ligands (e.g., a fatty acid, cholesterol, or retinoid) bound in its internal water-filled cavity.

US20160319003 to President and Fellows of Harvard College and titled “Anti-AP2 Antibodies and Antigen Binding Agents to Treat Metabolic Disorders” describes monoclonal antibodies directed to FABP4 for use in treating disorders such as diabetes, obesity, cardiovascular disease, fatty liver disease, and/or cancer, among others. The described antibodies generally have a K_{d} greater than about 2.9 μM. US20160319003, which discloses and describes the CA33 (a-Ab) monoclonal antibody and derivatives thereof, is incorporated herein by reference in its entirety.

The human FABP4 protein is a 14.7 kDa intracellular and extracellular (secreted) lipid binding protein that consists of 132 amino acids comprising the amino acid sequence SEQ ID NO: 4 of Table 3. The cDNA sequence of human FABP4 was previously described in Baxa, C. A., Sha, R. S., Buelt, M. K., Smith, A. J., Matarese, V., Chinander, L. L., Boundy, K. L., Bernlohr, A. Human adipocyte lipid-binding protein: purification of the protein and cloning of its complementary DNA. Biochemistry 28: 8683-8690, 1989, and is provided in SEQ ID NO: 7. The human protein is registered in Swiss-Prot under the number P15090.

TABLE 3
FABP4 Protein and cDNA Sequences.

| | SEQ ID | |
|---|---|---|
| Protein or cDNA | NO: | SEQUENCE |
| Fatty acid-binding protein, | 4 | MCDAFVGTWKLVSSENFDDYMKEVGVGF |
| adipocyte | | ATRKVAGMAKPNMIISVNGDVITIKSES |
| (FABP4/FABP4) [H. | | TFKNTEISFILGQEFDEVTADDRKVKST |
| sapiens] UniProtKB - | | ITLDGGVLVHVQKWDGKSTTIKRKREDD |
| P15090 | | KLVVECVMKGVTSTRVYERA |
| (FABP4_HUMAN) | | |
| Fatty acid-binding protein, | 5 | MCDAFVGTWKLVSSENFDDYMKEVGVGF |
| adipocyte | | ATRKVAGMAKPNMIISVNGDLVTIRSES |
| (FABP4/FABP4 [M. | | FTKNTEISFKLGVEFDEITADDRKVKSI |
| musculus]) | | ITLDGGALVQVQKWDGKSTTIKRKRDGD |
| | | KLVVECVMKGVTSTRVYERA |
| FABP4 nuclear | 6 | KEVGVGFATRK |
| localization amino acid | | |
| sequence | | |
| FABP4 fatty acid binding | | RVY |
| domain amino acid | | |
| sequence | | |
| Fatty acid-binding protein, | 7 | ATGTGTGATGCTTTTGTAGGTACCTGGA |
| adipocyte | | AACTTGTCTCCAGTGAAAACTTTGATGA |
| (FABP4/FABP4) [H. | | TTATATGAAAGAAGTAGGAGTGGGCTTT |
| sapiens] cDNA | | GCCACCAGGAAAGTGGCTGGCATGGCCA |
| | | AACCTAACATGATCATCAGTGTGAATGG |
| | | GGATGTGATCACCATTAAATCTGAAAGT |
| | | ACCTTTAAAAATACTGAGATTTCCTTCA |
| | | TACTGGGCCAGGAATTTGACGAAGTCAC |
| | | TGCAGATGACAGGAAAGTCAAGAGCACC |
| | | ATAACCTTAGATGGGGGTGTCCTGGTAC |
| | | ATGTGCAGAAATGGGATGGAAAATCAAC |
| | | CACCATAAAGAGAAAACGAGAGGATGAT |
| | | AAACTGGTGGTGGAATGCGTCATGAAAG |
| | | GCGTCACTTCCACGAGAGTTTATGAGAG |
| | | AGCATAA |
| Fatty acid-binding protein, | 8 | ATGTGTGATGCCTTTGTGGGAACCTGGA |
| adipocyte | | AGCTTGTCTCCAGTGAAAACTTCGATGA |
| (FABP4/FABP4 [M. | | TTACATGAAAGAAGTGGGAGTGGGCTTT |
| musculus]) cDNA | | GCCACAAGGAAAGTGGCAGGCATGGCCA |
| | | AGCCCAACATGATCATCAGCGTAAATGG |
| | | GGATTTGGTCACCATCCGGTCAGAGAGT |
| | | ACTTTTAAAAACACCGAGATTTCCTTCA |
| | | AACTGGGCGTGGAATTCGATGAAATCAC |
| | | CGCAGACGACAGGAAGGTGAAGAGCATC |
| | | ATAACCCTAGATGGCGGGGCCCTGGTGC |
| | | AGGTGCAGAAGTGGGATGGAAAGTCGAC |
| | | CACAATAAAGAGAAAACGAGATGGTGAC |
| | | AAGCTGGTGGTGGAATGTGTTATGAAAG |
| | | GCGTGACTTCCACAAGAGTTTATGAAAG |
| | | GGCATGA |

### Purinergic P2Y Receptors

P2Y receptors are G protein-coupled receptors (GPCRs) that are activated by adenine and uridine nucleotides and nucleotide sugars. There are eight subtypes of P2Y receptors (P2Y1, P2Y2, P2Y4, P2Y6, P2Y11, P2Y12, P2Y13, and P2Y14), which activate intracellular signaling cascades to regulate a variety of cellular processes, including proliferation, differentiation, phagocytosis, secretion, nociception, cell adhesion, and cell migration. These signaling cascades operate mainly by the sequential activation or deactivation of heterotrimeric and monomeric G proteins, phospholipases, adenylyl and guanylyl cyclases, protein kinases, and phosphodiesterases. In addition, there are numerous ion channels, cell adhesion molecules, and receptor tyrosine kinases that are modulated by P2Y receptors and operate to transmit an extracellular signal to an intracellular response. P2Y receptors are G-protein-coupled receptors (GPCRs) for extracellular nucleotides. P2Y receptors belong to the superfamily of heptahelical receptors found exclusively in eukaryotes that operate by binding or sensing signals outside the cell and then activating intracellular processes by coupling to heterotrimeric G proteins.

The human P2Y1 receptor is a widely expressed 373 amino acid purinergic G protein coupled receptor for extracellular adenine nucleotides such as ADP and ATP. The amino acid sequence is provided by UnitProtKB P47900 (P2RY1 HUMAN).

Compounds that Attenuate NDPK-ADK/FABP4 Complex Activity

In one aspect of the invention, methods for modulating NDPK-ADK/FABP4 complex activity are provided which include administering to a subject an effective amount of a compound that neutralizes the effect of FABP4 on NDPK-ADK modulation of GPCRs by either inhibiting the formation of the NDPK-ADK/FABP4 complex or inhibiting the NDPK-ADK/FABP4 complex from inhibiting GPCR modulation by the NDPK-ADK complex and allowing unbound NDPK-ADK complex to modulate GPCRs. Compounds useful in the methods described herein include those that directly target the NDPK-ADK/FABP4 complex or inhibit FABP4 binding to the NDPK-ADK complex, effectively neutralizing FABP4's ability to regulate the modulation of GPCRs by the NDPK-ADK complex. In some embodiments, the compound is an anti-NDPK, anti-ADK antibody, anti-FABP4 antibody, or anti-NDPK-ADK complex antibody or antigen binding agent that inhibits FABP4 binding to NDPK-ADK complex but does not inhibit the ability of NDPK-ADK complex to modulate GPCRs. In some embodiments, the compound is an anti-NDPK-ADK/FABP4 complex antibody or antigen binding agent that sequesters the NDPK-ADK/FABP4 complex, allowing the NDPK-ADK complex to modulate GPCRs. In some embodiments, the compound is a monoclonal antibody, antibody fragment, or antigen binding agent. In some embodiments, the compound is a humanized monoclonal antibody or antigen binding agent. In some embodiments, the antibody, antibody fragment, or antigen binding agent preferentially binds to NDPK-ADK/FABP4 complex over FABP4, NDPK, ADK, or NDPK-ADK in complex.

In some embodiments, the monoclonal antibody or antigen binding agent prevents the formation of an NDPK-ADK/FABP4 complex, wherein the antibody preferentially binds FABP4 over NDPK and ADK, either separately or when NDPK-ADK are in a complex, wherein the antibody is capable of increasing the ratio of extracellular ADP to ATP or capable of inducing glucose-stimulated insulin section in a human pancreatic islet β-cell or insulin secreting cell line assay in the presence of glucose, FABP4, NDPK, and ADK. In some embodiments, the monoclonal antibody or antigen binding agent binds to FABP4 at a K_{d} of less than 10⁻⁷. In some embodiments, the monoclonal antibody or antigen binding agent binds to FABP4 at a K_{d} of less than about 10⁻⁸. In some embodiments, the monoclonal antibody or antigen binding agent binds to FABP4 at a K_{d} of between 10⁻⁹ and 10¹². In some embodiments, the antibody binds to FABP4 at a K_{d} of less than about 100 nM, less than about 50 nM, less than about 10 nM, less than about 1 nM, or less than about 0.5 nM. In some embodiments, the monoclonal antibody or antigen binding agent is administered in an effective amount to a subject with an FABP4-mediated disorder. In some embodiments, the subject has dysregulated insulin secretion and elevated blood glucose levels, and administration of the antibody results in an increase of GSIS compared to when the subject is not administered the antibody. In some embodiments, the subject's blood glucose level is greater than about 100 mg/dL when challenged with an oral glucose tolerance test (oGTT) at fasting conditions prior to administration of the monoclonal antibody or antigen binding agent.

In some embodiments, the monoclonal antibody or antigen binding agent inhibits FABP4's ability to downregulate or inhibit NDPK-ADK complex activity by binding to nucleoside diphosphate kinase (NDPK), wherein the antibody binds to NDPK at a K_{d} of less than about 10⁻⁸. In some embodiments, the monoclonal antibody or antigen binding agent binds to NDPK at a K_{d} of between 10⁻⁹ and 10⁻¹². In some embodiments, the monoclonal antibody or antigen binding agent binds to NDPK at a K_{d} of less than 100 nM, and wherein the antibody is capable of increasing the extracellular ADP to ATP ratio or capable of inducing glucose-stimulated insulin secretion in a human pancreatic islet β-cell or insulin secreting cell line assay in the presence of glucose, adipocyte binding protein 2 (FABP4), NDPK, and adenosine kinase (ADK). In some embodiments, the antibody or antigen binding agent binds to NDPK at a K_{d} of less than about 5 nM. In some embodiments, the antibody binds to NDPK at a K_{d} of less than about 50 nM, less than about 10 nM, less than about 5 nM, less than about 3 nM, less than about 2 nM, less than about 1 nM, or less than about 0.5 nM or less. In some embodiments, the antibody or antigen binding agent binds to NDPK-A isoform. In some embodiments, the antibody or antigen binding agent binds to NDPK-B isoform. In some embodiments, the antibody or antigen binding agent does not bind to FABP4 alone or uncomplexed. In some embodiments, the monoclonal antibody or antigen binding agent is administered in an effective amount to a subject with an FABP4-mediated disorder. In some embodiments, the monoclonal antibody or antigen binding agent is administered in an effective amount to a subject with dysregulated insulin secretion and elevated blood glucose levels and results in an increase of glucose-stimulated insulin secretion (GSIS) compared to when the subject is not administered the antibody. In some embodiments, the subject's blood glucose level is greater than about 100 mg/dL when challenged with an oral glucose tolerance test (oGTT) at fasting conditions prior to administration of the monoclonal antibody or antigen binding agent.

In some embodiments, the monoclonal antibody or antigen binding agent binds to NDPK, wherein the antibody when bound to NDPK inhibits the ability of FABP4 to modulate NDPK activity, wherein the antibody does not inhibit the ability of NDPK to complex with ADK and modulate glucose-stimulated insulin secretion from an insulin secreting cell. In some embodiments, the monoclonal antibody or antigen binding agent binds to NDPK in complex with ADK. In some embodiments, the monoclonal antibody or antigen binding agent binds to NDPK or NDPK in complex with ADK at a K_{d} of less than about 100 nM. In some embodiments, the monoclonal antibody or antigen binding agent binds to NDPK or NDPK in complex with ADK at a K_{d} of less than about 5 nM. In some embodiments, the antibody or antigen binding agent binds to NDPK at a K_{d} of less than about 5 nM. In some embodiments, the antibody binds to NDPK or NDPK in complex with ADK at a K_{d} of less than about 50 nM, less than about 10 nM, less than about 5 nM, less than about 3 nM, less than about 2 nM, less than about 1 nM, or less than about 0.5 nM or less. In some embodiments, the antibody or antigen binding agent binds to NDPK-A isoform. In some embodiments, the antibody or antigen binding agent binds to NDPK-B isoform. In some embodiments, the monoclonal antibody or antigen binding agent is capable of increasing the extracellular ADP to ATP ratio or is capable of inducing GSIS in a human pancreatic islet β-cell or insulin secreting cell line assay in the presence of glucose, FABP4, NDPK, and ADK. In some embodiments, the monoclonal antibody or antigen binding agent is administered in an effective amount to a subject with an FABP4-mediated disorder. In some embodiments, the monoclonal antibody or antigen binding agent is administered in an effective amount to a subject with dysregulated insulin secretion and elevated blood glucose levels and results in an increase of glucose-stimulated insulin secretion (GSIS) compared to when the subject is not administered the antibody. In some embodiments, the subject's blood glucose level is greater than about 100 mg/dL when challenged with an oral glucose tolerance test (oGTT) at fasting conditions prior to administration of the monoclonal antibody or antigen binding agent.

In some embodiments, the monoclonal antibody or antigen binding agent prevents the formation of an NDPK-ADK/FABP4 complex, wherein the antibody binds to a NDPK-ADK complex, wherein the antibody does not bind to FABP4 alone or uncomplexed. In some embodiments, the monoclonal antibody or antigen binding agent binds to the NDPK-ADK complex at a K_{d} of less than about 500 nM. In some embodiments, the antibody binds to the NDPK-ADK complex at a K_{d} of less than about 500 nM, less than about 400 nM, less than about 300 nM, less than about 200 nM, less than about 100 nM, less than about 50 nM, less than about 10 nM, less than about 5 nM, less than about 3 nM, less than about 2 nM, less than about 1 nM, or less than about 0.5 nM or less. In some embodiments, the monoclonal antibody or antigen binding agent is capable of increasing the extracellular ADP to ATP ratio or is capable of inducing GSIS in a human pancreatic islet β-cell or insulin secreting cell line assay in the presence of glucose, FABP4, NDPK, and ADK. In some embodiments, the monoclonal antibody or antigen binding agent is administered in an effective amount to a subject with an FABPR4-mediated disorder. In some embodiments, the monoclonal antibody or antigen binding agent is administered in an effective amount to a subject with dysregulated insulin secretion and elevated blood glucose levels. and results in an increase of GSIS compared to when the subject is not administered the antibody. In some embodiments, the subject's blood glucose level is greater than about 100 mg/dL when challenged with an oral glucose tolerance test (oGTT) at fasting conditions prior to administration of the monoclonal antibody or antigen binding agent.

In some embodiments, the monoclonal antibody or antigen binding agent affects the modulation of a GPCR by an NDPK-ADK/FABP4 complex on a target cell, wherein the antibody binds to the NDPK-ADK/FABP4 complex, wherein the antibody does not bind to FABP4 alone or uncomplexed, and wherein the antibody is capable of increasing the ratio of extracellular ADP to ATP or capable of inducing GSIS in a human pancreatic islet β-cell or insulin secreting cell line assay in the presence of glucose, FABP4, NDPK, and ADK. In some embodiments, the monoclonal antibody or antigen binding agent binds to NDPK-ADK/FABP4 complex at a K_{d} of less than about 500 nM. In some embodiments, the monoclonal antibody or antigen binding agent binds to FABP4-NDPK-ADK complex at a K_{d} of less than about 5 nM. In some embodiments, the antibody binds to FABP4-NDPK-ADK complex at a K_{d} of less than about 500 nM, less than about 400 nM, less than about 300 nM, less than about 200 nM, less than about 100 nM, less than about 50 nM, less than about 10 nM, less than about 5 nM, less than about 3 nM, less than about 2 nM, less than about 1 nM, or less than about 0.5 nM or less. In some embodiments, the monoclonal antibody or antigen binding agent is capable of increasing the extracellular ADP to ATP ratio or is capable of inducing GSIS in a human pancreatic islet β-cell or insulin secreting cell line assay in the presence of glucose, FABP4, NDPK, and ADK. In some embodiments, the monoclonal antibody or antigen binding agent is administered in an effective amount to a subject with an FABPR4-mediated disorder. In some embodiments, the monoclonal antibody or antigen binding agent is administered in an effective amount to a subject with dysregulated insulin secretion and elevated blood glucose levels. and results in an increase of GSIS compared to when the subject is not administered the antibody. In some embodiments, the subject's blood glucose level is greater than about 100 mg/dL when challenged with an oral glucose tolerance test (oGTT) at fasting conditions prior to administration of the monoclonal antibody or antigen binding agent.

In some embodiments, the monoclonal antibody or antigen binding agent affects the modulation of a GPCR by an NDPK-ADK/FABP4 complex on a target cell, wherein the antibody binds to the NDPK-ADK/FABP4 complex, wherein the antibody preferentially binds the NDPK-ADK/FABP4 complex over FABP4, NDPK, or ADK alone or uncomplexed, and wherein the antibody is capable of increasing the ratio of extracellular ADP to ATP or capable of inducing GSIS in a human pancreatic islet β-cell or insulin secreting cell line assay in the presence of glucose, FABP4, NDPK, and ADK. In some embodiments, the monoclonal antibody or antigen binding agent binds to FABP4-NDPK-ADK complex at a K_{d} of less than about 500 nM. In some embodiments, the monoclonal antibody or antigen binding agent binds to FABP4-NDPK-ADK complex at a K_{d} of less than about 5 nM. In some embodiments, the antibody binds to FABP4-NDPK-ADK complex at a K_{d} of less than about 500 nM, less than about 400 nM, less than about 300 nM, less than about 200 nM, less than about 100 nM, less than about 50 nM, less than about 10 nM, less than about 5 nM, less than about 3 nM, less than about 2 nM, less than about 1 nM, or less than about 0.5 nM or less. In some embodiments, the monoclonal antibody or antigen binding agent is administered in an effective amount to a subject with an FABP4-mediated disorder. In some embodiments, the monoclonal antibody or antigen binding agent is administered in an effective amount to a subject with dysregulated insulin secretion and elevated blood glucose levels. and results in an increase of GSIS compared to when the subject is not administered the antibody. In some embodiments, the subject's blood glucose level is greater than about 100 mg/dL when challenged with an oral glucose tolerance test (oGTT) at fasting conditions prior to administration of the monoclonal antibody or antigen binding agent.

In some embodiments, the monoclonal antibody or antigen binding agent inhibits the modulation of a GPCR by an NDPK-ADK/FABP4 complex on a target cell, wherein the antibody binds to FABP4-NDPK in complex, wherein the antibody preferentially binds the FABP4-NDPK complex over FABP4 and NDPK alone or uncomplexed, and wherein the antibody is capable of increasing the ratio of extracellular ADP to ATP or capable of inducing GSIS in a human pancreatic islet β-cell or insulin secreting cell line assay in the presence of glucose, FABP4, NDPK, and ADK. In some embodiments, the monoclonal antibody or antigen binding agent binds to the FABP4-NDPK complex at a K_{d} of less than 500 nM. In some embodiments, the monoclonal antibody or antigen binding agent binds to the FABP4-NDPK complex at a K_{d} of less than 5 nM. In some embodiments, the antibody binds to the FABP4-NDPK in complex at a K_{d} of less than about 500 nM, less than about 400 nM, less than about 300 nM, less than about 200 nM, less than about 100 nM, less than about 50 nM, less than about 10 nM, less than about 5 nM, less than about 3 nM, less than about 2 nM, less than about 1 nM, or less than about 0.5 nM or less. In some embodiments, the monoclonal antibody or antigen binding agent is administered in an effective amount to a subject with an FABP4-mediated disorder. In some embodiments, the monoclonal antibody or antigen binding agent is administered in an effective amount to a subject with dysregulated insulin secretion and elevated blood glucose levels. and results in an increase of GSIS compared to when the subject is not administered the antibody. In some embodiments, the subject's blood glucose level is greater than about 100 mg/dL when challenged with an oral glucose tolerance test (oGTT) at fasting conditions prior to administration of the monoclonal antibody or antigen binding agent.

In some embodiments, the monoclonal antibody or antigen binding agent binds to an NDPK-ADK complex, wherein the antibody when bound to NDPK-ADK in complex inhibits the ability of FABP4 to bind to the NDPK-ADK complex, wherein the antibody does not inhibit the ability of NDPK-ADK to modulate glucose-stimulated insulin secretion from a targeted cell, wherein the antibody does not bind to FABP4 alone or uncomplexed, and wherein the antibody is capable of increasing the ratio of extracellular ADP to ATP or inducing GSIS in a human pancreatic islet β-cell or insulin secreting cell line assay in the presence of glucose, FABP4, NDPK, and ADK. In some embodiments, the monoclonal antibody or antigen binding agent binds to the NDPK-ADK complex at a K_{d} of less than 500 nM. In some embodiments, the monoclonal antibody or antigen binding agent binds to the NDPK-ADK complex at a K_{d} of less than 5 nM. In some embodiments, the antibody binds to the FABP4-NDPK in complex at a K_{d} of less than about 500 nM, less than about 400 nM, less than about 300 nM, less than about 200 nM, less than about 100 nM, less than about 50 nM, less than about 10 nM, less than about 5 nM, less than about 3 nM, less than about 2 nM, less than about 1 nM, or less than about 0.5 nM or less. In some embodiments, the monoclonal antibody or antigen binding agent is administered in an effective amount to a subject with an FABP4-mediated disorder. In some embodiments, the monoclonal antibody or antigen binding agent is administered in an effective amount to a subject with dysregulated insulin secretion and elevated blood glucose levels. and results in an increase of GSIS compared to when the subject is not administered the antibody. In some embodiments, the subject's blood glucose level is greater than about 100 mg/dL when challenged with an oral glucose tolerance test (oGTT) at fasting conditions prior to administration of the monoclonal antibody or antigen binding agent.

Methods of producing antibodies, antibody fragments, or antigen binding agents are known in the art. See, e.g., US2011/0129464. For example, polyclonal antibodies are preferably raised in animals by multiple subcutaneous (SC) or intraperitoneal (IP) injections of the relevant antigen and an adjuvant, for example, FABP4, NDPK, ADK, NDPK-ADK complex, or NDPK-ADK in complex with FABP4 (NDPK-ADK/FABP4). It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC12, or R1N═C═NR, where R and R1 are different alkyl groups.

For example, animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen but conjugated to a different protein and/or through a different cross-linking reagent. Conjugates also can be made in recombinant cell culture as protein fusions. Also, aggregating agents such as alum are suitably used to enhance the immune response.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et. al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (MA) or enzyme-linked immunosorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be sub-cloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the sub-clones are suitably separated from the culture medium, ascites fluid, or serum by conventional antibody purification procedures such as, for example, protein A-Sepharose, hydroxyapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy chain and light chain constant domains in place of the homologous murine sequences (U.S. Pat. No. 4,816,567; and Morrison, et al., Proc. Natl. Acad. Sci. USA, 81:6851(1984)), or by covalently joining to the immunoglobulin coding sequence all or part of the coding sequence for a non-immunoglobulin polypeptide.

Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Methods for humanizing non-human antibodies have been described in the art, for example, methods to prepare human and humanized antibodies are provided in a number of publications, including U.S. Pat. Nos. 7,223,392, 6,090,382, 5,859,205, 6,090,382, 6,054,297, 6,881,557, 6,284,471, and 7,070,775. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding. Various forms of the humanized antibody or affinity matured antibody are contemplated. For example, the humanized antibody or affinity matured antibody may be an antibody fragment, such as a Fab, which is optionally conjugated with one or more cytotoxic agent(s) in order to generate an immunoconjugate. Alternatively, the humanized antibody or affinity matured antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immunol., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S. and Chiswell, David J., Current Opinion in Structural Biology 3:564-571(1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894; and 5,587,458. The antibody fragment may also be a “linear antibody”, e.g., as described in U.S. Pat. No. 5,641,870 for example. Such linear antibody fragments may be monospecific or bispecific.

Techniques for generating antibodies have been described above. One may further select antibodies with certain biological characteristics, as desired, for example, preferential binding to the NDPK-ADK/FABP4 complex over FABP4, NDPK, ADK, and NDPK-ADK complex.

To identify an antibody which inhibits FABP4 complexing to NDPK-ADK complex, the ability of the antibody to inhibit FABP4 binding to the NDPK-ADK complex may be determined. For example, cells endogenously expressing, or transfected to express, GPCRs may be incubated with the antibody and then exposed to labeled FABP4 and NDPK-ADK complex. The ability of antibody to block binding of FABP4 to NDPK-ADK complex or the ability of NDPK-ADK complex to modulate GPCRs may then be evaluated.

Alternatively, or additionally, the ability of the antibody or antigen binding agent to restore the ability of the NDPK-ADK complex to modulate GPCRs in the presence of FABP4 may be assessed. For example, cells endogenously expressing GPCRs or transfected to express GPCRs may be incubated with the antibody and then assayed for NDPK-ADK complex modulation activity. Modulation can be measured by activation or inhibition of adenylate cyclase and/or phospholipase C, by measuring an increase or decrease in cAMP, by measuring insulin secretion if using pancreatic islet β-cells or insulin-secreting cell lines, by measuring the phosphorylation of the endoplasmic reticulum (ER) calcium efflux transporter IP3R, wherein a decrease in phosphorylation of IP3R indicates a compound capable of inhibiting formation of NDPK-ADK/FABP4 complex, by measuring calcium efflux from the ER, wherein a decrease in calcium efflux from the ER indicates a compound capable of inhibiting formation of NDPK-ADK/FABP4 complex, by measuring extracellular calcium influx in response to the addition of glucose, wherein an increase in extracellular calcium influx in response to the addition of glucose indicates a compound capable of inhibiting formation of NDPK-ADK/FABP4 complex, by measuring pPKA substrate phosphorylation downstream of cAMP, wherein a decrease in pPKA substrate phosphorylation downstream of cAMP indicates a compound capable of inhibiting formation of NDPK-ADK/FABP4 complex. In some embodiments, the cell population is INS1 cells. In some embodiments, the cell population is human islet cells.

In one aspect, the antibodies and fragments for administration are humanized.

Construction of CDR-grafted antibodies is generally described in European Patent Application EP-A-0239400, which discloses a process in which the CDRs of a mouse monoclonal antibody are grafted onto the framework regions of the variable domains of a human immunoglobulin by site directed mutagenesis using long oligonucleotides and is incorporated herein. The CDRs determine the antigen binding specificity of antibodies and are relatively short peptide sequences carried on the framework regions of the variable domains.

The human variable heavy and light chain germline subfamily classification can be derived from the Kabat germline subgroup designations: VH1, VH2, VH3, VH4, VH5, VH6 or VH7 for a particular VH sequence and JH1, JH2, JH3, JH4, JH5, and JH6 for a for a particular variable heavy joining group for framework 4; VK1, VK2, VK3, VK4, VK5 or VK6 for a particular VL kappa sequence for framework 1, 2, and 3, and JK1, JK2, JK3, JK4, or JK5 for a particular kappa joining group for framework 4; or VL1, VL2, VL3, VL4, VL5, VL6, VL7, VL8, VL9, or VL10 for a particular VL lambda sequence for framework 1, 2, and 3, and JL1, JL2, JL3, or JL7 for a particular lambda joining group for framework 4.

The general framework of the light chain comprises the structures selected from FR1-CDRL1-FR2-CDRL2-FR3-CDRL3-FR4 and FR1-CDRL1-FR2-CDRL2-FR3-CDRL3-FR4-CL, and variations thereof, wherein the framework regions are selected from either an immunoglobulin kappa light chain variable framework region, or an immunoglobulin lambda light chain variable framework region, and an immunoglobulin light chain constant region from either a kappa light chain constant region when the framework region is a kappa light chain variable framework region, or a lambda light chain constant region when the framework region is a lambda light chain variable framework region.

In some embodiments, the general framework of the heavy chain regions contemplated herein comprises the structures selected from FR1-CDRH1-FR2-CDRH2-FR3-CDRH3-FR4, FR1-CDRH1-FR2-CDRH2-FR3-CDRH3-FR4-CH1, FR1-CDRH1-FR2-CDRH2-FR3-CDRH3-FR4-CH1-Hinge-CH2 for IgG, IgD, and IgA immunoglobulin classes and FR1-CDRH1-FR2-CDRH2-FR3-CDRH3-FR4-CH1-CH2 for IgM and IgE immunoglobulin classes, FR1-CDRH1-FR2-CDRH2-FR3-CDRH3-FR4-CH1-Hinge-CH2-CH3 for IgG, IgD, and IgA immunoglobulin classes, FR1-CDRH1-FR2-CDRH2-FR3-CDRH3-FR4-CH1-CH2-CH3 for IgM and IgE immunoglobulin classes, and FR1-CDRH1-FR2-CDRH2-FR3-CDRH3-FR4-CH1-CH2-CH3-CH4 for IgM and IgE immunoglobulin classes, and variations thereof, wherein the framework regions are selected from heavy chain variable framework regions, and the heavy chain constant regions. IgA and IgM classes can further comprise a joining polypeptide that serves to link two monomer units of IgM or IgA together, respectively. In the case of IgM, the J chain-joined dimer is a nucleating unit for the IgM pentamer, and in the case of IgA it induces larger polymers.

The constant region domains of the antibody molecule for administration, if present, may be selected having regard to the proposed function of the antibody molecule, and in particular the effector functions which may be required. For example, the constant region domains may be human IgA, IgD, IgE, IgG or IgM domains. In particular embodiments, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required.

The antibody fragment administered may include Fab, Fab′, F(ab′)2, scFv, diabody, scFAb, dFv, single domain light chain antibodies, dsFv, a peptide comprising CDR, and the like.

In some embodiments, the human acceptor light chain framework is derived from an amino acid sequence encoded by a human IGKV (VL kappa) gene for framework 1, 2, and 3, and an IGKJ gene for framework 4. In some embodiments, the human acceptor light chain framework is derived from an amino acid sequence encoded by a human IGLV (VL lambda) gene for framework 1, 2, and 3, and an IGLJ gene for framework 4. Non-limiting examples of human light chain IGKV and IGKJ acceptor framework regions are provided, for example, in Table 4, and non-limiting examples of human light chain IGLV and IGLJ acceptor framework regions are provide, for example, Table 5.

TABLE 4
Human IGKV and IGKJ Framework Regions

| Variable | | |
|---|---|---|
| Light κ | SEQ | |
| Chain FR | ID | |
| Region | NO: | Sequence |
| O12 FR1 | 9 | DIQMTQSPSSLSASVGDRVTITC |
| O12 FR2 | 10 | WYQQKPGKAPKLLIY |
| O12 FR3 | 11 | GVPSRFSGSGSGTDFTLTISSLQPEDFATYYC |
| O2 FR1 | 12 | DIQMTQSPSSLSASVGDRVTITC |
| O2 FR2 | 13 | WYQQKPGKAPKLLIY |
| O2 FR3 | 14 | GVPSRFSGSGSGTDFTLTISSLQPEDFATYYC |
| O18 FR1 | 15 | DIQMTQSPSSLSASVGDRVTITC |
| O18 FR2 | 16 | WYQQKPGKAPKLLIY |
| O18 FR3 | 17 | GVPSRFSGSGSGTDFTFTISSLQPEDIATYYC |
| O8 FR1 | 18 | DIQMTQSPSSLSASVGDRVTITC |
| O8 FR2 | 19 | WYQQKPGKAPKLLIY |
| O8 FR3 | 20 | GVPSRFSGSGSGTDFTFTISSLQPEDIATYYC |
| A20 FR1 | 21 | DIQMTQSPSSLSASVGDRVTITC |
| A20 FR2 | 22 | WYQQKPGKVPKLLIY |
| A20 FR3 | 23 | GVPSRFSGSGSGTDFTLTISSLQPEDVATYYC |
| A30 FR1 | 24 | DIQMTQSPSSLSASVGDRVTITC |
| A30 FR2 | 25 | WYQQKPGKAPKRLIY |
| A30 FR3 | 26 | GVPSRFSGSGSGTEFTLTISSLQPEDFATYYC |
| L14 FR1 | 27 | NIQMTQSPSAMSASVGDRVTITC |
| L14 FR2 | 28 | WFQQKPGKVPKHLIY |
| L14 FR3 | 29 | GVPSRFSGSGSGTEFTLTISSLQPEDFATYYC |
| L1 FR1 | 30 | DIQMTQSPSSLSASVGDRVTITC |
| L1 FR2 | 31 | WFQQKPGKAPKSLIY |
| L1 FR3 | 32 | GVPSRFSGSGSGTDFTLTISSLQPEDFATYYC |
| L15 FR1 | 33 | DIQMTQSPSSLSASVGDRVTITC |
| L15 FR2 | 34 | WYQQKPEKAPKSLIY |
| L15 FR3 | 35 | GVPSRFSGSGSGTDFTLTISSLQPEDFATYYC |
| L4 FR1 | 36 | AIQLTQSPSSLSASVGDRVTITC |
| L4 FR2 | 37 | WYQQKPGKAPKLLIY |
| L4 FR3 | 38 | GVPSRFSGSGSGTDFTLTISSLQPEDFATYYC |
| L18 FR1 | 39 | AIQLTQSPSSLSASVGDRVTITC |
| L18 FR2 | 40 | WYQQKPGKAPKLLIY |
| L18 FR3 | 41 | GVPSRFSGSGSGTDFTLTISSLQPEDFATYYC |
| L5 FR1 | 42 | DIQMTQSPSSVSASVGDRVTITC |
| L5 FR2 | 43 | WYQQKPGKAPKLLIY |
| L5 FR3 | 44 | GVPSRFSGSGSGTDFTLTISSLQPEDFATYYC |
| L19 FR1 | 45 | DIQMTQSPSSVSASVGDRVTITC |
| L19 FR2 | 46 | WYQQKPGKAPKLLIY |
| L19 FR3 | 47 | GVPSRFSGSGSGTDFTLTISSLQPEDFATYYC |
| L8 FR1 | 48 | DIQLTQSPSFLSASVGDRVTITC |
| L8 FR2 | 49 | WYQQKPGKAPKLLIY |
| L8 FR3 | 50 | GVPSRFSGSGSGTEFTLTISSLQPEDFATYYC |
| L23 FR1 | 51 | AIRMTQSPFSLSASVGDRVTITC |
| L23 FR2 | 52 | WYQQKPAKAPKLFIY |
| L23 FR3 | 53 | GVPSRFSGSGSGTDYTLTISSLQPEDFATYYC |
| L9 FR1 | 54 | AIRMTQSPSSFSASTGDRVTITC |
| L9 FR2 | 55 | WYQQKPGKAPKLLIY |
| L9 FR3 | 56 | GVPSRFSGSGSGTDFTLTISCLQSEDFATYYC |
| L24 FR1 | 57 | VIWMTQSPSLLSASTGDRVTISC |
| L24 FR2 | 58 | WYQQKPGKAPELLIY |
| L24 FR3 | 59 | GVPSRFSGSGSGTDFTLTISCLQSEDFATYYC |
| L11 FR1 | 60 | AIQMTQSPSSLSASVGDRVTITC |
| L11 FR2 | 61 | WYQQKPGKAPKLLIY |
| L11 FR3 | 62 | GVPSRFSGSGSGTDFTLTISSLQPEDFATYYC |
| L12 FR1 | 63 | DIQMTQSPSTLSASVGDRVTITC |
| L12 FR2 | 64 | WYQQKPGKAPKLLIY |
| L12 FR3 | 65 | GVPSRFSGSGSGTEFTLTISSLQPDDFATYYC |
| O11 FR1 | 66 | DIVMTQTPLSLPVTPGEPASISC |
| O11 FR2 | 67 | WYLQKPGQSPQLLIY |
| O11 FR3 | 68 | GVPDRFSGSGSGTDFTLKISRVEAEDVGVYYC |
| O1 FR1 | 69 | DIVMTQTPLSLPVTPGEPASISC |
| O1 FR2 | 70 | WYLQKPGQSPQLLIY |
| O1 FR3 | 71 | GVPDRFSGSGSGTDFTLKISRVEAEDVGVYYC |
| A17 FR1 | 72 | DVVMTQSPLSLPVTLGQPASISC |
| A17 FR2 | 73 | WFQQRPGQSPRRLIY |
| A17 FR3 | 74 | GVPDRFSGSGSGTDFTLKISRVEAEDVGVYYC |
| A1 FR1 | 75 | DVVMTQSPLSLPVTLGQPASISC |
| A1 FR2 | 76 | WFQQRPGQSPRRLIY |
| A1 FR3 | 77 | GVPDRFSGSGSGTDFTLKISRVEAEDVGVYYC |
| A18 FR1 | 78 | DIVMTQTPLSLSVTPGQPASISC |
| A18 FR2 | 79 | WYLQKPGQSPQLLIY |
| A18 FR3 | 80 | GVPDRFSGSGSGTDFTLKISRVEAEDVGVYYC |
| A2 FR1 | 81 | DIVMTQTPLSLSVTPGQPASISC |
| A2 FR2 | 82 | WYLQKPGQPPQLLIY |
| A2 FR3 | 83 | GVPDRFSGSGSGTDFTLKISRVEAEDVGVYYC |
| A19 FR1 | 84 | DIVMTQSPLSLPVTPGEPASISC |
| A19 FR2 | 85 | WYLQKPGQSPQLLIY |
| A19 FR3 | 86 | GVPDRFSGSGSGTDFTLKISRVEAEDVGVYYC |
| A3 FR1 | 87 | DIVMTQSPLSLPVTPGEPASISC |
| A3 FR2 | 88 | WYLQKPGQSPQLLIY |
| A3 FR3 | 89 | GVPDRFSGSGSGTDFTLKISRVEAEDVGVYYC |
| A23 FR1 | 90 | DIVMTQTPLSSPVTLGQPASISC |
| A23 FR2 | 91 | WLQQRPGQPPRLLIY |
| A23 FR3 | 92 | GVPDRFSGSGAGTDFTLKISRVEAEDVGVYYC |
| A27 FR1 | 93 | EIVLTQSPGTLSLSPGERATLSC |
| A27 FR2 | 94 | WYQQKPGQAPRLLIY |
| A27 FR3 | 95 | GIPDRFSGSGSGTDFTLTISRLEPEDFAVYYC |
| A11 FR1 | 96 | EIVLTQSPATLSLSPGERATLSC |
| A11 FR2 | 97 | WYQQKPGLAPRLLIY |
| A11 FR3 | 98 | GIPDRFSGSGSGTDFTLTISRLEPEDFAVYYC |
| L2 FR1 | 99 | EIVMTQSPATLSVSPGERATLSC |
| L2 FR2 | 100 | WYQQKPGQAPRLLIY |
| L2 FR3 | 101 | GIPARFSGSGSGTEFTLTISSLQSEDFAVYYC |
| L16 FR1 | 102 | EIVMTQSPATLSVSPGERATLSC |
| L16 FR2 | 103 | WYQQKPGQAPRLLIY |
| L16 FR3 | 104 | GIPARFSGSGSGTEFTLTISSLQSEDFAVYYC |
| L6 FR1 | 105 | EIVLTQSPATLSLSPGERATLSC |
| L6 FR2 | 106 | WYQQKPGQAPRLLIY |
| L6 FR3 | 107 | GIPARFSGSGSGTDFTLTISSLEPEDFAVYYC |
| L20 FR1 | 108 | EIVLTQSPATLSLSPGERATLSC |
| L20 FR2 | 109 | WYQQKPGQAPRLLIY |
| L20 FR3 | 110 | GIPARFSGSGPGTDFTLTISSLEPEDFAVYYC |
| L25 FR1 | 111 | EIVMTQSPATLSLSPGERATLSC |
| L25 FR2 | 112 | WYQQKPGQAPRLLIY |
| L25 FR3 | 113 | GIPARFSGSGSGTDFTLTISSLQPEDFAVYYC |
| B3 FR1 | 114 | DIVMTQSPDSLAVSLGERATINC |
| B3 FR2 | 115 | WYQQKPGQPPKLLIY |
| B3 FR3 | 116 | GVPDRFSGSGSGTDFTLTISSLQAEDVAVYYC |
| B2 FR1 | 117 | ETTLTQSPAFMSATPGDKVNISC |
| B2 FR2 | 118 | WYQQKPGEAAIFIIQ |
| B2 FR3 | 119 | GIPPRFSGSGYGTDFTLTINNIESEDAAYYFC |
| A26 FR1 | 120 | EIVLTQSPDFQSVTPKEKVTITC |
| A26 FR2 | 121 | WYQQKPDQSPKLLIK |
| A26 FR3 | 122 | GVPSRFSGSGSGTDFTLTINSLEAEDAATYYC |
| A10 FR1 | 123 | EIVLTQSPDFQSVTPKEKVTITC |
| A10 FR2 | 124 | WYQQKPDQSPKLLIK |
| A10 FR3 | 125 | GVPSRFSGSGSGTDFTLTINSLEAEDAATYYC |
| A14 FR1 | 126 | DVVMTQSPAFLSVTPGEKVTITC |
| A14 FR2 | 127 | WYQQKPDQAPKLLIK |
| A14 FR3 | 128 | GVPSRFSGSGSGTDFTFTISSLEAEDAATYYC |
| JK1 FR4 | 129 | FGQGTKVEIK |
| JK2 FR4 | 130 | FGQGTKLEIK |
| JK3 FR4 | 131 | FGPGTKVDIK |
| JK4 FR4 | 132 | FGGGTKVEIK |
| JK5 FR4 | 133 | FGQGTRLEIK |

TABLE 5
Human IGLV and IGLJ Framework Regions

| Variable | | |
|---|---|---|
| Light λ | SEQ | |
| Chain FR | ID | |
| Region | NO: | Sequence |
| 1a FR1 | 134 | QSVLTQPPSVSEAPRQRVTISC |
| 1a FR2 | 135 | WYQQLPGKAPKLLIY |
| 1a FR3 | 136 | GVSDRFSGSKSG--TSASLAISGLQSEDEADYYC |
| 1e FR1 | 137 | QSVLTQPPSVSGAPGQRVTISC |
| 1e FR2 | 138 | WYQQLPGTAPKLLIY |
| 1e FR3 | 139 | GVPDRFSGSKSG--TSASLAITGLQAEDEADYYC |
| 1c FR1 | 140 | QSVLTQPPSASGTPGQRVTISC |
| 1c FR2 | 141 | WYQQLPGTAPKLLIY |
| 1c FR3 | 142 | GVPDRFSGSKSG--TSASLAISGLQSEDEADYYC |
| 1g FR1 | 143 | QSVLTQPPSASGTPGQRVTISC |
| 1g FR2 | 144 | WYQQLPGTAPKLLIY |
| 1g FR3 | 145 | GVPDRFSGSKSG--TSASLAISGLRSEDEADYYC |
| 1b FR1 | 146 | QSVLTQPPSVSAAPGQKVTISC |
| 1b FR2 | 147 | WYQQLPGTAPKLLIY |
| 1b FR3 | 148 | GIPDRFSGSKSG--TSATLGITGLQTGDEADYYC |
| 2c FR1 | 149 | QSALTQPPSASGSPGQSVTISC |
| 2c FR2 | 150 | WYQQHPGKAPKLMIY |
| 2c FR3 | 151 | GVPDRFSGSKSG--NTASLTVSGLQAEDEADYYC |
| 2e FR1 | 152 | QSALTQPRSVSGSPGQSVTISC |
| 2e FR2 | 153 | WYQQHPGKAPKLMIY |
| 2e FR3 | 154 | GVPDRFSGSKSG--NTASLTISGLQAEDEADYYC |
| 2a2 FR1 | 155 | QSALTQPASVSGSPGQSITISC |
| 2a2 FR2 | 156 | WYQQHPGKAPKLMIY |
| 2a2 FR3 | 157 | GVSNRFSGSKSG--NTASLTISGLQAEDEADYYC |
| 2d FR1 | 158 | QSALTQPPSVSGSPGQSVTISC |
| 2d FR2 | 159 | WYQQPPGTAPKLMIY |
| 2d FR3 | 160 | GVPDRFSGSKSG--NTASLTISGLQAEDEADYYC |
| 2b2 FR1 | 161 | QSALTQPASVSGSPGQSITISC |
| 2b2 FR2 | 162 | WYQQHPGKAPKLMIY |
| 2b2 FR3 | 163 | GVSNRFSGSKSG--NTASLTISGLQAEDEADYYC |
| 3r FR1 | 164 | SYELTQPPSVSVSPGQTASITC |
| 3r FR2 | 165 | WYQQKPGQSPVLVIY |
| 3r FR3 | 166 | GIPERFSGSNSG--NTATLTISGTQAMDEADYYC |
| 3j FR1 | 167 | SYELTQPLSVSVALGQTARITC |
| 3j FR2 | 168 | WYQQKPGQAPVLVIY |
| 3j FR3 | 169 | GIPERFSGSNSG--NTATLTISRAQAGDEADYYC |
| 3p FR1 | 170 | SYELTQPPSVSVSPGQTARITC |
| 3p FR2 | 171 | WYQQKSGQAPVLVIY |
| 3p FR3 | 172 | GIPERFSGSSSG--TMATLTISGAQVEDEADYYC |
| 3a FR1 | 173 | SYELTQPPSVSVSLGQMARITC |
| 3a FR2 | 174 | WYQQKPGQFPVLVIY |
| 3a FR3 | 175 | GIPERFSGSSSG--TIVTLTISGVQAEDEADYYC |
| 3l FR1 | 176 | SSELTQDPAVSVALGQTVRITC |
| 3l FR2 | 177 | WYQQKPGQAPVLVIY |
| 3l FR3 | 178 | GIPDRFSGSSSG--NTASLTITGAQAEDEADYYC |
| 3h FR1 | 179 | SYVLTQPPSVSVAPGKTARITC |
| 3h FR2 | 180 | WYQQKPGQAPVLVIY |
| 3h FR3 | 181 | GIPERFSGSNSG--NTATLTISRVEAGDEADYYC |
| 3e FR1 | 182 | SYELTQLPSVSVSPGQTARITC |
| 3e FR2 | 183 | WYQQKPGQAPELVIY |
| 3e FR3 | 184 | GIPERFSGSTSG--NTTTLTISRVLTEDEADYYC |
| 3m FR1 | 185 | SYELMQPPSVSVSPGQTARITC |
| 3m FR2 | 186 | WYQQKPGQAPVLVIY |
| 3m FR3 | 187 | GIPERFSGSSSG--TTVTLTISGVQAEDEADYYC |
| 2-19 FR1 | 188 | SYELTQPSSVSVSPGQTARITC |
| 2-19 FR2 | 189 | WFQQKPGQAPVLVIY |
| 2-19 FR3 | 190 | GIPERFSGSSSG--TTVTLTISGAQVEDEADYYC |
| 4c FR1 | 191 | LPVLTQPPSASALLGASIKLTC |
| 4c FR2 | 192 | WYQQRPGRSPQYIMK |
| 4c FR3 | 193 | GIPDRFMGSSSG--ADRYLTFSNLQSDDEAEYHC |
| 4a FR1 | 194 | QPVLTQSSSASASLGSSVKLTC |
| 4a FR2 | 195 | WHQQQPGKAPRYLMK |
| 4a FR3 | 196 | GVPDRFSGSSSG--ADRYLTISNLQLEDEADYYC |
| 4b FR1 | 197 | QLVLTQSPSASASLGASVKLTC |
| 4b FR2 | 198 | WHQQQPEKGPRYLMK |
| 4b FR3 | 199 | GIPDRFSGSSSG--AERYLTISSLQSEDEADYYC |
| 5e FR1 | 200 | QPVLTQPPSSSASPGESARLTC |
| 5e FR2 | 201 | WYQQKPGSPPRYLLY |
| 5e FR3 | 202 | GVPSRFSGSKDASANTGILLISGLQSEDEADYYC |
| 5c FR1 | 203 | QAVLTQPASLSASPGASASLTC |
| 5c FR2 | 204 | WYQQKPGSPPQYLLR |
| 5c FR3 | 205 | GVPSRFSGSKDASANAGILLISGLQSEDEADYYC |
| 5b FR1 | 206 | QPVLTQPSSHSASSGASVRLTC |
| 5b FR2 | 207 | WYQQKPGNPPRYLLY |
| 5b FR3 | 208 | GVPSRFSGSNDASANAGILRISGLQPEDEADYYC |
| 6a FR1 | 209 | NFMLTQPHSVSESPGKTVTISC |
| 6a FR2 | 210 | WYQQRPGSSPTTVIY |
| 6a FR3 | 211 | GVPDRFSGSIDSSSNSASLTISGLKTEDEADYYC |
| 7a FR1 | 212 | QTVVTQEPSLTVSPGGTVTLTC |
| 7a FR2 | 213 | WFQQKPGQAPRALIY |
| 7a FR3 | 214 | WTPARFSGSLLG--GKAALTLSGVQPEDEAEYYC |
| 7b FR1 | 215 | QAVVTQEPSLTVSPGGTVTLTC |
| 7b FR2 | 216 | WFQQKPGQAPRTLIY |
| 7b FR3 | 217 | WTPARFSGSLLG--GKAALTLSGAQPEDEAEYYC |
| 8a FR1 | 218 | QTVVTQEPSFSVSPGGTVTLTC |
| 8a FR2 | 219 | WYQQTPGQAPRTLIY |
| 8a FR3 | 220 | GVPDRFSGSILG--NKAALTITGAQADDESDYYC |
| 9a FR1 | 221 | QPVLTQPPSASASLGASVTLTC |
| 9a FR2 | 222 | WYQQRPGKGPRFVMR |
| 9a FR3 | 223 | GIPDRFSVLGSG--LNRYLTIKNIQEEDESDYHC |
| 10a FR1 | 224 | QAGLTQPPSVSKGLRQTATLTC |
| 10a FR2 | 225 | WLQQHQGHPPKLLSY |
| 10a FR3 | 226 | GISERLSASRSG--NTASLTITGLQPEDEADYYC |
| JL1 FR4 | 227 | FGTGTKVTVL |
| JL2 FR4 | 228 | FGGGTKLTVL |
| JL3 FR4 | 229 | FGGGTKLTVL |
| JL7 FR4 | 230 | FGGGTQLTVL |

The immunoglobulin constant light chain region for use in the present invention is determined by the variable light chain the CDRs are grafted into. For example, if the variable light chain FR regions are derived from the immunoglobulin kappa light chain variable region, then a constant light chain region from an immunoglobulin kappa light chain constant region (IGKC) can be used to produce a light chain VL-CL chain. An IGKC that may be used in the present invention includes SEQ ID NO: 231 in Table 6 below. Conversely, when the framework region is immunoglobulin lambda light chain variable region, then an immunoglobulin lambda light chain constant region (IGLC) may be used to produce a lambda VL-CL light chain. An immunoglobulin lambda light chain constant region that may be used in the present invention includes (SEQ ID NO: 232) in Table 6 below, and allelic variants thereof, which are generally known in the art, for example as identified in OMIM entry 147200 for IGKC variants and 147220 for IGLC variants.

TABLE 6
Sequence of Human Immunoglobulin
Light Chain Constant Regions

| Ig Light | | |
|---|---|---|
| Chain | SEQ | |
| Constant | ID | |
| Region | NO: | Sequence |
| Ig Kappa | 231 | TVAAPSVFIFPPSDEQLKSGTASVVCLLNN |
| Constant | | FYPREAKVQWKVDNALQSGNSQESVTEQDS |
| Region | | KDSTYSLSSTLTLSKADYEKHKVYACEVTH |
| (IGKC) | | QGLSSPVTKSFNRGEC |
| Ig Lambda | 232 | QPKAAPSVTLFPPSSEELQANKATLVCLIS |
| Constant | | DFYPGAVTVAWKADSSPVKAGVETTTPSKQ |
| Region) | | SNNKYAASSYLSLTPEQWKSHRSYSCQVTH |
| (IGLC) | | EGSTVEKTVAPTECS |

In some embodiments, the human acceptor heavy chain framework is derived from an amino acid sequence encoded by a human IGHV gene for framework 1, 2, and 3, and an IGHJ gene for framework 4. Non-limiting examples of human heavy chain IGHV and IGHJ acceptor framework regions are provided, for example, in Table 7.

TABLE 7
Sequences of Human Immunoglobulin
Heavy Chain Variable Regions

| Heavy | | |
|---|---|---|
| Chain | | |
| Variable | SEQ | |
| FR | ID | |
| Regions | NO: | Sequence |
| 1-02 FR1 | 233 | QVQLVQSGAEVKKPGASVKVSCKAS |
| 1-02 FR2 | 234 | WVRQAPGQGLEWMG |
| 1-02 FR3 | 235 | RVTMTRDTSISTAYMELSRLRSDDTAVYYCAR |
| 1-03 FR1 | 236 | QVQLVQSGAEVKKPGASVKVSCKAS |
| 1-03 FR2 | 237 | WVRQAPGQRLEWMG |
| 1-03 FR3 | 238 | RVTITRDTSASTAYMELSSLRSEDTAVYYCAR |
| 1-08 FR1 | 239 | QVQLVQSGAEVKKPGASVKVSCKAS |
| 1-08 FR2 | 240 | WVRQATGQGLEWMG |
| 1-08 FR3 | 241 | RVTMTRNTSISTAYMELSSLRSEDTAVYYCAR |
| 1-18 FR1 | 242 | QVQLVQSGAEVKKPGASVKVSCKAS |
| 1-18 FR2 | 243 | WVRQAPGQGLEWMG |
| 1-18 FR3 | 244 | RVTMTTDTSTSTAYMELRSLRSDDTAVYYCAR |
| 1-24 FR1 | 245 | QVQLVQSGAEVKKPGASVKVSCKVS |
| 1-24 FR2 | 246 | WVRQAPGKGLEWMG |
| 1-24 FR3 | 247 | RVTMTEDTSTDTAYMELSSLRSEDTAVYYCAT |
| 1-45 FR1 | 248 | QMQLVQSGAEVKKTGSSVKVSCKAS |
| 1-45 FR2 | 249 | WVRQAPGQALEWMG |
| 1-45 FR3 | 250 | RVTITRDRSMSTAYMELSSLRSEDTAMYYCAR |
| 1-46 FR1 | 251 | QVQLVQSGAEVKKPGASVKVSCKAS |
| 1-46 FR2 | 252 | WVRQAPGQGLEWMG |
| 1-46 FR3 | 253 | RVTMTRDTSTSTVYMELSSLRSEDTAVYYCAR |
| 1-58 FR1 | 254 | QMQLVQSGPEVKKPGTSVKVSCKAS |
| 1-58 FR2 | 255 | WVRQARGQRLEWIG |
| 1-58 FR3 | 256 | RVTITRDMSTSTAYMELSSLRSEDTAVYYCAA |
| 1-69 FR1 | 257 | QVQLVQSGAEVKKPGSSVKVSCKAS |
| 1-69 FR2 | 258 | WVRQAPGQGLEWMG |
| 1-69 FR3 | 259 | RVTITADESTSTAYMELSSLRSEDTAVYYCAR |
| 1-e FR1 | 260 | QVQLVQSGAEVKKPGSSVKVSCKAS |
| 1-e FR2 | 261 | WVRQAPGQGLEWMG |
| 1-e FR3 | 262 | RVTITADKSTSTAYMELSSLRSEDTAVYYCAR |
| 1-f FR1 | 263 | EVQLVQSGAEVKKPGATVKISCKVS |
| 1-f FR2 | 264 | WVQQAPGKGLEWMG |
| 1-f FR3 | 265 | RVTITADTSTDTAYMELSSLRSEDTAVYYCAT |
| 2-05 FR1 | 266 | QITLKESGPTLVKPTQTLTLTCTFS |
| 2-05 FR2 | 267 | WIRQPPGKALEWLA |
| 2-05 FR3 | 268 | RLTITKDTSKNQVVLTMTNMDPVDTATYYCAHR |
| 2-26 FR1 | 269 | QVTLKESGPVLVKPTETLTLTCTVS |
| 2-26 FR2 | 270 | WIRQPPGKALEWLA |
| 2-26 FR3 | 271 | RLTISKDTSKSQVVLTMTNMDPVDTATYYCARI |
| 2-70 FR1 | 272 | QVTLKESGPALVKPTQTLTLTCTFS |
| 2-70 FR2 | 273 | WIRQPPGKALEWLA |
| 2-70 FR3 | 274 | RLTISKDTSKNQVVLTMTNMDPVDTATYYCARI |
| 3-07 FR1 | 275 | EVQLVESGGGLVQPGGSLRLSCAAS |
| 3-07 FR2 | 276 | WVRQAPGKGLEWVA |
| 3-07 FR3 | 277 | RFTISRDNAKNSLYLQMNSLRAEDTAVYYCAR |
| 3-09 FR1 | 278 | EVQLVESGGGLVQPGRSLRLSCAAS |
| 3-09 FR2 | 279 | WVRQAPGKGLEWVS |
| 3-09 FR3 | 280 | RFTISRDNAKNSLYLQMNSLRAEDTALYYCAKD |
| 3-11 FR1 | 281 | QVQLVESGGGLVKPGGSLRLSCAAS |
| 3-11 FR2 | 282 | WIRQAPGKGLEWVS |
| 3-11 FR3 | 283 | RFTISRDNAKNSLYLQMNSLRAEDTAVYYCAR |
| 3-13 FR1 | 284 | EVQLVESGGGLVQPGGSLRLSCAAS |
| 3-13 FR2 | 285 | WVRQATGKGLEWVS |
| 3-13 FR3 | 286 | RFTISRENAKNSLYLQMNSLRAGDTAVYYCAR |
| 3-15 FR1 | 287 | EVQLVESGGGLVKPGGSLRLSCAAS |
| 3-15 FR2 | 288 | WVRQAPGKGLEWVG |
| 3-15 FR3 | 289 | RFTISRDD SKNTLYLQMNSLKTEDTAVYYCTT |
| 3-20 FR1 | 290 | EVQLVESGGGVVRPGGSLRLSCAAS |
| 3-20 FR2 | 291 | WVRQAPGKGLEWVS |
| 3-20 FR3 | 292 | RFTISRDNAKNSLYLQMNSLRAEDTALYHCAR |
| 3-21 FR1 | 293 | EVQLVESGGGLVKPGGSLRLSCAAS |
| 3-21 FR2 | 294 | WVRQAPGKGLEWVS |
| 3-21 FR3 | 295 | RFTISRDNAKNSLYLQMNSLRAEDTAVYYCAR |
| 3-23 FR1 | 296 | EVQLLESGGGLVQPGGSLRLSCAAS |
| 3-23 FR2 | 297 | WVRQAPGKGLEWVS |
| 3-23 FR3 | 298 | RFTISRDNSKNTLYLQMNSLRAEDTAVYYCAK |
| 3-30 FR1 | 299 | QVQLVESGGGVVQPGRSLRLSCAAS |
| 3-30 FR2 | 300 | WVRQAPGKGLEWVA |
| 3-30 FR3 | 301 | RFTISRDNSKNTLYLQMNSLRAEDTAVYYCAK |
| 3-30.3 FR1 | 302 | QVQLVESGGGVVQPGRSLRLSCAAS |
| 3-30.3 FR2 | 303 | WVRQAPGKGLEWVA |
| 3-30.3 FR3 | 304 | RFTISRDNSKNTLYLQMNSLRAEDTAVYYCAR |
| 3-3 FR1 | 305 | QVQLVESGGGVVQPGRSLRLSCAAS |
| 3-30.5 FR2 | 306 | WVRQAPGKGLEWVA |
| 3-30.5 FR3 | 307 | RFTISRDNSKNTLYLQMNSLRAEDTAVYYCAK |
| 3-3 FR13 | 308 | QVQLVESGGGVVQPGRSLRLSCAAS |
| 3-33 FR2 | 309 | WVRQAPGKGLEWVA |
| 3-33 FR3 | 310 | RFTISRDNSKNTLYLQMNSLRAEDTAVYYCAR |
| 3-43 FR1 | 311 | EVQLVESGGVVVQPGGSLRLSCAAS |
| 3-43 FR2 | 312 | WVRQAPGKGLEWVS |
| 3-43 FR3 | 313 | RFTISRDNSKNSLYLQMNSLRTEDTALYYCAKD |
| 3-48 FR1 | 314 | EVQLVESGGGLVQPGGSLRLSCAAS |
| 3-48 FR2 | 315 | WVRQAPGKGLEWVS |
| 3-48 FR3 | 316 | RFTISRDNAKNSLYLQMNSLRDEDTAVYYCAR |
| 3-49 FR1 | 317 | EVQLVESGGGLVQPGRSLRLSCTAS |
| 3-49 FR2 | 318 | WFRQAPGKGLEWVG |
| 3-49 FR3 | 319 | RFTISRDGSKSIAYLQMNSLKTEDTAVYYCTR |
| 3-53 FR1 | 320 | EVQLVETGGGLIQPGGSLRLSCAAS |
| 3-53 FR2 | 321 | WVRQAPGKGLEWVS |
| 3-53 FR3 | 322 | RFTISRDNSKNTLYLQMNSLRAEDTAVYYCAR |
| 3-64 FR1 | 323 | EVQLVESGGGLVQPGGSLRLSCAAS |
| 3-64 FR2 | 324 | WVRQAPGKGLEYVS |
| 3-64 FR3 | 325 | RFTISRDNSKNTLYLQMGSLRAEDMAVYYCAR |
| 3-66 FR1 | 326 | EVQLVESGGGLVQPGGSLRLSCAAS |
| 3-66 FR2 | 327 | WVRQAPGKGLEWVS |
| 3-66 FR3 | 328 | RFTISRDNSKNTLYLQMNSLRAEDTAVYYCAR |
| 3-72 FR1 | 329 | EVQLVESGGGLVQPGGSLRLSCAAS |
| 3-72 FR2 | 330 | WVRQAPGKGLEWVG |
| 3-72 FR3 | 331 | RFTISRDDSKNSLYLQMNSLKTEDTAVYYCAR |
| 3-73 FR1 | 332 | EVQLVESGGGLVQPGGSLKLSCAAS |
| 3-73 FR2 | 333 | WVRQASGKGLEWVG |
| 3-73 FR3 | 334 | RFTISRDDSKNTAYLQMNSLKTEDTAVYYCTR |
| 3-74 FR1 | 335 | EVQLVESGGGLVQPGGSLRLSCAAS |
| 3-74 FR2 | 336 | WVRQAPGKGLVWVS |
| 3-74 FR3 | 337 | RFTISRDNAKNTLYLQMNSLRAEDTAVYYCAR |
| 3-d FR1 | 338 | EVQLVESRGVLVQPGGSLRLSCAAS |
| 3-d FR2 | 339 | WVRQAPGKGLEWVS |
| 3-d FR3 | 340 | RFTISRDNSKNTLHLQMNSLRAEDTAVYYCKK |
| 4-04 FR1 | 341 | QVQLQESGPGLVKPSGTLSLTCAVS |
| 4-04 FR2 | 342 | WVRQPPGKGLEWIG |
| 4-04 FR3 | 343 | RVTISVDKSKNQFSLKLSSVTAADTAVYYCAR |
| 4-28 FR1 | 344 | QVQLQESGPGLVKPSDTLSLTCAVS |
| 4-28 FR2 | 345 | WIRQPPGKGLEWIG |
| 4-28 FR3 | 346 | RVTMSVDTSKNQFSLKLSSVTAVDTAVYYCAR |
| 4-30.1 FR1 | 347 | QVQLQESGPGLVKPSQTLSLTCTVS |
| 4-30.1 FR2 | 348 | WIRQHPGKGLEWIG |
| 4-30.1 FR3 | 349 | RVTISVDTSKNQFSLKLSSVTAADTAVYYCAR |
| 4-3 FR1 | 350 | QLQLQESGSGLVKPSQTLSLTCAVS |
| 4-30.2 FR2 | 351 | WIRQPPGKGLEWIG |
| 4-30.2 FR3 | 352 | RVTISVDRSKNQFSLKLSSVTAADTAVYYCAR |
| 4-3 FR10.4 | 353 | QVQLQESGPGLVKPSQTLSLTCTVS |
| 4-30.4 FR2 | 354 | WIRQPPGKGLEWIG |
| 4-30.4 FR3 | 355 | RVTISVDTSKNQFSLKLSSVTAADTAVYYCAR |
| 4-3 FR1 | 356 | QVQLQESGPGLVKPSQTLSLTCTVS |
| 4-31 FR2 | 357 | WIRQHPGKGLEWIG |
| 4-31 FR3 | 358 | RVTISVDTSKNQFSLKLSSVTAADTAVYYCAR |
| 4-34 FR1 | 359 | QVQLQQWGAGLLKPSETLSLTCAVY |
| 4-34 FR2 | 360 | WIRQPPGKGLEWIG |
| 4-34 FR3 | 361 | RVTISVDTSKNQFSLKLSSVTAADTAVYYCAR |
| 4-39 FR1 | 362 | QLQLQESGPGLVKPSETLSLTCTVS |
| 4-39 FR2 | 363 | WIRQPPGKGLEWIG |
| 4-39 FR3 | 364 | RVTISVDTSKNQFSLKLSSVTAADTAVYYCAR |
| 4-59 FR1 | 365 | QVQLQESGPGLVKPSETLSLTCTVS |
| 4-59 FR2 | 366 | WIRQPPGKGLEWIG |
| 4-59 FR3 | 367 | RVTISVDTSKNQFSLKLSSVTAADTAVYYCAR |
| 4-61 FR1 | 368 | QVQLQESGPGLVKPSETLSLTCTVS |
| 4-61 FR2 | 369 | WIRQPPGKGLEWIG |
| 4-61 FR3 | 370 | RVTISVDTSKNQFSLKLSSVTAADTAVYYCAR |
| 4-b FR1 | 371 | QVQLQESGPGLVKPSETLSLTCAVS |
| 4-b FR2 | 372 | WIRQPPGKGLEWIG |
| 4-b FR3 | 373 | RVTISVDTSKNQFSLKLSSVTAADTAVYYCAR |
| 5-51 FR1 | 374 | EVQLVQSGAEVKKPGESLKISCKGS |
| 5-51 FR2 | 375 | WVRQMPGKGLEWMG |
| 5-51 FR3 | 376 | QVTISADKSISTAYLQWSSLKASDTAMYYCAR |
| 5-a FR1 | 377 | EVQLVQSGAEVKKPGESLRISCKGS |
| 5-a FR2 | 378 | WVRQMPGKGLEWMG |
| 5-a FR3 | 379 | HVTISADKSISTAYLQWSSLKASDTAMYYCAR |
| 6-01 FR1 | 380 | QVQLQQSGPGLVKPSQTLSLTCAIS |
| 6-01 FR2 | 381 | WIRQSPSRGLEWLG |
| 6-01 FR3 | 382 | RITINPDTSKNQFSLQLNSVTPEDTAVYYCAR |
| 7-4.1 FR1 | 383 | QVQLVQSGSELKKPGASVKVSCKAS |
| 7-4.1 FR2 | 384 | WVRQAPGQGLEWMG |
| 7-4.1 FR3 | 385 | RFVFSLDTSVSTAYLQICSLKAEDTAVYYCAR |
| JH1 FR4 | 386 | WGQGTLVTVSS |
| JH2 FR4 | 387 | WGRGTLVTVSS |
| JH3 FR4 | 388 | WGQGTMVTVSS |
| JH4 FR4 | 389 | WGQGTLVTVSS |
| JH5 FR4 | 390 | WGQGTLVTVSS |
| JH6 FR4 | 391 | WGQGTTVTVSS |

The immunoglobulin heavy chain constant region for use in the present invention is determinant on the immunoglobulin class desired. All classes of immunoglobulins—IgG, IgD, IgA, IgM and IgE—are herein contemplated. For example, if the desired immunoglobulin is IgG, then the amino acid sequence encoding the IgG heavy chain constant region (IGGH) may be used. Immunoglobulin heavy chain constant regions that may be used in the present invention include those of IGGH, IGDH, IGAH, IGMH, and IGEH (SEQ ID NOS: 392-427) provided in Table 8 below, and allelic variants thereof, which are generally known in the art, for example as identified in OMIM entry 147100 for IGGH1 variants, 147110 for IGGH2 variants, 147120 for IGGH3 variants, 147130 for IGGH4 variants, 146900 for IGAH1 variants, 147000 for IGAH2 variants, 147180 for IGEH variants, 147020 for IGMH variants, 147170 for IGDH variants, all of which are incorporated by reference herein. In certain embodiment, the hinge region of a particular immunoglobulin class may be used in constructing the antibody contemplated herein. In some embodiments, the hinge region can be derived from a natural hinge region amino acid sequence as described in Table 8 (SEQ ID NOS: 393, 397, 401, 409, 413, 417, and 421), or a variant thereof.

In some embodiments, the hinge region can be synthetically generated. Further contemplated herein are antibodies of immunoglobulin class IgA and IgM, which, in some embodiments, may be complexed with a joining polypeptide described in Table 9, or a variant thereof.

TABLE 8
Immunoglobulin Heavy Chain Constant Region

| Heavy | | |
|---|---|---|
| Chain | SEQ | |
| Constant | ID | |
| Region | NO: | Sequence |
| IGAH1 CH1 | 392 | ASPTSPKVFPLSLCSTQPDGNVVIACLVQGFFPQEPLSVTWSESGQ |
| | | GVTARNFPPSQDASGDLYTTSSQLTLPATQCLAGKSVTCHVKHYTN |
| | | PSQDVTVPCP |
| IGAH1 Hinge | 393 | PSTPPTPSPSTPPTPSPS |
| IGAH1 CH2 | 394 | CCHPRLSLHRPALEDLLLGSEANLTCTLTGLRDASGVTFTWTPSSG |
| | | KSAVQGPPERDLCGCYSVSSVLPGCAEPWNHGKTFTCTAAYPESKT |
| | | PLTATLSKS |
| IGAH1 CH3 | 395 | GNTFRPEVHLLPPPSEELALNELVTLTCLARGFSPKDVLVRWLQGS |
| | | QELPREKYLTWASRQEPSQGTTTFAVTSILRVAAEDWKKGDTFSCM |
| | | VGHEALPLAFTQKTIDRLA |
| IGAH2 CH1 | 396 | ASPTSPKVFPLSLDSTPQDGNVVVACLVQGFFPQEPLSVTWSESGQ |
| | | NVTARNFPPSQDASGDLYTTSSQLTLPATQCPDGKSVTCHVKHYTN |
| | | PSQDVTVPCP |
| IGAH2 Hinge | 397 | PPPPP |
| IGAH2 CH2 | 398 | CCHPRLSLHRPALEDLLLGSEANLTCTLTGLRDASGATFTWTPSSG |
| | | KSAVQGPPERDLCGCYSVSSVLPGCAQPWNHGETFTCTAAHPELKT |
| | | PLTANITKS |
| IGAH2 CH3 | 399 | GNTFRPEVHLLPPPSEELALNELVTLTCLARGFSPKDVLVRWLQGS |
| | | QELPREKYLTWASRQEPSQGTTTFAVTSILRVAAEDWKKGDTFSCM |
| | | VGHEALPLAFTQKTIDRLA |
| IGDH CH1 | 400 | APTKAPDVFPIISGCRHPKDNSPVVLACLITGYHPTSVTVTWYMGT |
| | | QSQPQRTFPEIQRRDSYYMTSSQLSTPLQQWRQGEYKCVVQHTASK |
| | | SKKEIFRWP |
| IGDH Hinge | 401 | ESPKAQASSVPTAQPQAEGSLAKATTAPATTRNTGRGGEEKKKEKE |
| | | KEEQEERETKTP |
| IGDH CH2 | 402 | ECPSHTQPLGVYLLTPAVQDLWLRDKATFTCFVVGSDLKDAHLTWE |
| | | VAGKVPTGGVEEGLLERHSNGSQSQHSRLTLPRSLWNAGTSVTCTL |
| | | NHPSLPPQRLMALREP |
| IGDH CH3 | 403 | AAQAPVKLSLNLLASSDPPEAASWLLCEVSGFSPPNILLMWLEDQR |
| | | EVNTSGFAPARPPPQPRSTTFWAWSVLRVPAPPSPQPATYTCVVSH |
| | | EDSRTLLNASRSLEVS |
| IGEH CH1 | 404 | ASTQSPSVFPLTRCCKNIPSNATSVTLGCLATGYFPEPVMVTCDTG |
| | | SLNGTTMTLPATTLTLSGHYATISLLTVSGAWAKQMFTCRVAHTPS |
| | | STDWVDNKTFS |
| IGEH CH2 | 405 | VCSRDFTPPTVKILQSSCDGGGHFPPTIQLLCLVSGYTPGTINITW |
| | | LEDGQVMDVDLSTASTTQEGELASTQSELTLSQKHWLSDRTYTCQV |
| | | TYQGHTFEDSTKKCA |
| IGEH CH3 | 406 | DSNPRGVSAYLSRPSPFDLFIRKSPTITCLVVDLAPSKGTVNLTWS |
| | | RASGKPVNHSTRKEEKQRNGTLTVTSTLPVGTRDWIEGETYQCRVT |
| | | HPHLPRALMRSTTKTS |
| IGEH CH4 | 407 | GPRAAPEVYAFATPEWPGSRDKRTLACLIQNFMPEDISVQWLHNEV |
| | | QLPDARHSTTQPRKTKGSGFFVFSRLEVTRAEWEQKDEFICRAVHE |
| | | AASPSQTVQRAVSVNP |
| IGGH1 CH1 | 408 | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKKV |
| IGGH1 Hinge | 409 | EPKSCDKTHTCPPCP |
| IGGH1 CH2 | 410 | APELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCK |
| | | VSNKALPAPIEKTISKAK |
| IGGH1 CH3 | 411 | GQPREPQVYTLPPSRDELTKNQVSLTCLVKGFYPSDIAVEWESNGQ |
| | | PENNYKTTPPVLDSDGSFFLYSKLTVDKSRWQQGNVFSCSVMHEAL |
| | | HNHYTQKSLSLSP |
| IGGH2 CH1 | 412 | ASTKGPSVFPLAPCSRSTSESTAALGCLVKDYFPEPVTVSWNSGAL |
| | | TSGVHTFPAVLQSSGLYSLSSVVTVPSSNFGTQTYTCNVDHKPSNT |
| | | KVDKTV |
| IGGH2 Hinge | 413 | ERKCCVECPPCP |
| IGGH2 CH2 | 414 | APPVAGPSVFLFPPKPKDTLMISRTPEVTCVVVDVSHEDPEVQFNW |
| | | YVDGVEVHNAKTKPREEQFNSTFRVVSVLTVVHQDWLNGKEYKCKV |
| | | SNKGLPAPIEKTISKTK |
| IGGH2 CH3 | 415 | GQPREPQVYTLPPSREEMTKNQVSLTCLVKGFYPSDIAVEWESNGQ |
| | | PENNYKTTPPMLDSDGSFFLYSKLTVDKSRWQQGNVFSCSVMHEAL |
| | | HNHYTQKSLSLSP |
| IGGH3 CH1 | 416 | ASTKGPSVFPLAPCSRSTSGGTAALGCLVKDYFPEPVTVSWNSGAL |
| | | TSGVHTFPAVLQSSGLYSLSSVVTVPSSSLGTQTYTCNVNHKPSNT |
| | | KVDKRV |
| IGGH3 Hinge | 417 | ELKTPLGDTTHTCPRCPEPKSCDTPPPCPRCPEPKSCDTPPPCPRC |
| | | PEPKSCDTPPPCPRCP |
| IGGH3 CH2 | 418 | APELLGGPSVFLFPPKPKDTLMISRTPEVTCVVVDVSHEDPEVQFK |
| | | WYVDGVEVHNAKTKPREEQYNSTFRVVSVLTVLHQDWLNGKEYKCK |
| | | VSNKALPAPIEKTISKTK |
| IGGH3 CH3 | 419 | GQPREPQVYTLPPSREEMTKNQVSLTCLVKGFYPSDIAVEWESSGQ |
| | | PENNYNTTPPMLDSDGSFFLYSKLTVDKSRWQQGNIFSCSVMHEAL |
| | | HNRFTQKSLSLSP |
| IGGH4 CH1 | 420 | ASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNT |
| | | KVDKRV |
| IGGH4 Hinge | 421 | ESKYGPPCPSCP |
| IGGH4 CH2 | 422 | APEFLGGPSVFLFPPKPKDTLMISRTPEVTCVVVDVSQEDPEVQFN |
| | | WYVDGVEVHNAKTKPREEQFNSTYRVVSVLTVLHQDWLNGKEYKCK |
| | | VSNKGLPSSIEKTISKAK |
| IGGH4 CH3 | 423 | GQPREPQVYTLPPSQEEMTKNQVSLTCLVKGFYPSDIAVEWESNGQ |
| | | PENNYKTTPPVLDSDGSFFLYSRLTVDKSRWQEGNVFSCSVMHEAL |
| | | HNHYTQKSLSLSL |
| IGMH CH1 | 424 | GSASAPTLFPLVSCENSPSDTSSVAVGCLAQDFLPDSITLSWKYKN |
| | | NSDISSTRGFPSVLRGGKYAATSQVLLPSKDVMQGTDEHVVCKVQH |
| | | PNGNKEKNVPLP |
| IGMH CH2 | 425 | VIAELPPKVSVFVPPRDGFFGNPRKSKLICQATGFSPRQIQVSWLR |
| | | EGKQVGSGVTTDQVQAEAKESGPTTYKVTSTLTIKESDWLGQSMFT |
| | | CRVDHRGLTFQQNASSMCVP |
| IGMH CH3 | 426 | DQDTAIRVFAIPPSFASIFLTKSTKLTCLVTDLTTYDSVTISWTRQ |
| | | NGEAVKTHTNISESHPNATFSAVGEASICEDDWNSGERFTCTVTHT |
| | | DLPSPLKQTISRPK |
| IGMH CH4 | 427 | GVALHRPDVYLLPPAREQLNLRESATITCLVTGFSPADVFVQWMQR |
| | | GQPLSPEKYVTSAPMPEPQAPGRYFAHSILTVSEEEWNTGETYTCV |
| | | AHEALPNRVTERTVDKST |

TABLE 9
Joining Polypeptide for IgA and
IgM Class Antibodies

| | SEQ | |
|---|---|---|
| | ID | |
| Ig Protein | NO: | Sequence |
| Joining | 428 | QEDERIVLVDNKCKCARITSRIIRSSEDPNEDI |
| Polypeptide | | VERNIRIIVPLNNRENISDPTSPLRTRFVYHLS |
| | | DLCKKCDPTEVELDNQIVTATQSNICDEDSATE |
| | | TCYTYDRNKCYTAVVPLVYGGETKMVETALTPD |
| | | ACYPD |

Methods of Identifying Compounds that Neutralize NDPK-ADK/FABP4 Complex Activity or Inhibit NDPK-ADK/FABP4 Complex Formation

One aspect provided herein relates to a method for identifying compounds which modulate/affect, and preferably neutralize, the ability of FABP4 to inhibit NDPK-ADK complex modulation of GPCRs. In some embodiments, the compounds interact with the NDPK-ADK/FABP4 complex, without inhibiting the ability of the NDPK-ADK complex unbound to FABP4 from modulating GPCRs. In some embodiments, the compound interferes with the ability of FABP4, NDPK, and ADK to form a NDPK-ADK/FABP4 complex. In some embodiments, the compound interferes with the ability of NDPK and ADK to form a NDPK-ADK complex. Compounds may include, by way of non-limiting example, peptides produced by expression of an appropriate nucleic acid sequence in a host cell or using synthetic organic chemistries (e.g., antibodies, antibody fragments, or antigen binding agents), or non-peptide small molecules produced using conventional synthetic organic chemistries well known in the art. Identifying assays may be automated in order to facilitate the identification of a large number of small molecules at the same time. The compounds identified can be used to modulate NDPK-ADK/FABP4 complex antagonism of GPCRs in a host, for example a human, to treat and or prevent an FABP4-mediated disorder.

Methods used for identifying compounds may be cell-based or cell-free. In some embodiments, the screen is cell free, and compounds are screened to determine their ability to interact or bind to FABP4, NDPK, ADK, NDK-ADK/FABP4, or NDK-ADK without inhibiting NDPK-ADK activity. For example, a compound is contacted with FABP4, NDPK, ADK, NDK-ADK/FABP4, or NDK-ADK and then an assay is performed to detect binding of the compound to the target. Assays to detect binding of compounds are well known in the art, for example as described in McFedries, et al., Methods for the Elucidation of Protein-Small Molecule Interactions. Chemistry & Biology (2013); Vol. 20(5):667-673; Pollard, A Guide to Simple and Informative Binding Assays, Mol. Biol. Cell (2010) Vol. 21, 4061-4067, both incorporated herein by reference in their entirety.

In further embodiments, the compound can be introduced into an ADP-kinase assay with FABP4, NDPK, ADK, ATP, and GTP, wherein measurement of ADP production is indicative of a compound capable of inhibiting the formation of the NDPK-ADK/FABP4 complex without inhibiting NDPK-ADK activity. Additional methods to determine NDPK-ADK activity are known in the art, for example as described in Hippe, et al., Activation of Heteromeric G Proteins by a High Energy Phosphate Transfer via Nucleoside Diphosphate Kinase (NDPK) B and Gβ Subunits. The Journal of Biological Chemistry (2003); Vol. 278 (9): 7227-7233.

In an alternative aspect, a method of identifying a compound capable of binding the NDPK-ADK complex and inhibiting NDPK-ADK/FABP4 complex formation without inhibiting NDPK-ADK complex activity is described. For example, a compound is contacted with the NDPK-ADK complex and then an assay is performed to detect binding of the compound to the NDPK-ADK complex. In further embodiments, the compound can be introduced into an ADP-kinase assay with the NDPK-ADK complex, FABP4, ATP, and GDP, wherein measurement of ADP production is indicative of a compound capable of binding the NDPK-ADK complex and inhibiting NDPK-ADK/FABP4 complex formation without inhibiting NDPK-ADK activity. In some embodiments the method further comprises introducing the compound into a cellular assay in the presence of FABP4 and NDPK-ADK complex, wherein the cellular assay includes a population of pancreatic islet β-cells expressing GPCRs or an insulin-secreting cell line and measuring insulin secretion. In some embodiments, the cell population is human pancreatic islet β-cells. In another embodiment, the cell population is an insulin-secreting cell line.

In an alternative aspect, a method of identifying a compound capable of binding the NDPK-ADK/FABP4 complex activity is described. For example, a compound is contacted with the NDPK-ADK/FABP4 complex and then an assay is performed to detect binding of the compound to the NDPK-ADK complex. In further embodiments, the compound can be introduced into an ADP-kinase assay with the NDPK-ADK/FABP4 complex, NDPK and ADK (or alternatively, NDPK-ADK), ATP, and GDP, wherein measurement of ADP production is indicative of a compound capable of inhibiting the interaction of FABP4 and the NDPK-ADK complex or is capable of sequestering the NDPK-ADK/FABP4 complex and allowing unbound NDPK-ADK complex to modulate GPCRs. In some embodiments the method further comprises introducing the compound into a cellular assay in the presence of NDPK-ADK complex, wherein the cellular assay includes a population of pancreatic islet β-cells expressing GPCRs or an insulin-secreting cell line and measuring insulin secretion. In some embodiments, the cell population is human pancreatic islet β-cells. In another embodiment, the cell population is an insulin-secreting cell line.

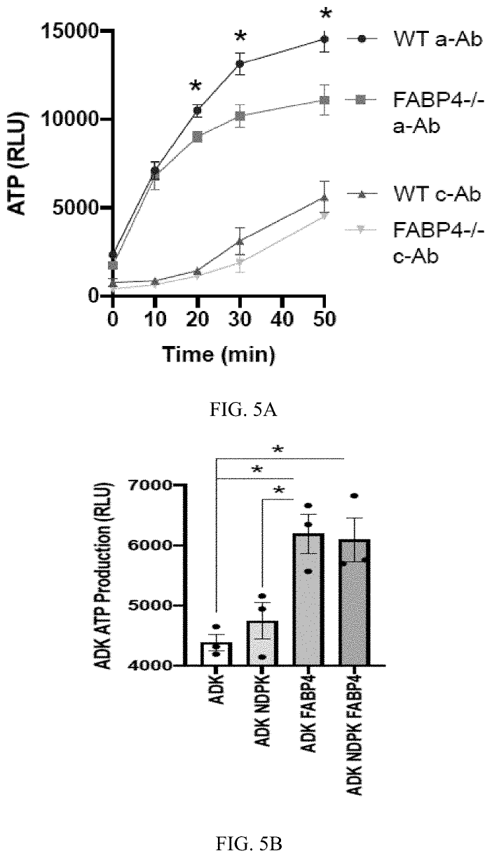

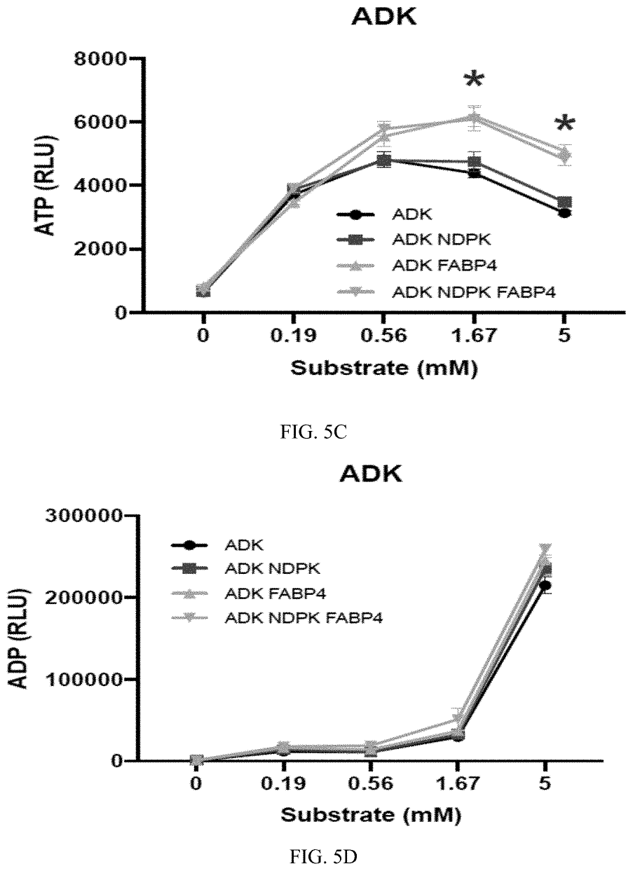

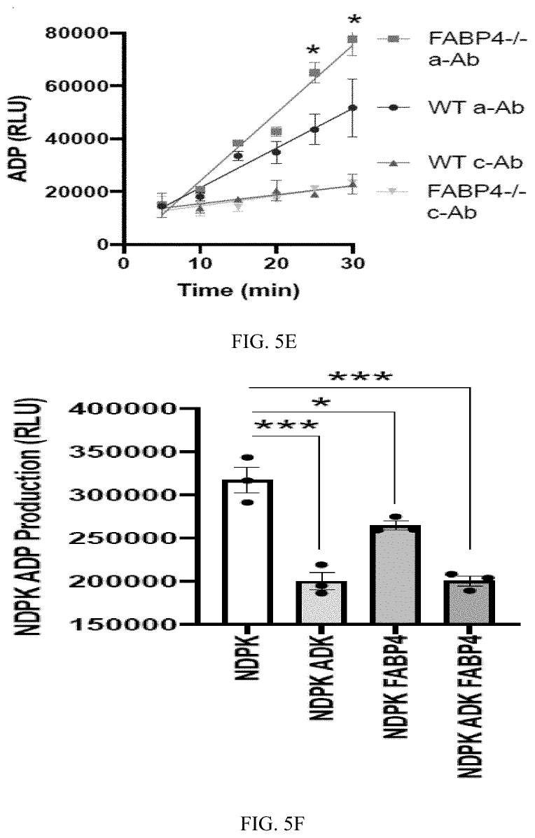

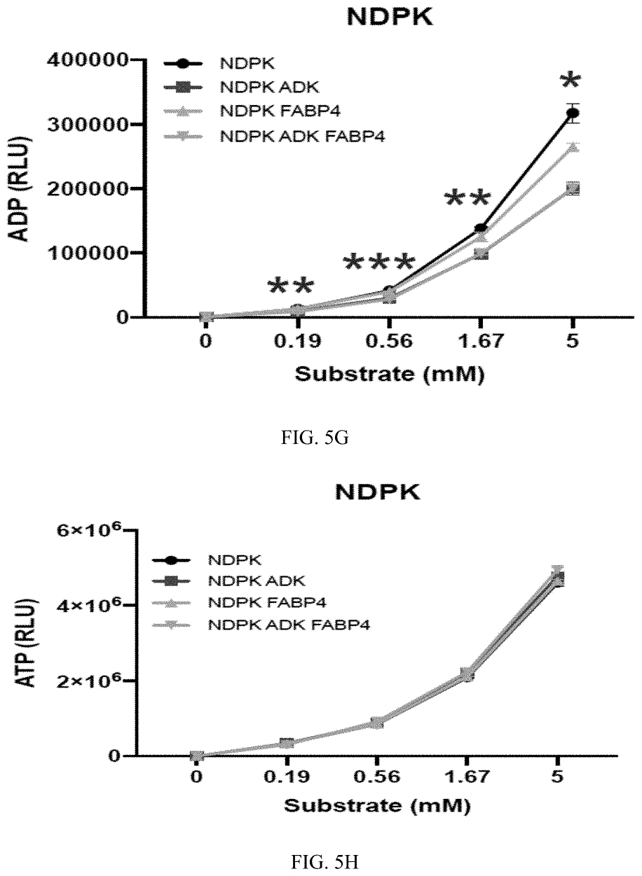

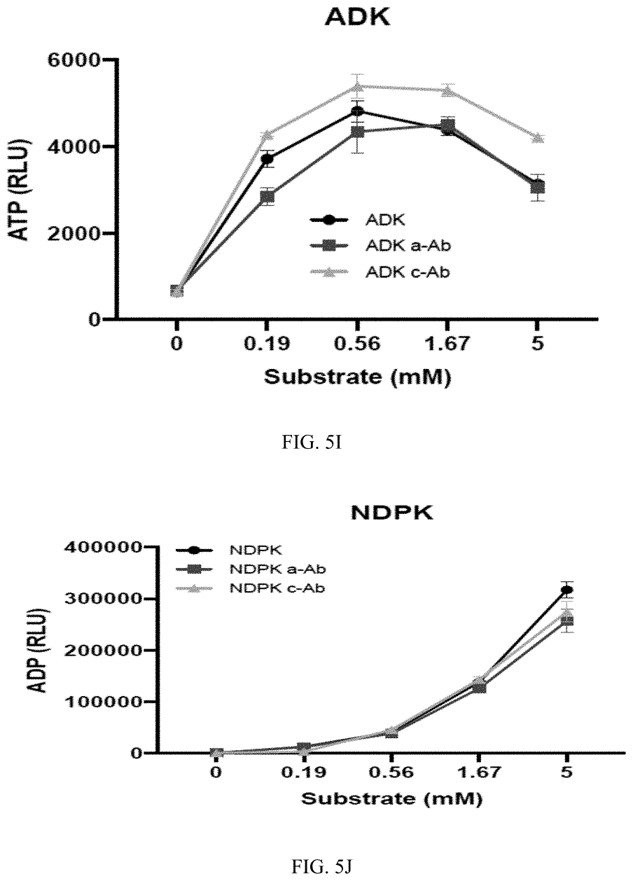

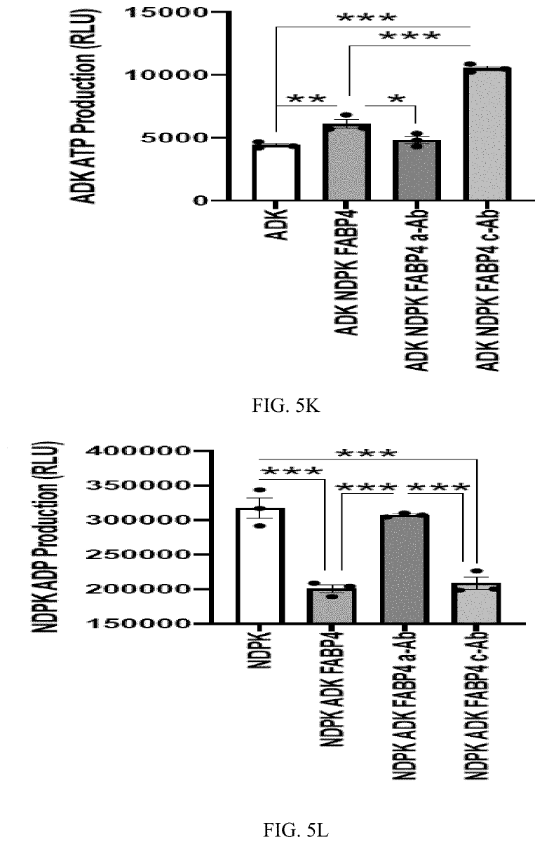

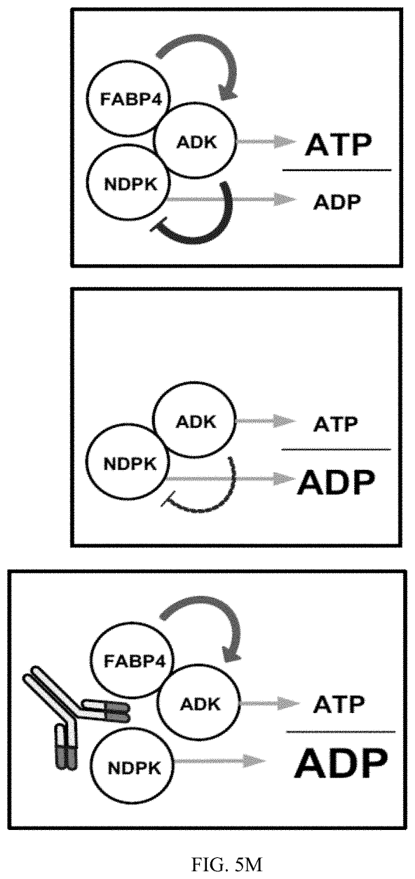

In an additional aspect, provided herein is a method of identifying a compound capable of increasing glucose-stimulated insulin secretion (GSIS) in the presence of FABP4 comprising contacting FABP4, NDPK, ADK, NDPK-ADK complex, and/or NDPK-ADK/FABP4 complex with pancreatic islet β-cells, which express GPCRs in the presence of a compound and contacting FABP4, NDPK, ADK, NDPK-ADK complex, and/or NDPK-ADK/FABP4 complex with pancreatic islet β-cells in the absence of the compound and measuring the level of insulin secretion in the presence of the compound and in the absence of the compound. An increase in the level of extracellular ADP to ATP or insulin secretion in the presence of the compound is indicative of a compound capable of neutralizing the binding of FABP4 to the NDPK-ADK complex to modulate GPCRs resulting in insulin secretion by the pancreatic islet β-cells. This is further exemplified, for example, in Example 4 below and FIGS. 5D-J).

In an additional aspect, provided herein is a method of identifying a compound capable of increasing glucose-stimulated insulin secretion (GSIS) in the presence of FABP4 comprising contacting FABP4, NDPK, ADK, NDPK-ADK complex, and/or NDPK-ADK/FABP4 complex with an insulin-secreting cell line in the presence of a compound and contacting FABP4, NDPK, ADK, NDPK-ADK complex, and/or NDPK-ADK/FABP4 complex with an insulin-secreting cell line in the absence of the compound and measuring the level of insulin secretion in the presence of the compound and in the absence of the compound. An increase in the GTP levels or the level of insulin secretion in the presence of the compound is indicative of a compound capable of increasing glucose-stimulated insulin secretion (GSIS) in the presence of FABP4.

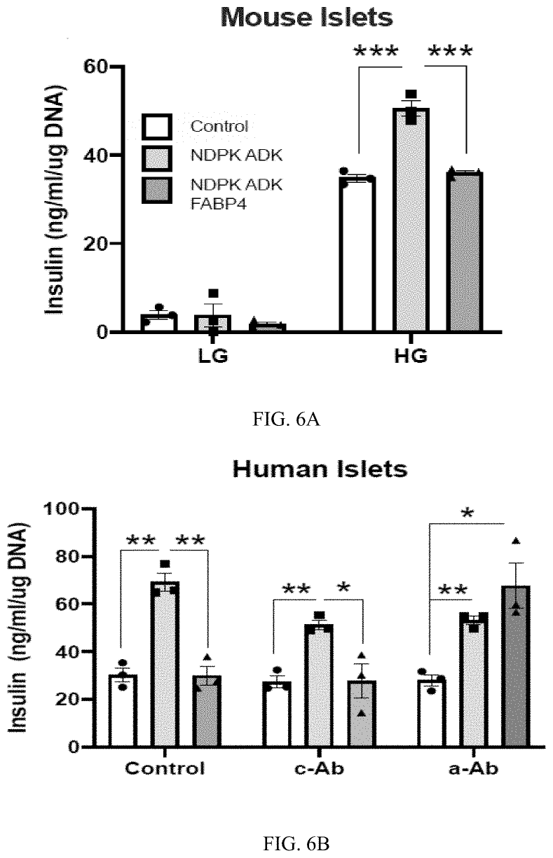

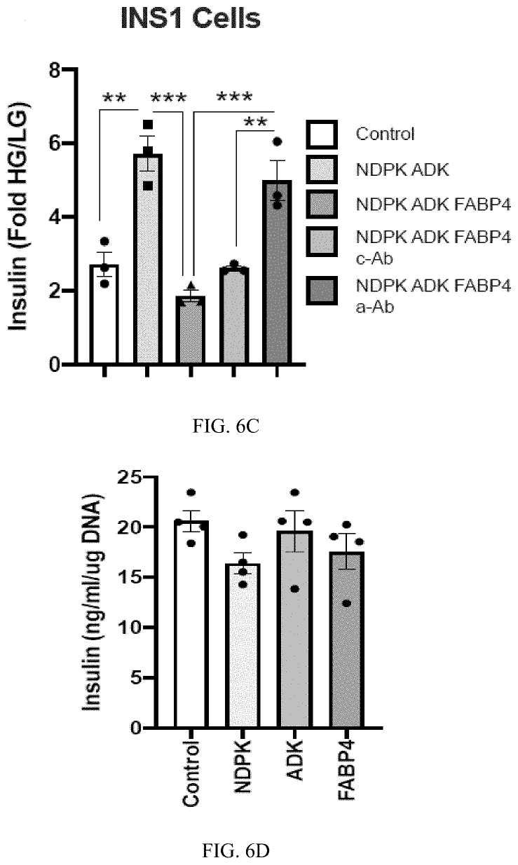

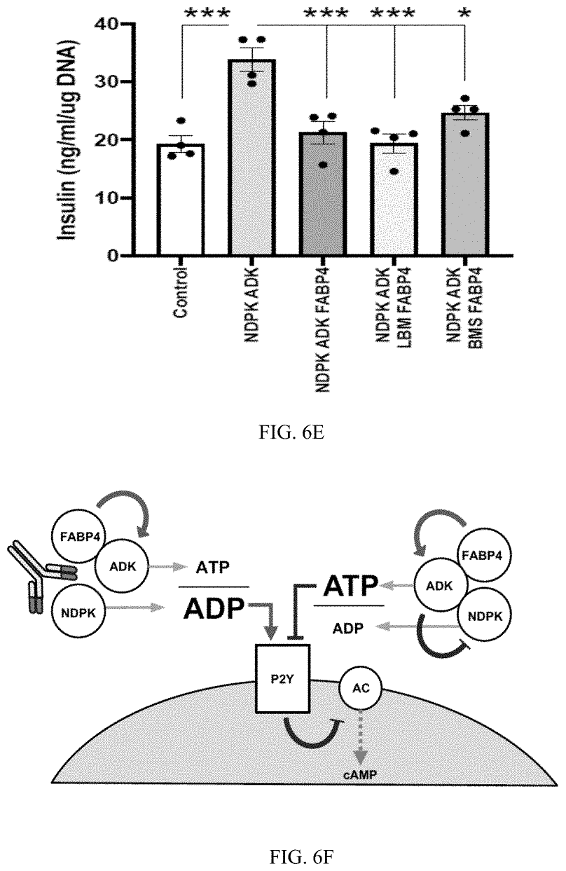

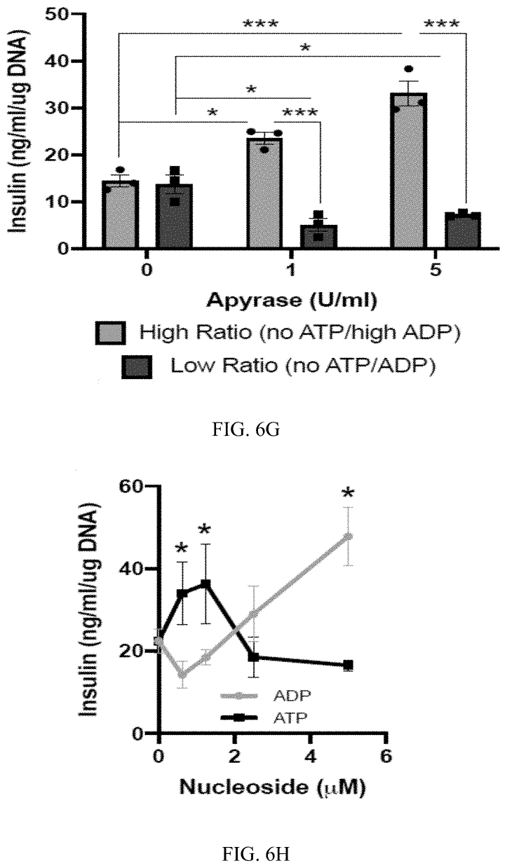

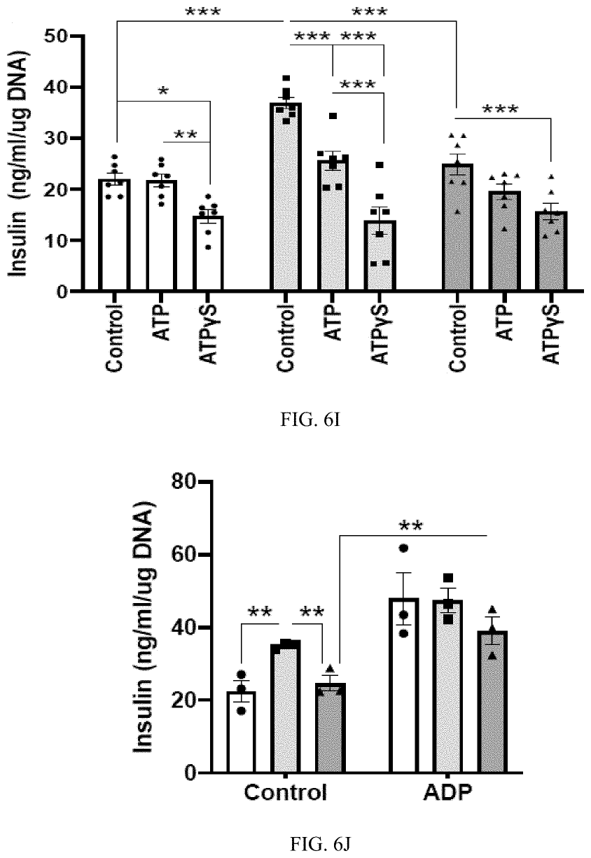

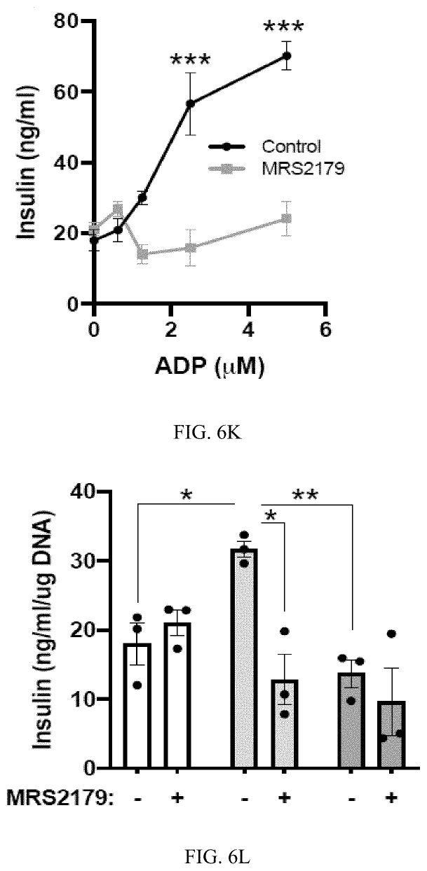

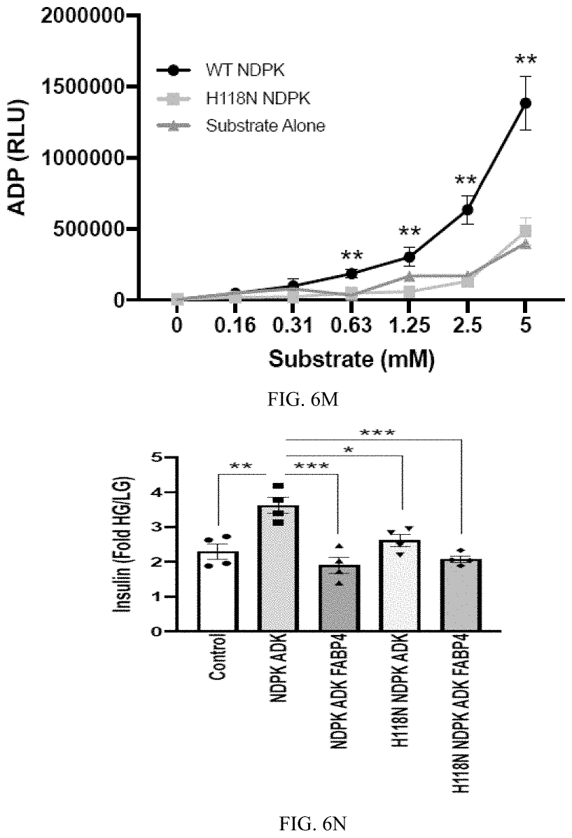

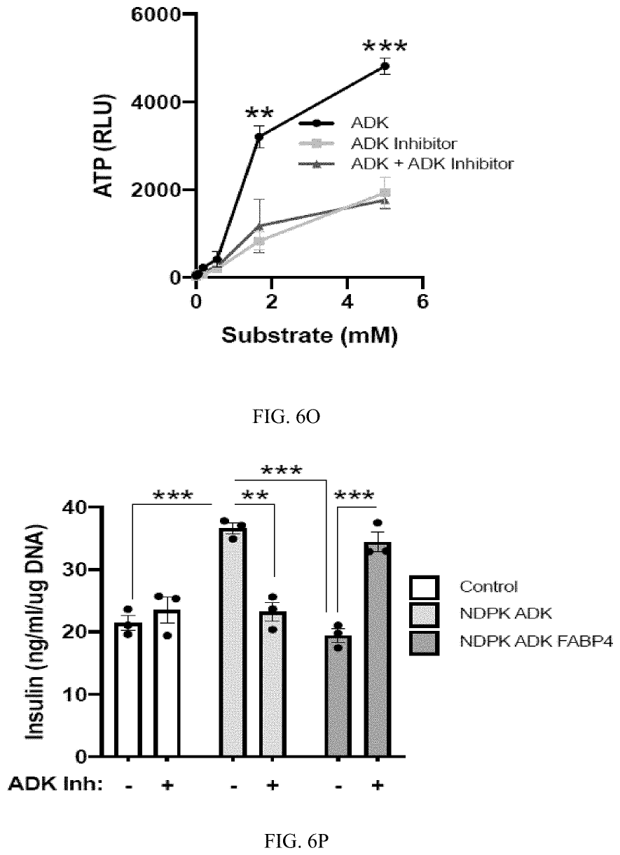

In an additional aspect, provided herein is a method of identifying a compound capable of increasing glucose-stimulated insulin secretion (GSIS) in the presence of FABP4 comprising contacting FABP4, NDPK, ADK, NDPK-ADK complex, and/or NDPK-ADK/FABP4 complex with an insulin-secreting cell line in the presence of a compound and contacting FABP4, NDPK, ADK, NDPK-ADK complex, and/or NDPK-ADK/FABP4 complex with an insulin-secreting cell line in the absence of the compound and measuring the level of insulin secretion in the presence of the compound and in the absence of the compound. An increase in the extracellular ratio of ADP to ATP or the level of insulin secretion in the presence of the compound is indicative of a compound capable of increasing glucose-stimulated insulin secretion (GSIS) in the presence of FABP4. See, for example, as exemplified in Example 5 and FIGS. 6A-6P.

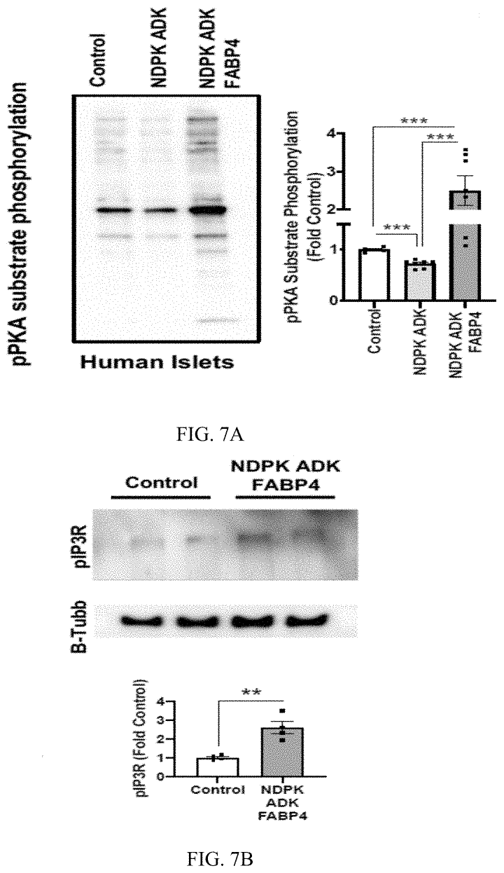

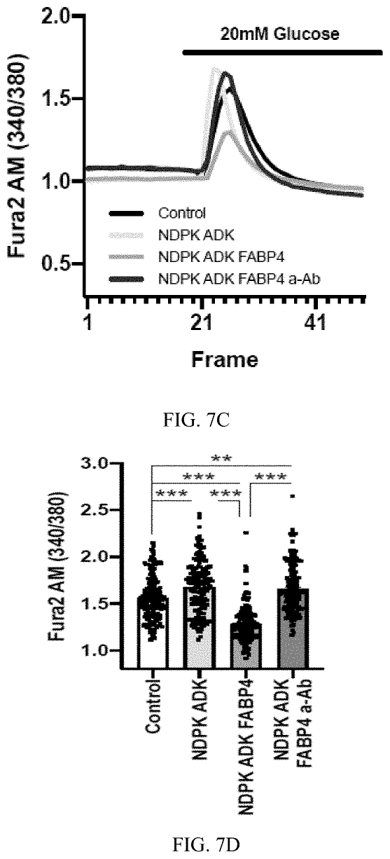

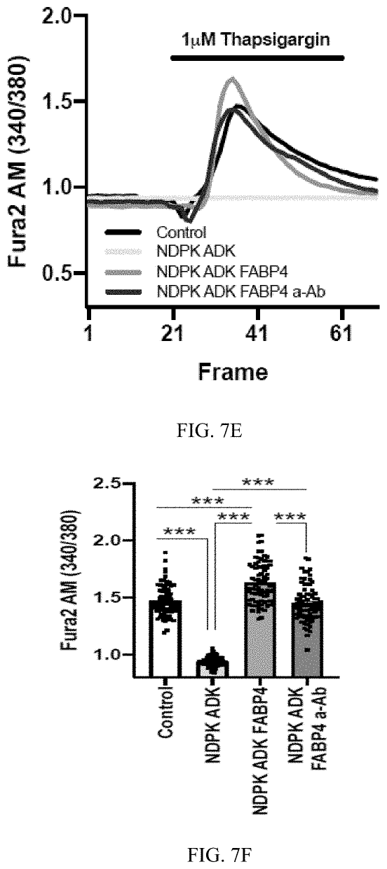

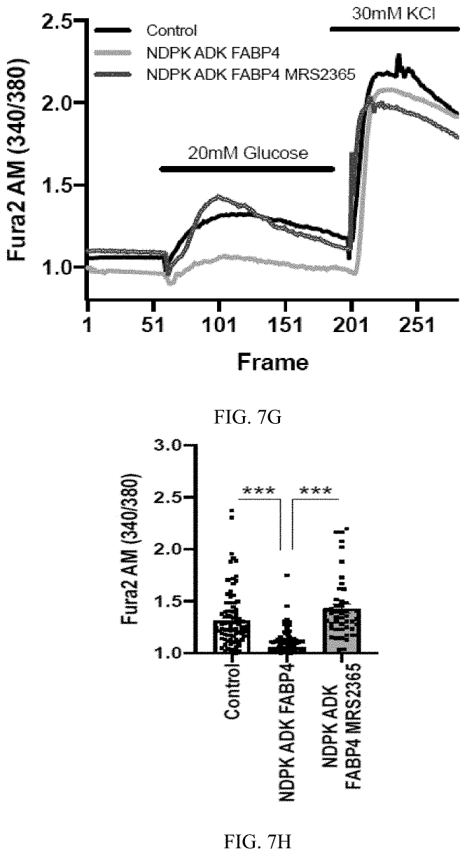

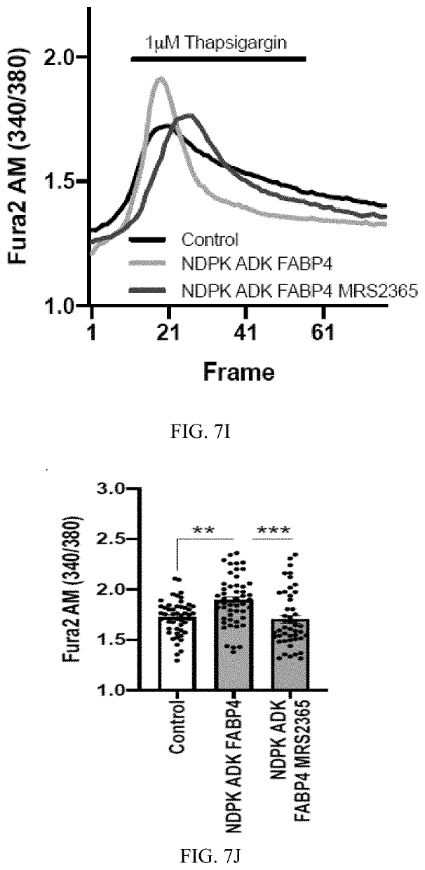

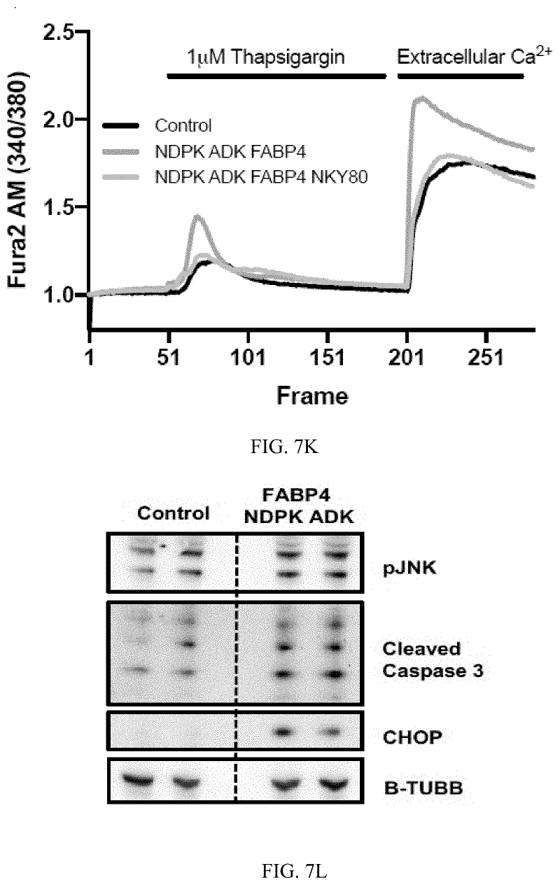

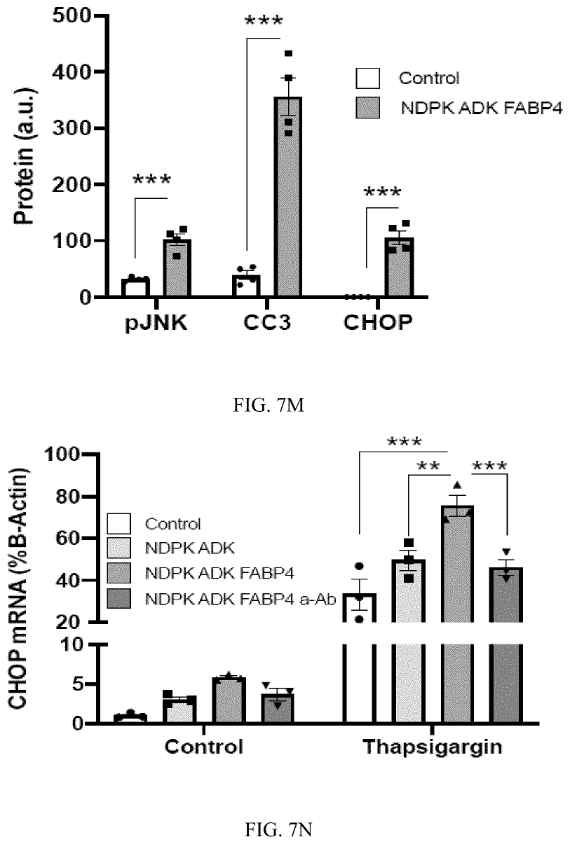

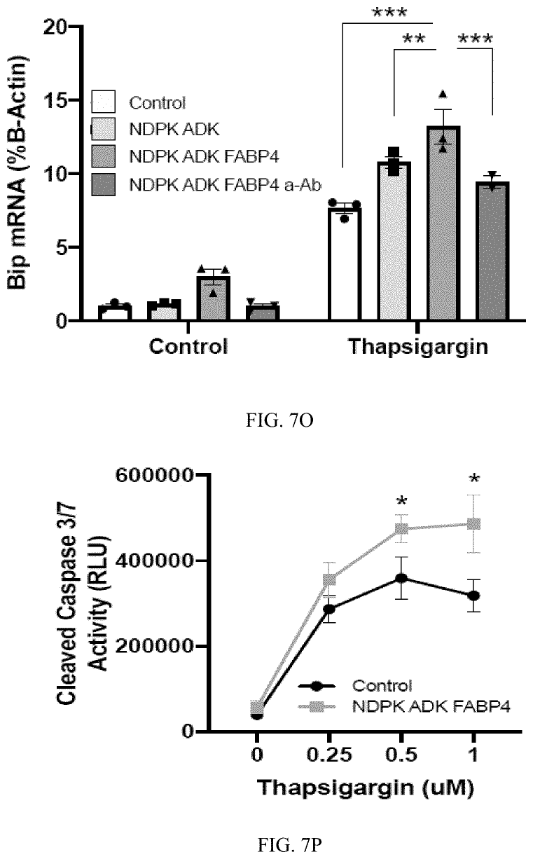

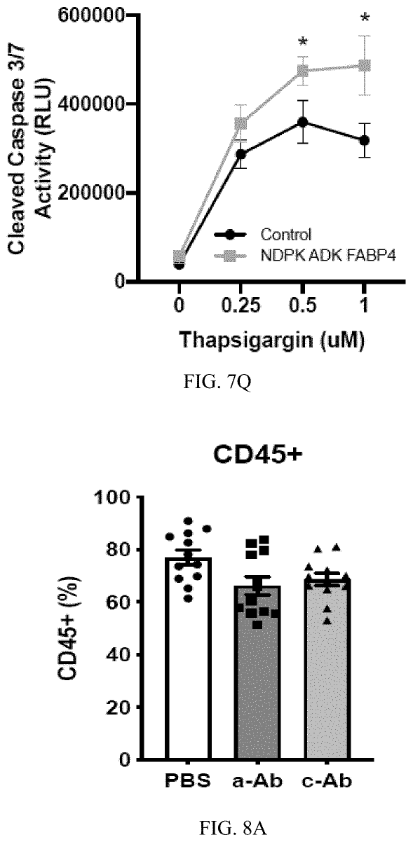

In an additional aspect, provided herein is a method of identifying a compound capable of preserving pancreatic islet beta cell activity in the presence of FABP4 comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the level of pPKA substrate phosphorylation in the presence of the compound and in the absence of the compound. A decrease in the level of pPKA substrate phosphorylation in the presence of the compound is indicative of a compound capable of reducing pPKA substrate phosphorylation in the presence of FABP4. See for example, as exemplified in Example 6 and FIG. 7A.

In an additional aspect, provided herein is a method of identifying a compound capable of preserving pancreatic islet beta cell activity in the presence of FABP4 comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the level of phosphorylation of the endoplasmic reticulum (ER) calcium efflux transporter IP3R in the presence of the compound and in the absence of the compound. A lower level of IPR3 phosphorylation in the presence of the compound is indicative of a compound capable preserving pancreatic islet beta cell activity in the presence of FABP4. See for example as exemplified in Example 6 and FIG. 7B.

In an additional aspect, provided herein is a method of identifying a compound capable of preserving pancreatic islet beta cell activity in the presence of FABP4 comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the level of cAMP formation in the presence of the compound and in the absence of the compound. A lower level of cAMP formation in the presence of the compound is indicative of a compound capable of preserving pancreatic islet beta cell activity. See for example as exemplified in Example 6 and FIG. 7A.

In an additional aspect, provided herein is a method of identifying a compound capable of preserving pancreatic islet beta cell activity in the presence of FABP4 comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the adenylyl cyclase activation in the presence of the compound and in the absence of the compound. A reduction of adenylyl cyclase activation in the presence of the compound is indicative of a compound capable of preserving pancreatic islet beta cell activity. See for example as exemplified in Example 6 and FIG. 7K.

In an additional aspect, provided herein is a method of identifying a compound capable of preserving pancreatic islet beta cell activity in the presence of FABP4 comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the intracellular Ca′ concentration in the presence of the compound and in the absence of the compound. A reduction in the intracellular Ca′ concentration in the presence of the compound is indicative of a compound capable of preserving pancreatic islet beta cell activity. See for example as exemplified in Example 6 and FIGS. 7C-7K. In an additional aspect, provided herein is a method of identifying a compound capable of preserving pancreatic beta cell activity comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the accumulation of misprocessed or misfolded proteins in the presence of the compound and in the absence of the compound. A lower level of misprocessed or misfolded proteins in the presence of the compound is indicative of a compound capable of capable of preserving pancreatic beta cell activity. See for example as exemplified in Example 6 and FIGS. 7N-O.

In an additional aspect, provided herein is a method of identifying a compound capable of preserving pancreatic beta cell activity comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the level of endoplasmic reticulum stress marker C/-EBP homologous protein (CHOP) in the presence of the compound and in the absence of the compound. A reduced level of CHOP in the presence of the compound is indicative of a compound capable of capable of preserving pancreatic beta cell activity. See for example as exemplified in Example 6 and FIGS. 7N-O. In an additional aspect, provided herein is a method of identifying a compound capable of preserving pancreatic beta cell activity comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the level of endoplasmic reticulum stress marker immunoglobulin heavy chain-binding protein (Bip (Grp78)) in the presence of the compound and in the absence of the compound. A lower level of Bip (Grp78) in the presence of the compound is indicative of a compound capable of capable of preserving pancreatic beta cell activity. See for example as exemplified in Example 6 and FIGS. 7N-O. In an additional aspect, provided herein is a method of identifying a compound capable of preserving pancreatic beta cell activity comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the activity of cleaved caspase 3 (CC3) in the presence of the compound and in the absence of the compound. A reduction in CC3 activity in the presence of the compound is indicative of a compound capable of capable of preserving pancreatic beta cell activity. See for example, as exemplified in Example 6 and FIGS. 6G and 7L.

In an additional aspect, provided herein is a method of identifying a compound capable of preserving pancreatic beta cell activity comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the level of c-Jun N-terminal kinase (JNK) phosphorylation in the presence of the compound and in the absence of the compound. A lower level JNK phosphorylation in the presence of the compound is indicative of a compound capable of capable of preserving pancreatic beta cell activity. See for example as exemplified in Example 6 and FIGS. 6P and 7L.

In an additional aspect, provided herein is a method of identifying a compound capable of preserving pancreatic beta cell activity comprising contacting FABP4, NDPK, ADK, NDPK-ADK complex, and/or NDPK-ADK/FABP4 complex with an insulin-secreting cell line in the presence of the compound and contacting FABP4, NDPK, ADK, NDPK-ADK complex, and/or NDPK-ADK/FABP4 complex with an insulin-secreting cell line in the absence of the compound and measuring the activity of cleaved caspase 3/7 (CC3/7) in the presence of the compound and in the absence of the compound. A reduction in CC3/7 activity in the presence of the compound is indicative of a compound capable of capable of preserving pancreatic beta cell activity. See for example as exemplified in Example 6 and FIG. 7P.

In addition, assays may measure the formation of complexes between NDPK, ADK, NDPK-ADK complex, and/or NDPK-ADK/FABP4 complex and the compound being tested or examine the degree to which NDPK-ADK complex can modulate GPCRs in the presence of FABP4 and the compound being tested. Thus, the present invention provides methods of identifying compounds comprising contacting a compound with NDPK, ADK, NDPK-ADK complex, and/or NDPK-ADK/FABP4 complex and assaying (i) for the presence of a complex between FABP4 and NDPK-ADK complex in the presence of the compound or (ii) for the ability of NDPK-ADK complex to modulate GPCRs in the presence of FABP4 and the compound. In such competitive binding assays, NDPK, ADK, NDPK-ADK complex, NDPK-ADK/FABP4 complex, and/or their substrates can be labelled. For example, free NDPK-ADK complex is separated from that present in a complex with FABP4 and the amount of free (i.e., uncomplexed) label is a measure of the ability of the compound to inhibit binding of FABP4 to the NDPK-ADK complex. Examples of competitive binding assays that can be utilized include biolayer interferometry with direct interaction of FABP4 with biotinylated NDPK, scintillation proximity assay, in which ¹²⁵I-NDPK interacted with biotinylated FABP4, isothermal titration calorimetry, which measures heat liberated from binding events in solution and microscale thermophoresis

The identification of a compound capable of neutralizing the ability of FABP4 to inhibit NDPK-ADK complex modulation of GPCRs can further be confirmed in additional assays, for example, cell based biological assays or cell-free phosphorylation assays. A sequence for facilitating the detection or purification of active NDPK-ADK or NDPK-ADK/FABP4, such as the sequence containing a histidine residue or a continuous sequence thereof (poly-His), a c-Myc partial peptide (Myc-tag), a hemagglutinin partial peptide (HA-tag), a Flag partial peptide (Flag-tag), a glutathione-S-transferase (GST), a maltose-binding protein (MBP), biotinylation, labeling with a fluorescent substance (such as a fluorescein), an Eu chelate, a chromophore, a luminophore, an enzyme, or a radioisotope (such as ¹²⁵I or tritium); or binding of a compound having a hydroxysuccinimide residue, a vinyl pyridine residue, etc. for facilitating the binding to a solid phase (such as a container or a carrier), may be introduced into the amino terminal, the carboxy terminal, or an intermediate region of the amino acid sequence of FABP4, NDPK, ADK, or the compound, if the compound is an antibody or fragment thereof, and such proteins can be used during the screen.

In some embodiments, the present invention provides a method of identifying compounds capable of neutralizing the ability of FABP4 to inhibit NDPK-ADK complex modulation of GPCRs utilizing pancreatic islet β-cells expressing GPCRs and analyzing the biological effects the compound has on NDPK-ADK complex modulation of the GPCRs in the presence of FABP4. Such cells, either in viable or fixed form, can be used for standard binding assays. For example, the assay may measure NDPK-ADK complex modulation of GPCRs in the presence of FABP4 and the compound or examine the degree to which biological activity of GPCRs in the presence of NDPK-ADK complex and FABP4 is increased in the presence of the compound. Thus, the present invention provides methods of identifying compounds comprising contacting a compound and FABP4, NDPK, ADK, NDPK-ADK complex, and/or NDPK-ADK/FABP4 complex and assaying (i) for the presence NDPK-ADK complex modulation of GPCRs or (ii) for modulation of GPCRs by measuring the biological effect of the GPCR. The influence of the compound on a biological activity of the GPCR can be determined by methods well known in the art. In such activity assays the biological activity of GPCR is typically monitored by provision of a reporter system. For example, this may involve provision of a natural or synthetic substrate that generates a detectable signal in proportion to the degree to which it is acted upon by the biological activity of GPCR modulation, for example, in increase of extracellular ADP compared to ATP, the measurement of insulin secretion in response to glucose, increase in GTP, intracellular free Ca2⁺ concentrations, cyclic AMP (cAMP) generation, inositol 1,4,5-trisphosphate (IP3) generation, level of cleaved caspase 3 (CC3), level of cleaved caspase 3/7 (CC3/7), level of c-Jun N-terminal kinase (JNK) phosphorylation, level of endoplasmic reticulum stress marker immunoglobulin heavy chain-binding protein (Bip (Grp78)), level of endoplasmic reticulum stress marker C/-EBP homologous protein (CHOP), the accumulation of misprocessed or misfolded proteins, and/or diacylglycerol (DAG) generation.

The cell-based assay includes a cell that expresses purinergic GPCR(s), either endogenously or recombinantly. The GPCR(s), as expressed, should be capable of eliciting a measurable biological affect upon modulation by the NDPK-ADK complex in the presence of its endogenous ligand. The GPCR(s) may be derived from any organism such as human beings, mice, rat, cattle, pig, or rabbit. In some embodiments, the GPCR(s) expressed are those naturally occurring in pancreatic islet β-cells. GPCRs expressed in islet β-cells are known in the art (see, e.g., Amisten S, Salehi A, Rorsman P, Jones P M, Persaud S J. An atlas and functional analysis of G-protein coupled receptors in human islets of Langerhans. Pharmacol Ther. 2013; 139(3):359-91. Epub 2013/05/23, incorporated herein by reference). The GPCR(s) for use in the assay may be extracted from a cell or tissue existing in nature and may be extracted from a cell or tissue which expresses the subunit by a genetic engineering procedure. The GPCR(s) may be purified or unpurified. The GPCR(s) produced by a genetic engineering procedure having a reported amino acid sequence or a variant amino acid sequence obtained by genetic mutation can be used as long as it substantially maintains the activity. In some embodiments, the GPCR is the human P2Y1R.

In some embodiments, the assay is a cell-free assay and the compound is brought into contact with the NDPK-ADK/FABP4 complex in a liquid phase, or alternatively the NDPK-ADK/FABP4 complex is fixed to a solid phase (such as a column) and then contacted with the compound. For example, the NDPK-ADK/FABP4 complex may be fixed to the solid phase by biotin/streptavidin, by using a reactable amino group, such as a hydroxysuccinimide group, by using a reactive carboxyl group on a surface, such as a hydrazine group, or by using a group reactable with a thiol group on a surface, such as a vinyl pyridine group. For example, NDPK-ADK/FABP4 complex may be fixed to the solid phase (such as a column) by attaching to a solid phase composed of a polystyrene resin or a glass using the electrostatic attractive force or the intermolecular force, by binding NDPK-ADK/FABP4 complex to a solid phase obtained by immobilizing an antibody against an amino acid sequence added to FABP4 and/or NDPK-ADK/FABP4 (such as poly-His, Myc-tag, HA-tag, Flag-tag, GST, or MBP), by NDPK-ADK/FABP4 attached with poly-His to a solid phase having on the surface a metal chelate, by binding NDPK-ADK/FABP4 attached with GST to a solid phase having on the surface a glutathione, or by binding NDPK-ADK/FABP4 attached with MBP to a solid phase having on the surface a sugar such as maltose. NDPK-ADK/FABP4 may also be fixed to the solid phase by another generally known method.

The contacting step of the compound with NDPK-ADK/FABP4 complex may be conducted, for example, by mixing a solution containing them. Alternatively, if, for example, NDPK-ADK/FABP4 complex or, alternatively the compound, is fixed to a solid phase such as a column, tube, or a multi-well plate, adding a solution containing the non-bound compound.

In some embodiments, the assay is a cell-based assay, wherein the method for identifying the compound by measuring the ability of the NDPK-ADK complex to modulate the GPCR(s) in the presence of FABP4 and the compound uses a cell, a tissue, or an extract thereof containing the GPCR(s). The cell or tissue substantially containing the GPCR(s) may be derived from any organism and may be any cell or tissue, although preferably a mammal cell or tissue, including a human cell or tissue. The cell or tissue may be one in which the GPCR(s) is endogenously expressed or is expressed by a genetic engineering procedure. In some embodiments, the cell is a pancreatic islet β-cell.

In some embodiments, a cell population expressing GPCR(s) is contacted with a solution comprising FABP4, NDPK, ADK, and/or NDPK-ADK complex, and the modulation of the GPCR(s) is measured in the presence and absence of a compound. GPCR modulation generally refers to any observable effect resulting from the interaction between the GPCR and its ligand. The biological activity may be NDPK-ADK complex modulating a GPCR, detection of GPCR-mediated intracellular signal transduction; or determination of an end-point physiological effect, for example insulin secretion. Representative, but non-limiting, examples of GPCR biological activity upon modulation by NDPK-ADK complex include, but are not limited to, signaling and regulation of the processes discussed herein, e.g., the measurement of ADP production, the measurement of insulin secretion in response to glucose, intracellular free Ca2+ concentrations, cyclic AMP (cAMP) generation, increase in GTP, inositol 1,4,5-trisphosphate (IP3) generation, level of cleaved caspase 3 (CC3), level of cleaved caspase 3/7 (CC3/7), level of c-Jun N-terminal kinase (JNK) phosphorylation, level of endoplasmic reticulum stress marker immunoglobulin heavy chain-binding protein (Bip (Grp78)), level of endoplasmic reticulum stress marker C/-EBP homologous protein (CHOP), the accumulation of misprocessed or misfolded proteins, and/or diacylglycerol (DAG) generation.

In some embodiments, the compound is a small molecule, a ligand, an antibody, antigen binding agent, or antibody fragment that binds to FABP4, NDPK, NDPK-ADK complex, and/or NDPK-ADK/FABP4 complex, and neutralizes the ability of FABP4 from inhibiting NDPK-ADK complex modulation of GPCRs. Methods of measuring biological effect of GPCR modulation are known in the art and non-limiting examples of assays to detect GPCR biological activity include, signaling and regulation of the processes discussed herein, e.g., the measurement of ADP production, the measurement of insulin secretion in response to glucose, intracellular free Ca2+ concentrations, cyclic AMP (cAMP) generation, increase in GTP, inositol 1,4,5-trisphosphate (IP3) generation, level of cleaved caspase 3 (CC3), level of cleaved caspase 3/7 (CC3/7), level of c-Jun N-terminal kinase (JNK) phosphorylation, level of endoplasmic reticulum stress marker immunoglobulin heavy chain-binding protein (Bip (Grp78)), level of endoplasmic reticulum stress marker C/-EBP homologous protein (CHOP), the accumulation of misprocessed or misfolded proteins and/or diacylglycerol (DAG) generation. In one non-limiting illustrative example, the cellular assay can be performed with varying concentrations of FABP4, NDPK, ADK, and/or NDPK-ADK complex, GPCR(s), and/or compound to confirm, for example, the efficacy of the ability of the compound to interfere in FABP4 inhibition of NDPK-ADK complex modulation of the GPCR(s). For example, as described above, a first cellular assay may be conducted as follows. 1 equivalent of the compound of interest is added to a solution of cells expressing GPCR(s) in the presence of 1 equivalent of FABP4 and 1 equivalent of NDPK-ADK complex. The modulation of the GPCR(s) is then measured using any method described herein or known in the art. In a typical embodiment, the concentration of the compound of interest is equal to or higher than that of FABP4 and NDPK-ADK complex in the cellular assay. In some embodiments, the concentration of the compound of interest is about 1, 2, 3, 4, 5, 10, 15, or 20 equivalents and the concentration of FABP4 and NDPK-ADK complex is about 1 equivalent. Methods to measure the modulation of GPCRs in the presence of the compound of interest include those described herein and discussed in the paper by Denis et al., “Probing heterotrimeric G protein activation: Applications to Biased Ligands”, Curr. Pharm. Des. (2012); 18 (2): 128-144.

In one non-limiting illustrative example, a second cellular assay may be conducted as follows. 1 equivalent of the compound of interest is added to a solution of cells expressing GPCR(s) in the presence of 20 equivalents of FABP4 and 20 equivalents of NDPK-ADK complex. The activity of GPCR(s) is then measured using any method described herein or known in the art. In a typical embodiment, the concentration of the compound of interest is less than that of FABP4 and NDPK-ADK complex in the cellular assay (i.e., FABP4 and NDPK-ADK complex are saturated with respect to the compound of interest). In some embodiments, the concentration of the FABP4 and NDPK is about 5, 10, 15, 20, 25, 30, 35, or 40 equivalents and the concentration of the compound of interest is 1 equivalent.

In some embodiments, the equivalency of the compound of interest to NDPK-ADK complex and FABP4 is not known and instead a concentration of compound is used.

In one non-limiting illustrative example, a cellular assay may be conducted as follows. 0.5 equivalent of the compound of interest is added to a solution of cells expressing GPCR(s) in the presence of 1 equivalent of FABP4 and 1 equivalent of NDPK-ADK complex. The activity of GPCR(s) is then measured using any method described herein or known in the art. Then the assay is serially repeated using 1 equivalent of the compound of interest, followed by 1.5 equivalents, 2 equivalents, etc. In some embodiments, the above procedure is conducted via serial dilution, starting with the highest concentration of compound and diluting it repeatedly to attain the lowest concentration. Methods to measure the activity of GPCR(s) in the presence of the compound of interest include those described herein and discussed in the paper by Denis et al., “Probing heterotrimeric G protein activation: Applications to Biased Ligands”, Curr. Pharm. Des. (2012); 18 (2): 128-144. In some embodiments, the concentration of the compound of interest is varied logarithmically for example 100 equivalents, 10 equivalents, 1 equivalent, and 0.1 equivalents of compound. In another embodiment, the equivalents of compound are not known and instead a concentration of the compound is varied, for example 100 mM, 10 mM, 1 mM, 100 nM, 10 nM, and 1 nM could be the concentrations used.

Methods for identifying a compound, for example an antibody, that selectively bind to NDPK-ADK/FABP4 over FABP4 alone or uncomplexed or NDPK alone or uncomplexed are also provided. Methods for identifying preferably binding antibodies are generally known in the field. In some embodiments, provided herein is a method of identifying an antibody that selectively binds NDPK-ADK/FABP4 over NDPK generally comprising administering to a non-human animal, for example a rabbit, mouse, rat, or goat, a heterologous NDPK-ADK/FABP4 protein complex, for example human NDPK-ADK/FABP4, in order to raise antibodies against the heterologous NDPK-ADK/FABP4 in complex, isolating said antibodies, subjecting said antibodies to one or more binding assays measuring the binding affinity to NDPK-ADK/FABP4 and FABP4 alone or uncomplexed and NDPK alone or uncomplexed, for example a competitive binding assay, wherein antibodies that preferably bind NDPK-ADK/FABP4 over FABP4 and NDPK are isolated for use to sequester NDPK-ADK/FABP4 and allow NDPK-ADK complex modulation of GPCRs. For example, antibodies to NDPK-ADK/FABP4 can be raised using hybridomas derived by standard procedures well known to those skilled in the field of immunology. Preferred methods for determining mAb specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference.

Fusion partner cell lines and methods for fusing and selecting hybridomas and screening for mAbs are well known in the art. The NDPK-ADK/FABP4 specific mAb can be produced in large quantities by injecting hybridoma or transfectoma cells secreting the antibody into the peritoneal cavity of mice and, after appropriate time, harvesting the ascites fluid which contains a high titer of the mAb, and isolating the mAb therefrom. For such in vivo production of the mAb with a non-murine hybridoma (e.g., rat or human), hybridoma cells are preferably grown in irradiated or athymic nude mice. Alternatively, the antibodies can be produced by culturing hybridoma or transfectoma cells in vitro and isolating secreted mAb from the cell culture medium or recombinantly, in eukaryotic or prokaryotic cells.

It should be noted that the methods for identifying the compounds above are considered to be illustrative and not restrictive.

Methods of Treating Disorders Associated with NDPK-ADK/FABP4 Modulation of GPCRs

Methods are provided for treating FABP4-mediated disorders by, for example, i) identifying a compound capable of neutralizing FABP4's ability to inhibit NDPK-ADK complex modulation of GPCRs, and ii) administering such compound to a subject in need thereof. Because of the prominent role the NDPK-ADK complex plays in inducing purinergic GPCRs as demonstrated by insulin secretion in response to glucose levels, neutralizing, either fully or partially, FABP4's negative effects on NDPK-ADK complex modulation of the GPCRs has the ability to modulate the severity of FABP4-mediated disorders and underlying conditions. In some embodiments, a compound which has been identified as capable of interfering with the formation of the NDPK-ADK/FABP4 complex or the ability of FABP4 to regulate NDPK-ADK complex modulation of GPCRs and administering to a subject having an underlying condition or disorder associated with excessive FABP4 expression.

In some embodiments, a compound, for example, an antibody, antigen-binding agent or antibody-binding fragment, is selected that targets the NDPK-ADK/FABP4 protein complex, including anti-NDPK-ADK/FABP4 protein complex humanized antibody, antigen-binding agent or antibody-binding fragments, and administered for the treatment of FABP4-mediated metabolic disorders involving dysregulated or elevated blood glucose levels, including, but not limited to, diabetes (type I and type II), obesity, and nonalcoholic fatty liver disease (NAFLD), nonalcoholic steatohepatitis (NASH), metabolic disorders, cardiovascular disease, atherosclerosis, fibrosis, cirrhosis, hepatocellular carcinoma, insulin resistance, dyslipidemia, hyperglycemia, hyperglucanemia, hyperinsulinemia, and asthma.

In one aspect, a method is provided for treating a disease or disorder caused by dysregulated insulin secretion and dysregulated or elevated glucose blood levels in a host by administering a compound, for example an antibody, antigen-binding agent or antibody-binding fragment, identified as described herein. In some embodiments, the disorder is a metabolic disorder. In some embodiments, the disorder is diabetes. In some embodiments, the disorder is type I diabetes. In some embodiments, the disorder is type II diabetes. In some embodiments, the disorder is hyperglycemia. In some embodiments, the disorder is obesity. In some embodiments, the disorder is dyslipidemia. In some embodiments, the disorder is nonalcoholic fatty liver disease (NAFLD).

In some embodiments, the disorder is nonalcoholic steatohepatitis (NASH). In some embodiments, the disorder is cardiovascular disease. In some embodiments, the disorder is atherosclerosis. In some embodiments, the disorder is fibrosis. In some embodiments, the disorder is cirrhosis. In some embodiments, the disorder is hepatocellular carcinoma. In some embodiments, the disorder is insulin resistance. In some embodiments, the disorder is dyslipidemia. In some embodiments, the disorder is hyperglucanemia. In some embodiments, the disorder is hyperinsulinemia. In some embodiments, the disorder is asthma.

Diabetes

Diabetes mellitus is the most common metabolic disease worldwide. Every day, 1700 new cases of diabetes are diagnosed in the United States, and at least one-third of the 16 million Americans with diabetes are unaware of it. Diabetes is the leading cause of blindness, renal failure, and lower limb amputations in adults and is a major risk factor for cardiovascular disease and stroke.

In one aspect, a method is provided for treating diabetes by administering to a host an effective amount of a compound, for example an antibody, antigen-binding agent or antibody-binding fragment, identified as described herein. In some embodiments, the disorder is type I diabetes. In some embodiments, the disorder is type II diabetes.

Type I diabetes results from autoimmune destruction of pancreatic beta cells causing insulin deficiency. Type II or non-insulin-dependent diabetes mellitus (NIDDM) accounts for >90% of cases and is characterized by a resistance to insulin action on glucose uptake in peripheral tissues, especially skeletal muscle and adipocytes, impaired insulin action to inhibit hepatic glucose production, and dysregulated insulin secretion.

In some embodiments, provided herein is a method of treating type I diabetes in a host by administering to the host an effective amount of a compound, for example an antibody, antigen-binding agent or antibody-binding fragment, identified as described herein in combination or alteration with insulin. In some embodiments, provided herein is a method of treating type I diabetes in a host by administering to the host of a compound, for example an antibody, antigen-binding agent or antibody-binding fragment, identified as described herein in combination or alteration with a synthetic insulin analog.

In some embodiments, provided herein is a method of preventing type I diabetes in a host by administering to the host an effective amount of an antibody, antigen-binding agent or antibody-binding fragment identified as described herein.

In some embodiments, provided herein is a method of preventing pancreatic islet β-cell dysfunction in a host by administering to the host an effective amount of an antibody, antigen-binding agent or antibody-binding fragment identified as described herein.

Some people who have type II diabetes can achieve their target blood sugar levels with diet and exercise alone, but many also need diabetes medications or insulin therapy. In some embodiments, provided herein is a method of treating type II diabetes in a host by administering to the host an effective amount of an antibody, antigen-binding agent or antibody-binding fragment described herein. In some embodiments, provided herein is a method of treating a disease or condition associated with diabetes by administering to a host an effective amount of an antibody, antigen-binding agent or antibody-binding fragment described herein. Diseases and conditions associated with diabetes mellitus can include, but are not restricted to, hyperglycemia, hyperlipidemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis and ulcerative colitis. Furthermore, diseases and conditions associated with diabetes mellitus comprise, but are not restricted to: coronary heart disease, hypertension, angina pectoris, myocardial infarction, stroke, skin and connective tissue disorders, foot ulcerations, metabolic acidosis, arthritis, osteoporosis and in particular conditions of impaired glucose tolerance.

### Body Weight Disorders

In some embodiments, a method is provided for treating obesity due to dysregulated insulin secretion or elevated blood glucose levels in a host by administering an effective amount of a compound, for example an antibody, antigen-binding agent or antibody-binding fragment, identified as described herein. Obesity represents the most prevalent of body weight disorders, affecting an estimated 30 to 50% of the middle-aged population in the western world.

In some embodiments, a method is provided for treating obesity in a host by administering a compound, for example an antibody, antigen-binding agent or antibody-binding fragment, identified as described herein. In some embodiments, a method is provided for reducing or inhibiting weight gain caused by dysregulated insulin secretion or elevated blood glucose in a host by administering a compound, for example an antibody, antigen-binding agent or antibody-binding fragment, identified as described herein.

### Nonalcoholic Fatty Liver Disease (NAFLD)

There is a need for compositions and methods for the treatment and prevention of the development of fatty liver and conditions stemming from fatty liver, such as nonalcoholic steatohepatitis (NASH), liver inflammation, cirrhosis and liver failure caused by dysregulation of glucose levels or elevated blood glucose levels and chronic hyperglycemia. In some embodiments, a method is provided for treating NAFLD in a host by administering a compound, for example an antibody, antigen-binding agent or antibody-binding fragment, identified as described herein.

### Nonalcoholic Steatohepatitis (NASH)

Nonalcoholic steatohepatitis (NASH), which is an advanced form of nonalcoholic fatty liver disease (NAFLD), refers to the accumulation of hepatic steatosis not due to excess alcohol consumption. NASH is a liver disease characterized by inflammation of the liver with concurrent fat accumulation. NASH is also frequently found in people with diabetes and obesity and is related to metabolic syndrome. NASH is the progressive form of the relatively benign non-alcoholic fatty liver disease, for it can slowly worsen causing fibrosis accumulation in the liver, which leads to cirrhosis (reviewed in Smith et al., (2011), Crit. Rev. Clin. Lab. Sci., 48(3):97-113). Currently, no approved therapies for NASH exist.

In some embodiments, a method is provided for treating NASH in a host by administering a compound, for example an antibody, antigen-binding agent or antibody-binding fragment, identified as described herein.

### Glucagonoma and Necrolytic Migratory Erythema

A glucagonoma is a rare tumor of the alpha cells of the pancreas that results in the overproduction of the hormone glucagon. The primary physiological effect of glucagonoma is an overproduction of the peptide hormone glucagon. Necrolytic migratory erythema (NME) is a classical symptom observed in patients with glucagonoma and is the presenting problem in 70% of cases (van Beek et al., (November 2004). “The glucagonoma syndrome and necrolytic migratory erythema: a clinical review”. Eur. J. Endocrinol. 151 (5): 531-7). Associated NME is characterized by the spread of erythematous blisters and swelling across areas subject to greater friction and pressure, including the lower abdomen, buttocks, perineum, and groin.

In some embodiments, a method is provided to treat glucagonoma and/or necrolytic migratory erythema (NME) in a host by administering a compound, for example an antibody, antigen-binding agent or antibody-binding fragment, identified as described herein.

### Metabolic Disorders

In one aspect, a method is provided for treating metabolic disorder in a host mediated by dysregulated insulin secretion or elevated blood glucose levels by administering an effective amount of an antibody, antigen-binding agent or antibody-binding fragment described herein. A metabolic disorder includes a disorder, disease, or condition which is caused or characterized by an abnormal metabolism (i.e., the chemical changes in living cells by which energy is provided for vital processes and activities) in a subject. Metabolic disorders include diseases, disorders, or conditions associated with hyperglycemia. Metabolic disorders can detrimentally affect cellular functions such as cellular proliferation, growth, differentiation, or migration, cellular regulation of homeostasis, inter- or intra-cellular communication; tissue function, such as liver function, renal function, or adipocyte function; systemic responses in an organism, such as hormonal responses (e.g., insulin response). Examples of metabolic disorders include obesity, diabetes, hyperphagia, endocrine abnormalities, triglyceride storage disease, Bardet-Biedl syndrome, Laurence-Moon syndrome, Prader-Labhart-Willi syndrome, and disorders of lipid metabolism.

In other aspects, methods are provided for reducing fasting blood glucose levels, reducing fat mass levels, reducing hepatic glucose production, reducing fat cell lipolysis, reducing liver steatosis, increasing glucose metabolism, increasing insulin sensitivity, increasing insulin secretion and production, preventing β-cell death, dysfunction, or loss comprising administering a compound, for example an antibody, antigen-binding agent or antibody-binding fragment, identified as described herein to a host, typically a human, in need thereof

Inflammatory Disorders

In one aspect, a method is provided for treating an inflammatory disorder in a host mediated by dysregulated insulin secretion or elevated blood glucose levels by administering a compound, for example an antibody, antigen-binding agent or antibody-binding fragment, identified as described herein. Inflammation is known to occur in many disorders which include, but are not limited to: severe acute respiratory syndrome coronavirus 2 (SARS-CoV-2), Systemic Inflammatory Response (SIRS); Alzheimer's Disease (and associated conditions and symptoms including: chronic neuroinflammation, glial activation; increased microglia; neuritic plaque formation; Amyotrophic Lateral Sclerosis (ALS), arthritis (and associated conditions and symptoms including, but not limited to: acute joint inflammation, antigen-induced arthritis, arthritis associated with chronic lymphocytic thyroiditis, collagen-induced arthritis, juvenile arthritis, rheumatoid arthritis, osteoarthritis, prognosis and streptococcus-induced arthritis, spondyloarthropathies, and gouty arthritis), asthma (and associated conditions and symptoms, including: bronchial asthma; chronic obstructive airway disease, chronic obstructive pulmonary disease, juvenile asthma and occupational asthma); cardiovascular diseases (and associated conditions and symptoms, including atherosclerosis, autoimmune myocarditis, chronic cardiac hypoxia, congestive heart failure, coronary artery disease, cardiomyopathy and cardiac cell dysfunction, including: aortic smooth muscle cell activation, cardiac cell apoptosis and immunomodulation of cardiac cell function); diabetes (and associated conditions, including autoimmune diabetes, insulin-dependent (Type I) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, cholesterol gallstones and hepatic fibrosis; HIV infection (and associated conditions, including—degenerative responses, neurodegenerative responses, and HIV associated Hodgkin's Disease); Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis); nephropathies (and associated diseases and conditions, including diabetic nephropathy, endstage renal disease, acute and chronic glomerulonephritis, acute and chronic interstitial nephritis, lupus nephritis, Goodpasture's syndrome, hemodialysis survival and renal ischemic reperfusion injury); neurodegenerative diseases or neuropathological conditions (and associated diseases and conditions, including acute neurodegeneration, induction of IL-I in aging and neurodegenerative disease, IL-I induced plasticity of hypothalamic neurons and chronic stress hyperresponsiveness, myelopathy); ophthalmopathies (and associated diseases and conditions, including diabetic retinopathy, Graves' ophthalmopathy, inflammation associated with corneal injury or infection including corneal ulceration, and uveitis), osteoporosis (and associated diseases and conditions, including alveolar, femoral, radial, vertebral or wrist bone loss or fracture incidence, postmenopausal bone loss, fracture incidence or rate of bone loss); otitis media (adult or pediatric); pancreatitis or pancreatic acinitis; periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis; immunological disorders, including autoimmune diseases, such as alopecia aerata, autoimmune myocarditis, Graves' disease, Graves ophthalmopathy, lichen sclerosis, multiple sclerosis, psoriasis, systemic lupus erythematosus, systemic sclerosis, thyroid diseases (e.g. goitre and struma lymphomatosa (Hashimoto's thyroiditis, lymphadenoid goitre); lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries), septicaemia, acute phase response (e.g. febrile response), general inflammatory response, acute respiratory distress response, acute systemic inflammatory response, wound healing, adhesion, immuno-inflammatory response, neuroendocrine response, fever development and resistance, acute-phase response, stress response, disease susceptibility, repetitive motion stress, tennis elbow, and pain management and response.

Cancer

In one aspect, a method is provided for treating a proliferative disorder such as a tumor, cancer or neoplasm in a host mediated by FABP4 by administering a compound, for example an antibody, antigen-binding agent or antibody-binding fragment, identified as described herein.

FABP4-mediated cancers include, but are not limited to, ovarian cancer, breast cancer, cervical cancer, head & neck cancer, urothelial cancer, prostate cancer, or glioblastoma.

Pancreatic Islet Beta-Cell Mass

In one aspect, a method is provided for reducing pancreatic islet beta-cell mass loss in a host mediated by dysregulated insulin secretion or elevated blood glucose levels by administering a compound, for example an antibody, antigen-binding agent or antibody-binding fragment, identified as described herein.

In one aspect, a method is provided for increasing pancreatic islet beta-cell mass in a host mediated by dysregulated insulin secretion or elevated blood glucose levels by administering a compound, for example an antibody, antigen-binding agent or antibody-binding fragment, identified as described herein.

Method of Diagnosing and Preventing Type I Diabetes

It has also been discovered that FABP4 serum levels are significantly elevated in human serum following the onset of type I diabetes (T1D). This elevation persists through the early phases of disease, despite significant reductions in body mass index (BMI), which has previously been associated with increased levels of serum FABP4 levels. In light of these findings, and the discovery that the onset of T1D diabetes may be inhibited by the methods described herein, the discovery provides a mechanism to monitor for, and inhibit the development of, T1D.

Accordingly, in one aspect, provided herein is a method of monitoring and inhibiting the development of the onset of type I diabetes in a subject comprising:

a. measuring an FABP4 baseline level in serum of a type I diabetes pre-onset subject at a first time point;

b. measuring the FABP4 level in serum of the subject at one or more subsequent time points;

c. if the subject's FABP4 levels in the one or more subsequent time points is significantly greater than the subject's FABP4 level at a first time point, administering to the subject an effective amount of a compound that neutralizes the ability of FABP4 to associate with the NDPK-ADK complex, prevents or inhibits the NDPK-ADK/FABP4 complex from generating ATP to antagonize purinergic G protein-coupled receptors (GPCRs) or channels on target cells, or generates ADP to agonize P2Y1Rs on pancreatic islet β-cells.

Methods of analyzing serum samples FABP4 levels, include using a commercially available enzyme-linked immunosorbent assay (ELISA) kit and a human FABP4 standard curve (R&D DY3150-05, USA).

### EXAMPLES

### Example 1. Targeting FABP4 Increases Beta Cell Mass and Insulin Secretion

Given the essential role for appropriate beta cell function and insulin secretion in the pathogenesis of diabetes, the pancreata of FABP4 deficient (FABP4^{-/-}) mice were examined for beta cell mass and insulin content. Animal care and experimental procedures were performed with approval from animal care committees of Harvard University. All mice were housed on a 12-hour light/dark cycle with ad libitum access to chow (PicoLab 5058 LabDiet) and water. FABP4−/− and WT mice were littermates obtained from heterozygous breeding. High fat diet (HFD) fed mice were provided ad libitum access to HFD (60% kcal from fat; Research Diets Inc., D12492i) for 14 weeks beginning at 4 weeks of age. Pancreata and isolated islets were obtained from 7-8-week-old male mice. Blood was collected every other week for 6 hour fasting plasma insulin and FABP4 levels. For acute FABP4 treatment, 10 μg of human recombinant FABP4 (produced in-house) or sterile PBS was injected intraperitoneally. Blood was collected from the tail vein at 0, 10, 20, 30, 60, 120, 240- and 360-minutes post injection for measurement of blood glucose, plasma insulin, and FABP4 levels.

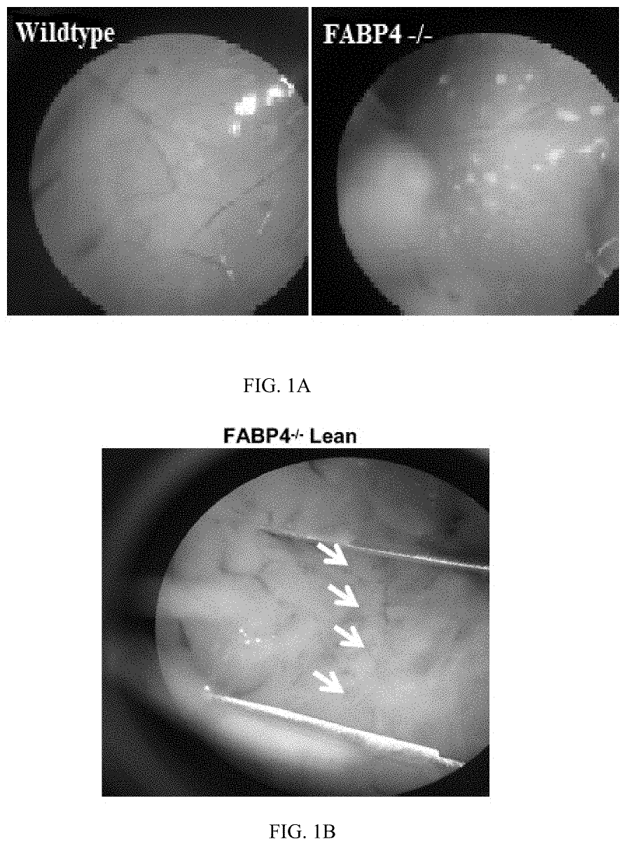

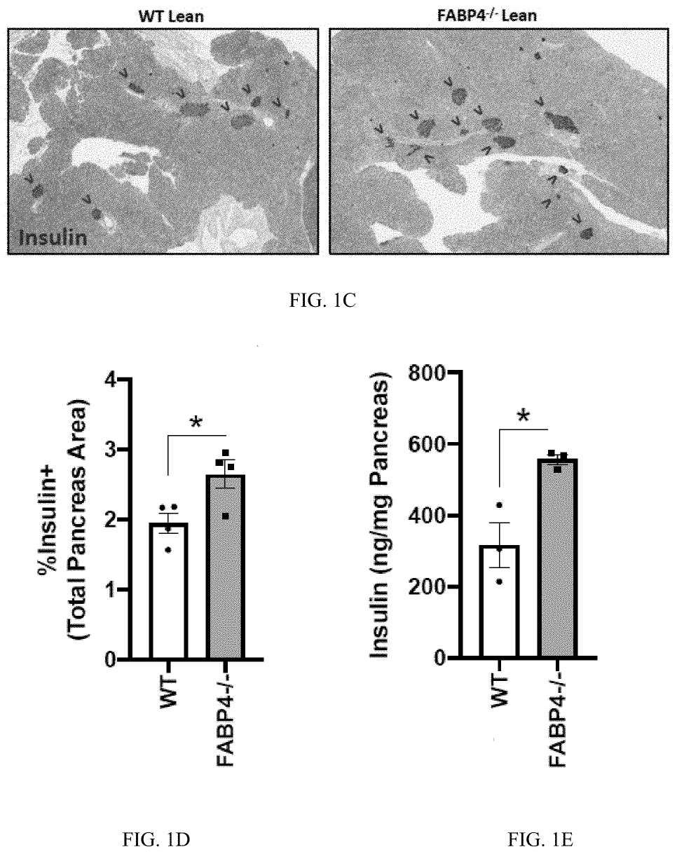

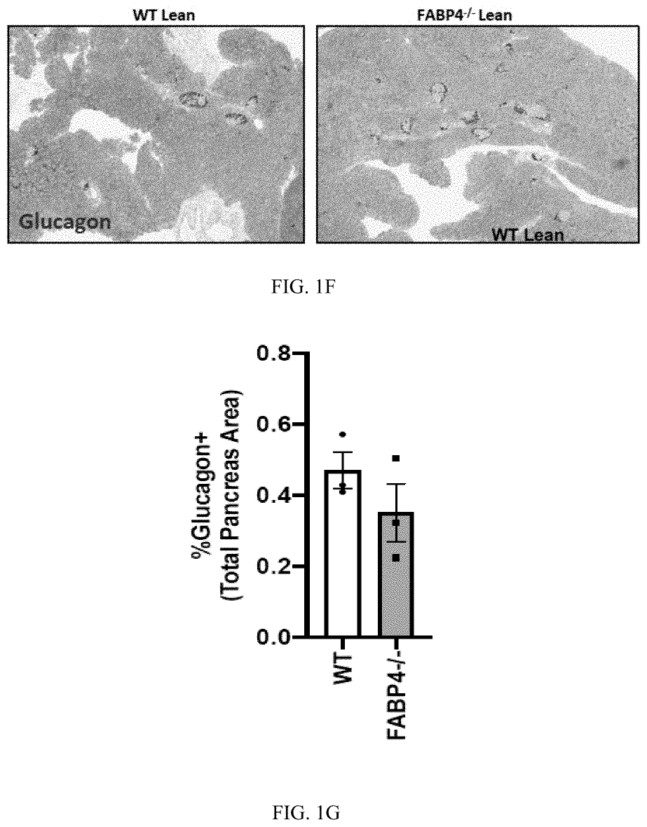

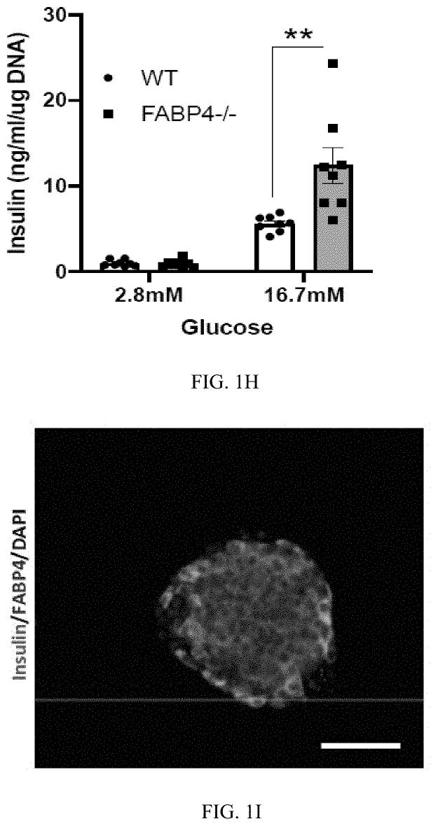

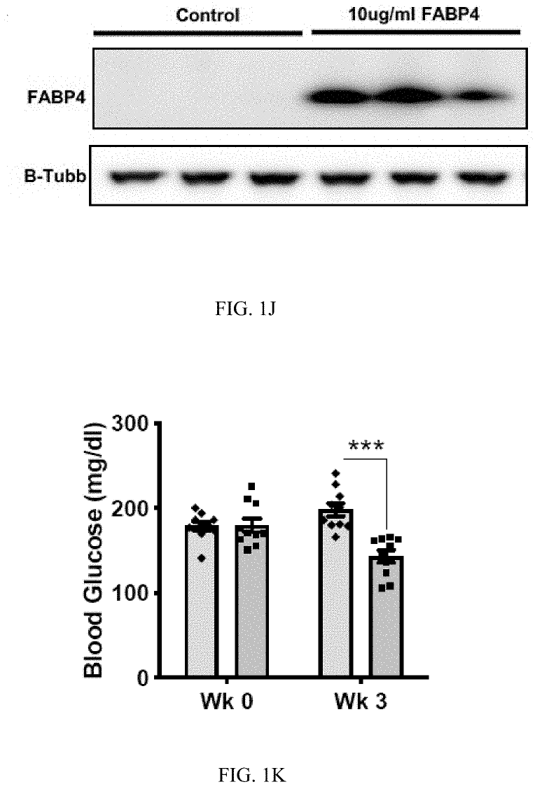

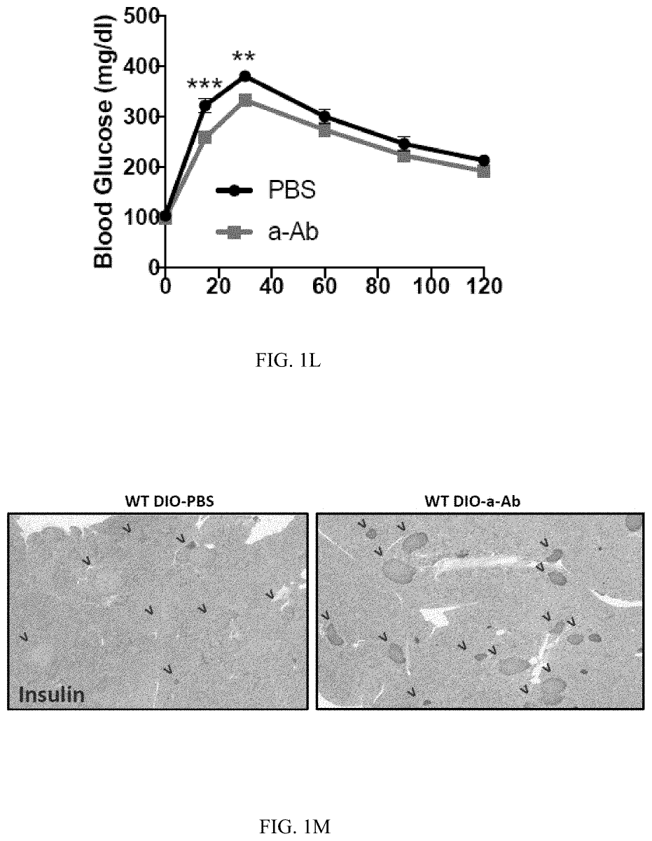

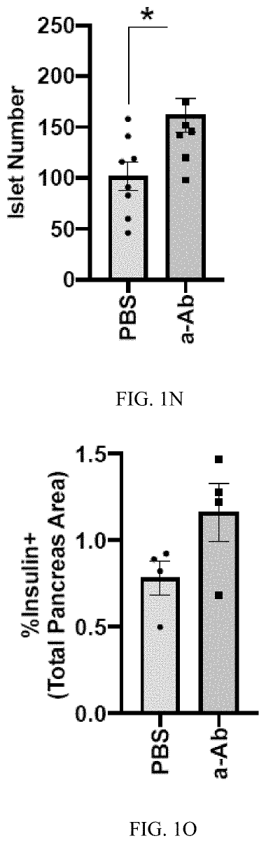

Remarkably, a difference in islet number could be observed even by gross visual examination of the pancreata of lean FABP4^{-/-} in vivo (FIG. 1A), with the presence of islets being confirmed by dithizone staining (FIG. 1B). Detailed examination revealed that lean FABP4^{-/-} mice exhibited significantly higher beta cell mass and pancreatic insulin content compared to wild type (WT) littermates, as evaluated by immunohistochemistry and quantification of total pancreatic insulin content (FIG. 1C-E).

Total pancreatic insulin content was evaluated from 7-week-old mice. Pancreata were isolated, weighed, and flash frozen in liquid nitrogen. Samples were then ground with a mortar and pestle in liquid nitrogen, collected into 1.5 ml tubes and lml acid ethanol added. Samples were stored overnight at −20° C., centrifuged for 15 min at 4° C., and ethanol evaporated by SpeedVac. Samples were resuspended to 0.5 ml in ultrapure water and insulin quantified by HTRF.

For the immunohistochemistry, pancreata were dissected and fixed in 10% zinc buffered formalin for 24-48 hrs before being washed and stored in 70% ethanol. Samples were paraffin embedded, and sectioned in steps 250 μm apart. Immunostaining for insulin and glucagon was performed as described previously (Prentice et al., 2014; Rezania et al., 2014). Images of each section were acquired using Aperio Imagescope at 20× magnification. The beta- or alpha-cell area was calculated by using positive pixel count analysis (Aperio ImageScope). Islet number and size was determined by manually circling insulin positive clusters in the Aperio ImageScope software. Two blinded individuals independently performed manual analysis and insulitis scoring.

This enhancement in beta cell mass was not associated with a general increase in endocrine cells, as there was no difference in glucagon positive area (FIGS. 1F, G). In addition to an increase in beta cell mass, primary islets isolated from FABP4^{-/-} mice demonstrated significantly increased glucose-stimulated insulin secretion (GSIS) (FIG. 1H). Importantly, FABP4 is not expressed in islet endocrine cells or clonal beta cell line INS1 (FIGS. 1I, 1J). Thus, this cell type provides an opportunity to examine the specific role of hormonal FABP4 and the mechanisms underlying its actions. A monoclonal antibody (previously reported as CA33 and active antibody (a-Ab)) to generate further support for the paracrine/endocrine role of FABP4 in vivo. This antibody was previously shown to improve metabolic parameters in the context of diet-induced obesity (Burak et al), but the impact on beta cells was not examined. The antibody sequence is shown in Table 10.

TABLE 10
Sequence of CA33 (a-Ab)
heavy chain and light chain

| | SEQ | |
|---|---|---|
| Antibody | ID | |
| region | NO: | Sequence |
| CA33 (a-Ab) | 446 | QSVEESGGRLVTPGTPLTLTCTVSGFSLST |
| heavy chain | | YYMSWVRQAPGKGLEWIGIIYPSGSTYCAS |
| | | WAKGRFTISKASTTVDLKITSPTTEDTATY |
| | | FCARPDNDGTSGYLSGFGLWGQGLVTVSSA |
| | | KTTPPSVYPLAPGSAAQTNSMVTLGCLVKG |
| | | YFPEPVTVTWNSGSLSSGVHTFPAVLQSDL |
| | | YTLSSSVTVPSSTWPSETVTCNVAHPASST |
| | | KVDKKIVPRDC |
| CA33 (a-Ab) | 447 | DVVMTQTPASVSEPVGGTVTIKCGASEDIS |
| light chain | | RYLVWYQQKPGQPPKRLIYKASTLASGVPS |
| | | RFKGSGSGTDFTLTISDLECDDAATYYCQC |
| | | TYGTYAGSFFYSFGGGTEVVVERTDAAPTV |
| | | SIFPPSSEQLTSGGASVVCFLNNFYPKDIN |
| | | VKWKIDGSERQNGVLNSWTDQDSKDCTYSM |
| | | SSTLTLTKDEYERHNSYTCEATHKTSTSPI |
| | | VKSFNRNEC |

Antibodies were stored at 4° c. at a stock concentration of 10 mg/ml in PBS. WT diet-induced obese (DIO) male mice were treated twice weekly with 33 mg/kg bodyweight of a-Ab or control antibody (c-Ab) in a final volume of 150 μl, injected subcutaneously for 3 weeks after 12 weeks of high fat breeding, as described (see US20160319003, incorporated herein by reference, and Burak et al., 2015). Treatment of wild-type DIO mice with a-Ab for three weeks resulted in improved glycemia, as evaluated by reduced 6 hr fasting glucose levels and improved glycemic excursion during glucose tolerance test (GTT) (FIGS. 1K, 1L). Glucose tolerance test were performed as previously described (Liu, Y. et al. Cell Rep 14, 2889-2900). Briefly, both chow and high fat diet fed mice were evaluated simultaneously. Before intraperitoneal glucose tolerance tests, mice were fasted 16 hr. overnight, before injection of 0.75 g/kg body weight of glucose. Blood glucose was evaluated at 0, 15, 30, 45, 60, 90, and 120-min post-injection. Examination of the pancreata of a-Ab treated DIO mice revealed an increased number of islets, consistent with what was observed in lean FABP4^{-/-} mice and a trend towards increased beta cell mass (FIGS. 1M-O). These data further support the hypothesis that hormonal FABP4 acts on beta cells to increase mass and function in vivo.

### Example 2. Hormonal FABP4 is Elevated in Type 1 Diabetes (T1D)

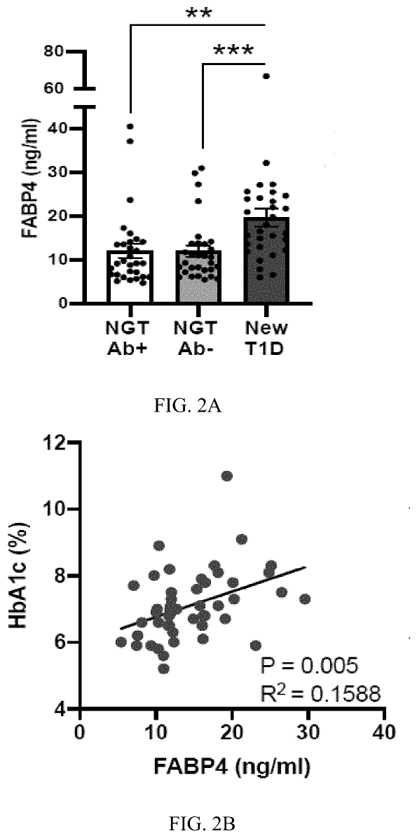

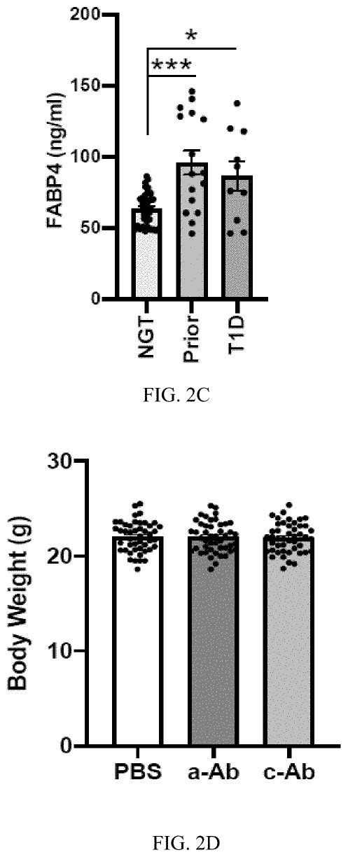

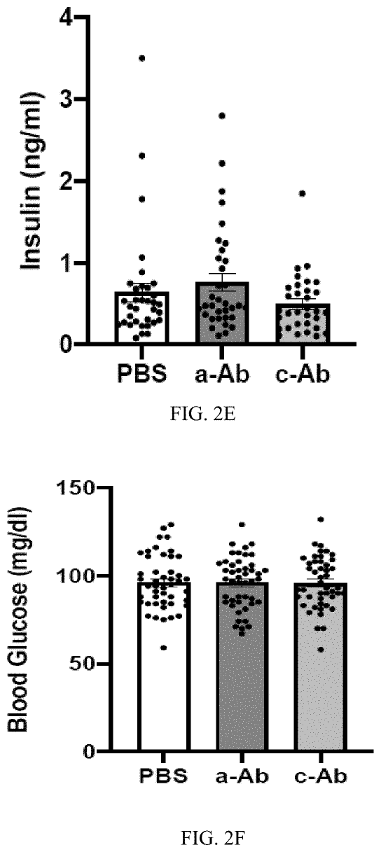

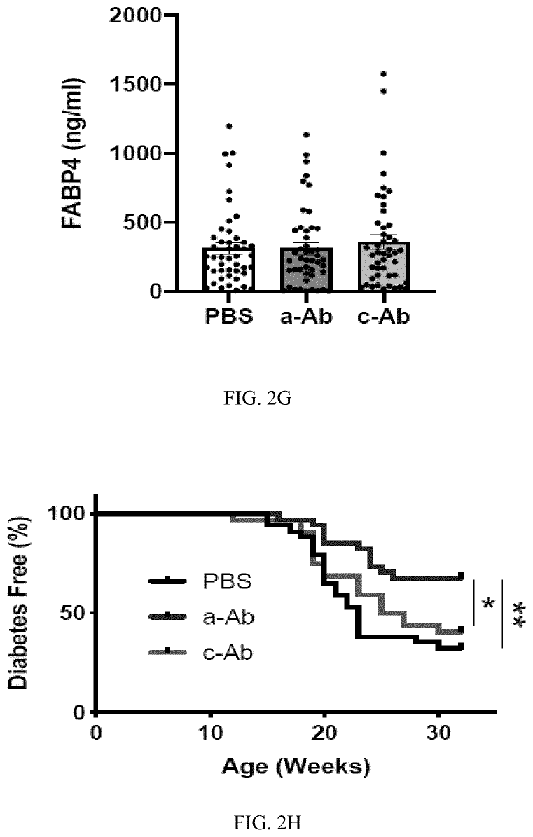

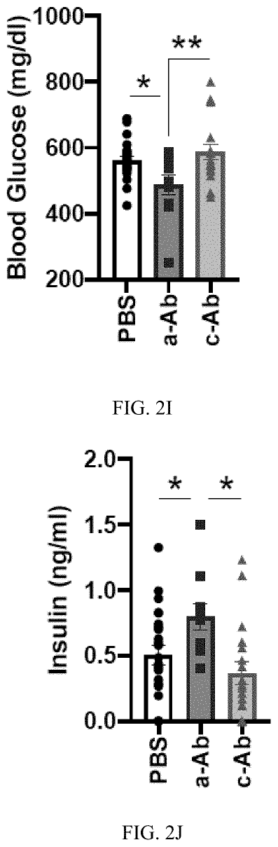

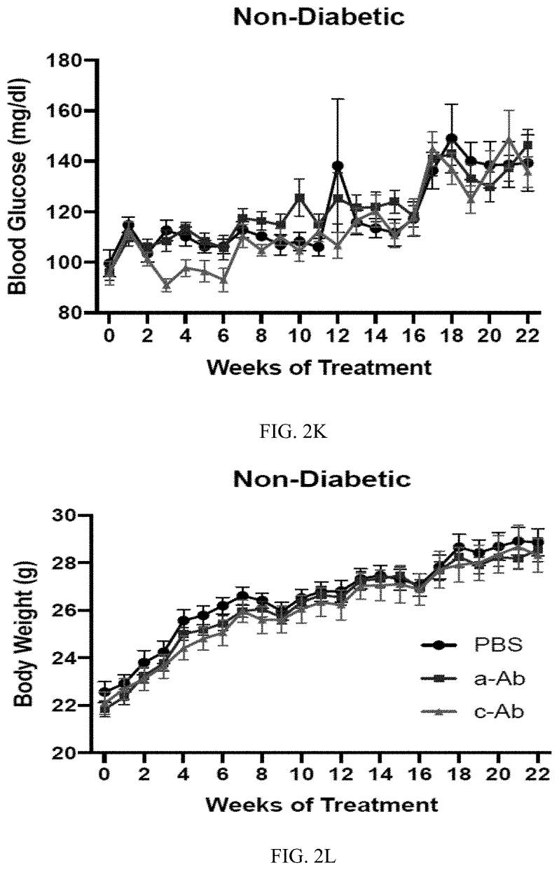

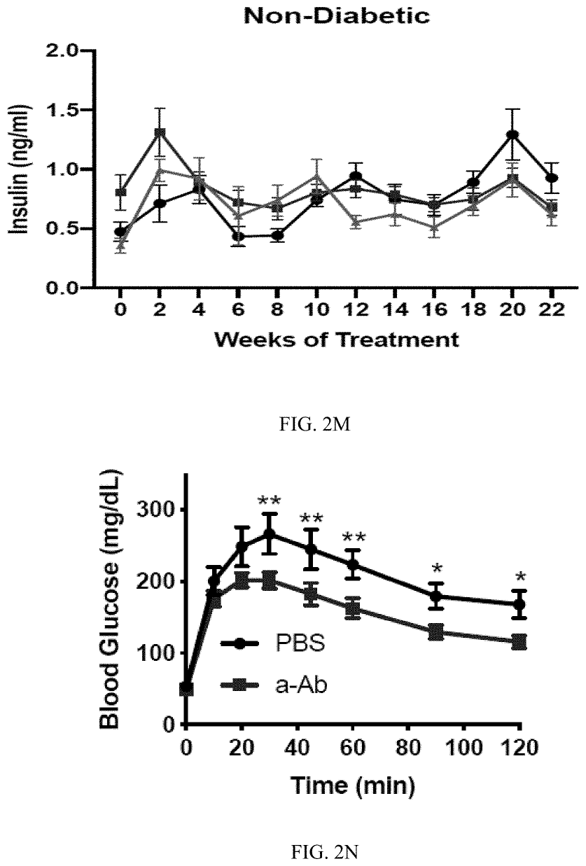

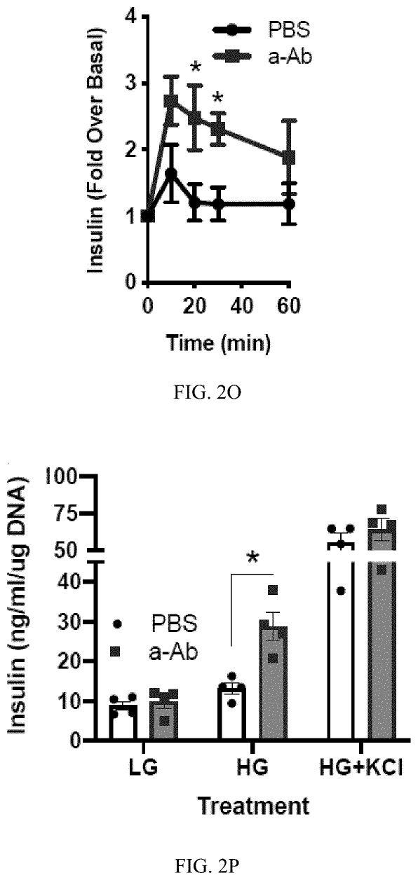

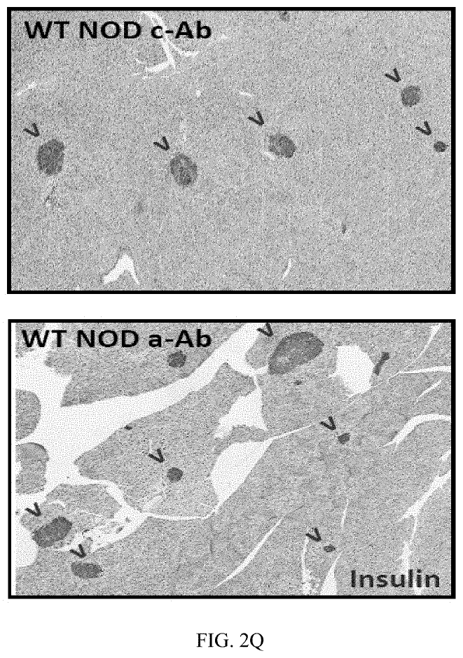

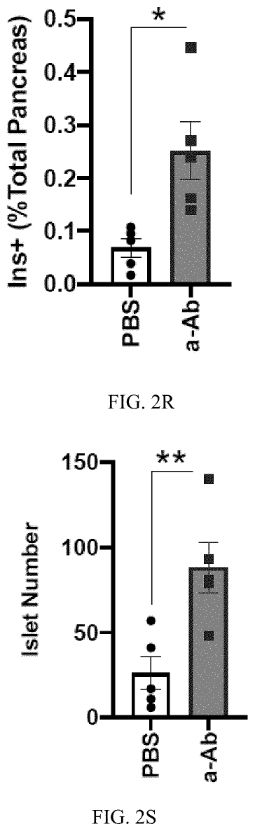

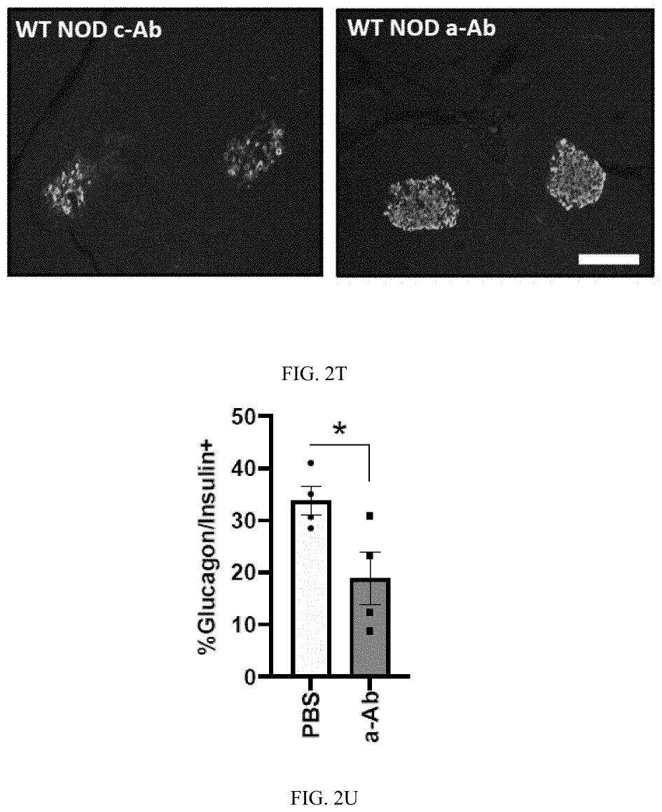

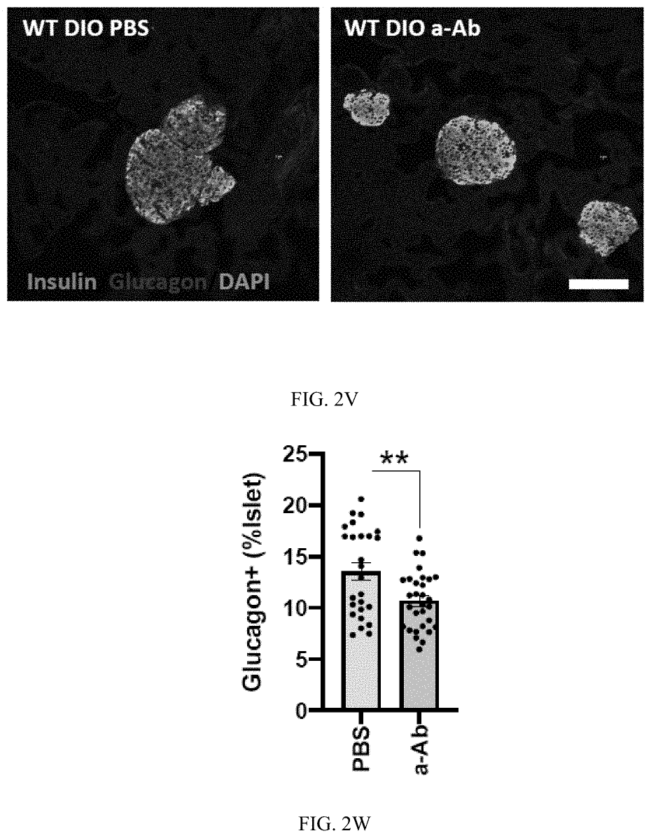

An endocrine link between adipose tissue and beta cells established through hormonal FABP4 may contribute to beta cell dysfunction and development of diabetes. It is well established that hormonal FABP4 is elevated in Type 2 diabetes (T2D). However, given the strong correlation between FABP4 and body mass index (BMI) there was an interest in determining if hormonal FABP4 is also regulated in T1D, independent of adiposity and insulin resistance, contributing to beta cell dysfunction directly. First, serum samples were evaluated from normoglycemic individuals with or without autoantibody positivity and new-onset T1D patients (<1 year following diagnosis) obtained from the BABYDIAB and DIMELLI cohorts (Thumer et al., 2010; Warncke et al., 2013; Ziegler et al., 1999) (Munich, Germany). In this study, 30 patients per group were matched for age and gender. Serum FABP4 was significantly elevated (-1.6 fold) in new-onset T1D individuals compared to both autoantibody positive and negative normoglycemic control groups (FIG. 2A, Table 11).

TABLE 11
Comparative data on T1D and control
groups (BABYDIAB and DIMELLI cohorts)

| | | | NGT | | NGT | |
|---|---|---|---|---|---|---|
| | T1D | | Positive | | Negative | |
| N | 30 | | 30 | | 30 | |
| Male, n (%) | 15 | (50) | 17 | (56.7) | 16 | (53) |
| Age (years), mean (SD) | 11.7 | (4.5) | 11.8 | (3.9) | 11.7 | (3.8) |
| Range | 3-18 | | 3-18 | | 3-18 | |
| BMI (kg/m²), | 17.9 | (4.0) | 20.2 | (3.7) | 18.3 | (3.2) |
| mean (SD) | | | | | | |
| Range | 12.8-28.2 | | 12.6-30.1 | | 14.1-27.7 | |
| FABP4 (ng/ml), | 19.6 | (11.2) | 12.1 | (8.5) | 12.1 | (7.0) |
| mean (SD) | | | | | | |
| Range | 6.0-67.0 | | 4.7-40.5 | | 5.5-31.0 | |
| Fasting Glucose (mg/dl), | 144.0 | (44.8) | 88.7 | (10.7) | 84.6 | (8.3) |
| mean (SD) | | | | | | |
| Range | 68-256 | | 67-114 | | 71-100 | |
| HbA1c (%), mean (SD) | 11.3 | (2.3) | 5.2 | (0.2) | 5.2 | (0.4) |

| | | | |
|---|---|---|---|
| Range | 7.6-15.7 | 4.7-5.5 | 4.6-6.2 |

Data are presented as mean ± standard deviation (SD), range, or numbers (n).
T1D, type 1 diabetes; BMI, body mass index; FABP4, fatty acid-binding protein 4; HbA1c, haemoglobin A1c.
Groups are matched for gender, age, and BMI.
Bold: Significant compared to NGT Positive (Independent Samples t-test).

In the second population (BRI Cohort) of older T1D patients with various duration of disease and NGT controls, a significant correlation was detected between serum FABP4 levels and HbA1c in diabetic patients (r²=0.16, p=0.005), suggesting FABP4 is associated with glycemic control (FIG. 2B, Tables 12-13). There was no significant difference in circulating FABP4 between diabetic and control subjects in this cohort (Tables 12-13).

TABLE 12
Comparative data on T1D and control group (BRI cohort)

| | T1D | | Control | | P-value^{a} |
|---|---|---|---|---|---|
| N | 50 | | 50 | | 1.0 |
| Male, n (%) | 21 | (42) | 21 | (42) | 1.0 |
| Age (years), mean (SD) | 34 | (9.7) | 34 | (9.7) | 1.0 |
| Range | 18-57 | | 18-57 | | — |
| BMI (kg/m²), mean (SD) | 25.4 | (3.6) | 25.4 | (3.6) | 1.0 |
| Range | 18.1-32.1 | | 17.8-32.0 | | — |
| FABP4 (ng/ml), mean (SD) | 16.7 | (11.9) | 14.2 | (7.6) | 0.217 |

| Range | 5.4-80.1 | | 4.0-41.5 | — |
|---|---|---|---|---|
| Glucose (mg/dl), mean (SD) | 157.4 | (55.5) | — | — |
| Range | 64.0-280.0 | | — | — |
| HbA1c (%), mean (SD) | 7.1 | (1.1) | — | — |

| | | | |
|---|---|---|---|
| Range | 5.2-11.0 | — | — |

Data are presented as mean ± standard deviation (SD), range, or numbers (n).
T1D, type 1 diabetes; BMI, body mass index; FABP4, fatty acid-binding protein 4; HbA1c, haemoglobin A1c.
Groups are matched for gender, age, and BMI.
^{a}Between groups comparison (Pearson Chi-square test or Independent Samples t-test).

TABLE 13
Spearman correlations of circulating FABP4 in the BRI cohort

| | Age | BMI | Glucose | HbA1c |
|---|---|---|---|---|
| | (years) | (kg/m²) | (mg/dl) | (%) |
| Spearman's rho, | 0.106 | 0.450 | 0.120 | 0.333 |
| FABP4 (ng/ml) vs. | | | | |
| P-value | 0.293 | <0.001 | 0.418 | 0.018 |
| N | 100 | 100 | 48 | 50 |

FABP4, fatty acid-binding protein 4; BMI, body mass index; HbA1c, haemoglobin A1c

To complement these analyses, human plasma samples from the BABYDIAB and

DIMELLI cohorts were collected and stored as previously described. Samples from patients were matched for age, gender, and BMI, and T1D individuals were within one year of diagnosis. Samples from the BRI cohort were matched for gender and BMI with varying duration of T1D. Samples were analyzed for serum FABP4 levels, quantified by an in-house developed FABP4 ELISA using monoclonal antibodies G9 and H3, and a human FABP4 standard curve (R&D DY3150-05, USA). Samples were blinded at the time of measurement, and biometric data was provided following the completion of assays. Serum FABP4 levels were significantly increased despite a reduction in BMI in the new onset diabetes group.

Additionally, FABP4 was quantified in serum from WT female non-obese diabetic (NOD) mice, comparing levels one week before T1D onset and at the time of diagnosis (second consecutive blood glucose measurement >250 mg/d1) to age-matched non-diabetic controls. FABP4 was significantly increased both shortly before diabetes onset and in new-onset T1D mice (FIG. 2C). Together, these findings demonstrate that circulating FABP4 are regulated before and immediately after impairment of glycemic control, suggesting that hormonal FABP4 may have a role in beta cell failure and diabetes pathogenesis.

WT female NOD mice matched for age, weight, blood glucose, plasma insulin, and circulating FABP4 levels were utilized to explore the effect of antibody-mediated FABP4 targeting in the context of T1D, (FIGS. 2D-G). Mice were treated twice weekly with a-Ab, PBS, or a control antibody of the same isotype (c-Ab) starting at 10 weeks of age, when insulitis had already been established. Administration of the a-Ab significantly protected against the development of T1D (FIG. 2H). Among the mice that developed diabetes, a-Ab-treated animals had significantly reduced blood glucose and higher plasma insulin levels, suggesting a less severe diabetes phenotype (FIGS. 2I, 2J). Throughout the study there were no significant differences in body weight, plasma insulin, or blood glucose levels among the non-diabetic mice (FIGS. 2K-M). Consistent with improved beta cell mass and function in FABP4−/− mice and DIO mice treated with a-Ab, non-diabetic NOD mice treated with a-Ab exhibited improved glycemic control during GTT, corresponding to improved GSIS both in vivo and ex vivo (FIGS. 2N-P), and a significant increase in islet number and beta cell mass compared to c-Ab-treated controls in vivo, with denser insulin staining were observed (FIGS. 2Q-S). Evaluation of alpha cell area in pancreatic sections from both NOD and DIO mice treated with a-Ab revealed significant reduction in glucagon positive area in both models, suggesting a normalization of islet endocrine cell populations that may also contribute to improved glycemia (FIGS. 2T-W). Collectively, a-Ab-mediated protection against T1D and improvement of glycemic control in a DIO model of T2D are both associated with improved beta cell mass and function.

### Example 3. Discovery of a FABP4-ADK-NDPK Complex

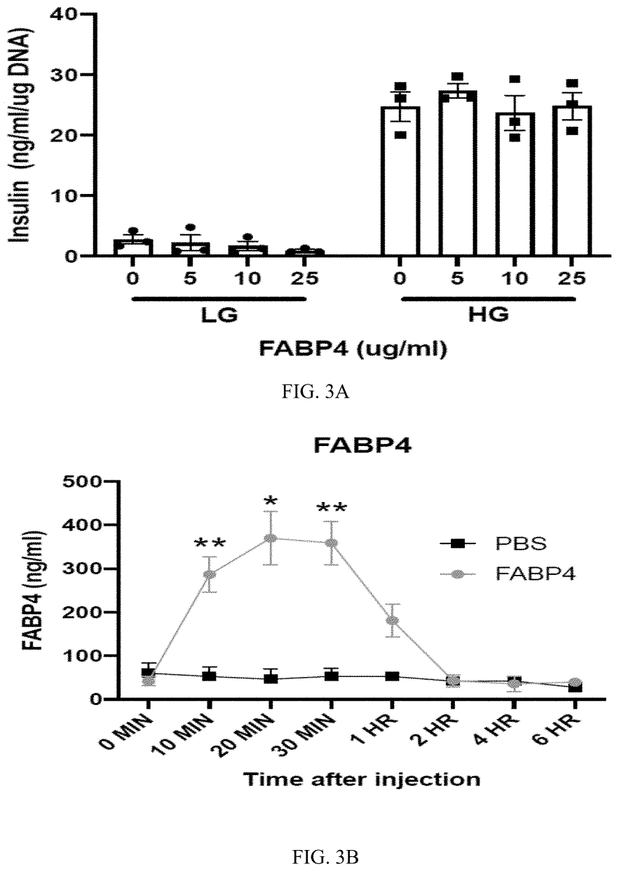

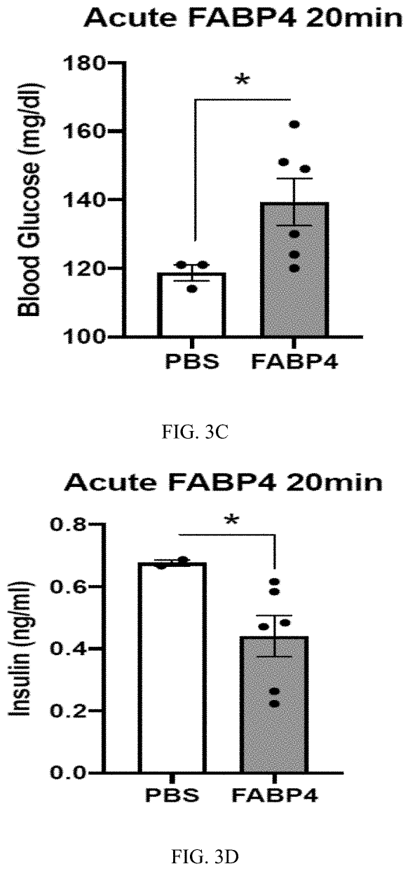

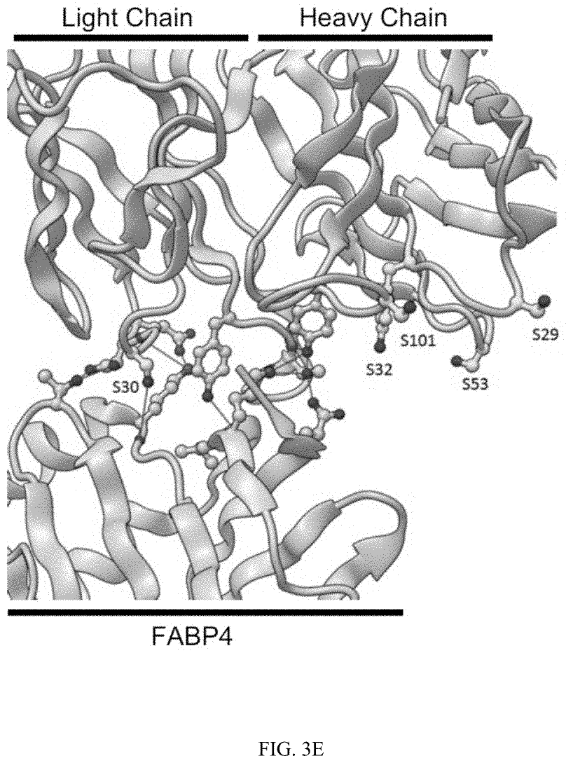

Despite the influence of hormonal FABP4 on beta cell mass and function in vivo, addition of recombinant FABP4 to primary mouse islets had no effect on GSIS, even at super-physiological doses (FIG. 3A). This is in line with previous studies which have been unable to demonstrate a consistent impact of recombinant FABP4 alone on GSIS in vitro. Intriguingly, acute FABP4 administration to WT NOD mice resulted in significantly increased blood glucose levels and a significant reduction in plasma insulin compared to vehicle-injected controls at the peak of plasma FABP4 levels, 20 minutes post-injection (FIGS. 3B-D). This raised the possibility that hormonal FABP4 may partner with other proteins in circulation to generate its biological functions. This prospect was further supported by the examination of structural properties of the interaction between FABP4 and a-Ab. Compared to traditional therapeutic antibodies, a-Ab exhibits a relatively weak binding affinity for recombinant FABP4. Further, examination of the crystal structure of a-Ab revealed that its binding to FABP4 is mediated through a limited number of interactions on the light chain only (FIG. 3E), unlike prototypical antibody-antigen interactions which are mediated by both the heavy and light chains, or predominantly the heavy chain. Thus, the heavy chain of a-Ab remains available for potential additional binding partners. Understanding the components of such a complex would define a novel mechanism of hormone action and shed light into the diverse metabolic functions of FABP4 hormone, including its potential effects on beta cells.

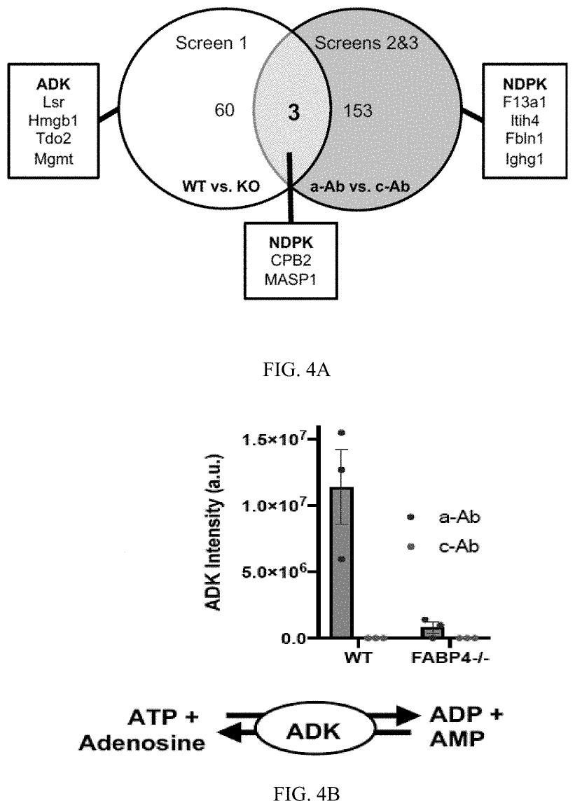

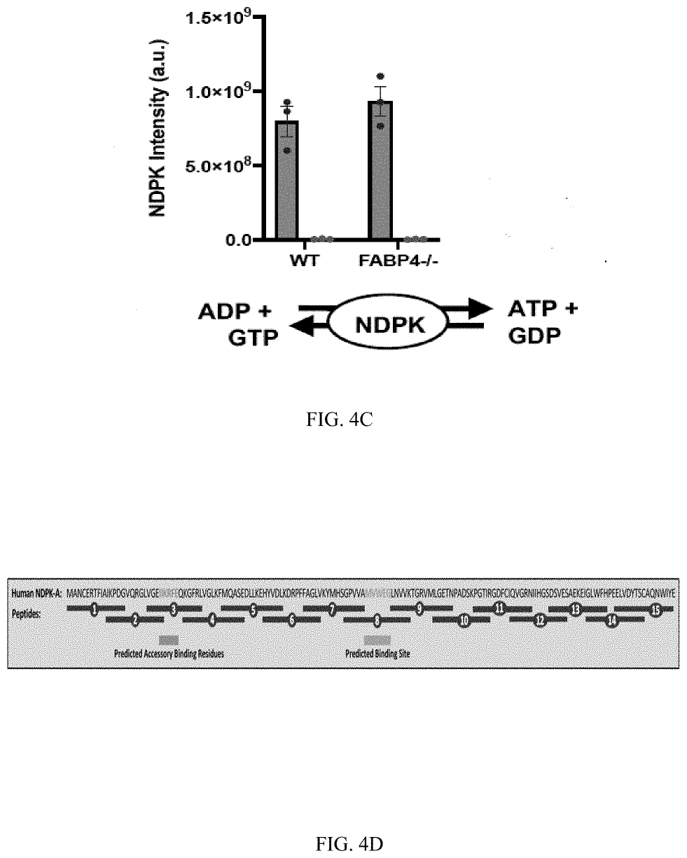

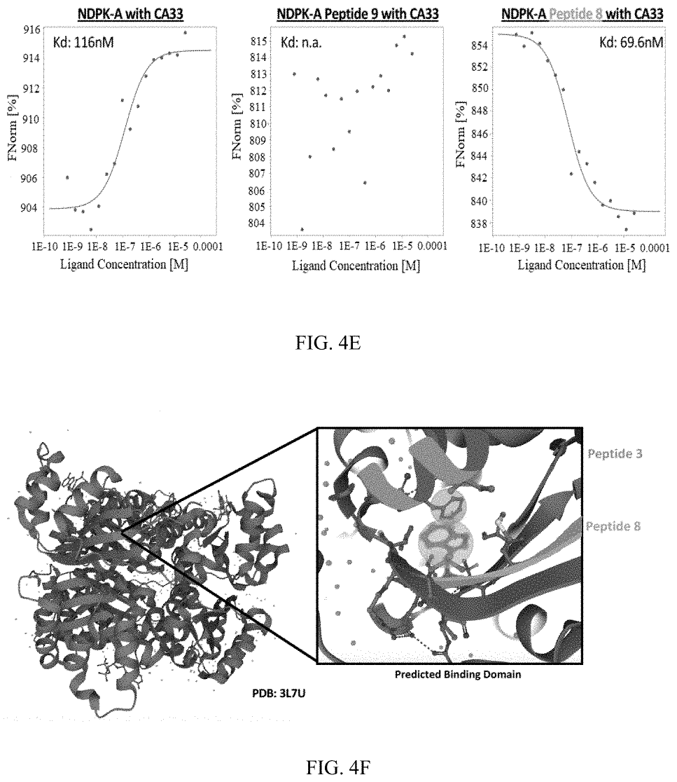

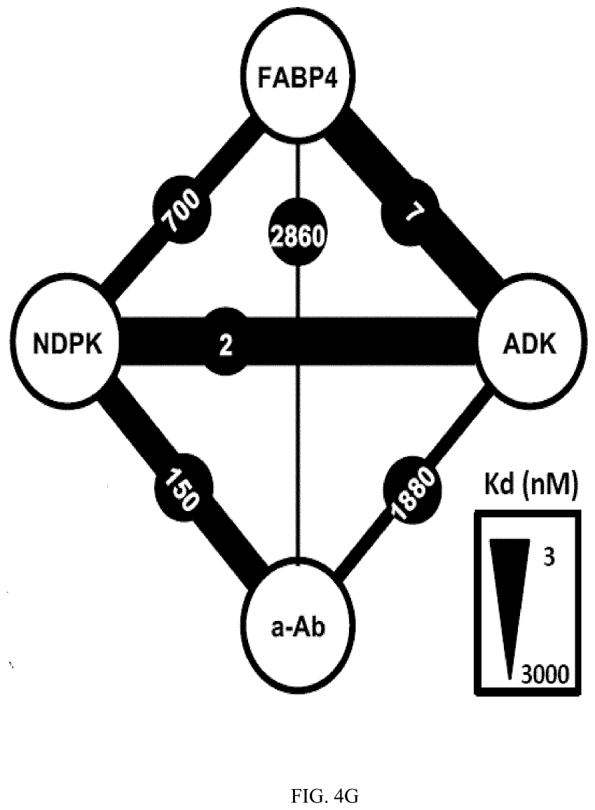

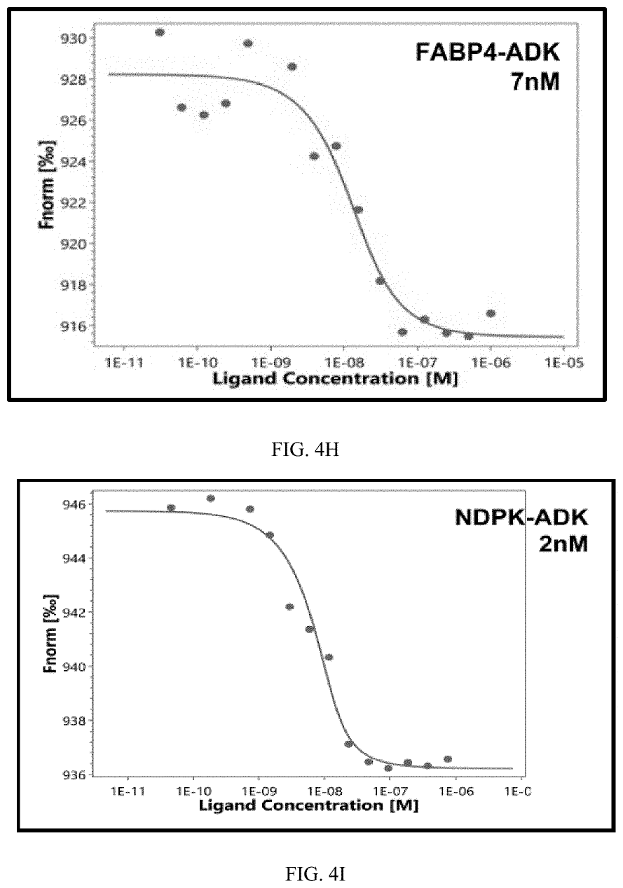

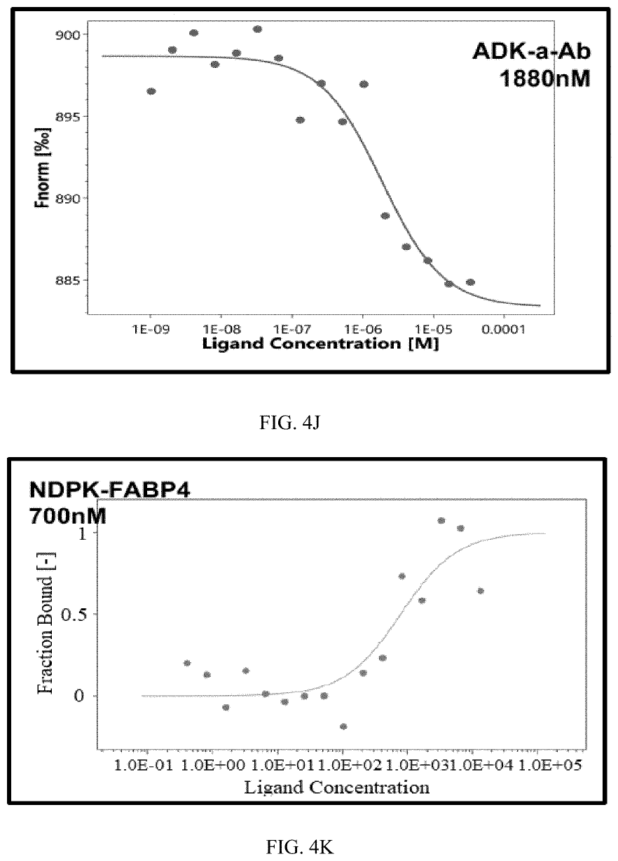

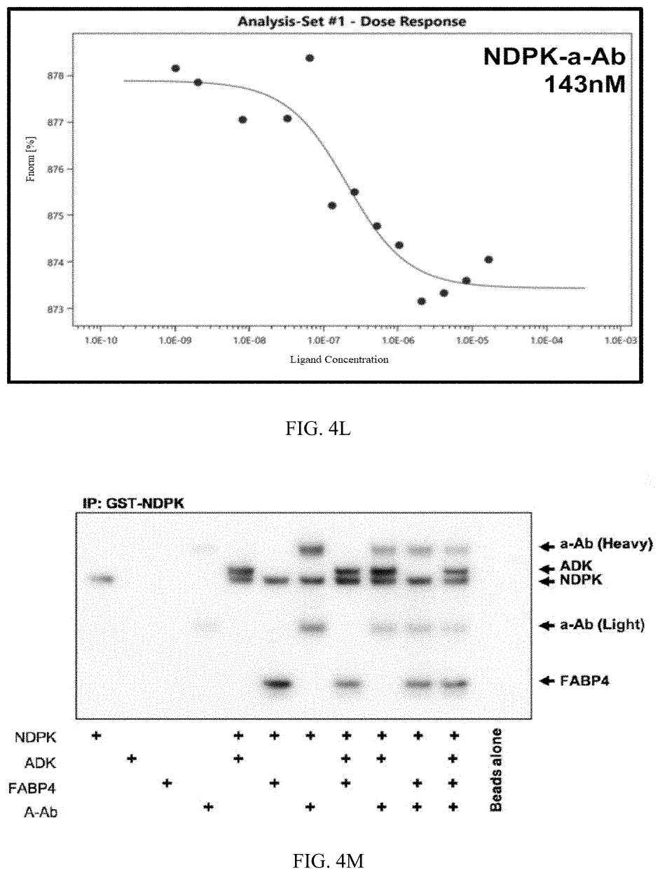

To identify such potential FABP4 binding partner(s), To identify such potential FABP4 binding partner(s), serum pulldown and mass spectrometry (MS) was performed using serum from DIO, glucose intolerant WT mice where clear efficacy of a-Ab treatment has been shown, and matched FABP4−/− controls. Comparing proteins pulled down specifically by a-Ab in WT serum, but not in FABP4−/− serum, Adenosine Kinase (ADK) was identified as a top hit (FIGS. 4A-B), suggesting that FABP4 is required for ADK interaction with a-Ab. The link between ADK and FABP4 is intriguing, as genetic models of beta cell-specific ADK deficiency describe an islet phenotype similar to FABP4−/− mice. Additional pulldown experiments comparing a-Ab with c-Ab identified another nucleoside kinase, Nucleoside Diphosphate Kinase (NDPK, also known as nm23/nme23/nme1/nme2) as the top hit binding a-Ab (FIGS. 4A, 4C). NDPK was bound to a-Ab, but not c-Ab, in both WT and FABP4−/− serum, suggesting that the antibody independently recognizes this protein.

Additionally, one-to-one recombinant protein interaction assays were performed using Microscale Thermophoresis (MST) to independently validate the interactions between each of the proposed complex components. MST is a technology for the interaction analysis of biomolecules. MST is the directed movement of particles in a microscopic temperature gradient. Any change of the hydration shell of biomolecules due to changes in their structure/conformation results in a relative change of the movement along the temperature gradient and is used to determine binding affinities. MST allows measurement of interactions directly in solution without the need of immobilization to a surface (immobilization-free technology).

To perform this technique, proteins were desalted and buffer exchanged into PBS prior to labeling. Desalting was performed using Zeba Spin Desalting Columns (Thermo Scientific, product number 89882, 0.5 ml volume) in a volume of 0.1 ml according to the manufacturer's instructions. Human NDPK-A (Acro Biosystems, product number NM1-H5147) was prepared in PBS to a stock concentration of 0.4 mg/ml. Human ADK (Novus Biologicals, product number NBP1-44382 or R&D Systems, product number 8024-AK-025) was purchased at a stock concentration of 0.5 mg/ml and buffer exchanged into PBS. Protein concentration used for labeling was 20 uM. Experiments were conducted independently with each of NDPK and ADK labeled with each kit (i.e., experiments were performed with lysine-labeled, cysteine-labeled, and His-labeled versions of each protein to confirm the site of labeling was not influencing detected interaction). Labeling kits were: Monolith Protein Labeling Kit RED-NETS (Amine Reactive; Product number MO-L001), Monolith Protein Labeling Kit RED-MALEIMIDE (Cysteine Reactive; Product number: MO-L004), and Monolith His-Tag Labeling Kit RED-tris-NTA (Product number: MO-L008). Antibodies and FABP4 were prepared in PBS buffer and not labeled in MST experiments. MST assays were performed in PBS buffer (with calcium and magnesium) with 5 mM MgCl₂ and 0.05% Tween-20. Concentration of labeled proteins ranged from 12 nM to 20 nM, as determined by MST quantification to obtain a fluorescence intensity of ˜350 from each capillary tube. For experiments involving ADK, premium coated capillaries were used. For all other interactions standard capillary tubes were used. The crystal structure for the FABP4-a-Ab interaction is available in the Protein Data Bank (PDB) database with the identifier SCON.

Using this technique, the strongest interactions were observed between FABP4 and ADK (K_{d}˜7 nM) and ADK and NDPK (K_{d}˜1.8 nM) (FIGS. 4G-K). ADK, however, has a relatively weak affinity for a-Ab directly (K_{d}˜1.9 μM; FIGS. 4G, 4I), consistent with the minimal level of detection in FABP4−/− serum pulldown experiments. NDPK binding to FABP4 was also detected with an affinity of ˜700 nM (FIGS. 4G, 4K) and to a-Ab with a K_{d} of ˜150 nM (FIGS. 4G, 4L). To further confirm the formation of the proposed complex, GST-tagged NDPK (GST-NDPK) was generated and immunoprecipitation experiments were performed to investigate the interaction of each proposed component with NDPK. Interaction between all three components of the proposed complex, as well as with the complex and a-Ab (FIG. 4M) were readily identified. These data provide strong evidence that FABP4 forms a complex with ADK and NDPK driven by the strong affinities of FABP4 to ADK and ADK to NDPK.

To define the binding site of a-Ab on NDPK, a nested series of peptides were generated covering human NDPK-A and interaction of each peptide with a-Ab was evaluated by Microscale Thermophoresis (MST) (FIG. 4D). MST is a technology for the interaction analysis of biomolecules. MST is the directed movement of particles in a microscopic temperature gradient. Any change of the hydration shell of biomolecules due to changes in their structure/conformation results in a relative change of the movement along the temperature gradient and is used to determine binding affinities. MST allows measurement of interactions directly in solution without the need of immobilization to a surface (immobilization-free technology). Low affinity binding was observed between peptide 2 and peptide 3 with a-Ab (uM range). High affinity binding (nM) was observed between peptide 8 and a-Ab, comparable to full-length NDPK-A protein (Table 14).

TABLE 14
Binding affinity as determined by
MicroScale Thermophoresis (MST)

| | | | Binding | |
|---|---|---|---|---|
| | | | Affinity | |
| | | SEQ | (Ka) to | |
| Peptide | Sequence | ID | CA33 | |
| Number | (3′-5′)** | NO | (a-Ab) | |
| 1 | MANCERTFIAIKPDG | 429 | NBD | |
| 2 | IKPDGVQRGLVGEII | 430 | 15.94 | μM |
| 3 | VGEIIKRFEQKGFRL | 431 | 8.98 | μM |
| 4 | KGFRLVGLKFMQASE | 432 | NBD | |
| 5 | MQASEDLLKEHYVDL | 433 | NBD | |
| 6 | HYVDLKDRPFFAGLV | 434 | NBD | |
| 7 | FAGLVKYMHSGPVVA | 435 | NBD | |
| 8 | GPVVAMVWEGLNVVK | 436 | 69.6 | nM |
| 9 | LNVVKTGRVMLGETN | 437 | NBD | |
| 10 | LGETNPADSKPGTIR | 438 | NBD | |
| 11 | PGTIRGDFCIQVGRN | 439 | NBD | |
| 12 | QVGRNIIHGSDSVES | 440 | NBD | |
| 13 | DSVESAEKEIGLWFH | 441 | NBD | |
| 14 | GLWFHPEELVDYTSC | 442 | NBD | |
| 15 | ELVDYTSCAQNWIYE | 443 | NBD | |
| Full | | | 116 | nM |
| Length | | | | |

**All peptides and full-length protein have N-Term 6xHis Tag
NBD: No Binding Detected
Predicted Binding Site - MVWEG on peptide 8
Predicted Accessory Binding Residues - IIKRFE on peptide ¾

No binding was detected (NBD) between a-Ab and any other peptide examined. The lack of binding between peptide 7 or peptide 9 and a-Ab indicated that the central 5 amino acids of peptide 8, non-overlapping in sequence with either peptide 7 or 9, is likely the primary epitope for a-Ab binding (Table 14). Examination of the published crystal structure for NDPK (PDB: 3L7U) indicates the proposed binding region of peptide 8 is on the surface of the protein, amenable for a-Ab interaction (FIG. 4F). Furthermore, the tertiary folding of NDPK-A places peptides 3 and peptide 8 in close proximity, suggesting binding of a-Ab may be primarily occurring through residues in peptide 8, and partially facilitated through interaction with residues in peptides 2 and 3. Taken together, these findings support the presence of a potential complex between FABP4-ADK-NDPK in vivo, which is targeted by a-Ab through interaction with both FABP4 and NDPK.

### Example 4. Function of the ADK-NDPK/FABP4 Complex and the Impact of FABP4 on its Activity In Vivo

An on-bead kinase assay was developed to measure the activity of endogenous NDPK and ADK pulled down from serum using a-Ab or c-Ab conjugated beads. Both NDPK and ADK are bi-directional kinases that will produce ATP or ADP depending on the substrates provided (FIGS. 4B-C). Kinase activity assays were performed in 384-well plates in reaction buffer (PBS with 0.1% BSA, 0.01% F-127, 5 mM MgCl2). Experiments with recombinant proteins were performed at final concentrations of 40 μg/ml for ADK and FABP4, and 20 ug/ml for NDPK, and a reaction volume of 10 μl. Antibodies were used at a final concentration of 80 ug/ml. Substrates were added in equimolar ratios depending on the kinase being assayed and the direction of the reaction in serial dose responses, with the highest concentration of 5 μM. Proteins and substrates were incubated for 1 hr at room temperature, and ADP and ATP production were monitored using ADP-Glo and Kinase-Glo kits, respectively, according the manufacturer instructions (V6930 and V6711 Promega, USA). Luminescence was quantified using a SpectraMax Paradigm plate reader 30 min after the addition of substrate. Consistent with the MS results, ADK activity, as measured by generation of ATP, was specifically observed in samples pulled down with a-Ab but not c-Ab (FIG. 5A). Furthermore, ADK activity was significantly lower in pull-down samples from FABP4−/− serum than WT, supporting the hypothesis that ADK binds to FABP4 as part of the complex, and that presence (or absence) of FABP4 in this complex may regulate the kinase activity (FIG. 5A). To test this possibility, the ATP and ADP generating activity of recombinant ADK was assessed in the presence of the proposed complex components. Interestingly, FABP4 both alone and in combination with NDPK significantly increased ADK activity to generate ATP, while NDPK alone had no effect (FIGS. 5B-C). There was no difference in the capacity to generate ADP, suggesting the alteration in activity is uni-directional (FIG. 5D).

The ADP-producing capacity of NDPK was also evaluated, beginning with serum pulldown samples in WT and FABP4^{-/-} serum using antibody conjugated beads as described above. When the ADP-producing capacity of NDPK was evaluated in the on-bead serum pulldown assay, similar to ADK, NDPK activity was observed only in samples pulled down with a-AB, but not c-Ab (FIG. 5E). Furthermore, increased NDPK activity was observed in FABP4−/− serum as compared to WT, despite no difference in abundance of NDPK. This supports the notion that FABP4 plays an important role in regulating this complex activity.

Endogenous protein activity assays were performed by conjugating magnetic beads with a-Ab or c-Ab overnight with rotation at 4° C. according to manufacturer instructions (Thermo Scientific Dynabeads 10002D). Beads were washed and suspended in buffer (PBS with 0.05% Tween20, 5 mM MgCl2). A volume of beads equivalent to 1 μg of antibody were then incubated with 40 ul of serum from lean WT or FABP4^{-/-} mice, and rotated at room temperature for 1 hr. Samples were then aliquoted into 96-well plates placed on a magnet and washed three times with reaction buffer. 150 μl of reaction buffer containing 1 mM ATP and 1 mM GDP was added, and 5 μl aliquots collected every 5 minutes for 30 minutes to evaluate NDPK activity to produce ADP using the ADP-Glo assay (Promega, USA). Beads were then washed three times with reaction buffer, and 100 μl of reaction buffer containing 1 mM ATP and 1 mM adenosine was added to evaluate ADK activity to produce ADP. 5 μl samples were collected at 10, 20, 30 and 60 minutes to evaluate ADP production, as above.

Consistent with a high affinity interaction between NDPK and ADK, addition of recombinant ADK significantly reduced the activity of NDPK to generate ADP, which was not further influenced by the addition of FABP4, suggesting that ADK is the primary component regulating NDPK activity (FIGS. 5F-G). There was no effect on the capacity of NDPK to produce ATP with addition of FABP4 or ADK, suggesting the effect is also unidirectional (FIG. 5H)

Finally, the effect of a-Ab on the capacity of both ADK and NDPK to generate ATP and ADP was evaluated, respectively, to elucidate in vivo mechanism of action. Addition of a-Ab to either ADK or NDPK alone had no effect on activity of either kinase (FIGS. 5I-J, indicating the functional importance of a-Ab on the entire NDPK-ADK/FABP4 complex in vivo and the impact of antibody-mediated targeting (Burak et al., 2015). Addition of a-Ab to the NDPK-ADK/FABP4 complex also had no effect on ADK activity to produce ATP compared to the complex alone (FIG. 5K). Conversely, a-Ab rescued the ADP producing capacity of NDPK in the presence of the complex components, while c-Ab had no effect (FIG. 5L). This suggests that a-Ab is preventing ADK suppression of NDPK activity. A model was developed based on these activity assays combined with the mass spectrometry analysis (Illustrated in FIG. 5M) regarding the function of the NDPK-ADK/FABP4 complex. Under WT conditions, the NDPK-ADK/FABP4 complex results in increased capacity for ATP production (via ADK) and reduced ADP production (via NDPK). In the absence of FABP4, ADK activity is reduced, resulting in lower ATP production and higher ADP production. This relative increase in ADP compared to ATP is phenocopied by the antibody-mediated therapeutic targeting of the complex using a-Ab.

### Example 5. FABP4-ADK-NDPK Impacts Glucose Stimulated Insulin Secretion (GSIS) Via P2Y1

Primary islet isolation from mice was performed as previously described (Batchuluun et al., 2018; Prentice et al., 2014) and GSIS was assessed following acute treatment with NDPK-ADK in the presence or absence of FABP4. Insulin secretion assays were performed as previously described (Prentice et al., 2014). For primary islets, 20 islets were hand-picked into 1.5 ml tubes, washed twice with Krebs Ringer Buffer (KRB) without glucose, and pre-incubated in 500 μl low glucose (LG; 2.8 mM) KRB for 1 hour at 37° C. KRB was then removed and 250 μl of fresh LG KRB added. Islets were incubated for 20 minutes at 37° C. Supernatant was collected and 250 μl high glucose (HG; 16.7 mM) KRB added. Islets were incubated for 20 minutes at 37° C. In conditions where KCl was used, HG KRB was collected, and 250 μl KC1 (16.7 mM glucose, 30 mM KCl) KRB added. Islets were incubated for 20 minutes at 37° C., and supernatant collected. Following the stimulation protocol, cells were lysed in 100 μl acid ethanol (70% ethanol, 1% HCl in water) and stored at 4° C. overnight. Samples were evaporated using a SpeedVac until dried, and resuspended in 60 μl ultrapure water. DNA was quantified by Nanodrop (ThermoFisher, USA). Insulin from supernatant was quantified using insulin HTRF (Insulin Ultrasensitive Assay kit 62IN2PEH; Cisbio, USA).

To quantify primary islet immunofluorescence, primary mouse islets were isolated as described above, and cultured overnight at 37° C. in RPMI-1640 media containing 10% FBS and 1% Penicillin/Streptomycin. 20 islets were picked into each chamber of a multi-chamber coverslip (ibidi 80827), washed with PBS and fixed for 25 min at room temperature (RT) in 4% paraformaldehyde (VWR, 157-4-100). Cells were washed twice, permeabilized for 30 min at RT (Permeabilization buffer PBS with 0.2% BSA, 0.1% Saponin, 0.1% Triton-X 100), and blocked for 30 min at RT (PBS with 3% BSA and 0.1% Saponin). Cells were incubated in primary antibody overnight at 4° C. in PBS with 1% BSA. Cells were washed 3×5 min in PBS, and incubated in secondary antibody for 1 hour at RT. Cells were washed 3×5 min in PBS, and mounted in VECTASHIELD® Hard Set Mounting Medium with DAPI (cat #H-1500). All immunofluorescence slides were imaged using a spinning disk confocal system: Yokogawa CSU-X1 (Andor Technology, South Windsor, Conn.) with a Nikon Ti-E inverted microscope (Nikon Instruments, Melville, N.Y.), with a 60× objective lens equipped with Zyla cMOS camera. NIS elements software was used for acquisition parameters, shutters, filter positions and focus control.

Unlike recombinant FABP4 which had no effect on GSIS in vitro, addition of NDPK-ADK significantly enhanced GSIS, comparable to the increase observed in FABP4^{-/-} islets. Further, addition of FABP4 to NDPK-ADK significantly blunted this response (FIG. 6A). This finding was recapitulated in both primary human islets from multiple non-diabetic donors (FIG. 6B), as well as INS1 cells (FIG. 6C). Human islets from review board approved healthy donors were isolated using the Edmonton protocol and provided by the IsletCore and Clinical Islet Laboratory (University of Alberta, Canada). Donors with a BMI<25 and HbA1c<6.0% were used for experiments. Islets were hand-picked three times and cultured at 37° C. in RPMI media with 10% FBS and 1% Penicillin/Streptomycin overnight before use. For INS1 cells, cells were plated at 8000 cells/well in 96-well plates and incubated overnight prior to experimentation. Insulin secretion assay was performed as above using 200 μl/well for each treatment. Cells were lysed using 80 μl ultrapure water and flash freezing. For siRNA experiments, INS1 cells were allowed to reach confluency before transfection of siRNA (Horizon Discovery; L-090381-02-0005) using Lipofectamine 3000 (Thermo Fischer; L3000015) according to manufacturer protocol for 48 hours prior to assays.

Importantly, addition of any of the proposed complex components or the antibodies alone had no effect on GSIS (FIGS. 6B-D). To further support the regulation of this complex activity being involved in metabolic health and beta cell function, the effect of the antibody treatment was examined under these treatment conditions. In both primary human islets and INS1 cells, addition of a-Ab rescued GSIS in response to the NDPK-ADK/FABP4 complex to levels observed with NDPK-ADK alone, while c-AB had no effect (FIGS. 6B-C). This highly conserved response, in mouse, rat, and human beta cells supports the relevance of targeting this complex to human biology.

As additional controls, it was explored whether lipid binding was required for FABP4 action. Cells were stimulated with ATP (5 μM; Promega #V915A) or ADP (2.5 μM; Promega #V916A) acutely with the addition of LG and HG KRB during the serial stimulation. Cells were treated with 100 ng/ml recombinant human FABP4 (produced in-house), 100 ng/ml lipid binding mutant FABP4 (as described in Cao et al., 2013), 200 ng/ml recombinant human ADK (Novus Biologicals, product number NBP1-44382 or R&D Systems, product number 8024-AK-025), 100 ng/ml recombinant human NDPK-A (Acro Biosystems, product number NM1-H5147), where indicated, for the pre-incubation period, as well as during the glucose stimulation. FABP4 inhibitor studies were performed using BMS-309403 (10 nM; Sigma #BM0015).

As a fatty acid binding protein, at least some of the functions of intracellular FABP4 have been attributed to its function capacity as a lipid chaperonel8. Importantly, the activity of FABP4 to inhibit GSIS was not dependent on lipid binding, as both a genetically-modified lipid binding mutant (LBM) version of FABP4, and FABP4 treated with the lipid-binding inhibitor BMS-309403, which out-competes fatty acid interactions, exhibited the same effect on suppressing NDPK-ADK-potentiated GSIS as WT recombinant FABP4 (FIG. 6E). Therefore, hormonal FABP4 exhibits a distinct biology independent of its intracellular lipid-binding function, which would not be captured by the use of inhibitors targeting lipid binding.

Extracellular ATP and ADP are known to signal through cell-surface purinergic G-protein coupled receptors (GPCRs). While all cell types express numerous purinergic receptors and channels, the predominant receptor on beta cells is P2Y1. P2Y1 receptors are potently agonized by ADP and antagonized by ATP, and are described as having critical relevance to the regulation of cAMP generation, calcium flux and insulin secretion in both rodents and humans (Khan et al., 2014; Leon et al., 1997; Petit et al., 2009) (outlined in FIG. 6F). The importance of extracellular ATP and ADP on the regulation of insulin secretion is made clear upon treatment with apyrases, which selectively degrade endogenous extracellular ATP (high ratio; producing excess ADP), or ATP and ADP (low ratio; producing excess AMP). Increasing the extracellular ADP concentration while eliminating ATP (high ratio apyrase) resulted in a potent enhancement in GSIS from WT mouse islets, while degradation of both ATP and ADP (low ratio apyrase) significantly blunted GSIS (FIG. 6G). Complementary to these findings, addition of exogenous ADP potentiated insulin secretion from INS1 cells, while ATP suppressed GSIS (FIG. 6H), as previously reported (Bauer et al., 2019).

To determine if modulating the extracellular nucleoside concentrations could influence the activity of the complex on beta cell function, the complex was supplemented with ATP or ADP. Addition of exogenous ATP blunted the increase in GSIS observed with the addition of NDPK-ADK to levels observed with exposure to NDPK-ADK/FABP4 (FIG. 6I). This inhibition was more pronounced upon addition of ATPgS, a non-metabolizable ATP analog that retains affinity for the P2Y1 receptor, consistent with metabolism of ATP as part of complex activity. Conversely, supplementation with ADP potentiated GSIS in the presence of the NDPK-ADK/FABP4 complex to levels observed with NDPK-ADK alone (FIG. 6J).

To further elucidate the role of purinergic receptors in this signaling process, a potent and selective P2Y1 inhibitor, MRS2179 (Baurand et al., 2001) was utilized. Cells were pre-treated for P2Y1 inhibitor (100 nM; MRS2179, Tocris #0900) during the pre-incubation period. Cells were stimulated with ATP (5 μM; Promega #V915A) or ADP (2.5 μM; Promega #V916A) acutely with the addition of LG and HG KRB during the serial stimulation. Cells were treated with 100 ng/ml recombinant human FABP4 (produced in-house), 200 ng/ml recombinant human ADK (Novus Biologicals, product number NBP1-44382 or R&D Systems, product number 8024-AK-025), 100 ng/ml recombinant human NDPK-A (Acro Biosystems, product number NM1-H5147) where indicated, for the pre-incubation period, as well as during the glucose stimulation.

To confirm activity of this inhibitor, INS1 cells were treated with increasing concentrations of ADP in the presence or absence of MRS2179. As anticipated, increasing concentrations of ADP stimulated GSIS, while this increase was completely inhibited in the presence of MRS2179 (FIG. 6K). This supports the hypothesis that the ADP produced by the NDPK-ADK complex signals through the P2Y1 receptor, treatment of INS1 cells with MRS2179 abolished the increase in GSIS associated with addition of NDPK-ADK, with no effect in the presence of NDPK-ADK/FABP4, consistent with the receptor being inhibited by ATP under these conditions (FIG. 6L). Finally, to confirm that the kinase activity of both NDPK and ADK are indeed required to mediate the effect on GSIS, we generated a kinase-dead H118N NDPK mutant that exhibited no capacity to generate ADP (FIG. 3m). Treatment of primary mouse islets with H118N NDPK and WT ADK had no effect on GSIS, confirming the requirement of NDPK kinase activity for the biology (FIG. 6N). Additionally, it was modeled that the inhibitory effect of the NDPK-ADK/FABP4 complex on GSIS is dependent on the production of ATP by ADK, as activated by the presence of FABP4. To test this, an ADK inhibitor was utilized. Cells were pre-treated with ADK inhibitor (2 nM; Santa Cruz #sc-202900) during the pre-incubation period. Cells were stimulated with ATP (5 μM; Promega #V915A) or ADP (2.5 μM; Promega #V916A) acutely with the addition of LG and HG KRB during the serial stimulation. Cells were treated with 100 ng/ml recombinant human FABP4 (produced in-house), 200 ng/ml recombinant human ADK (Novus Biologicals, product number NBP1-44382 or R&D Systems, product number 8024-AK-025), and 100 ng/ml recombinant human NDPK-A (Acro Biosystems, product number NM1-H5147), for the pre-incubation period, as well as during the glucose stimulation.

To confirm the efficacy of the ADK inhibitor, the capacity of ADK to produce ATP was evaluated in the presence of the inhibitor with increasing substrate concentrations. Indeed, the ADK inhibitor completely blocked the ability of ADK to produce ATP (FIG. 6O). Pre-treatment of the NDPK-ADK/FABP4 complex with the ADK inhibitor improved GSIS equivalent to the NDPK-ADK complex alone, indicating that ATP production mediates this activity (FIG. 6P). Based on these findings, it can be inferred that when FABP4-ADK-NDPK are in complex, there is an increase in extracellular ATP which inhibits purinergic receptors, resulting in inhibition of GSIS. In the absence of FABP4 or in the presence of a-Ab, there is increased production of extracellular ADP by NDPK, resulting purinergic receptor activation and increased GSIS (FIG. 6F).

### Example 6. The NDPK-ADK/FABP4 Complex Alters Calcium Dynamics

The purinergic P2Y receptors activate two downstream signaling pathways; phospholipase C (PLC), which promotes the generation of IP3, as well as inhibition of cAMP generation through the activation of G proteins (Sabala et al., 2001; Seo et al., 2016). These pathways directly influence calcium flux, which regulates both insulin secretion and survival. To elucidate how the NDPK-ADK/FABP4 complex influences beta cell function to promote diabetes, the intracellular effects mediated by this complex were examined using western blotting and quantitative PCR. Western blotting and quantitative PCR (QPCR) were performed as previously described (Prentice et al., 2018). Briefly, following cell treatment, INS1 cells or islets (>100) were lysed in RIPA buffer (Cell Signaling, USA) containing protease inhibitor (Sigma P8340). Samples were homogenized by running through an insulin syringe 5-10 times, followed by centrifugation at 12,000 rpm for 10 minutes. Protein content was evaluated by BCA (Invitrogen, USA). Equal amounts of protein were then combined with sample buffer containing DTT, heated at 92° C. for 10 min, and loaded onto a 4-12% SDS-PAGE gradient gel (Invitrogen, USA) and run in IVIES or MOPS running buffer (Invitrogen, USA) at increasing voltages of 50 V for 30 minutes, 75 V for 30 minutes, and then 100 V for 30 minutes. Proteins were then transferred onto PVDF membrane using a Turbo Blotter (BioRad, USA).

First, cAMP signaling was evaluated in human islets and INS1 cells following acute treatment. Addition of NDPK-ADK reduced pPKA substrate phosphorylation downstream of cAMP, while NDPK-ADK/FABP4 enhanced the response (FIG. 7A). Further, INS1 cells treated with the NDPK-ADK/FABP4 complex exhibit increased phosphorylation of the endoplasmic reticulum (ER) calcium efflux transporter IP3R (FIG. 7B).

Cytosolic calcium dynamics were directly evaluated in INS1 cells. Consistent with reduced GSIS, acute treatment with the NDPK-ADK/FABP4 complex significantly decreased extracellular calcium influx in response to the addition of glucose compared to control, or cells treated with NDPK-ADK alone (FIGS. 7C-D). Indeed, addition of NDPK-ADK increased Ca²⁺ influx compared to control cells, consistent with enhanced GSIS. Activation of IP3R with FABP4-ADK-NDPK treatment (FIG. 7B) suggests there may be increased ER calcium efflux into the cytosol. To evaluate this, experiments were performed in the absence of extracellular calcium, such that changes in calcium could only be sourced from the ER. Upon the addition of Thapsigargin (Tg), a sarco/endoplasmic reticulum Ca²⁺ ATPase (SERCA) inhibitor that prevents calcium uptake from the cytosol into the ER, ER calcium efflux was significantly enhanced in cells acutely treated with the FABP4-ADK-NDPK complex compared to controls (FIGS. 7E-F).

In both cases, co-treatment with FABP4-NDPK-ADK and a-Ab was able to rescue calcium flux to control levels. To confirm the alterations in calcium flux associated with the FABP4-ADK-NDPK complex are due to purinergic signaling through P2Y1, cells were co-treated with the P2Y1 receptor agonist MRS2365 (mimicking elevated extracellular ADP). This rescued both calcium influx (FIGS. 7G-H) and ER calcium efflux to control levels (FIGS. 7I-J). The FABP4-ADK-NDPK-induced alteration in ER calcium efflux was also rescued by co-treatment with NKY80, an adenylyl cyclase inhibitor which blocks cAMP formation (FIG. 7K). Altogether FABP4-ADK-NDPK signals through P2Y1 purinergic receptors to modulate cAlVIP production, resulting in alterations in calcium flux and beta cell dysfunction.

Cells were pre-treated with P2Y1 activator (0.5 nM; MRS2365 Tocris #2157) during the pre-incubation period. Cells were stimulated with ATP (5 μM; Promega #V915A) or ADP (2.5 μM; Promega #V916A) acutely with the addition of LG and HG KRB during the serial stimulation. Cells were treated with 100 ng/ml recombinant human FABP4 (produced in-house), 200 ng/ml recombinant human ADK (Novus Biologicals, product number NBP1-44382 or R&D Systems, product number 8024-AK-025), or 100 ng/ml recombinant human NDPK-A (Acro Biosystems, product number NM1-H5147) where indicated, for the pre-incubation period, as well as during the glucose stimulation.

Calcium imaging experiments were performed as previously described (Basford et al., 2012; Prentice et al., 2014). Briefly, INS1 cells were plated in imaging disks at 5000 cells/dish and cultured in standard media overnight. Cells were washed with PBS, and incubated in imaging buffer without glucose, with or without calcium containing Fura-2 AM (2 mM; ThermoFisher F1201) for 30 minutes at 37° C. Cells were washed twice with fresh PBS, and placed in lml imaging buffer. Calcium flux was evaluated with the addition of 20 mM glucose, 30 mM KCl, or 1 μM Thapsigargin. Extracellular calcium was added at 1.5 mM. Calcium flux measurements were performed using an epifluorescence system coupled to a Ti-S/L100 inverted microscope L100 with fluorescent light source (X-Cite 120LED System) equipped with Lambda DG-4 Plus for UV ratiometric probes. Excitations at 340 nm and 380 nm were used and an emission of 510 nm. Analysis was performed using ImageJ software. Calcium flux was measured from 50 cells per coverslip.

ER calcium homeostasis is essential for a variety of processes and reduced ER calcium can induce ER stress through the accumulation or inappropriate secretion of misprocessed or misfolded proteins (Engin et al., 2014; Engin et al., 2013). A significant increase was observed in the ER stress marker CHOP with 24 hr NDPK-ADK/FABP4 treatment, as well as increased cleaved caspase 3 (CC3) and JNK phosphorylation (pJNK), indicating that chronic exposure to this complex induces beta cell stress and death (FIG. 6G and FIG. 7L). In the pathogenesis of diabetes, beta cells are exposed to numerous stressors including increased synthetic demand, ER stress, lipotoxic stress, and in the case of T1D, cytotoxic stress from invading immune cells, which collectively potentiate beta cell apoptosis. Upon combined stimulation with the NDPK-ADK/FABP4 complex and ER stress-inducer Tg, there was a significant increase in ER stress markers Chop and Bip (Grp78), which were rescued to control levels upon addition of a-Ab (FIGS. 7N-O). Induction of ER stress markers with FABP4-ADK-NDPK corresponded to higher cleaved caspase 3/7 activity, and thus potentiated cell death (FIG. 7P). To measure cleaved caspase activity, INS1 cells were plated at 6000 cells/well in 96-well plates and cultured overnight. Cells were treated for 2 hours with increasing doses of Thapsigargin or 18 hours with increasing doses of cytokines (1=TNFα (Peprotech #315-05-20 ug) long/ml, IFNγ (Peprotech #315-01A-20 ug) 100 ng/ml, IL-1β (R&D Systems #401-ML-005) 5 ng/ml), washed with PBS, and cleaved caspase 3/7 activity was determined using the Promega Caspase-Glo 3/7 Assay System, according to manufacturer's instructions.

Complementary to this, reduced expression of Chop was observed in FABP4^{-/-} islets compared to WT, with reduced induction upon Tg treatment. Cytokines produced by invading immune cell populations are a primary factor contributing to beta cell apoptosis in T1D. Therefore, we evaluated if the NDPK-ADK/FABP4 complex also impacted cytokine-induced cell death. Following 12 hr incubation with a cytokine cocktail containing TNFα, IL1ß, and IFNγ, a significant enhancement in cleaved caspase 3/7 activity was observed in the presence of the complex compared to cytokines alone (FIG. 6K). Thus, the NDPK-ADK/FABP4 complex alters ER calcium homeostasis, resulting in ER stress, increased sensitivity to environmental stressors, and potentiation of beta cell death in vitro.

### Example 7: A-Ab Preserves Beta Cell Mass In Vivo

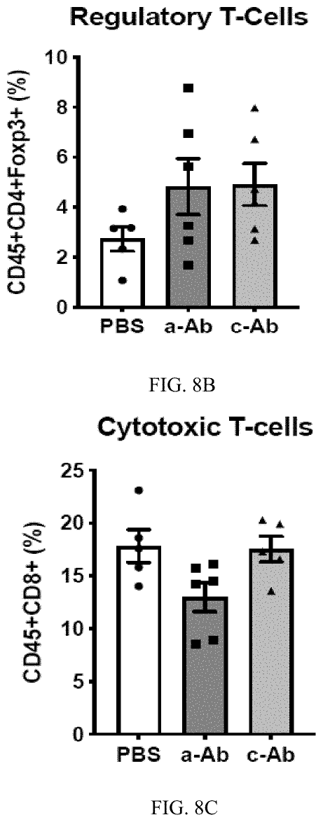

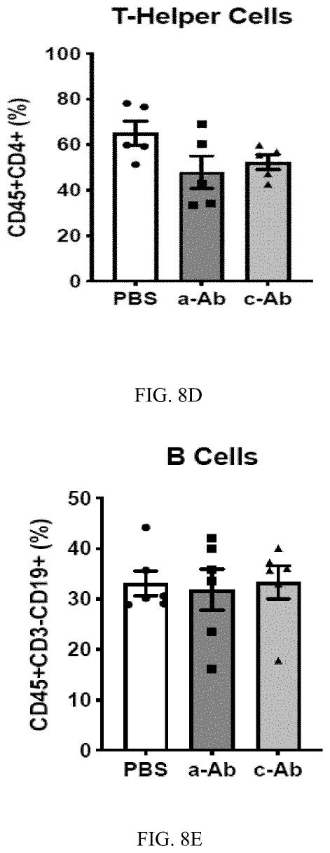

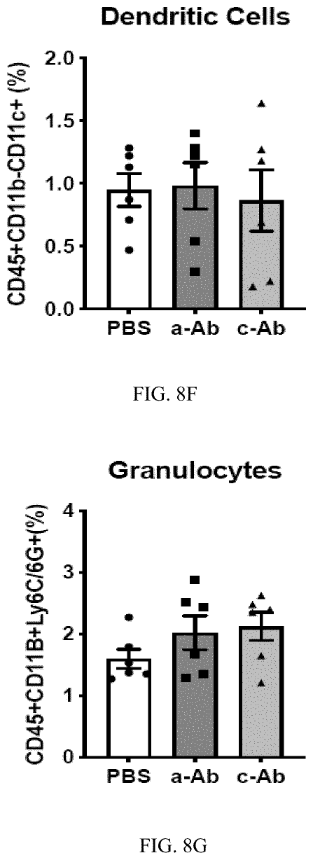

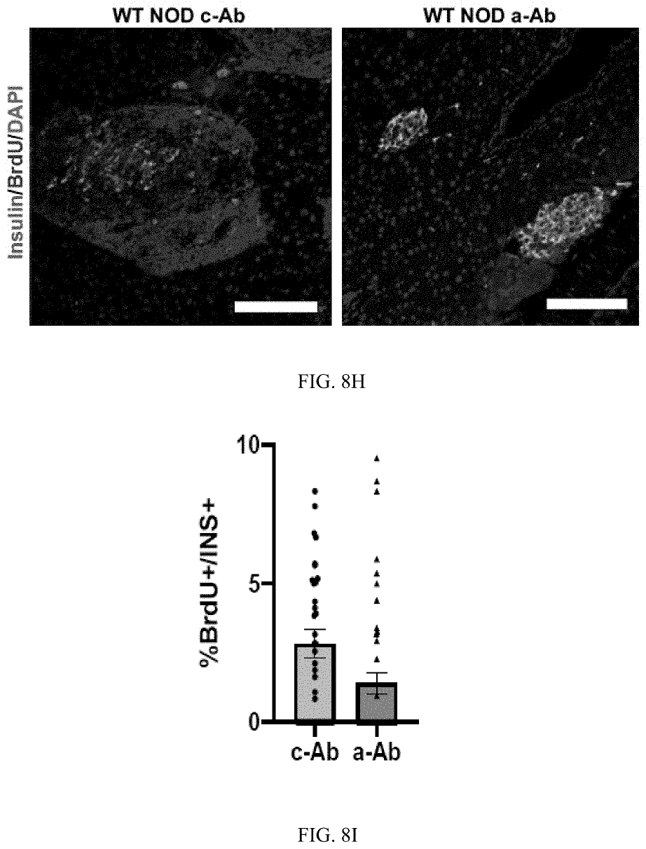

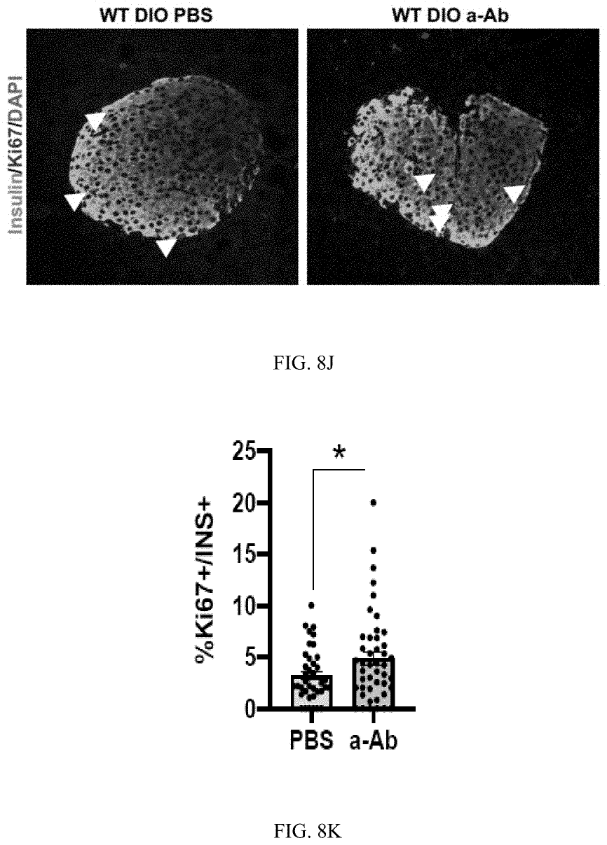

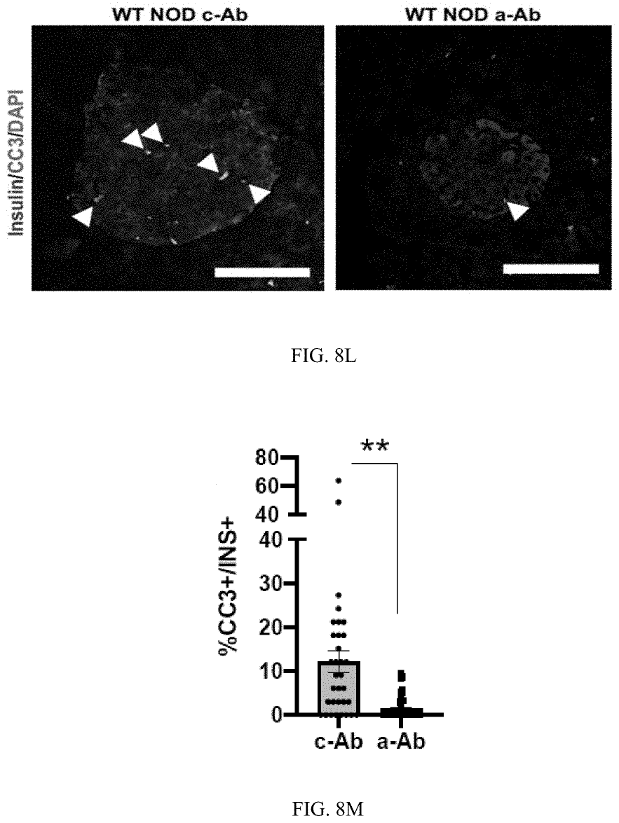

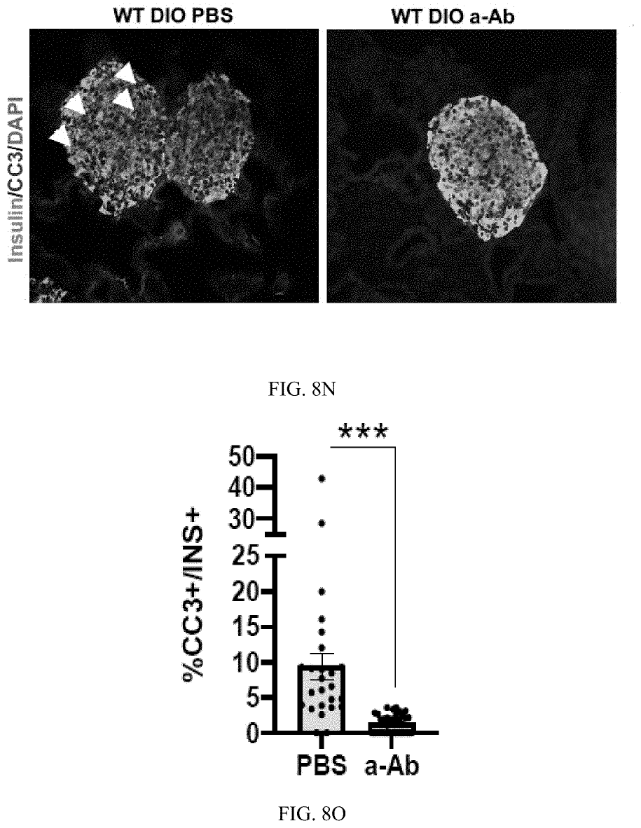

Given the findings that targeting the novel FABP4-ADK-NDPK complex with a-Ab improves beta cell stress resistance and functionality in vitro, the next step was to confirm that a-Ab treatment reduces beta cell death in vivo. In both the NOD model of T1D and the DIO model of T2D significant increases in islet number and beta cell mass with a-Ab treatment compared to controls were observed (FIGS. 1M-O, 2Q-S). The conserved phenotype between these models suggests a direct effect on beta cells, as there is limited immune influence on the disruption of beta cell mass and function in DIO mice. This is supported by the lack of significant impact on the immune cell profile in pancreata of non-diabetic a-Ab-treated NOD mice (FIGS. 8A-G, Table 14).

TABLE 14
Immunophenotyping of peripheral
tissues in non-diabetic NOD mice

| Tissue | Cell Type | PBS (%) | | a-Ab (%) | | c-Ab (%) | |
|---|---|---|---|---|---|---|---|
| Spleen | CD45+ | 97.8 | (0.42) | 96.2 | (0.85) | 97.9 | (0.31) |
| | B Cells | 50.7 | (3.73) | 51.3 | (2.25) | 48.7 | (1.34) |
| | T-Helper | 29.9 | (2.02) | 30.4 | (1.42) | 32.4 | (0.96) |
| | Cells | | | | | | |
| | Cytotoxic | 12.1 | (1.50) | 11.5 | (0.72) | 12.9 | (0.66) |
| | T-Cells | | | | | | |
| | Regulatory | 3.2 | (0.59) | 3.1 | (0.48) | 3.4 | (0.42) |
| | T-Cells | | | | | | |
| | Dendritic | 0.95 | (0.13) | 0.98 | (0.18) | 0.86 | (0.24) |
| | Cells | | | | | | |
| | Granulocytes | 1.6 | (0.15) | 2.0 | (0.28) | 2.1 | (0.22) |
| ILN | CD45+ | 99.0 | (0.16) | 98.8 | (0.31) | 99.0 | (0.15) |
| | B Cells | 21.1 | (4.2) | 19.0 | (0.91) | 17.7 | (2.64) |
| | T-Helper | 54.7 | (3.43) | 56.6 | (1.13) | 57.8 | (2.21) |
| | Cells | | | | | | |
| | Cytotoxic | 21.7 | (1.15) | 21.4 | (0.36) | 22.0 | (0.98) |
| | T-Cells | | | | | | |
| | Regulatory | 3.8 | (0.77) | 3.9 | (0.63) | 4.2 | (0.40) |
| | T-Cells | | | | | | |
| | Dendritic | 3.3 | (0.29) | 4.8 | (0.86) | 3.8 | (0.48) |
| | Cells | | | | | | |
| | Granulocytes | 3.2 | (0.85) | 2.3 | (0.16) | 2.5 | (0.54) |
| PLN | CD45+ | 97.9 | (0.68) | 97.4 | (1.10) | 97.8 | (0.52) |
| | B Cells | 44.5 | (3.01) | 38.1 | (7.17) | 35.9 | (3.93) |
| | T-Helper | 33.7 | (2.14) | 41.3 | (5.64) | 43.6 | (2.53) |
| | Cells | | | | | | |
| | Cytotoxic | 13.8 | (1.42) | 15.7 | (2.36) | 16.0 | (1.35) |
| | T-Cells | | | | | | |
| | Regulatory | 3.5 | (0.67) | 3.3 | (0.42) | 3.3 | (0.45) |
| | T-Cells | | | | | | |
| | Dendritic | 16.9 | (2.67) | 12.9 | (3.55) | 8.8 | (1.31) |
| | Cells | | | | | | |
| | Granulocytes | 5.5 | (0.42) | 5.0 | (0.59) | 4.6 | (0.46) |

Values are mean (SEM)

Immune phenotyping of whole pancreas from non-diabetic mice treated with PBS, a-Ab or c-Ab for 12 wks revealed that a-Ab-treated mice had a significantly reduced percentage of total immune cell numbers. To perform immune phenotyping, mice were euthanized with CO2 and cardiac perfusion performed with 40 ml of sterile PBS. Whole pancreas, pancreatic lymph nodes (PLN), inguinal lymph nodes (ILN), and spleen were harvested, diced into 1 mm pieces, placed in digestion buffer (RPMI 1640, lmg/ml Collagenase Type IV, 10 U/ml DNAsel, and 1% BSA), and incubated at 37° C. for 20 min shaking at 250 rpm. Pancreas samples were filtered through a 40 μm nylon strainer into a fresh 50 ml tube, and fresh media added up to 40 ml (RPMI 1640, 1% FBS, 1% Sodium Pyruvate, 1.56 ul β-mercaptoethanol). Tubes were then centrifuged at 1500 rpm for 10 min, supernatant removed, and cells resuspended in 2 ml fresh media. Resuspended cells were passed through a 40 μm nylon filter into a 15 ml round-bottomed polystyrene tube. For PLN, ILN, and spleen, digestion samples were filtered through a 40 μm nylon strainer into a 15 ml round bottom polystyrene tube, and lml of media was added. Spleen samples had supernatant removed, and lml ACK lysis buffer (Thermo Scientific A1049201) added for 2 min at room temperature. Samples were then centrifuged at 1500 rpm for 5 min, and resuspended in 1 ml media. All samples were centrifuged at 1500 rpm for 5 min, supernatant removed, and cells resuspended in FACS buffer (5% BSA in cold PBS; 0.5 ml for PLN and ILN, lml for pancreas and spleen). Staining and FACS analysis were performed as previously described (Engin et al., 2013).

To investigate the contribution of proliferation to the increase in beta cell mass, NOD mice were treated with BrdU along with a-Ab, c-Ab, or PBS for 5 weeks beginning at 10 weeks of age. There was no significant difference in the overall number of BrdU positive beta cells with a-Ab treatment compared to c-Ab (FIGS. 8H-I). In the DIO model, however, three weeks of a-Ab treatment was associated with a small but significant increase in beta cell proliferation, as assessed by Ki67 staining (FIGS. 8J-K). This difference is likely associated with the pro-proliferative environment of DIO as compared to the NOD model. Conversely, and consistent with the in vitro findings, a profound reduction in CC3 positive beta cells was observed upon a-Ab treatment compared to controls in both the NOD and DIO diabetes models (FIGS. 8L-O). Thus, targeting the FABP4-ADK-NDPK complex with a-Ab preserves beta cell mass and enhances beta cell function to protect against diabetes.

Pancreata were dissected and fixed in 10% zinc buffered formalin for 24-48 hrs before being washed and stored in 70% ethanol. Samples were paraffin embedded, and sectioned in steps 250 μm apart. Immunostaining for insulin, glucagon, BrdU and Cleaved Caspase 3 was performed as described previously (Prentice et al., 2014; Rezania et al., 2014). Images of each section were acquired using Aperio Imagescope at 20× magnification. The beta- or alpha-cell area was calculated by using positive pixel count analysis (Aperio ImageScope). Islet number and size was determined by manually circling insulin positive clusters in the Aperio ImageScope software. Two blinded individuals independently performed manual analysis and insulitis scoring.

This specification has been described with reference to embodiments of the invention. Given the teaching herein, one of ordinary skill in the art will be able to modify the invention for a desired purpose and such variations are considered within the scope of the invention.

We claim:

1. A method of identifying a compound capable of binding fatty acid binding protein 4 (FABP4) and inhibiting formation of nucleoside diphosphate kinase (NDPK)-adenosine kinase (ADK)/FABP4 complex comprising:
i. determining whether the compound binds to FABP4;
ii. contacting the compound with FABP4 and NDPK-ADK complex or NDPK and ADK; and,
iii. determining whether the compound inhibits the interaction of FABP4 with NDPK-ADK complex.

2. The method of claim 1, further comprising determining whether the compound inhibits FABP4's ability to modulate NDPK-ADK complex activity on purinergic G protein-coupled receptors and/or channels comprising:
i. introducing the compound into a cellular assay in the presence of i) FABP4 and the NDPK-ADK complex or ii) NDPK-ADK/FABP4 complex, wherein the cellular assay includes a population of cells expressing purinergic receptors and/or channels; and
ii. measuring an adenosine triphosphate (ATP) concentration and adenosine diphosphate (ADP) concentration, wherein an ADP concentration greater than an ATP concentration in the assay indicates a compound capable of inhibiting FABP4's ability to modulate NDPK-ADK complex activity on the purinergic G protein-coupled receptors and/or channels.

3. The method of claim 2, wherein the cell population expressing the purinergic receptors and/or channels comprises pancreatic islet β-cells.

4. The method of claim 3, wherein pancreatic islet β-cells are human cells.

5. A method of identifying a compound capable of neutralizing FABP4 regulation of NDPK-ADK complex modulation of purinergic G protein-coupled receptors and/or channels comprising:
i. introducing i) FABP4 and the NDPK-ADK complex or ii) NDPK-ADK/FABP4 complex into a first cellular assay comprising cells expressing the purinergic G protein-coupled receptors and/or channels;
ii. determining an ATP to ADP ratio in the first cellular assay;
iii. introducing i) FABP4 and the NDPK-ADK complex or ii) the NDPK-ADK/FABP4 complex into a second cellular assay comprising cells expressing the purinergic G protein-coupled receptors and/or channels, wherein i) FABP4 and the NDPK-ADK complex or ii) the NDPK-ADK/FABP4 complex are introduced in the presence of the compound;
iv. determining an ATP to ADP ratio in the second cellular assay; and
v. comparing the ATP to ADP ratio in the first cellular assay with the ATP to ADP ratio in the second cellular assay;
wherein a reduction in the ATP to ADP ratio in the second cellular assay compared to the ATP to ADP ratio in the first cellular assay is indicative of a compound that is capable of neutralizing FABP4 regulation of NDPK-ADK complex modulation of the purinergic G protein-coupled receptors and/or channels.

6. The method of claim 5, wherein the cell population expressing the purinergic G protein-coupled receptors and/or channels comprises pancreatic islet β-cells.

7. The method of claim 6, wherein the cell population expressing purinergic G protein-coupled receptors and/or channels comprises human cells.

8. A composition for use in an assay to identify a compound capable of treating or preventing a FABP4-mediated disorder comprising:
i) a recombinant FABP4 protein;
ii) a recombinant NDPK protein, and,
iii) a recombinant ADK protein.

9. The composition of claim 8, wherein the recombinant FABP4 protein is a recombinant human FABP4 protein.

10. The composition of claim 9, wherein the recombinant FABP4 protein comprises an amino acid sequence comprising SEQ ID NO: 4.

11. The composition of claim 8, wherein the recombinant NDPK protein is a recombinant human NDPK protein.

12. The composition of claim 11, wherein the recombinant NDPK protein is NDPK-A protein.

13. The composition of claim 12, wherein the recombinant NDPK-A protein comprises an amino acid sequence comprising SEQ ID NO: 1.

14. The composition of claim 11, wherein the recombinant NDPK protein is NDPK-B protein.

15. The composition of claim 14, wherein the recombinant NDPK-B protein comprises an amino acid sequence comprising SEQ ID NO: 2.

16. The composition of claim 8, wherein the recombinant ADK protein is a recombinant human ADK protein.

17. The composition of claim 16, wherein the recombinant ADK protein comprises an amino acid sequence comprising SEQ ID NO: 3.
